(19) United States
(12) Patent Application Publication
(10) Pub. No.: US 2025/0092017 A1
(43) Pub. Date: Mar. 20, 2025

(54) 2-PIPERIDYL OR 2-PYRAZOLYL SUBSTITUTED PYRIMIDINE COMPOUND SERVING AS EGFR INHIBITOR

(71) Applicant: SUZHOU PUHE BIOPHARMA CO., LTD., Suzhou (CN)

(72) Inventors: Bin Liu, Suzhou (CN); Feng Gao, Suzhou (CN); Shuai Yuan, Suzhou (CN); Hui Zhao, Suzhou (CN); Xingzhe Peng, Suzhou (CN); Chong Liu, Beijing (CN); Ning Shao, Beijing (CN); Yongqi Guo, Suzhou (CN); Yongyong Wu, Suzhou (CN); Zhuo Wu, Suzhou (CN)

(21) Appl. No.: 18/729,309

(22) PCT Filed: Oct. 28, 2022
(86) PCT No.: PCT/CN2022/128264

§ 371 Date: Jul. 16, 2024

(30) Foreign Application Priority Data
Jan. 17, 2022 (WO) PCT/CN2022/072306
May 12, 2022 (WO) PCT/CN2022/092375

(51) Int. Cl. C07D 401/14; A61K 31/506; A61K 31/517; A61K 31/675; A61P 35/00; C07D 405/14; C07D 417/14; C07D 471/04; C07F 9/6558

(52) U.S. Cl. CPC C07D 401/14; A61K 31/506; A61K 31/517; A61K 31/675; A61P 35/00; C07D 405/14; C07D 417/14; C07D 471/04; C07F 9/65583

(57) ABSTRACT

A 2-piperidyl or 2-pyrazolyl substituted pyrimidine compound, which is a compound represented by formula (I) or a pharmaceutically acceptable salt, an isotopic variant, a tautomer, a stereoisomer, a prodrug, a polymorph, a hydrate or a solvate thereof. A pharmaceutical composition comprising the compound, and a use of the pharmaceutical composition in the treatment of diseases mediated by an EGFR protein and mutants thereof.

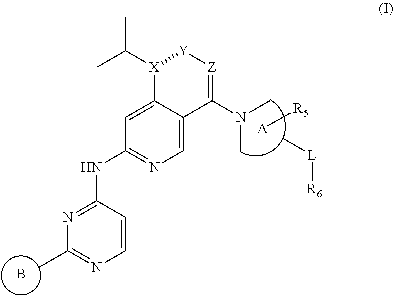

### TECHNICAL FIELD

The present disclosure belongs to the field of medicine, and specifically relates to pyrimidine compounds substituted with 2-piperidinyl or 2-pyrazolyl as EGFR inhibitors.

### BACKGROUND

Lung cancer is one of the most common malignant tumors, with approximately 1.6 million new cases of lung cancer worldwide each year. Lung cancer has two types: small cell lung cancer and non-small cell lung cancer (NSCLC), of which non-small cell lung cancer accounts for approximately 85% of all lung cancers (Nature Reviews Disease Primers, 2015, 1,15009). Epidermal growth factor receptor (EGFR) is the most common driver gene for non-small cell lung cancer, with a positive rate of 17% in all non-small cell lung cancers, close to 30% to 40% in domestic patients of China, and as high as about 60% in lung adenocarcinoma.

EGFR is a transmembrane glycoprotein that belongs to the ErbB family of tyrosine kinase receptors. EGFR is abnormally activated by various mechanisms, such as receptor overexpression, mutation, ligand-dependent receptor dimerization, and ligand-independent activation. The continuous activation of its kinase activity initiates downstream signal transduction of cell proliferation, differentiation, and survival. Small molecule inhibitors of EGFR kinase can inhibit the activation of tyrosine kinase, inhibit the proliferation of tumor cells, promote the apoptosis of tumor cells and other biological effects, and are a hot area for lung cancer development.

Osimertinib is a drug developed for first- and second-generation drug-resistant mutations of EGFR (del19 or L858R) accompanied by T790M mutations. It has shown very significant efficacy in clinical practice, but patients will also develop drug resistance as treatment continues. In 2015 (Nature Medicine, 2015, 21, 560-562), the drug resistance data of 15 patients taking osimertinib were first reported, among which EGFR C797S mutation was one of main mechanisms leading to drug resistance to osimertinib, accounting for about 40%. In addition, the latest literature reports show that among patients with resistance after second-line treatment with osimertinib, 22-25% have C797S mutations (Nature Cancer, 2021, 377-391). Therefore, there is an urgent clinical need to develop new small molecule inhibitors targeting C797S mutations to provide patients with safer and more effective fourth-generation EGFR inhibitors.

On the other hand, although the first-generation EGFR inhibitor gefitinib and the third-generation EGFR inhibitor osimertinib both produced good therapeutic effects in the first-line population with EGFR activating mutations, in subgroup analysis, the benefits of the L858R population were much lower than those of the del19 population, with PFS of 14.4 and 21.4 months, respectively (The new England journal of medicine, 2018, 378, 113-125).

### SUMMARY

In the present disclosure, we used the main mutation types that appear in the clinic, such as L858R/C797S, L858R/T790M/C797S and L858R, to carry out enzymatic evaluation and verify it at the constructed Ba/F3 cell level, and finally discovered a series of new chemical entities with strong biological activity.

In one aspect, the present disclosure provides a compound of formula (I), or a pharmaceutically acceptable salt, isotopic variant, tautomer, stereoisomer, prodrug, polymorph, hydrate or solvate thereof:

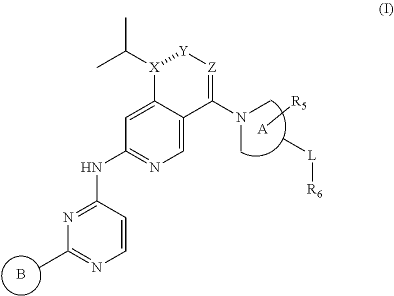

wherein:

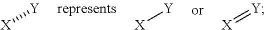

wherein when

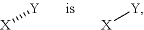

X is selected from CH and N, and Y is selected from CH₂, C═O and C═S; and when

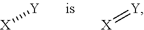

### X is C, and Y is CH;

Z is selected from CH and N;
ring A is 4- to 8-membered heterocyclyl;
ring B is selected from

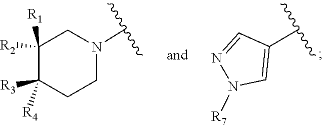

L is selected from a chemical bond, —S(═O)(=M)-, —N(R_{N})—S(═O)(=M)-, —S(═O)(=M)-N(R_{N})—, —N(R_{N})—C(═O)—, —C(═O)—N(R_{N})—, —CH(R_{N})—C(═O)—, —C(═O)—CH(R_{N})—, —CH(R_{N})—S(═O)(=M)- and —S(═O)(=M)-CH(R_{N})—;
R₁ and R₂ are independently selected from H, halogen, C₁₋₆ alkyl and C₁₋₆ haloalkyl;
R₃ and R₄ are independently selected from H, ORₐ, C₁₋₆ alkyl and C₁₋₆ haloalkyl;
R₅ is selected from H, halogen, C₁₋₆ alkyl and C₁₋₆ haloalkyl;
R₆ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl;
R₇ is selected from H, C₁₋₆ alkyl, C₁₋₆ haloalkyl and —S(═O)(=M)-R₈;
wherein M is O or NH;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
R₈ is selected from ORₐ, NR_{b}R_{c}, C₁₋₆ alkyl, —C₁₋₆ alkylene-C₃₋₆ cycloalkyl, C₁₋₆ haloalkyl, C₃₋₆ cycloalkyl, 4- to 6-membered heterocyclyl, C₆₋₁₀ aryl and 5- to 6-membered heteroaryl, which are optionally substituted with one or more Rₐ;
Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl;
provided that, when L is —CH(R_{N})—S(═O)(=M)-, and the ring where X, Y and Z are located is a benzene ring, B is

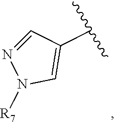

In another aspect, the present disclosure provides a pharmaceutical composition comprising a compound of the present disclosure, and optionally pharmaceutically acceptable excipients.

In another aspect, the present disclosure provides a pharmaceutical composition comprising a compound of the present disclosure and pharmaceutically acceptable excipients, which also contain other therapeutic agents.

In another aspect, the present disclosure provides use of the compound of the present disclosure in the manufacture of a medicament for treating and/or preventing a disease mediated by EGFR protein and mutants thereof.

In another aspect, the present disclosure provides a method for treating and/or preventing a disease mediated by EGFR protein and mutants thereof in a subject, comprising administering a compound or composition of the present disclosure to the subject.

In another aspect, the present disclosure provides a compound or a composition of the present disclosure, for use in treating and/or preventing a disease mediated by EGFR protein and mutants thereof.

In a specific embodiment, the EGFR mutant is one or more of the L858R mutant, del19 mutant, T790M mutant, and C797S mutant; optionally, the disease is selected from lung cancer, colon cancer, urothelial carcinoma, breast cancer, prostate cancer, brain cancer, ovarian cancer, gastric cancer, pancreatic cancer, head and neck cancer, bladder cancer and mesothelioma, alternatively non-small cell lung cancer.

Other objects and advantages of the present disclosure will be apparent to those skilled in the art from the following detailed embodiments, examples and claims.

### Definition

### Chemical Definitions

Definitions of specific functional groups and chemical terms are described in more detail below.

When a range of values is listed, it is intended to encompass each value and sub-range within the range. For example, “C₁₋₆ alkyl” is intended to include C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅ and C₅₋₆ alkyl.

“C₁₋₆ alkyl” refers to a radical of a straight or branched, saturated hydrocarbon group having 1 to 6 carbon atoms. In some embodiments, C₁₋₄ alkyl and C₁₋₂ alkyl are alternative. Examples of C₁₋₆ alkyl include methyl (C₁), ethyl (C₂), n-propyl (C₃), iso-propyl (C₃), n-butyl (C₄), tert-butyl (C₄), sec-butyl (C₄), iso-butyl (C₄), n-pentyl (C₅), 3-pentyl (C₅), pentyl (C₅), neopentyl (C₅), 3-methyl-2-butyl (C₅), tert-pentyl (C₅) and n-hexyl (C₆). The term “C₁₋₆ alkyl” also includes heteroalkyl, wherein one or more (e.g., 1, 2, 3 or 4) carbon atoms are substituted with heteroatoms (e.g., oxygen, sulfur, nitrogen, boron, silicon, phosphorus). Alkyl groups can be optionally substituted with one or more substituents, for example, with 1 to 5 substituents, 1 to 3 substituents or 1 substituent. Conventional abbreviations of alkyl include Me (—CH₃), Et (—CH₂CH₃), iPr (—CH(CH₃)₂), nPr (—CH₂CH₂CH₃), n-Bu (—CH₂CH₂CH₂CH₃) or i-Bu (—CH₂CH(CH₃)₂).

“C₂₋₆ alkenyl” refers to a radical of a straight or branched hydrocarbon group having 2 to 6 carbon atoms and at least one carbon-carbon double bond. In some embodiments, C₂₋₄ alkenyl is alternative. Examples of C₂₋₆ alkenyl include vinyl (C₂), 1-propenyl (C₃), 2-propenyl (C₃), 1-butenyl (C₄), 2-butenyl (C₄), butadienyl (C₄), pentenyl (C₅), pentadienyl (C₅), hexenyl (C₆), etc. The term “C₂₋₆ alkenyl” also includes heteroalkenyl, wherein one or more (e.g., 1, 2, 3 or 4) carbon atoms are replaced by heteroatoms (e.g., oxygen, sulfur, nitrogen, boron, silicon, phosphorus). The alkenyl groups can be optionally substituted with one or more substituents, for example, with 1 to 5 substituents, 1 to 3 substituents or 1 substituent.

“C₂₋₆ alkynyl” refers to a radical of a straight or branched hydrocarbon group having 2 to 6 carbon atoms, at least one carbon-carbon triple bond and optionally one or more carbon-carbon double bonds. In some embodiments, C₂₋₄ alkynyl is alternative. Examples of C₂₋₆ alkynyl include, but are not limited to, ethynyl (C₂), 1-propynyl (C₃), 2-propynyl (C₃), 1-butynyl (C₄), 2-butynyl (C₄), pentynyl (C₅), hexynyl (C₆), etc. The term “C₂₋₆ alkynyl” also includes heteroalkynyl, wherein one or more (e.g., 1, 2, 3 or 4) carbon atoms are replaced by heteroatoms (e.g., oxygen, sulfur, nitrogen, boron, silicon, phosphorus). The alkynyl groups can be substituted with one or more substituents, for example, with 1 to 5 substituents, 1 to 3 substituents or 1 substituent.

“C₁₋₆ alkylene” refers to a divalent group formed by removing another hydrogen of the C₁₋₆ alkyl, and can be substituted or unsubstituted. In some embodiments, C₁₋₄ alkylene, C₂₋₄ alkylene, and C₁₋₃ alkylene are alternative. The unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), etc. Examples of substituted alkylene groups, such as those substituted with one or more alkyl (methyl) groups, include, but are not limited to, substituted methylene (—CH(CH₃)—, —C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), etc.

“C₀₋₆ alkylene” refers to a chemical bond and the above-mentioned C₁₋₆ alkylene.

“C₂₋₆ alkenylene” refers to a C₂₋₆ alkenyl group wherein another hydrogen is removed to provide a divalent radical of alkenylene, and which may be substituted or unsubstituted. In some embodiments, C₂₋₄ alkenylene is yet alternative. Exemplary unsubstituted alkenylene groups include, but are not limited to, ethenylene (—CH═CH—) and propenylene (e.g., —CH═CHCH₂—, —CH₂—CH═CH—). Exemplary substituted alkenylene groups, e.g., substituted with one or more alkyl (methyl) groups, include but are not limited to, substituted ethylene (—C(CH₃)═CH—, —CH═C(CH₃)—), substituted propylene (e.g., —C(CH₃)═CHCH₂—, —CH═C(CH₃)CH₂—, —CH═CHCH(CH₃)—, —CH═CHC(CH₃)₂—, —CH(CH₃)—CH═CH—, —C(CH₃)₂—CH═CH—, —CH₂—C(CH₃)═CH—, —CH₂—CH═C(CH₃)—), and the like.

“C₂₋₆ alkynylene” refers to a C₂₋₆ alkynyl group wherein another hydrogen is removed to provide a divalent radical of alkynylene, and which may be substituted or unsubstituted. In some embodiments, C₂₋₄ alkynylene is yet alternative. Exemplary alkynylene groups include, but are not limited to, ethynylene (—C≡C—), substituted or unsubstituted propynylene (—C≡CCH₂—), and the like.

“Halo” or “halogen” refers to fluorine (F), chlorine (Cl), bromine (Br) and iodine (I).

Thus, “C₁₋₆ haloalkyl” refers to the above “C₁₋₆ alkyl”, which is substituted by one or more halogen. In some embodiments, C₁₋₄ haloalkyl is yet alternative, and still alternatively C₁₋₂ haloalkyl. Exemplary haloalkyl groups include, but are not limited to, —CF₃, —CH₂F, —CHF₂, —CHFCH₂F, —CH₂CHF₂, —CF₂CF₃, —CCl₃, —CH₂Cl, —CHCl₂, 2,2,2-trifluoro-1,1-dimethyl-ethyl, and the like. The haloalkyl can be substituted at any available point of attachment, for example, with 1 to 5 substituents, 1 to 3 substituents or 1 substituent.

“C₃₋₁₀ cycloalkyl” refers to a radical of a non-aromatic cyclic hydrocarbon group having from 3 to 10 ring carbon atoms and zero heteroatoms. In some embodiments, C₅₋₇ cycloalkyl, C₃₋₆ cycloalkyl and C₃₋₅ cycloalkyl are yet alternative, and still alternatively C₅₋₆ cycloalkyl. The cycloalkyl also includes a ring system in which the cycloalkyl described herein is fused with one or more aryl or heteroaryl groups, wherein the point of attachment is on the cycloalkyl ring, and in such case, the number of carbon atoms continues to represent the number of carbon atoms in the cycloalkyl system. Exemplary cycloalkyl groups include, but are not limited to, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), etc. The cycloalkyl can be substituted with one or more substituents, for example, with 1 to 5 substituents, 1 to 3 substituents or 1 substituent.

“3- to 12-membered heterocyclyl” refers to a radical of 3- to 12-membered non-aromatic ring system having ring carbon atoms and 1 to 5 ring heteroatoms, wherein each of the heteroatoms is independently selected from nitrogen, oxygen, sulfur, boron, phosphorus and silicon. In the heterocyclyl containing one or more nitrogen atoms, the point of attachment can be a carbon or nitrogen atom as long as the valence permits. In some embodiments, 4- to 8-membered heterocyclyl is alternative, which is a radical of 4- to 8-membered non-aromatic ring system having ring carbon atoms and 1 to 5 ring heteroatoms. In some embodiments, 5- to 8-membered heterocyclyl is alternative, which is a radical of 5- to 8-membered non-aromatic ring system having ring carbon atoms and 1 to 5 ring heteroatoms. In some embodiments, 3- to 8-membered heterocyclyl is alternative, which is a radical of 3- to 8-membered non-aromatic ring system having ring carbon atoms and 1 to 4 ring heteroatoms. 3- to 6-membered heterocyclyl is alternative, which is a radical of 3- to 6-membered non-aromatic ring system having ring carbon atoms and 1 to 3 ring heteroatoms. 4- to 7-membered heterocyclyl is alternative, which is a radical of 4- to 7-membered non-aromatic ring system having ring carbon atoms and 1 to 3 ring heteroatoms. 4- to 6-membered heterocyclyl is alternative, which is a radical of 4- to 6-membered non-aromatic ring system having ring carbon atoms and 1 to 3 ring heteroatoms. 5- to 6-membered heterocyclyl is more alternative, which is a radical of 5- to 6-membered non-aromatic ring system having ring carbon atoms and 1 to 3 ring heteroatoms. The heterocyclyl also includes a ring system wherein the heterocyclyl described above is fused with one or more cycloalkyl groups, wherein the point of attachment is on the cycloalkyl ring, or the heterocyclyl described above is fused with one or more aryl or heteroaryl groups, wherein the point of attachment is on the heterocyclyl ring; and in such cases, the number of ring members continues to represent the number of ring members in the heterocyclyl ring system. Exemplary 3-membered heterocyclyl groups containing one heteroatom include, but are not limited to, aziridinyl, oxiranyl and thiorenyl. Exemplary 4-membered heterocyclyl groups containing one heteroatom include, but are not limited to, azetidinyl, oxetanyl and thietanyl. Exemplary 5-membered heterocyclyl groups containing one heteroatom include, but are not limited to, tetrahydrofuranyl, dihydrofuranyl, tetrahydrothiophenyl, dihydrothienyl, pyrrolidinyl, dihydropyrrolyl and pyrrolyl-2,5-dione. Exemplary 5-membered heterocyclyl groups containing two heteroatoms include, but are not limited to, dioxolanyl, oxasulfuranyl, disulfuranyl, and oxazolidin-2-one. Exemplary 5-membered heterocyclyl groups containing three heteroatoms include, but are not limited to, triazolinyl, oxadiazolinyl, and thiadiazolinyl. Exemplary 6-membered heterocyclyl groups containing one heteroatom include, but are not limited to, piperidyl, tetrahydropyranyl, dihydropyridyl and thianyl. Exemplary 6-membered heterocyclyl groups containing two heteroatoms include, but are not limited to, piperazinyl, morpholinyl, dithianyl and dioxanyl. Exemplary 6-membered heterocyclyl groups containing three heteroatoms include, but are not limited to, triazinanyl. Exemplary 7-membered heterocycyl groups containing one heteroatom include, but are not limited to, azepanyl, oxepanyl and thiepanyl. Exemplary 5-membered heterocyclyl groups fused with a C₆ aryl (also referred as 5,6-bicyclic heterocyclyl herein) include, but are not limited to, indolinyl, isoindolinyl, dihydrobenzofuranyl, dihydrobenzothiophenyl, benzoxazolinonyl, etc. Exemplary 6-membered heterocyclyl groups fused with a C₆ aryl (also referred as 6,6-bicyclic heterocyclyl herein) include, but are not limited to, tetrahydroquinolinyl, tetrahydroisoquinolinyl, etc. The heterocyclyl can be substituted with one or more substituents, for example, with 1 to 5 substituents, 1 to 3 substituents or 1 substituent.

“C₆₋₁₀ aryl” refers to a radical of monocyclic or polycyclic (e.g., bicyclic) 4n+2 aromatic ring system having 6-10 ring carbon atoms and zero heteroatoms (e.g., having 6 or 10 shared R electrons in a cyclic array). In some embodiments, the aryl group has six ring carbon atoms (“C₆ aryl”; for example, phenyl). In some embodiments, the aryl group has ten ring carbon atoms (“C₁₀ aryl”; for example, naphthyl, e.g., 1-naphthyl and 2-naphthyl). The aryl group also includes a ring system in which the aryl ring described above is fused with one or more cycloalkyl or heterocyclyl groups, and the point of attachment is on the aryl ring, in which case the number of carbon atoms continues to represent the number of carbon atoms in the aryl ring system. The aryl can be substituted with one or more substituents, for example, with 1 to 5 substituents, 1 to 3 substituents or 1 substituent.

“5- to 14-membered heteroaryl” refers to a radical of 5- to 14-membered monocyclic or bicyclic 4n+2 aromatic ring system (e.g., having 6, 10 or 14 shared π electrons in a cyclic array) having ring carbon atoms and 1-4 ring heteroatoms, wherein each heteroatom is independently selected from nitrogen, oxygen and sulfur. In the heteroaryl group containing one or more nitrogen atoms, the point of attachment can be a carbon or nitrogen atom as long as the valence permits. Heteroaryl bicyclic systems may include one or more heteroatoms in one or two rings. Heteroaryl also includes ring systems wherein the heteroaryl ring described above is fused with one or more cycloalkyl or heterocyclyl groups, and the point of attachment is on the heteroaryl ring. In such case, the number the carbon atoms continues to represent the number of carbon atoms in the heteroaryl ring system. In some embodiments, 5- to 10-membered heteroaryl groups are alternative, which are radicals of 5- to 10-membered monocyclic or bicyclic 4n+2 aromatic ring systems having ring carbon atoms and 1-4 ring heteroatoms. In some embodiments, 5- to 9-membered heteroaryl groups are alternative, which are radicals of 5- to 9-membered monocyclic or bicyclic 4n+2 aromatic ring systems having ring carbon atoms and 1-4 ring heteroatoms. In other embodiments, 5- to 6-membered heteroaryl groups are yet alternative, which are radicals of 5- to 6-membered monocyclic 4n+2 aromatic ring systems having ring carbon atoms and 1-4 ring heteroatoms. Exemplary 5-membered heteroaryl groups containing one heteroatom include, but are not limited to, pyrrolyl, furyl and thienyl. Exemplary 5-membered heteroaryl groups containing two heteroatoms include, but are not limited to, imidazolyl, pyrazolyl, oxazolyl, isoxazolyl, thiazolyl, and isothiazolyl. Exemplary 5-membered heteroaryl groups containing three heteroatoms include, but are not limited to, triazolyl, oxadiazolyl (such as, 1,2,4-oxadiazoly), and thiadiazolyl. Exemplary 5-membered heteroaryl groups containing four heteroatoms include, but are not limited to, tetrazolyl. Exemplary 6-membered heteroaryl groups containing one heteroatom include, but are not limited to, pyridyl. Exemplary 6-membered heteroaryl groups containing two heteroatoms include, but are not limited to, pyridazinyl, pyrimidinyl, and pyrazinyl. Exemplary 6-membered heteroaryl groups containing three or four heteroatoms include, but are not limited to, triazinyl and tetrazinyl, respectively. Exemplary 7-membered heteroaryl groups containing one heteroatom include, but are not limited to, azepinyl, oxepinyl, and thiepinyl. Exemplary 5,6-bicyclic heteroaryl groups include, but are not limited to, indolyl, isoindolyl, indazolyl, benzotriazolyl, benzothiophenyl, isobenzothiophenyl, benzofuranyl, benzoisofuranyl, benzimidazolyl, benzoxazolyl, benzoisoxazolyl, benzoxadiazolyl, benzothiazolyl, benzoisothiazolyl, benzothiadiazolyl, indolizinyl and purinyl. Exemplary 6,6-bicyclic heteroaryl groups include, but are not limited to, naphthyridinyl, pteridinyl, quinolyl, isoquinolyl, cinnolinyl, quinoxalinyl, phthalazinyl and quinazolinyl. The heteroaryl can be substituted with one or more substituents, for example, with 1 to 5 substituents, 1 to 3 substituents or 1 substituent.

“Cycloalkylene”, “heterocyclylene”, “arylene” or “heteroarylene” is a divalent group formed by removing another hydrogen from “cycloalkyl”, “heterocyclyl”, “aryl” or “heteroaryl” as defined above, and may be substituted or unsubstituted. For example, “C₅₋₇ cycloalkylene” refers to a divalent group formed by removing another hydrogen from C₅₋₇ cycloalkyl, “5- to 8-membered heterocyclylene” refers to a divalent group formed by removing another hydrogen from 5- to 8-membered heterocyclyl, “C₆₋₁₀ arylene” refers to a divalent group formed by removing another hydrogen from C₆₋₁₀ aryl, and “5- to 6-membered heteroarylene” refers to a divalent group formed by removing another hydrogen from 5- to 6-membered heteroaryl.

Alkyl, alkenyl, alkynyl, cycloalkyl, heterocyclyl, aryl and heteroaryl as defined herein are optionally substituted groups.

Exemplary substituents on carbon atoms include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂—OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), alkyl, haloalkyl, alkenyl, alkynyl, cycloalkyl, heterocyclyl, aryl and heteroaryl, wherein each of the alkyl, alkenyl, alkynyl, cycloalkyl, heterocyclyl, aryl and heteroaryl is independently substituted with 0, 1, 2, 3, 4 or 5 R^{dd} groups;
or two geminal hydrogen on a carbon atom are replaced with ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb} or ═NOR^{cc} groups;
each of the R^{aa} is independently selected from alkyl, haloalkyl, alkenyl, alkynyl, cycloalkyl, heterocyclyl, aryl and heteroaryl, or two of the R^{aa} groups are combined to form a heterocyclyl or heteroaryl ring, wherein each of the alkyl, alkenyl, alkynyl, cycloalkyl, heterocyclyl, aryl and heteroaryl is independently substituted with 0, 1, 2, 3, 4 or 5 R^{dd} groups;
each of the R^{bb} is independently selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, alkyl, haloalkyl, alkenyl, alkynyl, cycloalkyl, heterocyclyl, aryl and heteroaryl, or two R^{bb} groups are combined to form a heterocyclyl or a heteroaryl ring, wherein each of the alkyl, alkenyl, alkynyl, cycloalkyl, heterocyclyl, aryl and heteroaryl is independently substituted with 0, 1, 2, 3, 4 or 5 R^{dd} groups;
each of the R^{cc} is independently selected from hydrogen, alkyl, haloalkyl, alkenyl, alkynyl, cycloalkyl, heterocyclyl, aryl and heteroaryl, or two R^{cc} groups are combined to form a heterocyclyl or a heteroaryl ring, wherein each of the alkyl, alkenyl, alkynyl, cycloalkyl, heterocyclyl, aryl and heteroaryl is independently substituted with 0, 1, 2, 3, 4 or 5 R^{dd} groups;
each of the R^{dd} is independently selected from halogen, —CN, —NO₂, —N₃, —SO₂H, —SO₃H, —OH, —OR^{cc}, —ON(R^{ff})₂, —N(R^{ff})₂, —N(R)₃+X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{E})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, alkyl, haloalkyl, alkenyl, alkynyl, cycloalkyl, heterocyclyl, aryl, and heteroaryl, wherein each of the alkyl, alkenyl, alkynyl, cycloalkyl, heterocyclyl, aryl and heteroaryl is independently substituted with 0, 1, 2, 3, 4 or 5 R^{gg} groups, or two geminal R^{dd} substituents can be combined to form ═O or ═S;
each of the R^{ee} is independently selected from alkyl, haloalkyl, alkenyl, alkynyl, cycloalkyl, aryl, heterocyclyl, and heteroaryl, wherein each of the alkyl, alkenyl, alkynyl, cycloalkyl, heterocyclyl, aryl and heteroaryl is independently substituted with 0, 1, 2, 3, 4 or 5 R^{gg} groups;
each of the R^{ff} is independently selected from hydrogen, alkyl, haloalkyl, alkenyl, alkynyl, cycloalkyl, heterocyclyl, aryl and heteroaryl, or two R^{ff} groups are combined to form a heterocyclyl or a heteroaryl ring, wherein each of the alkyl, alkenyl, alkynyl, cycloalkyl, heterocyclyl, aryl and heteroaryl is independently substituted with 0, 1, 2, 3, 4 or 5 R^{gg} groups;
each of the R^{gg} is independently selected from halogen, —CN, —N₀₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃+X⁻, —NH(C₁₋₆ alkyl)₂+X⁻, —NH₂(C₁₋₆ alkyl)⁺X⁻, —NH₃⁻X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂₀C₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ haloalkyl, C₂-C₆ alkenyl, C₂-C₆ alkynyl, C₃-C₇ cycloalkyl, C₆-C₁₀ aryl, C₃-C₇ heterocyclyl, and C₅-C₁₀ heteroaryl; or two geminal R^{gg} substituents may combine to form ═O or ═S; wherein X⁻ is a counter-ion.

Exemplary substituents on nitrogen atoms include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{cc}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, alkyl, haloalkyl, alkenyl, alkynyl, cycloalkyl, heterocyclyl, aryl and heteroaryl, or two R^{cc} groups attached to a nitrogen atom combine to form a heterocyclyl or a heteroaryl ring, wherein each of the alkyl, alkenyl, alkynyl, cycloalkyl, heterocyclyl, aryl and heteroaryl is independently substituted with 0, 1, 2, 3, 4 or 5 R^{dd} groups, and wherein R^{aa}, R^{bb}, R^{cc} and R^{dd} are as described herein.

### Other Definitions

The term “pharmaceutically acceptable salt” as used herein refers to those carboxylate and amino acid addition salts of the compounds of the present disclosure, which are suitable for the contact with patients' tissues within a reliable medical judgment, and do not produce inappropriate toxicity, irritation, allergy, etc. They are commensurate with a reasonable benefit/risk ratio, and are effective for their intended use. The term includes, if possible, the zwitterionic form of the compounds of the disclosure.

“Subjects” to which administration is contemplated include, but are not limited to, humans (e.g., males or females of any age group, e.g., paediatric subjects (e.g., infants, children, adolescents) or adult subjects (e.g., young adults, middle-aged adults or older adults) and/or non-human animals, such as mammals, e.g., primates (e.g., cynomolgus monkeys, rhesus monkeys), cattle, pigs, horses, sheep, goats, rodents, cats and/or dogs. In some embodiments, the subject is a human. In some embodiments, the subject is a non-human animal. The terms “human”, “patient” and “subject” can be used interchangeably herein.

“Disease,” “disorder,” and “condition” can be used interchangeably herein.

Generally, the “effective amount” of a compound refers to an amount sufficient to elicit a target biological response. As understood by those skilled in the art, the effective amount of the compound of the disclosure can vary depending on the following factors, such as the desired biological endpoint, the pharmacokinetics of the compound, the diseases being treated, the mode of administration, and the age, health status and symptoms of the subjects. The effective amount includes therapeutically effective amount and prophylactically effective amount.

“Combination” and related terms refer to the simultaneous or sequential administration of the compounds of the present disclosure and other therapeutic agents. For example, the compounds of the present disclosure can be administered simultaneously or sequentially in separate unit dosage with other therapeutic agents, or simultaneously in a single unit dosage with other therapeutic agents.

### DETAILED DESCRIPTION

“Compound(s) of the present disclosure” herein refers to the following compounds of formula (I) (including sub-formulae, such as formula (II), formula (V-1), etc.), a pharmaceutically acceptable salt, enantiomer, diastereomer, solvate, hydrate or isotopic variant thereof, and mixtures thereof.

In one embodiment, the present disclosure relates to a compound of formula (I), or a pharmaceutically acceptable salt, isotopic variant, tautomer, stereoisomer, prodrug, polymorph, hydrate or solvate thereof:

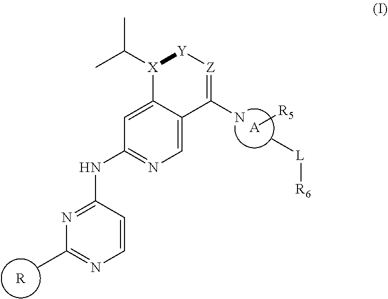

wherein

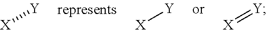

wherein when

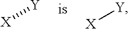

X is selected from CH and N, and Y is selected from CH₂, C═O and C═S; and when

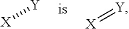

### X is C, and Y is CH;

Z is selected from CH and N;
ring A is 4- to 8-membered heterocyclyl;
ring B is selected from

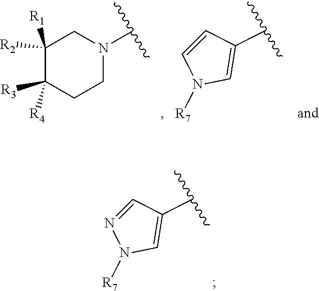

L is selected from a chemical bond, —S(═O)(=M)-, —N(R_{N})—S(═O)(=M)-, —S(═O)(=M)-N(R_{N})—, —N(R_{N})—C(═O)—, —C(═O)—N(R_{N})—, —CH(R_{N})—C(═O)—, —C(═O)—CH(R_{N})—, —CH(R_{N})—S(═O)(=M)- and —S(═O)(=M)-CH(R_{N})—;
R₁ and R₂ are independently selected from H, halogen, C₁₋₆ alkyl and C₁₋₆ haloalkyl;
R₃ and R₄ are independently selected from H, ORₐ, C₁₋₆ alkyl and C₁₋₆ haloalkyl;
R₅ is selected from H, halogen, C₁₋₆ alkyl and C₁₋₆ haloalkyl;
R₆ is selected from —C₀₋₆ alkylene-ORₐ, —C₀₋₆ alkylene-NR_{b}R_{c}, C₁₋₆ alkyl, C₁₋₆ haloalkyl, C₃-6 cycloalkyl, 4- to 6-membered heterocyclyl, C₆₋₁₀ aryl and 5- to 6-membered heteroaryl;
R₇ is selected from H, —C₁₋₆ alkylene-CN, —C₁₋₆ alkylene-ORₐ, —C₁₋₆ alkylene-NR_{b}R_{c}, C₁₋₆ alkyl, C₁₋₆ haloalkyl and —S(═O)(=M)-R₈;

wherein M is O or NH;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
R₈ is selected from —C₀₋₆ alkylene-ORₐ, —C₀₋₆ alkylene-NR_{b}R_{c}, C₁₋₆ alkyl, —C₁₋₆ alkylene-C₃₋₆ cycloalkyl, C₁₋₆ haloalkyl, C₃₋₆ cycloalkyl, 4- to 6-membered heterocyclyl, C₆₋₁₀ aryl and 5- to 6-membered heteroaryl, which are optionally substituted with one or more Rₐ;
Rₐ, R_{b} and R_{c} are independently selected from H, C₁₋₆ alkyl, C₁₋₆ haloalkyl, C₃₋₆ cycloalkyl and 4- to 6-membered heterocyclyl; or R_{b} and R_{c} together with the nitrogen atom to which they are attached form 4- to 7-membered heterocyclyl;
each group in X, Y, Z, ring A, ring B, L, R₁-R₈, R_{N}, Rₐ, R_{b} and R_{c} may be substituted by one or more deuterium atoms, up to full deuteration;
provided that, when L is —CH(R_{N})—S(═O)(=M)-, and the ring where X, Y and Z are located is a benzene ring, B is

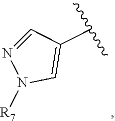

X, Y, Z

In one embodiment,

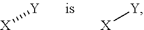

X is selected from CH and N, and Y is selected from CH₂, C═O and C═S, alternatively C═O; in another embodiment,

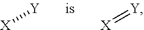

### is x, X is C, and Y is CH.

In one embodiment, Z is CH; in another embodiment, Z is N.

In a more specific embodiment, the ring where X, Y and Z are located is selected from:

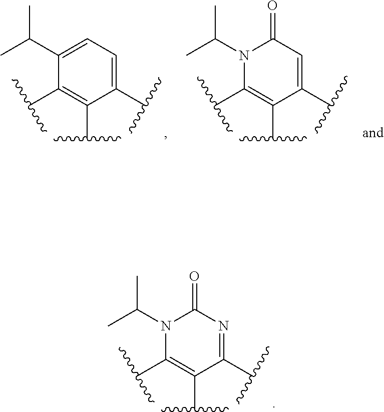 [SMILES: *c1ccc(C(C)C)c(*)c1* ; *c1cc(=O)n(C(C)C)c(*)c1* ; *c1nc(=O)n(C(C)C)c(*)c1*]

### Ring A

In one embodiment, ring A is 4- to 8-membered heterocyclyl; in another embodiment, ring A is selected from:

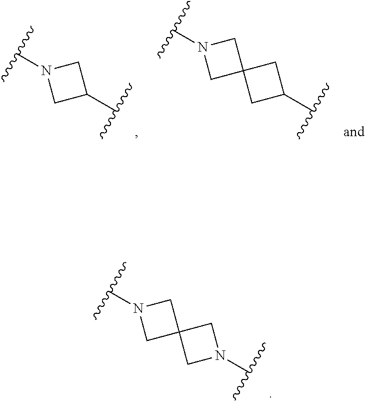 [SMILES: *C1CN(*)C1 ; *C1CC2(C1)CN(*)C2 ; *N1CC2(C1)CN(*)C2]

### Ring B

In one embodiment, ring B is

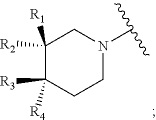

in another embodiment, ring B is

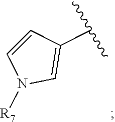

in another embodiment, ring B is

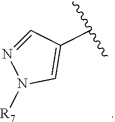

### L

In one embodiment, L is a chemical bond; in another embodiment, L is —S(═O)(=M)-; in another embodiment, L is —N(R_{N})—S(═O)(=M)-; in another embodiment, L is —S(═O)(=M)-N(R_{N})—; in another embodiment, L is —N(R_{N})—C(═O)—; in another embodiment, L is —C(═O)—N(R_{N})—; in another embodiment, L is —CH(R_{N})—C(═O)—; in another embodiment, L is —C(═O)—CH(R_{N})—; in another embodiment, L is —CH(R_{N})—S(═O)(=M)-; in another embodiment, L is —S(═O)(=M)-CH(R_{N})—.

R₁, R₂, R₃ and R₄

In one embodiment, R₁ and R₂ are independently selected from H, halogen, C₁₋₆ alkyl and C₁₋₆ haloalkyl; in another embodiment, one of R₁ and R₂ is halogen, and the other is selected from H, halogen and C₁₋₆ alkyl; in another embodiment, one of R₁ and R₂ is F, and the other is selected from H, F and Me; in another embodiment, one of R₁ and R₂ is F, and the other is H.

In one embodiment, one of R₃ and R₄ is ORₐ, and the other is selected from H and C₁₋₆ alkyl; in another embodiment, one of R₃ and R₄ is OH or OMe, and the other is selected from H and Me.

### R₅

In one embodiment, R₅ is H; in another embodiment, R₅ is halogen; in another embodiment, R₅ is C₁₋₆ alkyl; in another embodiment, R₅ is C₁₋₆ haloalkyl; in another embodiment, R₅ is selected from H and Me.

### R₆

In one embodiment, R₆ is —C₀₋₆ alkylene-ORₐ; in another embodiment, R₆ is OR; in another embodiment, R₆ is —C₀₋₆ alkylene-N_{b}R_{c}; in another embodiment, R₆ is NR_{b}R_{c}; in another embodiment, R₆ is C₁₋₆ alkyl; in another embodiment, R₆ is C₁₋₆ haloalkyl; in another embodiment, R₆ is C₃₋₆ cycloalkyl; in another embodiment, R₆ is 4- to 6-membered heterocyclyl; in another embodiment, R₆ is C₆₋₁₀ aryl; in another embodiment, R₆ is 5- to 6-membered heteroaryl.

### R₇

In one embodiment, R₇ is H; in another embodiment, R₇ is —C₁₋₆ alkylene-CN; in another embodiment, R₇ is —C₁₋₆ alkylene-ORₐ; in another embodiment, R₇ is —C₁₋₆ alkylene-NR_{b}R_{c}; in another embodiment, R₇ is C₁₋₆ alkyl; in another embodiment, R₇ is C₁₋₆ haloalkyl; in another embodiment, R₇ is —S(═O)(=M)-R₈.

### M

In one embodiment, M is O; in another embodiment, M is NH.

### R_{N}

In one embodiment, R_{N} is H; in another embodiment, R_{N} is C₁₋₆ alkyl; in another embodiment, R_{N} is C₁₋₆ haloalkyl; in another embodiment, R_{N} and R₆ are linked to form C₁₋₆ alkylene; in another embodiment, R_{N} and R₆ are linked to form C₂₋₆ alkenylene; in another embodiment, R_{N} and R₆ are linked to form C₂₋₆ alkynylene.

### R₈

In one embodiment, R₈ is —C₀₋₆ alkylene-ORₐ; in another embodiment, R₈ is OR; in another embodiment, R₈ is —C₀₋₆ alkylene-NR_{b}R_{c}; in another embodiment, R₈ is NR_{b}R_{c}; in another embodiment, R₈ is C₁₋₆ alkyl; in another embodiment, R₈ is —C₁₋₆ alkylene-C₃₋₆ cycloalkyl; in another embodiment, R₈ is C₁₋₆ haloalkyl; in another embodiment, R₈ is C₃₋₆ cycloalkyl; in another embodiment, R₈ is 4- to 6-membered heterocyclyl; in another embodiment, R₈ is C₆₋₁₀ aryl; in another embodiment, R₈ is 5- to 6-membered heteroaryl.

Any technical solution in any one of the above specific embodiments, or any combination thereof, may be combined with any technical solution in other specific embodiments or any combination thereof. For example, any technical solution of X or any combination thereof may be combined with any technical solution of Y, Z, ring A, ring B, L, R₅, R₆ or any combination thereof. The present disclosure is intended to include all combination of such technical solutions, which are not exhaustively listed here to save space.

In a more specific embodiment, the present disclosure provides a compound of formula (I), or a pharmaceutically acceptable salt, isotopic variant, tautomer, stereoisomer, prodrug, polymorph, hydrate or solvate thereof:

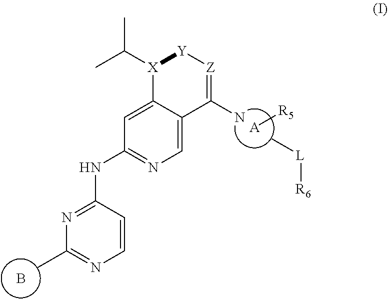

wherein:

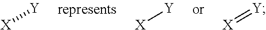

wherein when x is

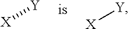

X is selected from CH and N, and Y is selected from CH₂, C═O and C═S; and when

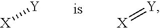

### X is C, and Y is CH;

Z is selected from CH and N;
ring A is 4- to 8-membered heterocyclyl;
ring B is selected from

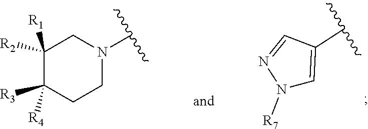

L is selected from a chemical bond, —S(═O)(=M)-, —N(R_{N})—S(═O)(=M)-, —S(═O)(=M)-N(R_{N})—, —N(R_{N})—C(═O)—, —C(═O)—N(R_{N})—, —CH(R_{N})—C(═O)—, —C(═O)—CH(R_{N})—, —CH(R_{N})—S(═O)(=M)- and —S(═O)(=M)-CH(R_{N})—;
R₁ and R₂ are independently selected from H, halogen, C₁₋₆ alkyl and C₁₋₆ haloalkyl;
R₃ and R₄ are independently selected from H, ORₐ, C₁₋₆ alkyl and C₁₋₆ haloalkyl;
R₅ is selected from H, halogen, C₁₋₆ alkyl and C₁₋₆ haloalkyl;
R₆ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl;
R₇ is selected from H, C₁₋₆ alkyl, C₁₋₆ haloalkyl and —S(═O)(=M)-R₈;
wherein M is O or NH;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
R₈ is selected from ORₐ, NR_{b}R_{c}, C₁₋₆ alkyl, —C₁₋₆ alkylene-C₃₋₆ cycloalkyl, C₁₋₆ haloalkyl, C₃₋₆ cycloalkyl, 4- to 6-membered heterocyclyl, C₆₋₁₀ aryl and 5- to 6-membered heteroaryl, which are optionally substituted with one or more Rₐ;
Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl;
provided that, when L is —CH(R_{N})—S(═O)(=M)-, and the ring where X, Y and Z are located is a benzene ring, B is

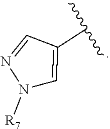

In a more specific embodiment, the present disclosure provides a compound of the above formula (I), or a pharmaceutically acceptable salt, isotopic variant, tautomer, stereoisomer, prodrug, polymorph, hydrate or solvate thereof, wherein the ring where X, Y and Z are located is selected from:

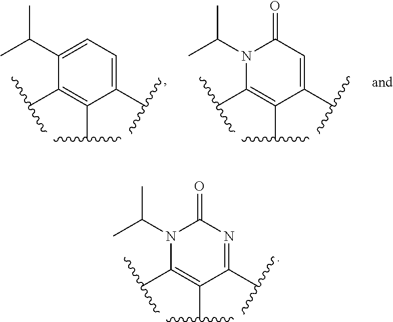 [SMILES: *c1ccc(C(C)C)c(*)c1* ; *c1cc(=O)n(C(C)C)c(*)c1* ; *c1nc(=O)n(C(C)C)c(*)c1*]

In a more specific embodiment, the present disclosure provides a compound of the above formula (I), or a pharmaceutically acceptable salt, isotopic variant, tautomer, stereoisomer, prodrug, polymorph, hydrate or solvate thereof, wherein ring A is selected from:

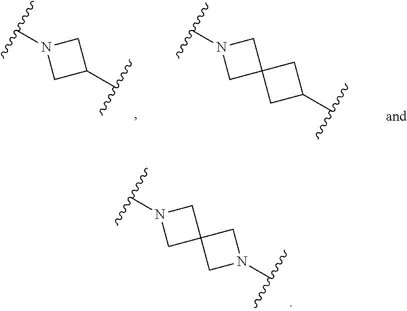 [SMILES: *C1CN(*)C1 ; *C1CC2(C1)CN(*)C2 ; *N1CC2(C1)CN(*)C2]

In a more specific embodiment, the present disclosure provides a compound of the above formula (I), or a pharmaceutically acceptable salt, isotopic variant, tautomer, stereoisomer, prodrug, polymorph, hydrate or solvate thereof, wherein ring B is

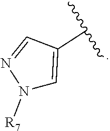

In a more specific embodiment, the present disclosure provides a compound of the above formula (I), or a pharmaceutically acceptable salt, isotopic variant, tautomer, stereoisomer, prodrug, polymorph, hydrate or solvate thereof, wherein ring B is

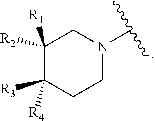

In a more specific embodiment, the present disclosure provides a compound of the above formula (I), or a pharmaceutically acceptable salt, isotopic variant, tautomer, stereoisomer, prodrug, polymorph, hydrate or solvate thereof, wherein L is selected from —S(═O)(=M)-, —N(R_{N})—S(═O)(=M)-, —N(R_{N})—C(═O)—, —CH(R_{N})—C(═O)— and —CH(R_{N})—S(═O)(=M)-; alternatively, L is —N(R_{N})—S(═O)(=M)- or —CH(R_{N})—S(═O)(=M)-.

In a more specific embodiment, the present disclosure provides a compound of the above formula (I), or a pharmaceutically acceptable salt, isotopic variant, tautomer, stereoisomer, prodrug, polymorph, hydrate or solvate thereof, wherein one of R₁ and R₂ is halogen, and the other is selected from H, halogen and C₁₋₆ alkyl; alternatively, one of R₁ and R₂ is F, and the other is selected from H, F and Me; alternatively, one of R₁ and R₂ is F, and the other is H.

In a more specific embodiment, the present disclosure provides a compound of the above formula (I), or a pharmaceutically acceptable salt, isotopic variant, tautomer, stereoisomer, prodrug, polymorph, hydrate or solvate thereof, wherein one of R₃ and R₄ is ORₐ, and the other is selected from H and C₁₋₆ alkyl; alternatively, one of R₃ and R₄ is OH or OMe, and the other is selected from H and Me.

In a more specific embodiment, the present disclosure provides a compound of the above formula (I), or a pharmaceutically acceptable salt, isotopic variant, tautomer, stereoisomer, prodrug, polymorph, hydrate or solvate thereof, wherein R₅ is selected from H and Me.

In a more specific embodiment, the present disclosure provides a compound of the above formula (I), or a pharmaceutically acceptable salt, isotopic variant, tautomer, stereoisomer, prodrug, polymorph, hydrate or solvate thereof, which has the following structure:

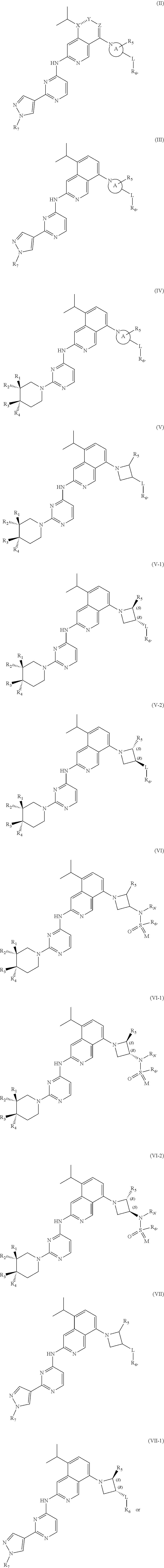

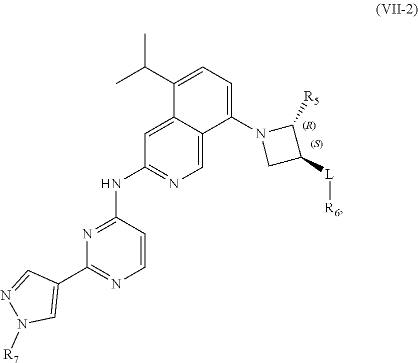

wherein each group is as defined above.

In a more specific embodiment, the present disclosure provides a compound of formula (II), or a pharmaceutically acceptable salt, isotopic variant, tautomer, stereoisomer, prodrug, polymorph, hydrate or solvate thereof:

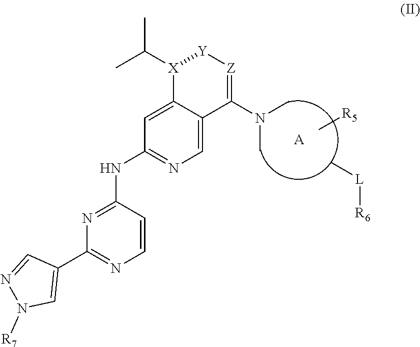

wherein:

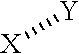

represents

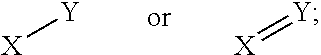

wherein when

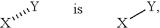

X is selected from CH and N, and Y is selected from CH₂, C═O and C═S; and when

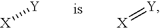

### X is C, and Y is CH;

Z is selected from CH and N;
ring A is 4- to 8-membered heterocyclyl;
L is selected from —S(═O)(=M)-, —N(R_{N})—S(═O)(=M)-, —S(═O)(=M)-N(R_{N})—, —N(R_{N})—C(═O)—, —C(═O)—N(R_{N})—, —CH(R_{N})—C(═O)—, —C(═O)—CH(R_{N})—, —CH(R_{N})—S(═O)(=M)- and —S(═O)(=M)-CH(R_{N})—;
R₅ is selected from H, halogen, C₁₋₆ alkyl and C₁₋₆ haloalkyl;
R₆ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl;
R₇ is selected from H, C₁₋₆ alkyl, C₁₋₆ haloalkyl and —S(═O)(=M)-R₈;
wherein M is O or NH;
R_{N} is selected from H, C₁₋₆ alkyl and C₁₋₆ haloalkyl;

or R_{N} and R₆ are linked to form C₁₋₆ alkylene, C₂₋₆ alkenylene or C₂₋₆ alkynylene;
R₈ is selected from ORₐ, NR_{b}R_{c}, C₁₋₆ alkyl, —CH₂—C₃₋₆ cycloalkyl, C₁₋₆ haloalkyl, C₃₋₆ cycloalkyl, 4- to 6-membered heterocyclyl, C₆₋₁₀ aryl and 5- to 6-membered heteroaryl, which are optionally substituted with one or more Rₐ;
Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl.

In a more specific embodiment, the present disclosure provides a compound of formula (III), or a pharmaceutically acceptable salt, isotopic variant, tautomer, stereoisomer, prodrug, polymorph, hydrate or solvate thereof:

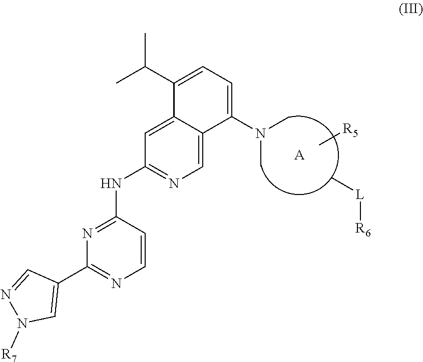

wherein:
ring A is 4- to 8-membered heterocyclyl;
L is selected from —S(═O)(=M)-, —N(R_{N})—S(═O)(=M)-, —S(═O)(=M)-N(R_{N})—, —N(R_{N})—C(═O)—, —C(═O)—N(R_{N})—, —CH(R_{N})—C(═O)—, —C(═O)—CH(R_{N})—, —CH(R_{N})—S(═O)(=M)- and —S(═O)(=M)-CH(R_{N})—;
R₅ is selected from H, halogen, C₁₋₆ alkyl and C₁₋₆ haloalkyl;
R₆ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl;
R₇ is selected from H, C₁₋₆ alkyl, C₁₋₆ haloalkyl and —S(═O)(=M)-R₈;
wherein M is O or NH;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
R₈ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl, which are optionally substituted with one or more Rₐ;
Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl.

In a more specific embodiment, the present disclosure provides the above-mentioned compound, or a pharmaceutically acceptable salt, isotopic variant, tautomer, stereoisomer, prodrug, polymorph, hydrate or solvate thereof, wherein:
ring A is selected from

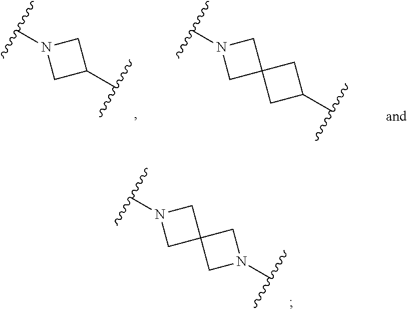 [SMILES: *C1CN(*)C1 ; *C1CC2(C1)CN(*)C2 ; *N1CC2(C1)CN(*)C2]

L is selected from —S(═O)(=M)-, —N(R_{N})—S(═O)(=M)-, —S(═O)(=M)-N(R_{N})—, —N(R_{N})—C(═O)—, —C(═O)—N(R_{N})—, —CH(R_{N})—C(═O)—, —C(═O)—CH(R_{N})—, —CH(R_{N})—S(═O)(=M)- and —S(═O)(=M)-CH(R_{N})—;
R₅ is selected from H and Me;
R₆ is selected from Me, Et, Pr, CH₂CF₃, cyclopropyl, OH, NH₂, NHMe, NMe₂, furan-2-yl and pyridin-4-yl;
R₇ is selected from H, trifluoroethyl and —S(═O)(=M)-R₈;
wherein M is O or NH;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
R₈ is selected from Me, Et, Pr, CH₂CF₃, cyclopropyl, OH, NH₂, NHMe, NMe₂, pyran-4-yl, furan-2-yl and pyridin-4-yl;
Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl.

In a more specific embodiment, the present disclosure provides the above-mentioned compound, or a pharmaceutically acceptable salt, isotopic variant, tautomer, stereoisomer, prodrug, polymorph, hydrate or solvate thereof, wherein:
ring A is 4- to 8-membered heterocyclyl;
L is selected from —S(═O)(=M)-, —CH(R_{N})—C(═O)— and —CH(R_{N})—S(═O)(=M)-;
R₅ is H, C₁₋₆ alkyl or C₁₋₆ haloalkyl;
R₆ is selected from ORₐ, NR_{b}R_{c}, C₁₋₆ alkyl, C₁₋₆ haloalkyl, C₃₋₆ cycloalkyl and 4- to 6-membered heterocyclyl;
R₇ is selected from H, trifluoroethyl and —S(═O)(=M)-R₈;
wherein M is O or NH;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
R₈ is selected from ORₐ, NR_{b}R_{c}, C₁₋₆ alkyl, C₁₋₆ haloalkyl, C₃₋₆ cycloalkyl and 4- to 6-membered heterocyclyl;
Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl.

In a more specific embodiment, the present disclosure provides the above-mentioned compound, or a pharmaceutically acceptable salt, isotopic variant, tautomer, stereoisomer, prodrug, polymorph, hydrate or solvate thereof, wherein:
ring A is 4- to 8-membered heterocyclyl;
L is selected from —S(═O)(=M)-, —CH(R_{N})—C(═O)— and —CH(R_{N})—S(═O)(=M)-;
R₅ is selected from H and C₁₋₆ alkyl;
R₆ is selected from NR_{b}R_{c}, C₁₋₆ alkyl and C₁₋₆ haloalkyl;
R₇ is selected from H, trifluoroethyl and —S(═O)(=M)-R₈;
wherein M is O or NH;
R_{N} is selected from H, C₁₋₆ alkyl and C₁₋₆ haloalkyl;
R₈ is selected from C₁₋₆ alkyl, C₁₋₆ haloalkyl, C₃₋₆ cycloalkyl and pyran-4-yl;
R_{b} and R_{c} are independently selected from H, C₁₋₆ alkyl and C₁₋₆ haloalkyl.

In a more specific embodiment, the present disclosure provides the above-mentioned compound, or a pharmaceutically acceptable salt, isotopic variant, tautomer, stereoisomer, prodrug, polymorph, hydrate or solvate thereof, wherein:
ring A is selected from

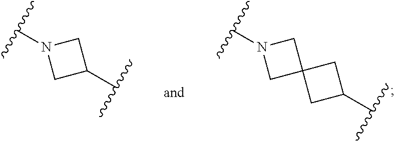 [SMILES: *C1CN(*)C1 ; *C1CC2(C1)CN(*)C2]

L is selected from —S(═O)(=M)-, —CH(R_{N})—C(═O)— and —CH(R_{N})—S(═O)(=M)-;
R₅ is H;
R₆ is selected from Me and NH₂;
R₇ is selected from H and —S(═O)(=M)-R₈;
wherein M is O;
R_{N} is selected from H, C₁₋₆ alkyl and C₁₋₆ haloalkyl;
R₈ is selected from Me, Et, Pr, cyclopropyl and pyran-4-yl.

In a more specific embodiment, the present disclosure provides a compound of formula (IV), or a pharmaceutically acceptable salt, isotopic variant, tautomer, stereoisomer, prodrug, polymorph, hydrate or solvate thereof:

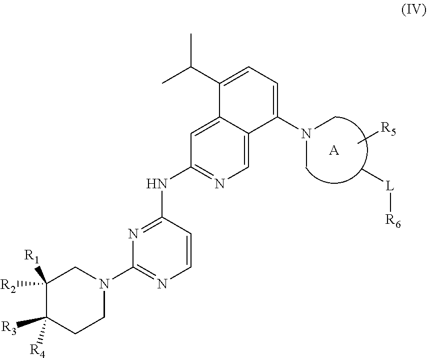

wherein:
ring A is 4- to 8-membered heterocyclyl;
L is selected from —S(═O)(=M)-, —N(R_{N})—S(═O)(=M)-, —S(═O)(=M)-N(R_{N})—, —N(R_{N})—C(═O)—, —C(═O)—N(R_{N})—, —CH(R_{N})—C(═O)— and —C(═O)—CH(R_{N})—;
R₁ and R₂ are independently selected from H, halogen, C₁₋₆ alkyl and C₁₋₆ haloalkyl;
R₃ and R₄ are independently selected from H, ORₐ, C₁₋₆ alkyl and C₁₋₆ haloalkyl;
R₅ is selected from H, halogen, C₁₋₆ alkyl and C₁₋₆ haloalkyl;
R₆ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl;
wherein M is O or NH;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
Rₐ, R_{b} and R, are independently selected from H, C₁₋₆ alkyl and C₁₋₆ haloalkyl; or R_{b} and R_{c} together with the nitrogen atom to which they are attached form 4- to 7-membered heterocyclyl.

In a more specific embodiment, the present disclosure provides the above-mentioned compound, or a pharmaceutically acceptable salt, isotopic variant, tautomer, stereoisomer, prodrug, polymorph, hydrate or solvate thereof, wherein:
ring A is selected from

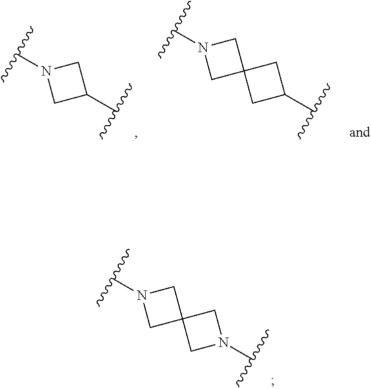 [SMILES: *C1CN(*)C1 ; *C1CC2(C1)CN(*)C2 ; *N1CC2(C1)CN(*)C2]

L is selected from —S(═O)(=M)-, —N(R_{N})—S(═O)(=M)-, —S(═O)(=M)-N(R_{N})—, —N(R_{N})—C(═O)—, —C(═O)—N(R_{N})—, —CH(R_{N})—C(═O)— and —C(═O)—CH(R_{N})—;
R₁ and R₂ are independently selected from H, halogen, C₁₋₆ alkyl and C₁₋₆ haloalkyl;
R₃ and R₄ are independently selected from H, ORₐ, C₁₋₆ alkyl and C₁₋₆ haloalkyl;
R₅ is selected from H, C₁₋₆ alkyl and C₁₋₆ haloalkyl;
R₆ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl;
wherein M is O or NH;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl.

In a more specific embodiment, the present disclosure provides a compound of formula (V), (V-1) or (V-2), or a pharmaceutically acceptable salt, isotopic variant, tautomer, stereoisomer, prodrug, polymorph, hydrate or solvate thereof:

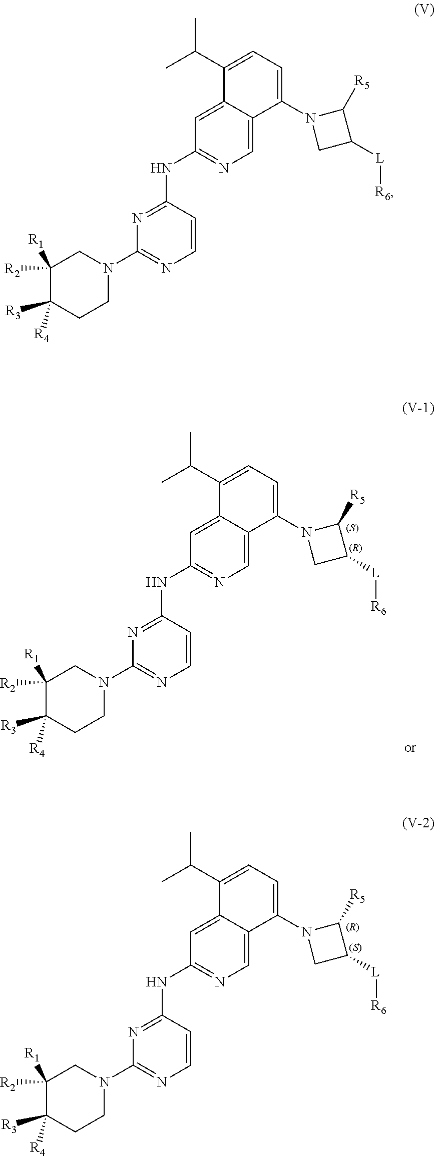

wherein:
L is selected from —S(═O)(=M)-, —N(R_{N})—S(═O)(=M)-, —S(═O)(=M)-N(R_{N})—, —N(R_{N})—C(═O)—, —C(═O)—N(R_{N})—, —CH(R_{N})—C(═O)— and —C(═O)—CH(R_{N})—;
R₁ and R₂ are independently selected from H, halogen, C₁₋₆ alkyl and C₁₋₆ haloalkyl;
R₃ and R₄ are independently selected from H, ORₐ, C₁₋₆ alkyl and C₁₋₆ haloalkyl;
R₅ is selected from H, halogen, C₁₋₆ alkyl and C₁₋₆ haloalkyl;
R₆ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl;
wherein M is O or NH;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl.

In a more specific embodiment, the present disclosure provides the above-mentioned compound, or a pharmaceutically acceptable salt, isotopic variant, tautomer, stereoisomer, prodrug, polymorph, hydrate or solvate thereof, wherein:
L is selected from —S(═O)(=M)-, —N(R_{N})—S(═O)(=M)-, —N(R_{N})—C(═O)— and —CH(R_{N})—C(═O)—;
one of R₁ and R₂ is halogen, and the other is selected from H, halogen and C₁₋₆ alkyl;
one of R₃ and R₄ is ORₐ, and the other is selected from H and C₁₋₆ alkyl;
R₅ is selected from H, C₁₋₆ alkyl and C₁₋₆ haloalkyl;
R₆ is selected from ORₐ, NR_{b}R_{c}, C₁₋₆ alkyl, C₁₋₆ haloalkyl, C₃₋₆ cycloalkyl and 5- to 6-membered heteroaryl;
wherein M is O or NH;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl.

In a more specific embodiment, the present disclosure provides a compound of formula (VI), (VI-1) or (VI-2), or a pharmaceutically acceptable salt, isotopic variant, tautomer, stereoisomer, prodrug, polymorph, hydrate or solvate thereof:

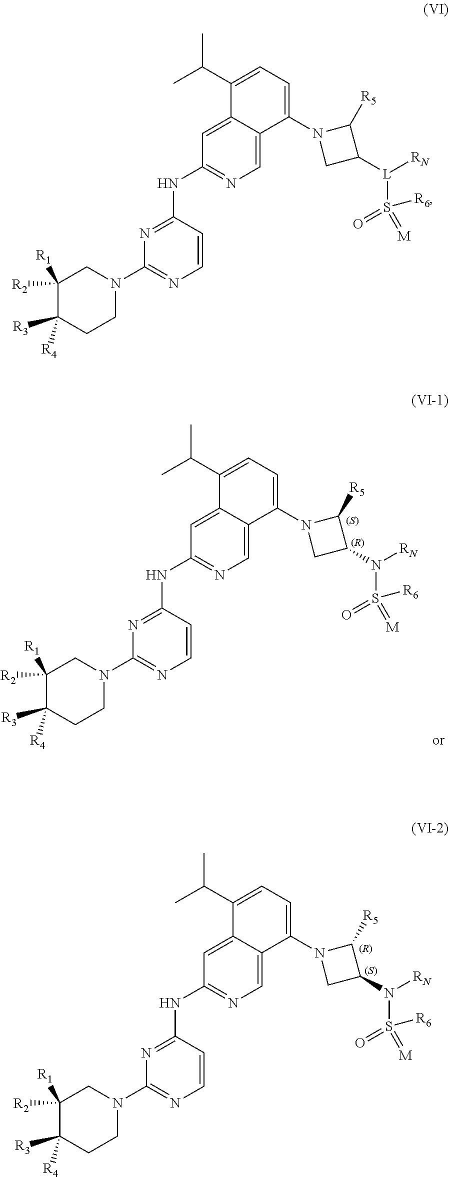

wherein:
M is O or NH;
R₁ and R₂ are independently selected from H, halogen, C₁₋₆ alkyl and C₁₋₆ haloalkyl;
R₃ and R₄ are independently selected from H, ORₐ, C₁₋₆ alkyl and C₁₋₆ haloalkyl;
R₅ is selected from H, halogen, C₁₋₆ alkyl and C₁₋₆ haloalkyl;
R₆ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
wherein Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl.

In a more specific embodiment, the present disclosure provides the above-mentioned compound, or a pharmaceutically acceptable salt, isotopic variant, tautomer, stereoisomer, prodrug, polymorph, hydrate or solvate thereof, wherein:
M is O or NH;
one of R₁ and R₂ is halogen, and the other is selected from H, halogen and C₁₋₆ alkyl;
one of R₃ and R₄ is ORₐ, and the other is selected from H and C₁₋₆ alkyl;
R₅ is selected from H and C₁₋₆ alkyl;
R₆ is selected from ORₐ, NR_{b}R_{c}, C₁₋₆ alkyl, C₁₋₆ haloalkyl, C₃₋₆ cycloalkyl and 5- to 6-membered heteroaryl;
R_{N} is selected from H and C₁₋₆ alkyl;
or R_{N} and R₆ are linked to form C₁₋₆ alkylene;
wherein Rₐ, R_{b} and R_{c} are independently selected from H, C₁₋₆ alkyl and C₁₋₆ haloalkyl.

In a more specific embodiment, the present disclosure provides the above-mentioned compound, or a pharmaceutically acceptable salt, isotopic variant, tautomer, stereoisomer, prodrug, polymorph, hydrate or solvate thereof, wherein:
M is O;
one of R₁ and R₂ is F, and the other is selected from H, F and Me;
one of R₃ and R₄ is OH or OMe, and the other is selected from H and Me;
R₅ is selected from H and Me;
R₆ is selected from Me, Et, Pr, CH₂CF₃, cyclopropyl, NMe₂, furan-2-yl and pyridin-4-yl;
R_{N} is selected from H and Me;
or R_{N} and R₆ are linked to form C₁₋₄ alkylene.

In a more specific embodiment, the present disclosure provides the above-mentioned compound, or a pharmaceutically acceptable salt, isotopic variant, tautomer, stereoisomer, prodrug, polymorph, hydrate or solvate thereof, wherein:
M is O or NH;
one of R₁ and R₂ is selected from halogen, C₁₋₆ alkyl and C₁₋₆ haloalkyl, and the other is H;
one of R₃ and R₄ is selected from ORₐ, C₁₋₆ alkyl and C₁₋₆ haloalkyl, and the other is H;
R₅ is selected from H, halogen, C₁₋₆ alkyl and C₁₋₆ haloalkyl;
R₆ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
wherein Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl.

In a more specific embodiment, the present disclosure provides the above-mentioned compound, or a pharmaceutically acceptable salt, isotopic variant, tautomer, stereoisomer, prodrug, polymorph, hydrate or solvate thereof, wherein:
M is O or NH;
one of R₁ and R₂ is selected from halogen and C₁₋₆ alkyl, and the other is H;
one of R₃ and R₄ is ORₐ, and the other is H;
R₅ is selected from H, C₁₋₆ alkyl and C₁₋₆ haloalkyl;
R₆ is selected from C₁₋₆ alkyl, C₁₋₆ haloalkyl, C₃₋₆ cycloalkyl and 4- to 6-membered heterocyclyl;
R_{N} is selected from H, C₁₋₆ alkyl and C₁₋₆ haloalkyl;
wherein Rₐ is selected from H, C₁₋₆ alkyl and C₁₋₆ haloalkyl.

In a more specific embodiment, the present disclosure provides the above-mentioned compound, or a pharmaceutically acceptable salt, isotopic variant, tautomer, stereoisomer, prodrug, polymorph, hydrate or solvate thereof, wherein:
M is O;
one of R₁ and R₂ is halogen (alternatively F), and the other is H;
one of R₃ and R₄ is ORₐ, and the other is H;
R₅ is selected from H and C₁₋₄ alkyl;
R₆ is selected from C₁₋₄ alkyl and C₁₋₄ haloalkyl;
R_{N} is selected from H, C₁₋₄ alkyl and C₁₋₄ haloalkyl; alternatively, C₁₋₄ alkyl;
wherein Rₐ is selected from H and C₁₋₄ alkyl.

In a more specific embodiment, the present disclosure provides a compound of formula (VII), (VII-1) or (VII-2), or a pharmaceutically acceptable salt, isotopic variant, tautomer, stereoisomer, prodrug, polymorph, hydrate or solvate thereof:

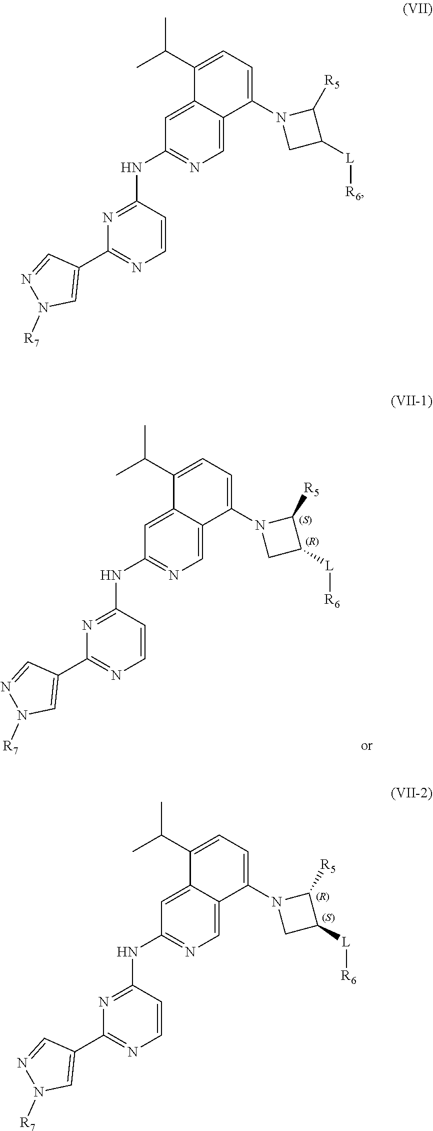

wherein:
L is selected from —S(═O)(=M)-, —N(R_{N})—S(═O)(=M)-, —S(═O)(=M)-N(R_{N})—, —N(R_{N})—C(═O)—, —C(═O)—N(R_{N})—, —CH(R_{N})—C(═O)—, —C(═O)—CH(R_{N})—, —CH(R_{N})—S(═O)(=M)- and —S(═O)(=M)-CH(R_{N})—;
R₅ is selected from H, halogen, C₁₋₆ alkyl and C₁₋₆ haloalkyl;
R₆ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl;
R₇ is selected from H, C₁₋₆ alkyl, C₁₋₆ haloalkyl and —S(═O)(=M)-R₈;
wherein M is O or NH;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
R₈ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl, which are optionally substituted with one or more Rₐ;
Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl.

In a more specific embodiment, the present disclosure provides the above-mentioned compound, or a pharmaceutically acceptable salt, isotopic variant, tautomer, stereoisomer, prodrug, polymorph, hydrate or solvate thereof, wherein:
wherein:
L is selected from —N(R_{N})—S(═O)(=M)- and —CH(R_{N})—S(═O)(=M)-;
R₅ is selected from C₁₋₆ alkyl and C₁₋₆ haloalkyl;
R₆ is selected from C₁₋₆ alkyl, C₁₋₆ haloalkyl, C₃₋₆ cycloalkyl and 4- to 6-membered heterocyclyl;
R₇ is —S(═O)(=M)-R₈;
wherein M is O or NH;
R_{N} is selected from H, C₁₋₆ alkyl and C₁₋₆ haloalkyl;
R₈ is selected from C₁₋₆ alkyl, C₁₋₆ haloalkyl, C₃₋₆ cycloalkyl and 4- to 6-membered heterocyclyl, which are optionally substituted with one or more Rₐ;
Rₐ is selected from H, C₁₋₆ alkyl and C₁₋₆ haloalkyl.

In a more specific embodiment, the present disclosure provides the above-mentioned compound, or a pharmaceutically acceptable salt, isotopic variant, tautomer, stereoisomer, prodrug, polymorph, hydrate or solvate thereof, wherein:
wherein:
L is selected from —N(R_{N})—S(═O)(=M)- and —CH₂—S(═O)(=M)-;
R₅ is selected from C₁₋₆ alkyl and C₁₋₆ haloalkyl;
R₆ is selected from C₁₋₆ alkyl and C₁₋₆ haloalkyl;
R₇ is —S(═O)(=M)-R₈;
wherein M is O or NH;
R_{N} is selected from C₁₋₆ alkyl and C₁₋₆ haloalkyl;
R₈ is selected from C₁₋₆ alkyl, C₁₋₆ haloalkyl, C₃₋₆ cycloalkyl and 4- to 6-membered heterocyclyl.

In a more specific embodiment, the present disclosure provides the above-mentioned compound, or a pharmaceutically acceptable salt, isotopic variant, tautomer, stereoisomer, prodrug, polymorph, hydrate or solvate thereof, wherein:
wherein:
L is selected from —N(R_{N})—S(═O)₂— and —CH₂—S(═O)₂—;
R₅ is selected from C₁₋₆ alkyl and C₁₋₆ haloalkyl;
R₆ is selected from C₁₋₆ alkyl and C₁₋₆ haloalkyl;
R₇ is —S(═O)₂—R₈;
wherein R_{N} is selected from C₁₋₆ alkyl and C₁₋₆ haloalkyl;
R₈ is selected from C₁₋₆ alkyl, C₁₋₆ haloalkyl and C₃₋₆ cycloalkyl.

In a more specific embodiment, the present disclosure provides the above-mentioned compound, or a pharmaceutically acceptable salt, isotopic variant, tautomer, stereoisomer, prodrug, polymorph, hydrate or solvate thereof, wherein:
wherein:
L is selected from —N(R_{N})—S(═O)₂— and —CH₂—S(═O)₂—;
R₅ is C₁₋₄ alkyl;
R₆ is C₁₋₄ alkyl;
R₇ is —S(═O)₂—R₈;
wherein R_{N} is C₁₋₄ alkyl;
R₈ is selected from C₁₋₄ alkyl and C₃₋₆ cycloalkyl.

In a more specific embodiment, the present disclosure provides a compound, or a tautomer, stereoisomer, prodrug, crystalline form, pharmaceutically acceptable salt, hydrate or solvate thereof, wherein the compound is selected from:

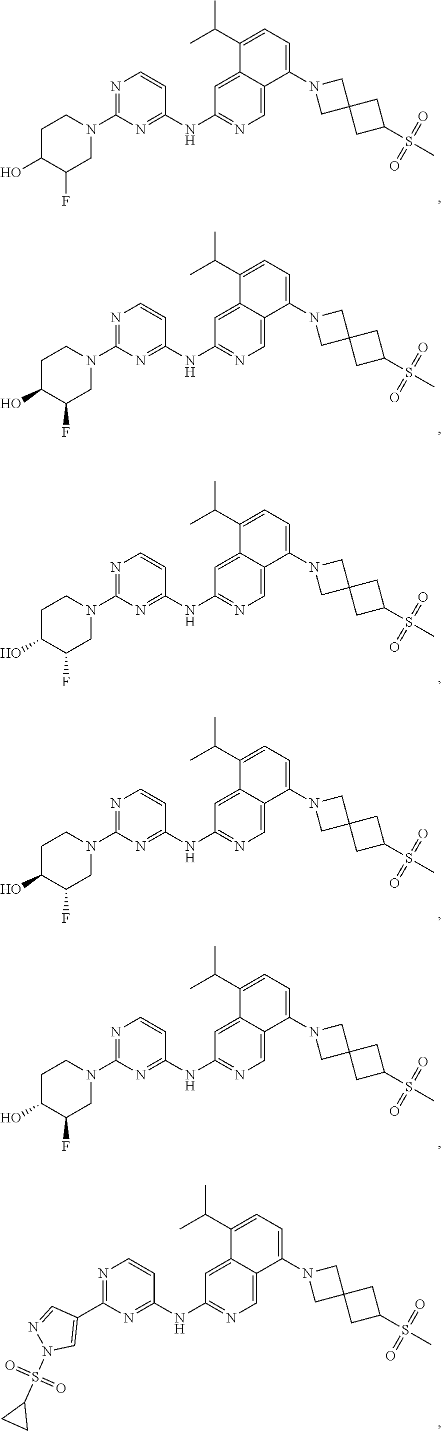 [SMILES: CC(C)c1ccc(N2CC3(CC(S(C)(=O)=O)C3)C2)c2cnc(Nc3ccnc(N4CCC(O)C(F)C4)n3)cc12 ; CC(C)c1ccc(N2CC3(CC(S(C)(=O)=O)C3)C2)c2cnc(Nc3ccnc(N4CC[C@H](O)[C@H](F)C4)n3)cc12 ; CC(C)c1ccc(N2CC3(CC(S(C)(=O)=O)C3)C2)c2cnc(Nc3ccnc(N4CC[C@@H](O)[C@@H](F)C4)n3)cc12 ; CC(C)c1ccc(N2CC3(CC(S(C)(=O)=O)C3)C2)c2cnc(Nc3ccnc(N4CC[C@H](O)[C@@H](F)C4)n3)cc12 ; CC(C)c1ccc(N2CC3(CC(S(C)(=O)=O)C3)C2)c2cnc(Nc3ccnc(N4CC[C@@H](O)[C@H](F)C4)n3)cc12 ; CC(C)c1ccc(N2CC3(CC(S(C)(=O)=O)C3)C2)c2cnc(Nc3ccnc(-c4cnn(S(=O)(=O)C5CC5)c4)n3)cc12]

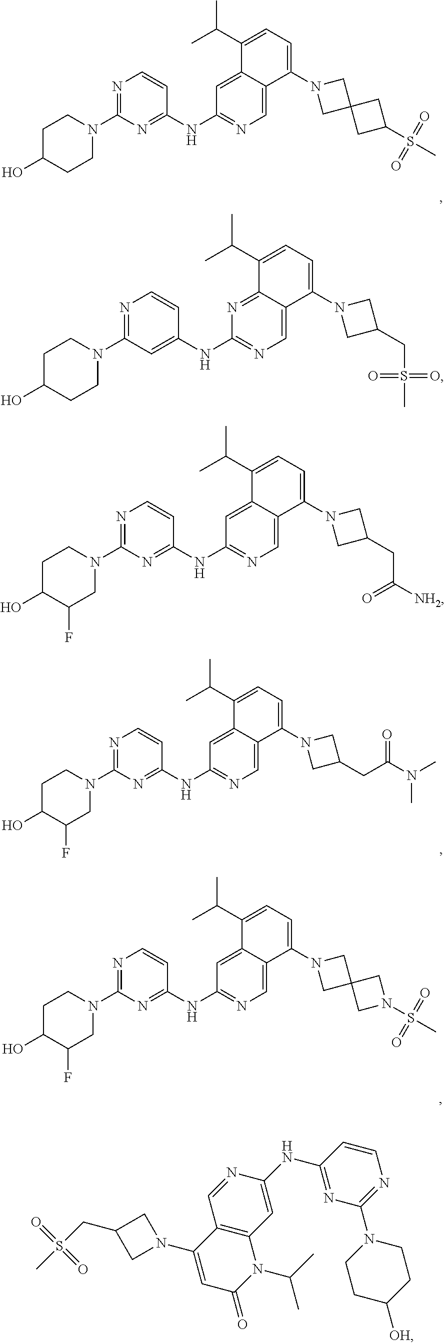 [SMILES: CC(C)c1ccc(N2CC3(CC(S(C)(=O)=O)C3)C2)c2cnc(Nc3ccnc(N4CCC(O)CC4)n3)cc12 ; CC(C)c1ccc(N2CC(CS(C)(=O)=O)C2)c2cnc(Nc3ccnc(N4CCC(O)CC4)c3)nc12 ; CC(C)c1ccc(N2CC(CC(N)=O)C2)c2cnc(Nc3ccnc(N4CCC(O)C(F)C4)n3)cc12 ; CC(C)c1ccc(N2CC(CC(=O)N(C)C)C2)c2cnc(Nc3ccnc(N4CCC(O)C(F)C4)n3)cc12 ; CC(C)c1ccc(N2CC3(C2)CN(S(C)(=O)=O)C3)c2cnc(Nc3ccnc(N4CCC(O)C(F)C4)n3)cc12 ; CC(C)n1c(=O)cc(N2CC(CS(C)(=O)=O)C2)c2cnc(Nc3ccnc(N4CCC(O)CC4)n3)cc21]

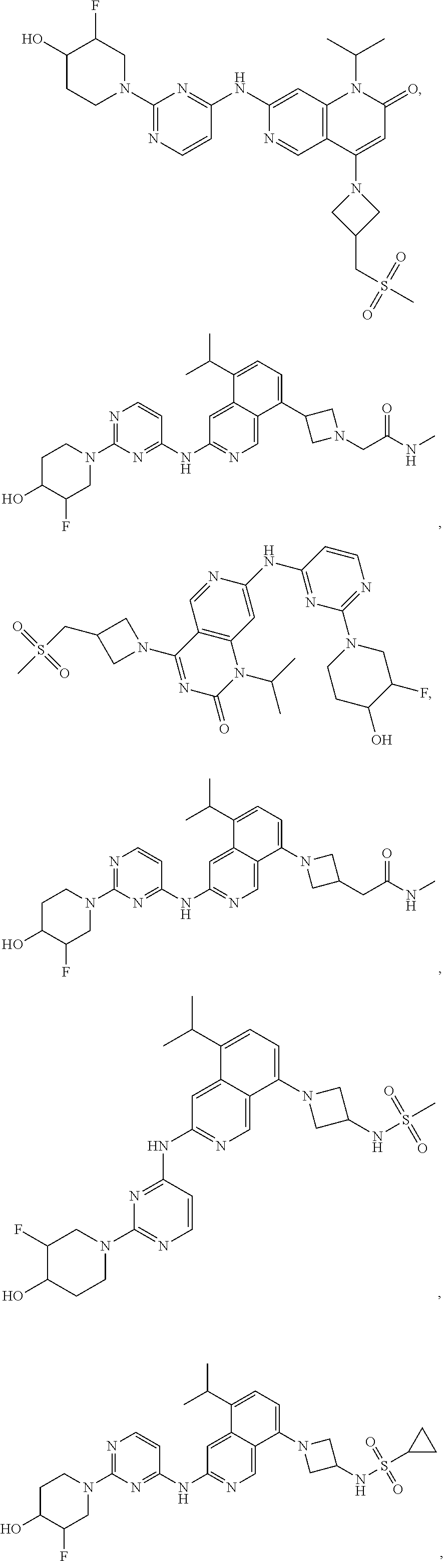 [SMILES: CC(C)n1c(=O)cc(N2CC(CS(C)(=O)=O)C2)c2cnc(Nc3ccnc(N4CCC(O)C(F)C4)n3)cc21 ; CNC(=O)CN1CC(c2ccc(C(C)C)c3cc(Nc4ccnc(N5CCC(O)C(F)C5)n4)ncc23)C1 ; CC(C)n1c(=O)nc(N2CC(CS(C)(=O)=O)C2)c2cnc(Nc3ccnc(N4CCC(O)C(F)C4)n3)cc21 ; CNC(=O)CC1CN(c2ccc(C(C)C)c3cc(Nc4ccnc(N5CCC(O)C(F)C5)n4)ncc23)C1 ; CC(C)c1ccc(N2CC(NS(C)(=O)=O)C2)c2cnc(Nc3ccnc(N4CCC(O)C(F)C4)n3)cc12 ; CC(C)c1ccc(N2CC(NS(=O)(=O)C3CC3)C2)c2cnc(Nc3ccnc(N4CCC(O)C(F)C4)n3)cc12]

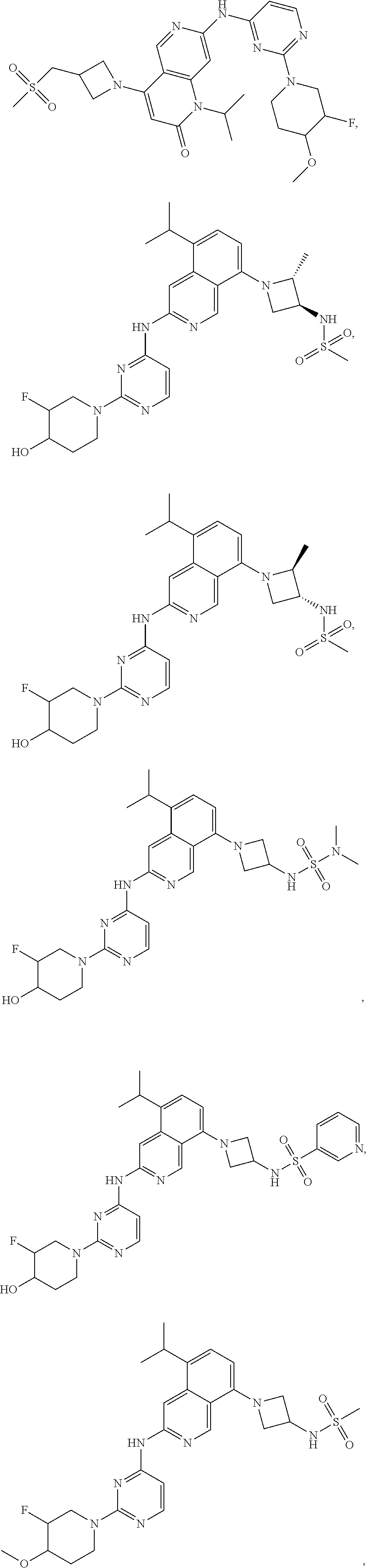

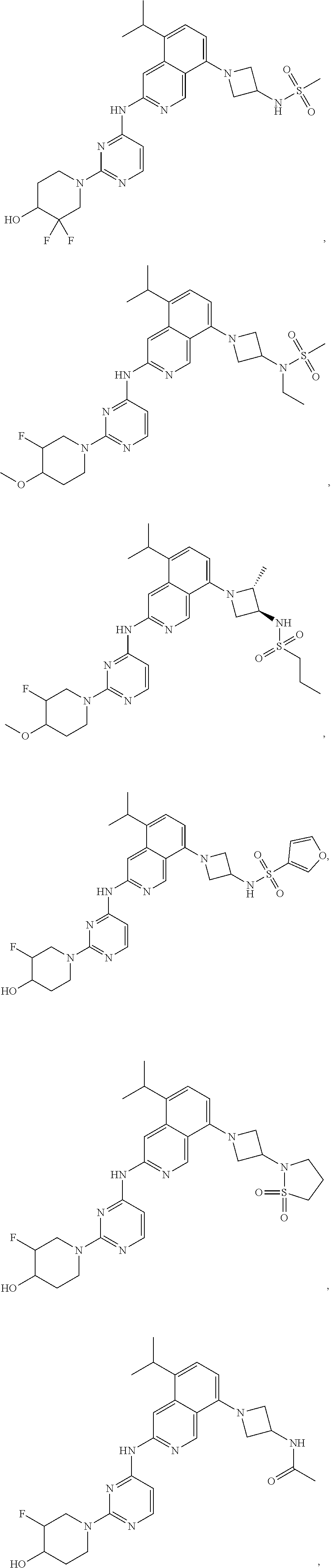

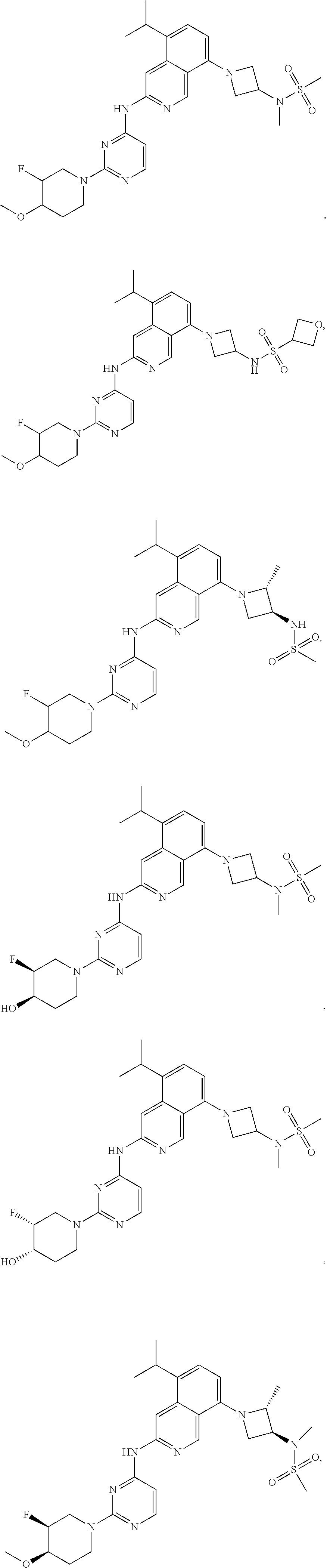

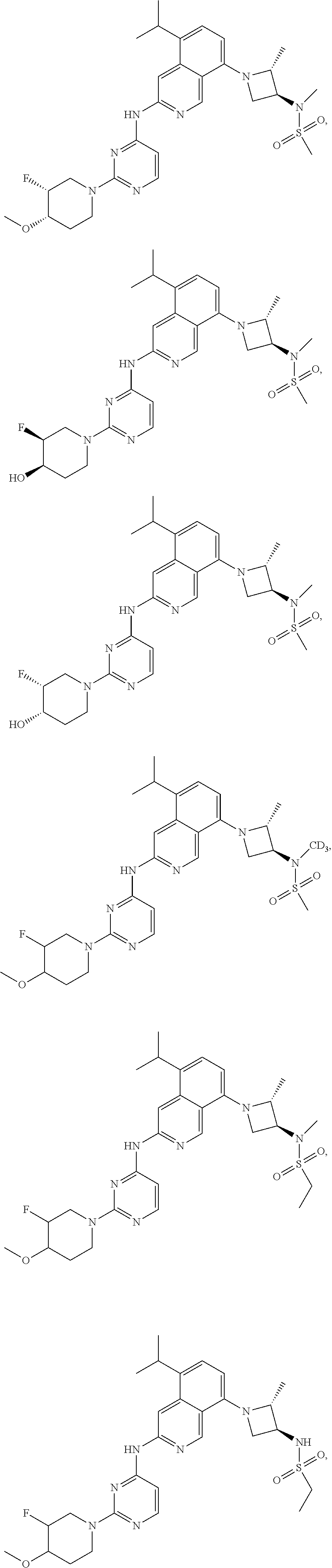

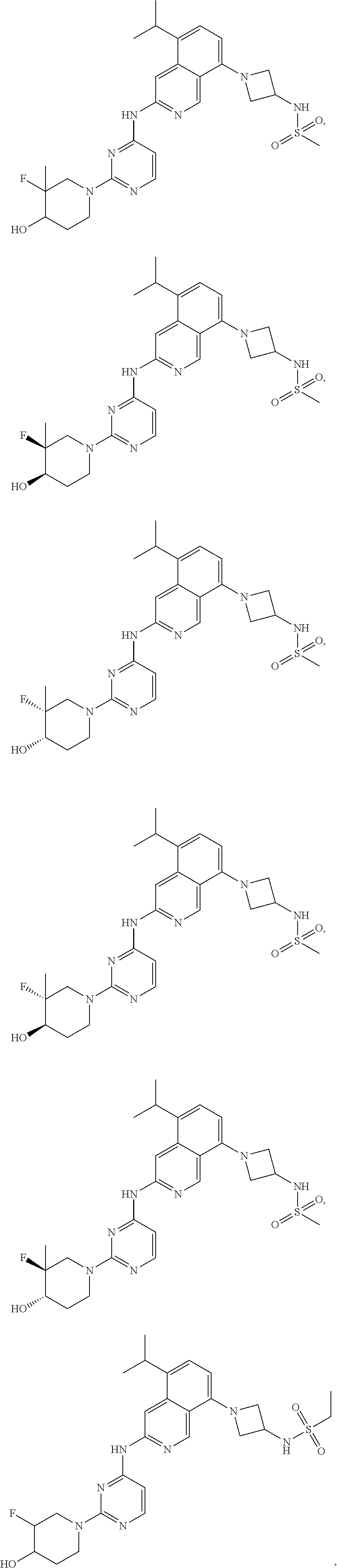 [SMILES: CC(C)c1ccc(N2CC(NS(C)(=O)=O)C2)c2cnc(Nc3ccnc(N4CCC(O)C(C)(F)C4)n3)cc12 ; CC(C)c1ccc(N2CC(NS(C)(=O)=O)C2)c2cnc(Nc3ccnc(N4CC[C@@H](O)[C@@](C)(F)C4)n3)cc12 ; CC(C)c1ccc(N2CC(NS(C)(=O)=O)C2)c2cnc(Nc3ccnc(N4CC[C@H](O)[C@](C)(F)C4)n3)cc12 ; CC(C)c1ccc(N2CC(NS(C)(=O)=O)C2)c2cnc(Nc3ccnc(N4CC[C@@H](O)[C@](C)(F)C4)n3)cc12 ; CC(C)c1ccc(N2CC(NS(C)(=O)=O)C2)c2cnc(Nc3ccnc(N4CC[C@H](O)[C@@](C)(F)C4)n3)cc12 ; CCS(=O)(=O)NC1CN(c2ccc(C(C)C)c3cc(Nc4ccnc(N5CCC(O)C(F)C5)n4)ncc23)C1]

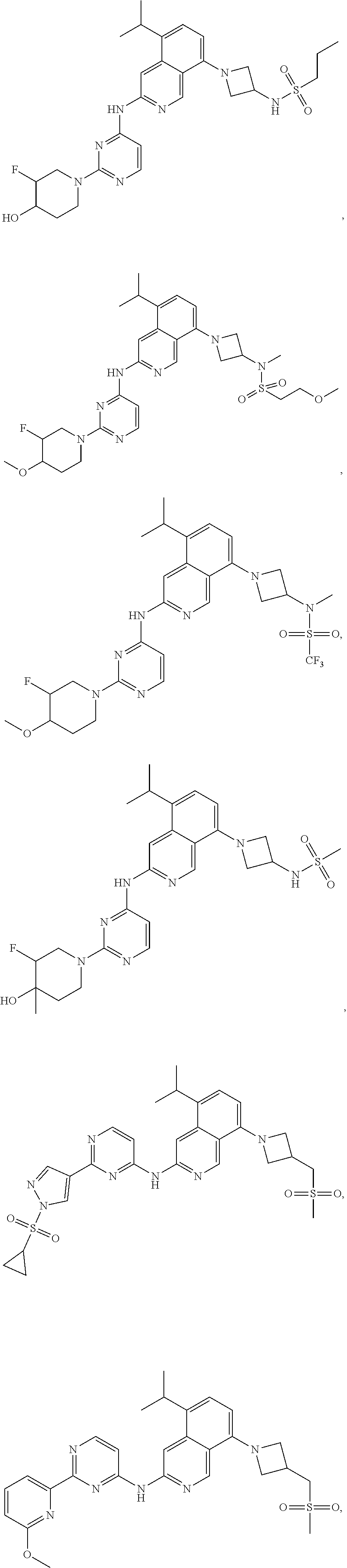

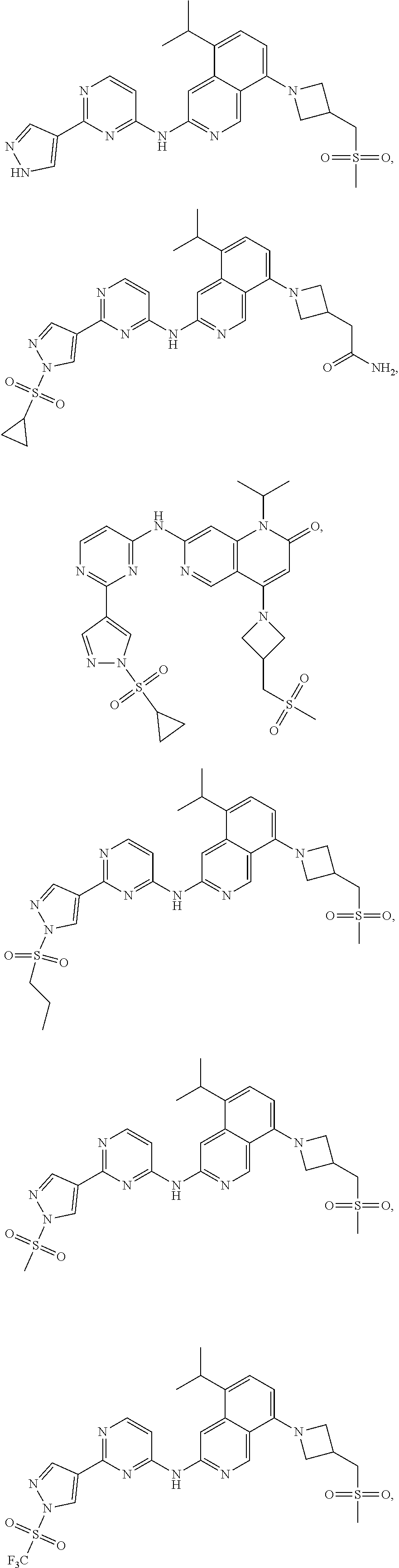 [SMILES: CC(C)c1ccc(N2CC(CS(C)(=O)=O)C2)c2cnc(Nc3ccnc(-c4cn[nH]c4)n3)cc12 ; CC(C)c1ccc(N2CC(CC(N)=O)C2)c2cnc(Nc3ccnc(-c4cnn(S(=O)(=O)C5CC5)c4)n3)cc12 ; CC(C)n1c(=O)cc(N2CC(CS(C)(=O)=O)C2)c2cnc(Nc3ccnc(-c4cnn(S(=O)(=O)C5CC5)c4)n3)cc21 ; CCCS(=O)(=O)n1cc(-c2nccc(Nc3cc4c(C(C)C)ccc(N5CC(CS(C)(=O)=O)C5)c4cn3)n2)cn1 ; CC(C)c1ccc(N2CC(CS(C)(=O)=O)C2)c2cnc(Nc3ccnc(-c4cnn(S(C)(=O)=O)c4)n3)cc12 ; CC(C)c1ccc(N2CC(CS(C)(=O)=O)C2)c2cnc(Nc3ccnc(-c4cnn(S(=O)(=O)C(F)(F)F)c4)n3)cc12]

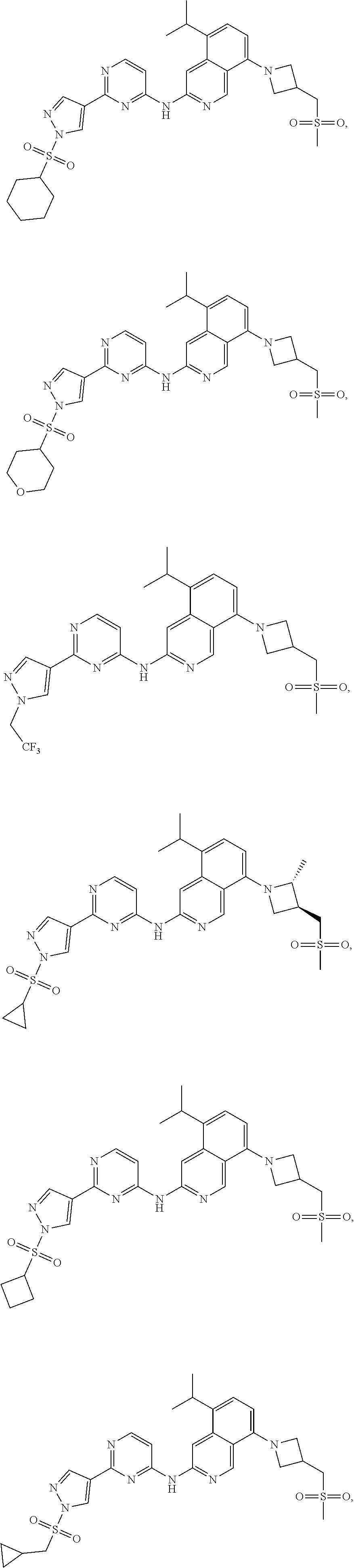 [SMILES: CC(C)c1ccc(N2CC(CS(C)(=O)=O)C2)c2cnc(Nc3ccnc(-c4cnn(S(=O)(=O)C5CCCCC5)c4)n3)cc12 ; CC(C)c1ccc(N2CC(CS(C)(=O)=O)C2)c2cnc(Nc3ccnc(-c4cnn(S(=O)(=O)C5CCOCC5)c4)n3)cc12 ; CC(C)c1ccc(N2CC(CS(C)(=O)=O)C2)c2cnc(Nc3ccnc(-c4cnn(CC(F)(F)F)c4)n3)cc12 ; CC(C)c1ccc(N2C[C@H](CS(C)(=O)=O)[C@H]2C)c2cnc(Nc3ccnc(-c4cnn(S(=O)(=O)C5CC5)c4)n3)cc12 ; CC(C)c1ccc(N2CC(CS(C)(=O)=O)C2)c2cnc(Nc3ccnc(-c4cnn(S(=O)(=O)C5CCC5)c4)n3)cc12 ; CC(C)c1ccc(N2CC(CS(C)(=O)=O)C2)c2cnc(Nc3ccnc(-c4cnn(S(=O)(=O)CC5CC5)c4)n3)cc12]

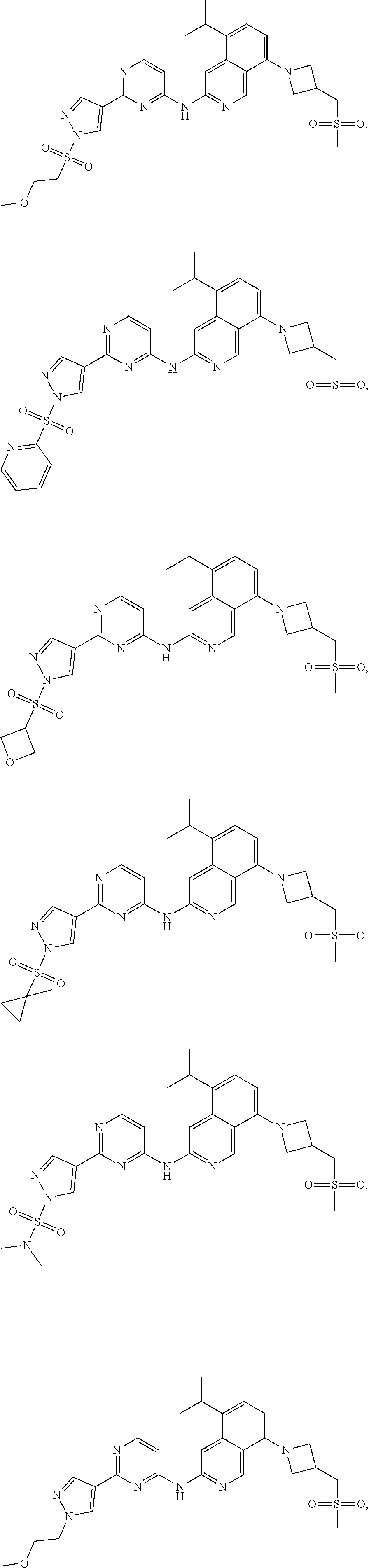 [SMILES: COCCS(=O)(=O)n1cc(-c2nccc(Nc3cc4c(C(C)C)ccc(N5CC(CS(C)(=O)=O)C5)c4cn3)n2)cn1 ; CC(C)c1ccc(N2CC(CS(C)(=O)=O)C2)c2cnc(Nc3ccnc(-c4cnn(S(=O)(=O)c5ccccn5)c4)n3)cc12 ; CC(C)c1ccc(N2CC(CS(C)(=O)=O)C2)c2cnc(Nc3ccnc(-c4cnn(S(=O)(=O)C5COC5)c4)n3)cc12 ; CC(C)c1ccc(N2CC(CS(C)(=O)=O)C2)c2cnc(Nc3ccnc(-c4cnn(S(=O)(=O)C5(C)CC5)c4)n3)cc12 ; CC(C)c1ccc(N2CC(CS(C)(=O)=O)C2)c2cnc(Nc3ccnc(-c4cnn(S(=O)(=O)N(C)C)c4)n3)cc12 ; COCCn1cc(-c2nccc(Nc3cc4c(C(C)C)ccc(N5CC(CS(C)(=O)=O)C5)c4cn3)n2)cn1]

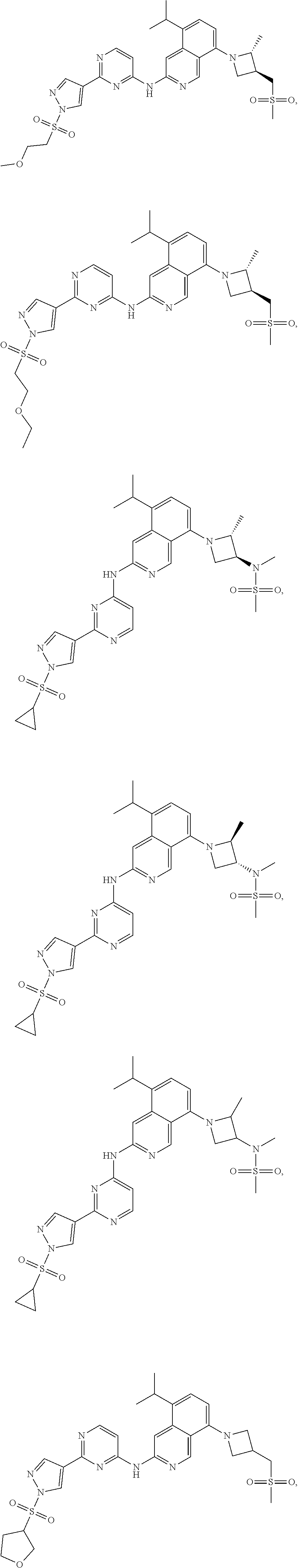

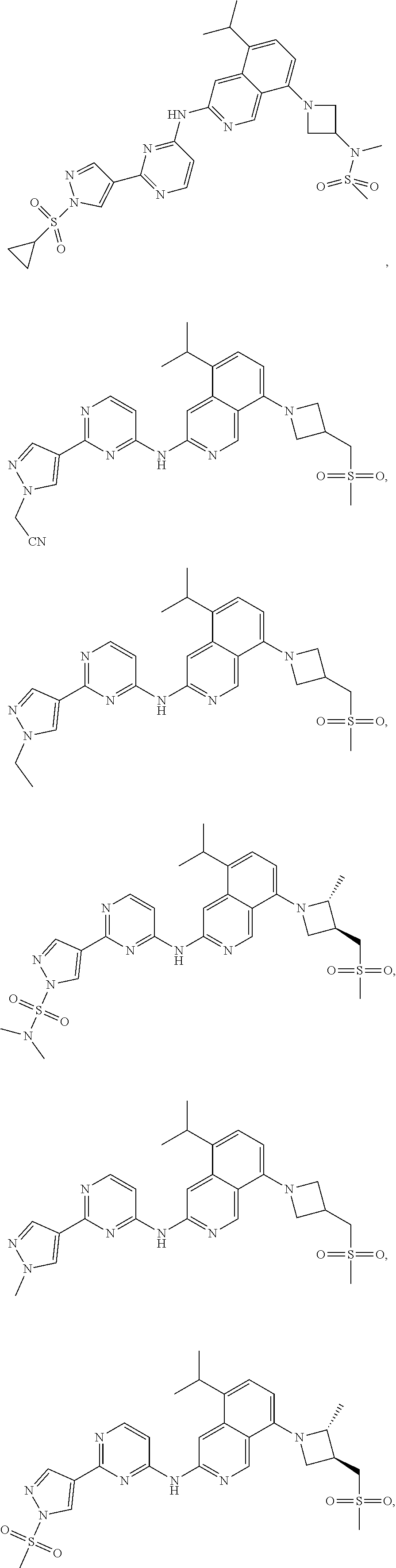

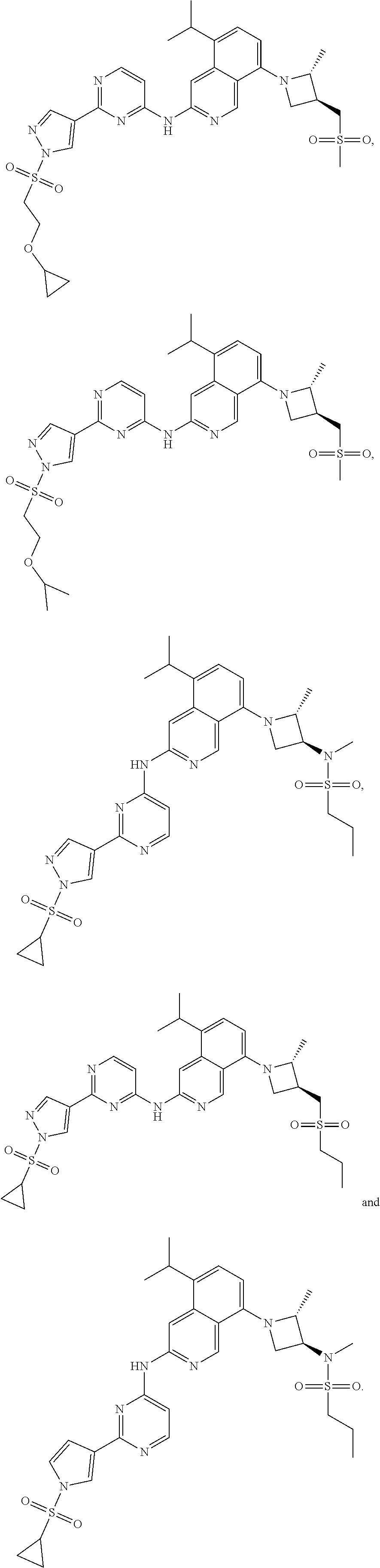 [SMILES: CC(C)c1ccc(N2C[C@H](CS(C)(=O)=O)[C@H]2C)c2cnc(Nc3ccnc(-c4cnn(S(=O)(=O)CCOC5CC5)c4)n3)cc12 ; CC(C)OCCS(=O)(=O)n1cc(-c2nccc(Nc3cc4c(C(C)C)ccc(N5C[C@H](CS(C)(=O)=O)[C@H]5C)c4cn3)n2)cn1 ; CCCS(=O)(=O)N(C)[C@H]1CN(c2ccc(C(C)C)c3cc(Nc4ccnc(-c5cnn(S(=O)(=O)C6CC6)c5)n4)ncc23)[C@@H]1C ; CCCS(=O)(=O)C[C@H]1CN(c2ccc(C(C)C)c3cc(Nc4ccnc(-c5cnn(S(=O)(=O)C6CC6)c5)n4)ncc23)[C@@H]1C ; CCCS(=O)(=O)N(C)[C@H]1CN(c2ccc(C(C)C)c3cc(Nc4ccnc(-c5ccn(S(=O)(=O)C6CC6)c5)n4)ncc23)[C@@H]1C]

The compounds of the present disclosure may include one or more asymmetric centers, and thus may exist in a variety of stereoisomeric forms, for example, enantiomers and/or diastereomers. For example, the compounds of the present disclosure may be in the form of an individual enantiomer, diastereomer or geometric isomer (e.g., cis- and trans-isomers), or may be in the form of a mixture of stereoisomers, including racemic mixture and a mixture enriched in one or more stereoisomers. The isomers can be separated from the mixture by the methods known to those skilled in the art, including chiral high-pressure liquid chromatography (HPLC) and the formation and crystallization of chiral salts; or alternative isomers can be prepared by asymmetric synthesis.

The compounds of the present disclosure may also exist as tautomers. For compounds that exist in different tautomeric forms, a compound is not limited to any specific tautomer, but is intended to encompass all tautomeric forms.

It will be understood by those skilled in the art that the organic compounds can form complexes with solvents in which they are reacted or from which they are precipitated or crystallized. These complexes are known as “solvates.” Where the solvent is water, the complex is known as “hydrate.” The present disclosure encompasses all solvates of the compounds of the present disclosure.

The term “solvate” refers to forms of a compound or a salt thereof, which are associated with a solvent, usually by a solvolysis reaction. This physical association may include hydrogen bonding. Conventional solvents include water, methanol, ethanol, acetic acid, DMSO, THF, diethyl ether, etc. The compounds described herein can be prepared, for example, in crystalline form, and can be solvated. Suitable solvates include pharmaceutically acceptable solvates and further include both stoichiometric solvates and non-stoichiometric solvates. In some cases, the solvates will be capable of isolation, for example, when one or more solvent molecules are incorporated into the crystal lattice of a crystalline solid. “Solvate” includes both solution-phase and isolatable solvates. Representative solvates include hydrates, ethanolates and methanolates.

The term “hydrate” refers to a compound that is associated with water. Generally, the number of water molecules contained in a hydrate of a compound is in a definite ratio to the number of the compound molecules in the hydrate. Therefore, hydrates of a compound can be represented, for example, by a general formula R-x H₂O, wherein R is the compound, and x is a number greater than 0. Given compounds can form more than one type of hydrates, including, for example, monohydrates (x is 1), lower hydrates (x is a number greater than 0 and smaller than 1, for example, hemihydrates (R-0.5 H₂O)) and polyhydrates (x is a number greater than 1, for example, dihydrates (R-2 H₂O) and hexahydrates (R-6 H₂O)).

Compounds of the present disclosure may be in an amorphous or a crystalline form (polymorph). Furthermore, the compounds of the present disclosure may exist in one or more crystalline forms. Therefore, the present disclosure includes all amorphous or crystalline forms of the compounds of the present disclosure within its scope. The term “polymorph” refers to a crystalline form of a compound (or a salt, hydrate or solvate thereof) in a particular crystal packing arrangement. All polymorphs have the same elemental composition. Different crystalline forms generally have different X-ray diffraction patterns, infrared spectra, melting points, density, hardness, crystal shapes, optical and electrical properties, stability, and solubility. Recrystallization solvents, rate of crystallization, storage temperatures, and other factors may cause one crystalline form to dominate. Various polymorphs of a compound can be prepared by crystallization under different conditions.

The present disclosure also comprises compounds that are labeled with isotopes (isotope variants), which are equivalent to those described in formula (I), but one or more atoms are replaced by atoms having an atom mass or mass number that are different from that of atoms that are common in nature. Examples of isotopes which may be introduced into the compounds of the disclosure include isotopes of hydrogen, carbon, nitrogen, oxygen, phosphorus, sulfur, fluorine and chlorine, such as ²H, ³H, ¹³C, ¹¹C, ¹⁴C, ¹⁵N, ¹⁸O, ¹⁷O, ³¹P, ³²P, 35S, ¹⁸F and ³⁶Cl, respectively. Compounds of the present disclosure that comprise the above isotopes and/or other isotopes of other atoms, prodrugs thereof and pharmaceutically acceptable salts of said compounds or prodrugs all are within the scope of the present disclosure. Certain isotope-labeled compounds of the present disclosure, such as those incorporating radioactive isotopes (e.g., ³H and ¹⁴C), can be used for the measurement of the distribution of drug and/or substrate in tissue. Tritium, which is ³H and carbon-14, which is ¹⁴C isotope, are yet alternative, because they are easy to prepare and detect. Furthermore, replaced by heavier isotopes, such as deuterium, which is ²H, may provide therapeutic benefits due to the higher metabolic stability, such as prolonging the half-life in vivo or decreasing the dosage requirements, and thus may be alternative in some cases. Isotope-labeled compounds of formula (I) of the present disclosure and prodrugs thereof can be prepared generally by using readily available isotope-labeled reagents to replace non-isotope-labeled reagents in the following schemes and/or the procedures disclosed in the examples and preparation examples.

In addition, prodrugs are also included within the context of the present disclosure. The term “prodrug” as used herein refers to a compound that is converted into an active form that has medical effects in vivo by, for example, hydrolysis in blood. Pharmaceutically acceptable prodrugs are described in T. Higuchi and V. Stella, Prodrugs as Novel Delivery Systems, A.C.S. Symposium Series, Vol. 14, Edward B. Roche, ed., Bioreversible Carriers in Drug Design, American Pharmaceutical Association and Pergamon Press, 1987, and D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

### Pharmaceutical Compositions and Kits

In another aspect, the present disclosure provides a pharmaceutical composition comprising a compound of the present disclosure (also referred to as the “active ingredient”) and a pharmaceutically acceptable excipient. In certain embodiments, the pharmaceutical composition comprises an effective amount of the compound of the present disclosure. In certain embodiments, the pharmaceutical composition comprises a therapeutically effective amount of the compound of the present disclosure. In certain embodiments, the pharmaceutical composition comprises a prophylactically effective amount of the compound of the present disclosure.

A pharmaceutically acceptable excipient for use in the present disclosure refers to a non-toxic carrier, adjuvant or vehicle which does not destroy the pharmacological activity of the compound formulated together. Pharmaceutically acceptable carriers, adjuvants, or vehicles that may be used in the compositions of the present disclosure include, but are not limited to, ion exchangers, alumina, aluminum stearate, lecithin, serum proteins (e.g., human serum albumin), buffer substances (such as phosphate), glycine, sorbic acid, potassium sorbate, a mixture of partial glycerides of saturated plant fatty acids, water, salt or electrolyte (such as protamine sulfate), disodium hydrogen phosphate, potassium hydrogen phosphate, sodium chloride, zinc salt, silica gel, magnesium trisilicate, polyvinyl pyrrolidone, cellulose-based materials, polyethylene glycol, sodium carboxymethyl cellulose, polyacrylate, wax, polyethylene-polyoxypropylene block polymers, polyethylene glycol and lanolin.

The present disclosure also includes kits (e.g., pharmaceutical packs). Kits provided may include a compound disclosed herein, other therapeutic agents, and a first and a second containers (e.g., vials, ampoules, bottles, syringes, and/or dispersible packages or other materials) containing the compound disclosed herein or other therapeutic agents. In some embodiments, kits provided can also optionally include a third container containing a pharmaceutically acceptable excipient for diluting or suspending the compound disclosed herein and/or other therapeutic agent. In some embodiments, the compound disclosed herein provided in the first container and the other therapeutic agents provided in the second container is combined to form a unit dosage form.

### Administration

The pharmaceutical composition provided by the present disclosure can be administered by a variety of routes including, but not limited to, oral administration, parenteral administration, inhalation administration, topical administration, rectal administration, nasal administration, oral administration, vaginal administration, administration by implant or other means of administration. For example, parenteral administration as used herein includes subcutaneous administration, intradermal administration, intravenous administration, intramuscular administration, intra-articular administration, intraarterial administration, intrasynovial administration, intrasternal administration, intracerebroventricular administration, intralesional administration, and intracranial injection or infusion techniques.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

When used to prevent the disorder disclosed herein, the compounds provided herein will be administered to a subject at risk for developing the condition, typically on the advice and under the supervision of a physician, at the dosage levels described above. Subjects at risk for developing a particular condition generally include those that have a family history of the condition, or those who have been identified by genetic testing or screening to be particularly susceptible to developing the condition.

The pharmaceutical compositions provided herein can also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc, or may be continued indefinitely, for example, for the rest of the subject's life. In certain embodiments, the chronic administration is intended to provide a constant level of the compound in the blood, e.g., within the therapeutic window over the extended period of time.

The pharmaceutical compostions of the present disclosure may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or alternatively from about 1 to about 40% by weight) with the remainder being various vehicles or excipients and processing aids helpful for forming the desired dosing form.

With oral dosing, one to five and especially two to four and typically three oral doses per day are representative regimens. Using these dosing patterns, each dose provides from about 0.01 to about 20 mg/kg of the compound provided herein, with alternative doses each providing from about 0.1 to about 10 mg/kg, and especially about 1 to about 5 mg/kg.

Transdermal doses are generally selected to provide similar or lower blood levels than are achieved using injection doses, generally in an amount ranging from about 0.01 to about 20% by weight, alternatively from about 0.1 to about 20% by weight, alternatively from about 0.1 to about 10% by weight, and still alternatively from about 0.5 to about 15% by weight.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable excipients known in the art. As before, the active compound in such compositions is typically a minor component, often being from about 0.05 to 10% by weight with the remainder being the injectable excipient and the like.

Transdermal compositions are typically formulated as a topical ointment or cream containing the active ingredient(s). When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or Formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds of the present disclosure can also be administered in sustained release forms or from sustained release drug delivery systems. A description of representative sustained release materials can be found in Remington's Pharmaceutical Sciences.

The present disclosure also relates to the pharmaceutically acceptable formulations of a compound of the present disclosure. In one embodiment, the formulation comprises water. In another embodiment, the formulation comprises a cyclodextrin derivative. The most common cyclodextrins are α-, β- and γ-cyclodextrins consisting of 6, 7 and 8 α-1,4-linked glucose units, respectively, optionally comprising one or more substituents on the linked sugar moieties, which include, but are not limited to, methylated, hydroxyalkylated, acylated, and sulfoalkylether substitution. In certain embodiments, the cyclodextrin is a sulfoalkyl ether β-cyclodextrin, e.g., for example, sulfobutyl ether β-cyclodextrin, also known as Captisol. See, e.g., U.S. Pat. No. 5,376,645. In certain embodiments, the formulation comprises hexapropyl-β-cyclodextrin (e.g., 10-50% in water).

### Examples

The reagents used in the present disclosure are commercial reagents purchased directly or synthesized by common methods well known in the art.

The specific reaction routes or steps exemplified below are used in the present disclosure:

### Example 1

### Preparation of Key Intermediates

### Synthesis of Intermediate a1

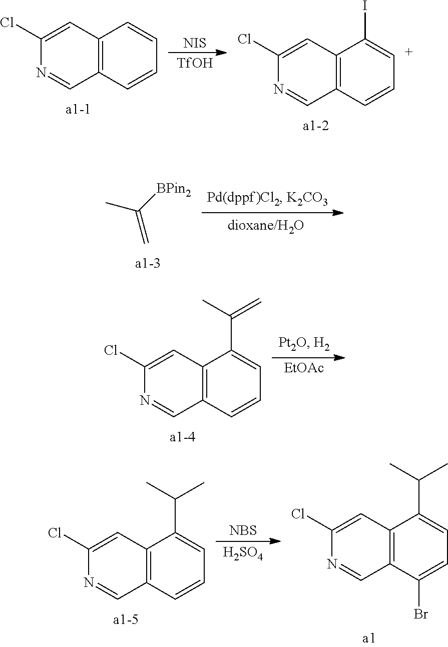

Step 1: 3-Chloroisoquinoline a1-1 (3.5 g, 21.4 mmol) was dissolved in 50 mL of trifluoromethanesulfonic acid at −10° C. under nitrogen protection. N-iodosuccinimide NIS (7.21 g, 32.1 mmol) was added in batches and the mixture was stirred for 4 hours. The reaction was completed by TLC monitoring. The reaction solution was quenched by slowly adding 100 mL of ammonia water, extracted with ethyl acetate, washed with saturated brine, dried over anhydrous sodium sulfate, and filtered. The filtrate was concentrated. The residue was separated by column chromatography (PE/EA=100/1) to obtain a white solid a1-2 (4.1 g), yield: 60%, LCMS: ESI-MS (m/z): 289.8 [M+H]⁺.

1H NMR (400 MHz, CDCl₃) δ 8.95 (s, 1H), 8.26 (dd, J=7.4, 1.1 Hz, 1H), 7.98-7.94 (m, 1H), 7.93 (d, J=0.7 Hz, 1H), 7.33 (dd, J=8.2, 7.4 Hz, 1H).

Step 2: Under nitrogen protection, the intermediate a1-2 (4.1 g, 14.2 mmol) from the previous step and K₂CO₃ (5.87 g, 42.3 mmol) were mixed in 110 mL of a mixture of 1,4-dioxane and water (v/v=10/1), and Pd(dppf)Cl₂ (1.04 g, 1.42 mmol) was added. The mixture was heated to 90° C. Isopropenylboronic acid pinacol ester a1-3 (3.6 g, 21.2 mmol) was added to the reaction solution, and the reaction solution was reacted at 60° C. for another 3 hours. The reaction was stopped. The reaction solution was filtered, and the filtrate was concentrated. 50 mL of water was added to the system, and the mixture was extracted with ethyl acetate, dried over anhydrous sodium sulfate, and filtered. The filtrate was concentrated, and separated by flash column chromatography to obtain a yellow solid a1-4 (1.9 g), yield: 53%, LC-MS: M+H⁺=204.0.

1H NMR (400 MHz, DMSO-d6) δ 9.21 (s, 1H), 8.09 (dd, J=6.2, 3.1 Hz, 1H), 7.85 (s, 1H), 7.68-7.65 (m, 2H), 5.49 (t, J=1.6 Hz, 1H), 5.03 (s, 1H), 2.14 (s, 3H).

Step 3: 3-Chloro-5-(prop-1-en-2-yl)isoquinoline a1-4 (3.5 g, 17.2 mmol) from the previous step was dissolved in 50 mL of ethyl acetate, and platinum dioxide (0.78 g, 3.4 mmol) was added. The mixture was reacted at room temperature for 6 hours under hydrogen atmosphere. The reaction solution was filtered. The filtrate was concentrated, and separated by column chromatography to obtain an oily intermediate a1-5 (2.1 g), yield: 54%, LC-MS: ESI-MS (m/z): 206.1 [M+H]⁺.

1H NMR (400 MHz, DMSO-d₆) δ 9.21 (s, 1H), 8.12 (s, 1H), 8.03 (d, J=8.1 Hz, 1H), 7.76 (d, J=6.7 Hz, 1H), 7.69 (d, J=7.9 Hz, 1H), 3.76-3.65 (m, 1H), 1.32 (d, J=6.8 Hz, 6H).

Step 4: The intermediate a1-5 (1.8 g, 8.75 mmol) from the previous step was dissolved in 24 mL of concentrated sulfuric acid at −10° C. under nitrogen protection. N-bromosuccinimide NBS (2.02 g, 11.4 mmol) was added in batches and the mixture was stirred for 8 hours. The reaction was completed by LC-MS monitoring. 200 mL of ice water was slowly added to the reaction solution to quench the reaction, and the mixture was adjusted to pH of about 8 with ammonia water. The mixture was extracted with ethyl acetate, washed with saturated brine, dried over anhydrous sodium sulfate, and filtered. The filtrate was concentrated. The residue was separated by column chromatography (PE/EA=50/1) to obtain a white solid a1 (1.0 g), yield: 36%, LCMS: ESI-MS (m/z): 283.9 [M+H]⁺.

1H NMR (400 MHz, DMSO-d₆) δ 9.34 (s, 1H), 8.22 (s, 1H), 7.99 (d, J=7.9 Hz, 1H), 7.66 (d, J=7.9 Hz, 1H), 3.72 (dt, J=13.7, 6.8 Hz, 1H), 1.31 (d, J=6.8 Hz, 6H).

### Synthesis of Intermediate a2

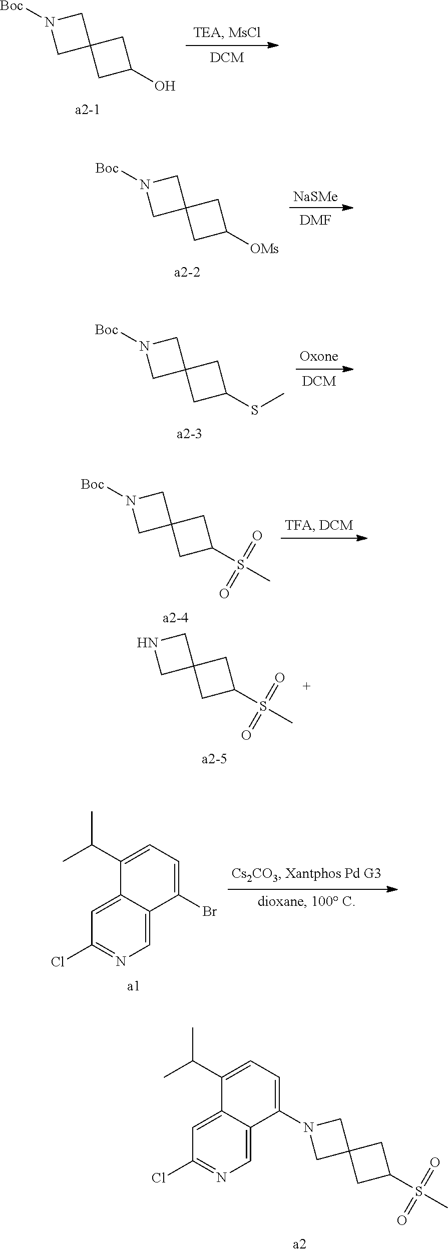

Step 1: Tert-butyl 6-hydroxy-2-azaspiro[3.3]heptane-2-carboxylate a2-1 (4.8 g, 22 mmol) and triethylamine (4.53 g, 44 mmol) were dissolved in 50 mL of dichloromethane in an ice bath and under nitrogen protection, and methylsulfonyl chloride (3.08 g, 26 mmol) was added dropwise. After the addition was completed, the mixture was stirred for another 2 hours. 10 mL of aqueous saturated sodium bicarbonate solution was added to the reaction solution to quench the reaction, and the mixture was extracted with dichloromethane. The combined organic layer was dried over anhydrous sodium sulfate and concentrated to obtain a crude compound a2-2 (6.24 g), yield: 86%.

Step 2: Under nitrogen protection, the intermediate a2-2 (6.24 g, 21 mmol) from the previous step and sodium thiomethoxide (20% aqueous solution) (15.0 g, 42 mmol) were dissolved in 50 mL of DMF, and the solution was heated to 60° C. and stirred for 12 hours. The reaction was completed by LC-MS monitoring. 200 mL of water was added to the system, and the mixture was extracted with ethyl acetate, and washed with saturated brine. The combined organic layer was dried over anhydrous sodium sulfate, and filtered. The filtrate was concentrated to obtain a colorless oil a2-3 (3.98 g), yield: 69%.

Step 3: The intermediate a2-3 (3.98 g, 16 mmol) from the previous step was dissolved in 200 mL of dichloromethane, and the oxidant Oxone (50.1 g, 81 mmol) was added. The mixture was reacted at room temperature for 48 hours. The reaction was stopped, and the mixture was filtered. The filtrate was concentrated to obtain compound a2-4 (4.18 g), yield: 83%.

1H NMR (400 MHz, CDCl₃) δ 3.95 (d, J=5.7 Hz, 4H), 3.72-3.52 (m, 1H), 2.80 (s, 3H), 2.76-2.65 (m, 2H), 2.59-2.49 (m, 2H), 1.43 (s, 9H).

Step 4: Compound a2-4 (3.72 g, 13 mmol) was dissolved in 40 mL of dichloromethane, and trifluoroacetic acid (4.62 g, 41 mmol) was added dropwise. The mixture was stirred at room temperature for 2 hours. The solvent was removed by evaporation under reduced pressure to obtain crude product a2-5 (1.95 g). LCMS: ESI-MS (m/z): 176.0 [M+H]⁺.

Step 5: Under nitrogen protection, the intermediate a2-5 (1.6 g, 5.5 mmol) from the previous step and Cs₂CO₃ (5.38 g, 16.5 mmol) were mixed in 25 mL of 1,4-dioxane, and Xantphos Pd G3 (520 mg, 0.5 mmol) was added. The mixture was heated to 100° C. The intermediate a1 (1.57 g, 5.5 mmol) was added to the reaction solution, and the reaction solution was reacted at 100° C. for another 3 hours. The reaction was stopped, and the mixture was filtered. The filtrate was concentrated, and separated by flash column chromatography to obtain a yellow solid a2 (820 mg), yield: 35%, LCMS: ESI-MS (m/z): 379.1 [M+H]⁺.

### Synthesis of Intermediates a3, a5, a9

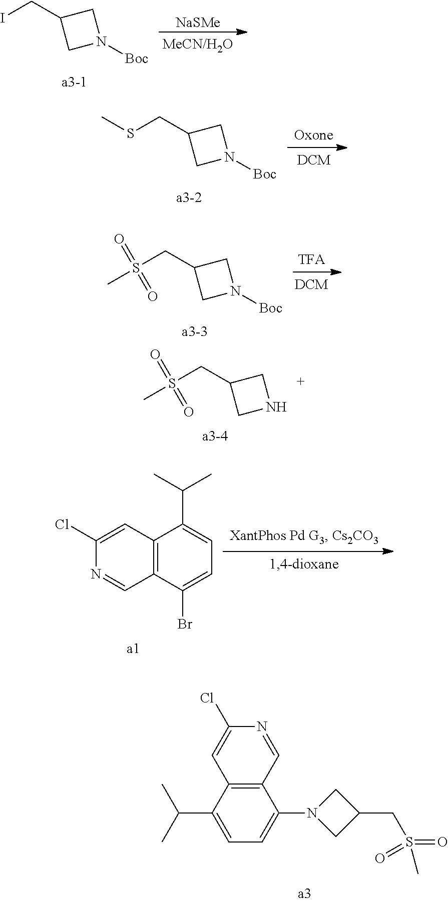

Step 1: Under nitrogen protection, tert-butyl [3-(iodomethyl)azetidin-1-yl]carboxylate a3-1 (6.0 g, 16 mmol) and sodium thiomethoxide (20% aqueous solution) (6.0 g, 16 mmol) were dissolved in 50 mL of a mixture of acetonitrile and water (v/v=3/1). The mixture was heated to 60° C. and stirred for 12 hours. The reaction was completed by LC-MS monitoring. 100 mL of water was added to the reaction solution, and the mixture was extracted with ethyl acetate, washed with saturated brine, dried over anhydrous sodium sulfate, and filtered. The filtrate was concentrated to obtain a yellow oil a3-2 (2.43 g), yield: 81%, LC-MS: [M+H]=162.0.

Step 2: Under nitrogen protection, the oil a3-2 (2.43 g, 10 mmol) from the previous step was dissolved in 50 mL of dichloromethane, and the oxidant Oxone (34.4 g, 56 mmol) was added. The mixture was reacted at room temperature for 48 hours. The reaction was completed by LC-MS monitoring. The reaction solution was filtered and the filtrate was concentrated to obtain a white solid a3-3 (2.99 g), yield: 95%, LC-MS: [M+H]=250.0.

Step 3: The intermediate a3-3 (2.99 g, 12 mmol) from the previous step was dissolved in 20 mL of dichloromethane, and 5 mL of trifluoroacetic acid was added. The mixture was stirred at room temperature for 2 hours. The reaction solution was concentrated under reduced pressure to obtain a crude product a3-4, LC-MS: [M+H]=150.0.

Step 4: Under nitrogen protection, the crude product a3-4 (1.39 g, 5.27 mmol) from the previous step, intermediate at (500 mg, 1.76 mmol) and Cs₂CO₃ (2.86 g, 8.79 mmol) were mixed in 10 mL of 1,4-dioxane, and XantPhos Pd G3 (167 mg, 0.18 mmol) was added. The mixture was heated to 100° C. and reacted for 3 hours. The reaction was stopped, and filtered. The filtrate was concentrated, and separated by flash column chromatography to obtain a green solid a3 (410 mg), yield: 60%, LC-MS: M+H⁺=353.0.

1H NMR (400 MHz, CDCl₃) δ 9.11 (d, J=0.5 Hz, 1H), 7.84 (s, 1H), 7.46 (d, J=8.0 Hz, 1H), 6.52 (d, J=8.0 Hz, 1H), 4.45 (t, J=7.5 Hz, 2H), 4.02 (dd, J=7.6, 5.3 Hz, 2H), 3.50-3.40 (m, 4H), 2.98 (s, 3H), 1.33 (d, J=6.8 Hz, 6H).

Referring to the synthetic route of intermediate a3, the following intermediates were synthesized using a similar skeleton structure.

| Replacement of structure | | | Target intermediate | | LC-MS [M + H]⁺ |
|---|---|---|---|---|---|
| Replace a3-4 with | 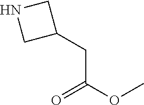 | a5-1 | 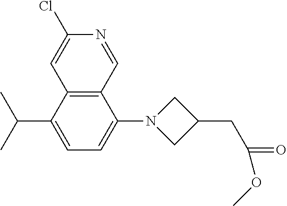 [SMILES: COC(=O)CC1CN(c2ccc(C(C)C)c3cc(Cl)ncc23)C1] | a5 | 333 |
| Replace a3-4 with | 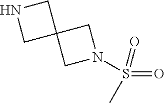 | a9-1 | 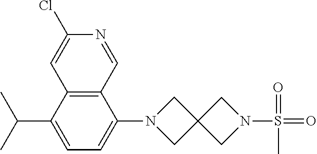 [SMILES: CC(C)c1ccc(N2CC3(C2)CN(S(C)(=O)=O)C3)c2cnc(Cl)cc12] | a9 | 380 |

### Synthesis of Intermediate a4

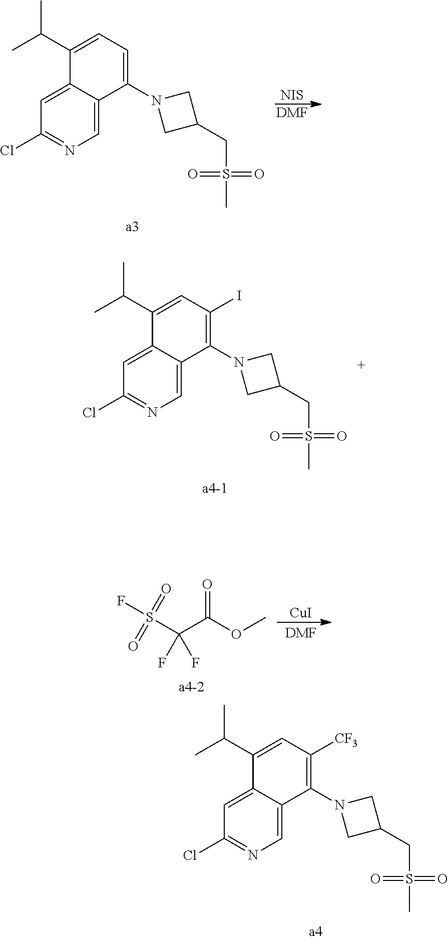

Step 1: The intermediate a3 (200 mg, 0.57 mmol) was dissolved in 20 mL of DMF, and N-iodosuccinimide NIS (380 mg, 1.7 mmol) was added. The mixture was reacted at room temperature for 2 hours. The reaction was completed by LC-MS monitoring. 100 mL of water was added to the reaction solution. The mixture was extracted with ethyl acetate, washed with saturated sodium thiosulfate solution, dried with anhydrous sodium sulfate, and filtered. The filtrate was concentrated to obtain a yellow solid a4-1 (200 mg), yield: 59%, LC-MS: M+H⁺=478.9.

Step 2: Under nitrogen protection, the intermediate a4-1 (20 mg, 0.042 mmol), methyl 2,2-difluoro-2-(fluorosulfonyl)acetate a4-2 (40 mg, 0.21 mmol) and CuI (24 mg, 0.12 mmol) were mixed in 5 mL of DMF. The mixture was heated to 90° C. and reacted for 1 hour. The reaction was completed by LC-MS monitoring. 50 mL of water was added to the reaction solution. The mixture was extracted with ethyl acetate, dried over anhydrous sodium sulfate, and filtered. The filtrate was concentrated, and separated by column chromatography (DCM/MeOH=25/1) to obtain a yellow solid a4 (15 mg), yield: 77%, LC-MS: M+H⁺=421.0.

### Synthesis of Intermediate a6

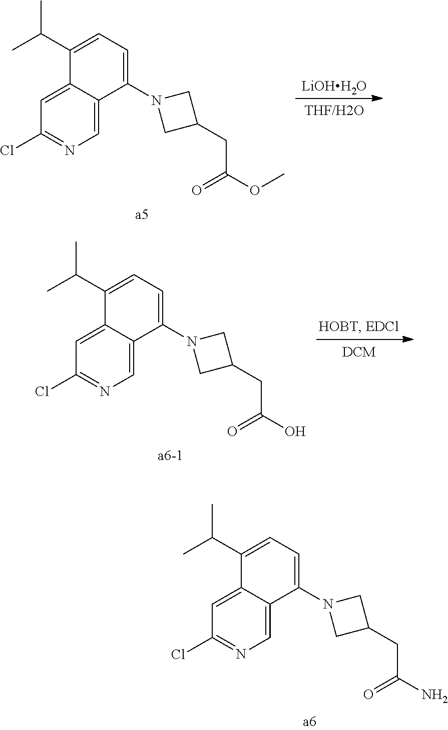

Step 1: Methyl 2-(1-(3-chloro-5-isopropylisoquinolin-8-yl)azetidin-3-yl)acetate a5 (120 mg, 0.36 mmol) was dissolved in 6 mL of a mixture of tetrahydrofuran and water (v/v=5/1), and lithium hydroxide monohydrate (23 mg, 0.54 mmol) was added. The mixture was stirred at room temperature for 12 hours. The reaction was stopped, and acetic acid was slowly added to the reaction solution to adjust the pH value to about 6. The mixture was extracted with ethyl acetate. The organic phases were combined, dried over anhydrous sodium sulfate, and filtered. The filtrate was concentrated under reduced pressure to obtain a crude intermediate a6-1 (100 mg), yield: 18%, LC-MS:ESI-MS (m/z): 319.1 [M+H]⁺.

Step 2: The crude product a6-1 (50 mg, 0.16 mmol) from the previous step was dissolved in 3 mL of dichloromethane. HOBT (6 mg, 0.047 mmol), EDCI (36 mg, 0.19 mmol) and DIPEA (40 mg, 0.31 mmol) were added. The mixture was stirred at room temperature for 1 hour. 0.2 mL of ammonia water was added to the reaction solution, and the mixture was reacted at room temperature for another 2 hours. The reaction was stopped, and 20 mL of water was added to the reaction solution. The mixture was extracted with ethyl acetate. The organic phases were combined, dried over anhydrous sodium sulfate, filtered, and separated by thin layer chromatography to obtain intermediate a6 (20 mg), yield: 36%, LC-MS:ESI-MS (m/z): 318.1 [M+H]⁺.

### Synthesis of Intermediate a7

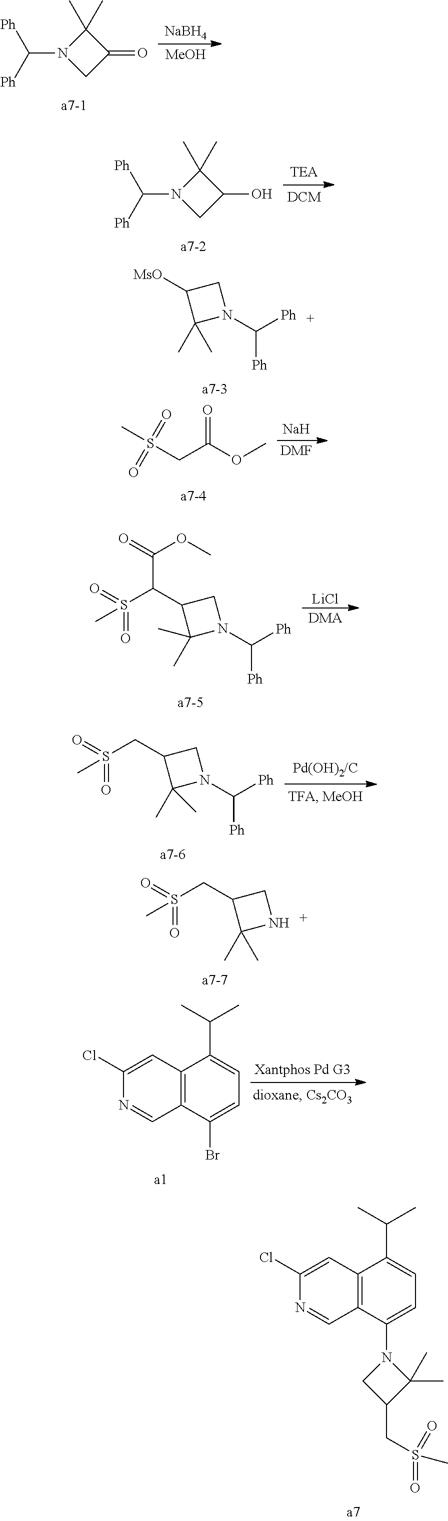

Step 1: In an ice bath, the raw material 1-(diphenylmethyl)-2,2-dimethylazetidin-3-one a7-1 (3.2 g, 12.1 mmol) was dissolved in 50 mL of methanol, and sodium borohydride (912 mg, 24.1 mmol) was added. The mixture was heated to 70° C. and reacted for 16 hours. The reaction was stopped, and the solvent was removed by evaporation under reduced pressure. 100 mL of water was added to the residue, and the mixture was extracted with ethyl acetate. The organic phase was washed with brine, dried over anhydrous sodium sulfate, and filtered. The filtrate was concentrated, and separated by flash column chromatography (PE/EA=3/1) to obtain a white solid a7-2 (2.0 g), yield: 59%, LC-MS: ESI-MS (m/z): 268.1 [M+H]⁺. 1H NMR (400 MHz, CDCl₃) δ 7.46 (dd, J=18.3, 7.4 Hz, 4H), 7.24-7.08 (m, 6H), 4.58 (s, 1H), 4.05-3.95 (m, 1H), 3.40-3.28 (m, 1H), 2.72 (dd, J=7.7, 5.9 Hz, 1H), 1.69 (d, J=7.5 Hz, 1H), 1.08 (s, 3H), 1.01 (s, 3H).

Step 2: In an ice bath, the intermediate a7-2 (1.0 g, 3.74 mmol) from the previous step and triethylamine (605 mg, 5.98 mmol) were dissolved in 20 mL of dichloromethane, and methylsulfonyl chloride (554 mg, 4.86 mmol) was added. The mixture was stirred at room temperature for 1 hour. The reaction was stopped. 50 mL of water was added to the system. The mixture was extracted with dichloromethane and concentrated to obtain a crude product a7-3 (1.1 g), which was directly used in the next step.

Step 3: In an ice bath, methyl 2-methanesulfonylacetate a7-4 (745 mg, 4.89 mmol) was dissolved in 20 mL of DMF, and NaH (181 mg, 4.52 mmol, 60%) was added. The reaction solution was stirred at 0° C. for 15 minutes, and the crude product a7-3 (1.1 g) from the previous step was added to the system. The mixture was heated to 80° C. and reacted for 16 hours. 50 mL of saturated aqueous ammonium chloride solution was added to the system to quench the reaction. The mixture was extracted with ethyl acetate. The organic phase was washed with brine, dried over anhydrous sodium sulfate, and filtered. The filtrate was concentrated, and separated by flash column chromatography (DCM/MeOH=20/1) to obtain a white solid a7-5 (900 mg), yield: 54%, LC-MS: ESI-MS (m/z): 402.0 [M+H]⁺.

Step 4: The intermediate a7-5 (900 mg, 2.24 mmol) from the previous step was dissolved in 20 mL of DMA, and LiCl (950 mg, 22.42 mmol) was added. The mixture was heated to 150° C. and reacted for 1 hour. The reaction was stopped, and 80 mL of water was added to the system. The mixture was extracted with ethyl acetate. The organic phase was washed with brine, dried over anhydrous sodium sulfate, and filtered. The filtrate was concentrated, and separated by flash column chromatography (DCM/MeOH=20/1) to obtain a white solid a7-6 (200 mg), yield: 27%, LC-MS: ESI-MS (m/z): 344.0 [M+H]⁺.

Step 5: Under a hydrogen balloon atmosphere, the intermediate a7-6 (2200 mg, 0.64 mmol) from the previous step was dissolved in 12 mL of methanol. 0.5 mL of trifluoroacetic acid and Pd(OH)₂/C (20 mg) were added. The mixture was reacted at room temperature for 16 hours. The reaction was stopped, and filtered. The filtrate was concentrated under reduced pressure to obtain a yellow trifluoroacetate a7-7 (150 mg), yield: 92%, LC-MS: ESI-MS (m/z): 178.2 [M+H]⁺.

Step 6: Under nitrogen protection, the trifluoroacetate salt a7-7 (92 mg, 0.31 mmol) from the previous step, intermediate a1 (60 mg, 0.21 mmol) and Cs₂CO₃ (171 mg, 0.52 mmol) were mixed in 3 mL of 1,4-dioxane. XantPhos Pd G3 (65 mg, 0.063 mmol) was added. The mixture was heated to 100° C. and reacted for 6 hours. The reaction was stopped, and filtered. The filtrate was concentrated, and separated by flash column chromatography to obtain a yellow solid a7 (35 mg), yield: 39%, LC-MS: ESI-MS (m/z): 381.1 [M+H]⁺.

### Synthesis of Intermediate a8

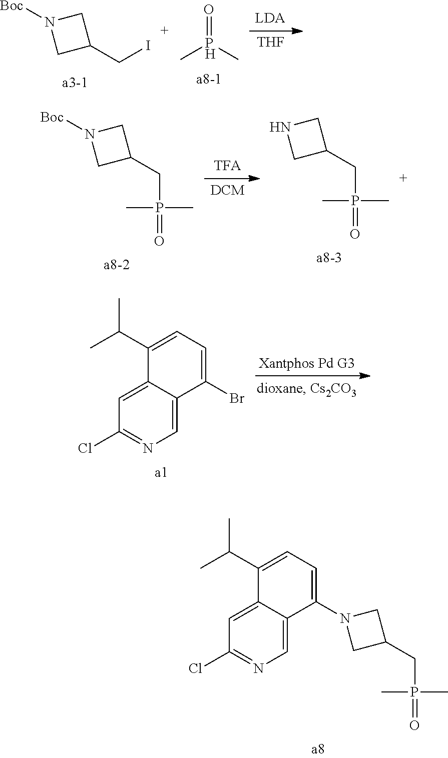

Step 1: Dimethylphosphine oxide a8-1 (1.0 g, 12.8 mmol) was dissolved in 20 mL of anhydrous tetrahydrofuran at −20° C. under nitrogen protection, and LDA (1M, 38.4 mL) was added dropwise. After stirring for 20 minutes, a solution of intermediate a3-1 (5.72 g, 19.2 mmol) in tetrahydrofuran (10 mL) was added dropwise. After the addition was completed, the mixture was slowly warmed to room temperature, and the mixture was reacted at room temperature for 5 hours. The reaction was stopped. 50 mL of saturated aqueous ammonium chloride solution was added to the system to quench the reaction. The mixture was extracted with ethyl acetate, dried over anhydrous sodium sulfate, and filtered. The filtrate was concentrated, and separated by flash column chromatography to obtain a brown solid a8-2 (0.8 g), yield: 24%, LC-MS: ESI-MS (m/z): 248.1 [M+H]⁺.

Step 2: The intermediate a8-2 (800 mg, 3.23 mmol) was dissolved in 6 mL of dichloromethane, and 3 mL of trifluoroacetic acid was added. The mixture was stirred at room temperature overnight. The reaction solution was concentrated under reduced pressure to obtain a crude product a8-3, which was directly used in the next step. LC-MS: ESI-MS (m/z): 148.1 [M+H]⁺.

Step 3: Under nitrogen protection, the trifluoroacetate salt a8-3, intermediate a1 (774 mg, 2.7 mmol) and Cs₂CO₃ (2.66 g, 8.2 mmol) were mixed in 15 mL of 1,4-dioxane. XantPhos Pd G3 (258 mg, 0.27 mmol) was added. The mixture was heated to 100° C. and reacted for 16 hours. The reaction was stopped, and filtered. The filtrate was concentrated, and separated by flash column chromatography to obtain a yellow solid a8 (200 mg), LC-MS: ESI-MS (m/z): 351.0 [M+H]⁺.

Synthesis of Intermediates a10, a17

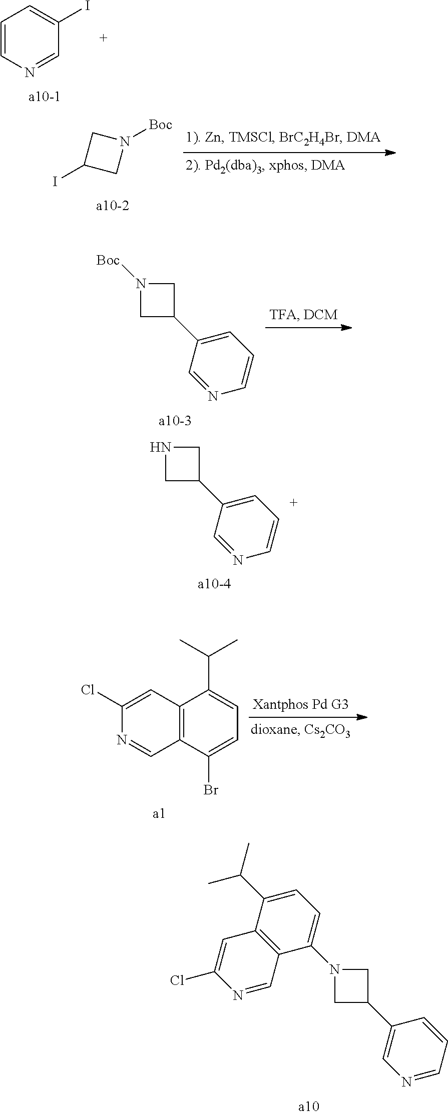 [SMILES: Ic1cccnc1 ; CC(C)(C)OC(=O)N1CC(I)C1 ; CC(C)(C)OC(=O)N1CC(c2cccnc2)C1 ; c1cncc(C2CNC2)c1 ; CC(C)c1ccc(Br)c2cnc(Cl)cc12 ; CC(C)c1ccc(N2CC(c3cccnc3)C2)c2cnc(Cl)cc12]

Step 1: Activated zinc powder (0.6 g, 9.16 mmol) was dissolved in 4 mL of DMA, and the mixture was heated to 65° C. Trimethylsilyl chloride (0.13 g, 0.13 mL) and 1,2-dibromoethane (0.1 mL, 1.3 mmol) were added dropwise to the system. After the addition was completed, the mixture was stirred for another 40 minutes. A solution of a10-2 (2.0 g, 7.06 mmol) in DMF (4 mL) was added to the reaction solution, and the mixture was reacted for another 30 minutes. The mixture was allowed to cool down to room temperature for use.

Under nitrogen protection, 3-iodopyridine a10-1 (1.45 g, 7.1 mmol), Pd₂(dba)₃ (647 mg, 0.71 mmol) and Xphos (673 mg, 1.41 mmol) were dissolved in 4 mL of DMA in another reaction bottle. The solution prepared from the previous step was added dropwise. The mixture was heated to 85° C. and reacted for 16 hours. The reaction was stopped, and the mixture was cooled to room temperature. The reaction solution was poured into 30 mL of ice water, and the mixture was filtered by suction. The filter cake was dissolved in ethyl acetate, concentrated, and separated by flash column chromatography (PE/EA=1/1) to obtain compound a10-3 (200 mg), yield 11%, LC-MS: ESI-MS (m/z): 235.2 [M+H]⁺.

Step 2: The intermediate a10-3 (200 mg, 0.85 mmol) was dissolved in 4 mL of dichloromethane, and 1.5 mL of trifluoroacetic acid was added dropwise. The mixture was reacted at room temperature for 3 hours. The reaction was stopped. The solvent was removed by evaporation under reduced pressure to obtain trifluoroacetate a10-4 (160 mg) with a yield of 69%, LC-MS: ESI-MS (m/z): 135.1 [M+H]⁺.

Step 3: Under nitrogen protection, the trifluoroacetate salt a10-4 (31 mg, 0.23 mmol) from the previous step, intermediate a1 (65 mg, 0.23 mmol) and Cs₂CO₃ (223 mg, 0.69 mmol) were mixed in 3 mL of 1,4-dioxane. XantPhos Pd G3 (22 mg, 0.023 mmol) was added. The mixture was heated to 100° C. and reacted for 16 hours. The reaction was stopped, and filtered. The filtrate was concentrated, and separated by flash column chromatography to obtain a yellow solid a10 (50 mg), LC-MS: ESI-MS (m/z): 338.0 [M+H]⁺.

Referring to the synthetic route of compound a10, the following intermediates were synthesized using a similar skeleton structure.

| Replacement of structure | | | Target intermediate | | LC-MS [M + H]⁺ |
|---|---|---|---|---|---|
| Replace a10-1 with: | 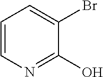 | a17-1 | 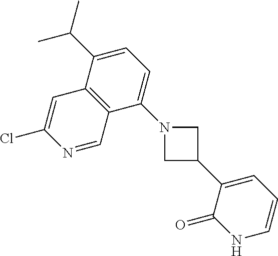 [SMILES: CC(C)c1ccc(N2CC(c3ccc[nH]c3=O)C2)c2cnc(Cl)cc12] | a17 | 354 |

### Synthesis of Intermediate a11

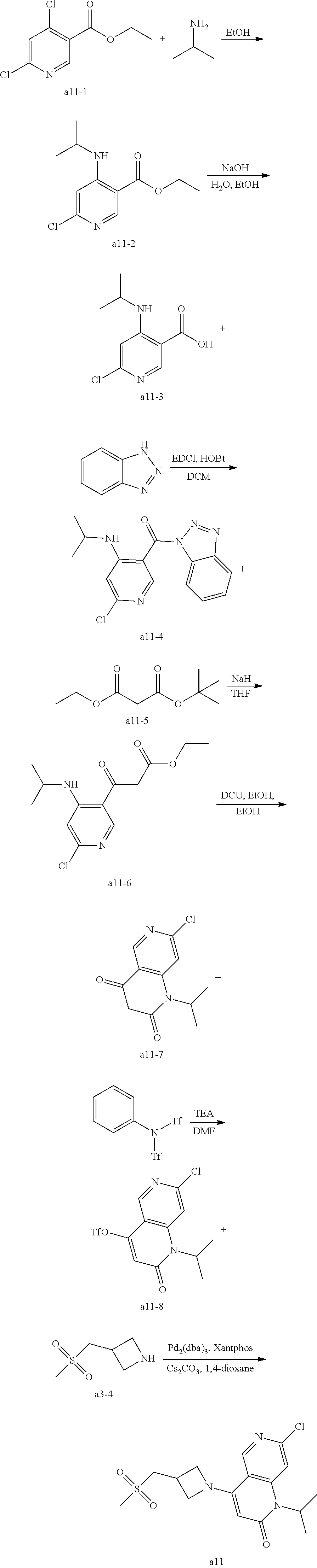

Step 1: Ethyl 4,6-dichloronicotinate all-1 (3.0 g, 0.013 mol) was dissolved in 50 mL of ethanol, and isopropylamine (2.41 g, 0.040 mol) was added. The reaction solution was stirred at 80° C. for 12 hours. The reaction was completed by LC-MS monitoring. The solvent was removed by evaporation under reduced pressure, and the residue was separated by column chromatography (PE/EA=10/1) to obtain a white solid all-2 (3.31 g), yield: 91%, LCMS: ESI-MS (m/z): 243.1[M+H]⁺.

Step 2: The intermediate all-2 (3.3 g, 13 mmol) was dissolved in 40 mL of ethanol, and 20 mL of 1N aqueous NaOH solution was added. The mixture was heated to 80° C. and reacted for 2 hours. The reaction was stopped. The solvent ethanol was removed by distillation in vacuum, and the residue was adjusted to pH of 3-4 with 1M hydrochloric acid, with a large amount of white solid precipitated. The mixture was filtered by suction. The filter cake was washed with water, and dried to obtain a white solid all-3 (1.6 g), yield: 50%, LC-MS: ESI-MS (m/z): 215.0 [M+H]⁺.

Step 3: The intermediate all-3 (1.6 g, 7.48 mmol) from the previous step was dissolved in 30 mL of dichloromethane. Benzotriazole (1.07 g, 8.99 mmol), EDCI (1.73 g, 8.99 mmol) and HOBT (0.3 g, 2.2 mmol) were added. The mixture was reacted at room temperature for 2 hours. The reaction was stopped. 50 mL of water was added to the reaction solution. The mixture was extracted with dichloromethane, dried over anhydrous sodium sulfate, and filtered. The filtrate was concentrated, and separated by column chromatography (PE/EA=10/1) to obtain an oil all-4 (1.65 g), yield: 63%, LC-MS: ESI-MS (m/z): 315.9 [M+H]⁺.

Step 4: In an ice bath, NaH (420 mg, 10. g mmol, 60%) was dissolved in 10 mL of anhydrous tetrahydrofuran under nitrogen protection, and tert-butyl ethyl malonate all-5 (1.96 g, 10.5 mmol) was slowly added. The mixture was stirred for 30 minutes, and a solution of intermediate all-4 (1.65 g, 5.24 mmol) in tetrahydrofuran (10 mL) was added. The mixture was stirred for another 2 hours in an ice bath. 30 mL of saturated aqueous ammonium chloride solution was added to the reaction solution to quench the reaction. Tetrahydrofuran was removed by distillation under reduced pressure, and the mixture was extracted with ethyl acetate. The organic phase was washed with saturated brine, dried over anhydrous sodium sulfate, and filtered. The filtrate was concentrated to obtain a crude product. The crude product was dissolved in dichloromethane, and 10 mL of trifluoroacetic acid was added. The mixture was stirred at room temperature for 2 hours. After the reaction was completed, 20 mL of toluene was added and the mixture was separated by column chromatography (PE/EA=10/1) to obtain a colorless oil all-6 (780 mg), yield: 48%, LCMS: ESI-MS (m/z): 285.1[M+H]⁺.

Step 5: The intermediate all-6 (780 mg, 2.75 mmol) from the previous step was dissolved in 10 mL of ethanol, and DBU (1.23 g, 8.22 mmol) was added. The mixture was heated to 80° C. and reacted for 2 hours. The reaction was stopped. Ethanol was removed by concentration under reduced pressure. 1M hydrochloric acid was added to the reaction solution to adjust the pH to about 3, with a white solid precipitated. The mixture was filtered by suction. The filter cake was washed with water, and dried to obtain intermediate all-7 (440 mg), yield: 61%, LC-MS: ESI-MS (m/z): 239.0 [M+H]⁺.

Step 6: The intermediate all-7 (440 mg, 1.67 mmol) from the previous step was dissolved in 10 mL of DMF. Triethylamine (507 mg, 5.02 mmol) and N-phenylbis(trifluoromethanesulfonyl)imide (718 mg, 2.0 mmol) were added, and the mixture was reacted at room temperature for 2 hours. The reaction was stopped. 50 mL of water was added to the reaction solution, and the mixture was extracted with ethyl acetate. The organic phases were combined, washed with saturated brine, dried over anhydrous sodium sulfate, and filtered. The filtrate was concentrated, and separated by thin layer chromatography (PE/EA=3/1) to obtain a white solid all-8 (620 mg), yield: 90%, LCMS: ESI-MS (m/z): 371.0 [M+H]⁺.

Step 7: Under nitrogen protection, the intermediate all-8 (210 mg, 0.57 mmol) from the previous step, intermediate a3-4 (140 mg, 0.53 mmol) and Cs₂CO₃ (556 mg, 1.71 mmol) were mixed in 10 mL of 1,4-dioxane. Pd₂(dba)₃ (52 mg, 0.057 mmol) and Xantphos (65 mg, 0.113 mmol) were added. The mixture was heated to 60° C. and reacted for 2 hours. The reaction was stopped, and filtered. The filtrate was concentrated, and separated by flash column chromatography to obtain a white solid all (30 mg), yield: 15%, LC-MS: ESI-MS (m/z): 370.0 [M+H]⁺.

### Synthesis of Intermediate a12

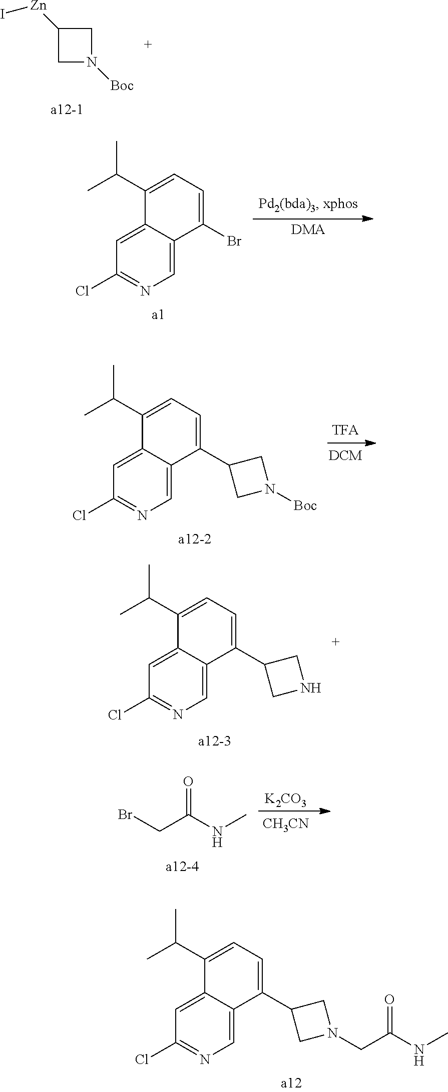

Step 1: Under nitrogen protection, the intermediate a1 (700 mg, 2.46 mmol), Pd₂(dba)₃ (225 mg, 0.25 mmol), Xphos (234 mg, 0.49 mmol) and the newly prepared raw material a12-1 (4 mL, 5.0 mmol) were dissolved in 4 mL of DMA. The mixture was heated to 85° C. and reacted for 15 hours. After cooling to room temperature, the reaction solution was poured into 15 mL of ice water, and a solid precipitated. The mixture was filtered by suction. The filter cake was dissolved in ethyl acetate and filtered. The filtrate was concentrated, and separated by flash column chromatography (PE/EA=1/1) to obtain a yellow solid a12-2 (280 mg), yield: 29%, LC-MS: ESI-MS (m/z): 361.1 [M+H]⁺.

Step 2: The intermediate a12-2 (85 mg, 0.24 mmol) from the previous step was dissolved in 6 mL of dichloromethane, and 2 mL of trifluoroacetic acid was added. The mixture was reacted at room temperature for 12 hours. The reaction was stopped. The reaction solution was concentrated to obtain trifluoroacetate a12-3 (20 mg), yield: 30%, LC-MS: ESI-MS (m/z): 261.1 [M+H]⁺.

Step 3: The intermediate a12-3 (20 mg, 0.08 mmol) from the previous step and K₂CO₃ (43 mg, 0.31 mmol) were mixed in 5 mL of acetonitrile, and 2-bromo-N-methylacetamide a12-4 (9 mg, 0.061 mmol) was added. The mixture was reacted at room temperature for 3 hours. The reaction was stopped. 20 mL of water was added to the reaction solution. The mixture was extracted with ethyl acetate, washed with saturated brine, dried over anhydrous sodium sulfate, and filtered. The filtrate was concentrated, and separated by flash column chromatography (DCM/MeOH=10/1) to obtain a white solid a12 (35 mg), yield: 96%, LC-MS: ESI-MS (m/z): 332.2 [M+H]⁺.

### Synthesis of Intermediate a13

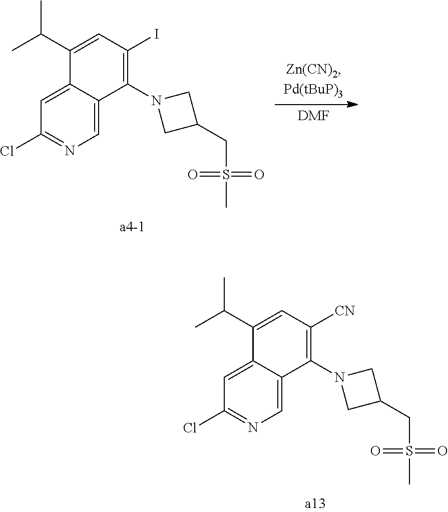

Under nitrogen protection, the intermediate a4-1 (50 mg, 0.10 mmol) was dissolved in 5 mL of DMF, Pd(^{t}BuP)₃ (16 mg, 0.03 mmol) and zinc cyanide (18 mg, 0.16 mmol) were added. The mixture was heated to 100° C. and reacted for 1 hour. The reaction was completed by LC-MS monitoring. The reaction was stopped. 30 mL of aqueous saturated sodium bicarbonate solution was added to the reaction solution. The mixture was extracted with ethyl acetate, dried over anhydrous sodium sulfate, and filtered. The filtrate was concentrated, and separated by thin layer chromatography to obtain a yellow solid a13 (30 mg), yield: 69%, LCMS: ESI-MS (m/z): 378.0 [M+H]⁺.

### Synthesis of Intermediate a14

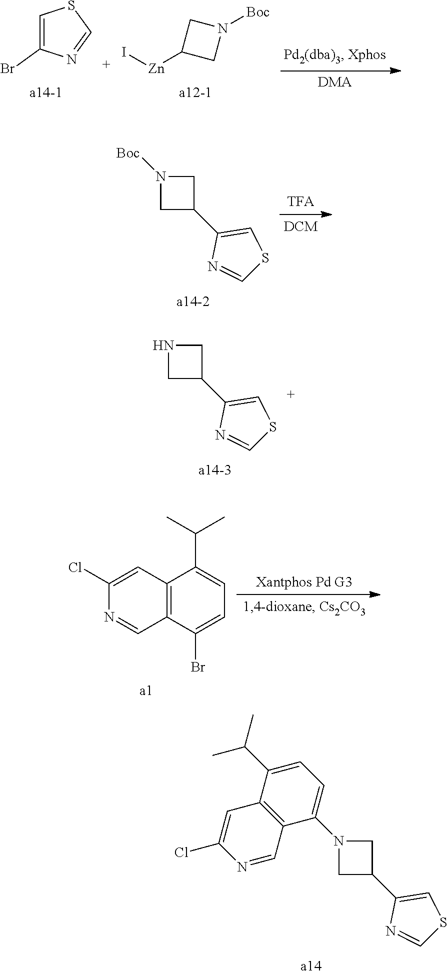

Step 1: Under nitrogen protection, the intermediate a14-1 (1.0 g, 6.11 mmol), Pd₂(dba)₃ (558 mg, 0.61 mmol), Xphos (581 mg, 1.22 mmol) and the newly prepared raw material a12-1 (8 mL, 12.0 mmol) were dissolved in 6 mL of DMA. The mixture was heated to 85° C. and reacted for 12 hours. After cooling to room temperature, the reaction solution was poured into 35 mL of ice water. The mixture was extracted with ethyl acetate, concentrated, and separated by flash column chromatography (PE/EA=2/1) to obtain a yellow solid a14-2 (120 mg), yield: 8%, LC-MS: ESI-MS (m/z): 185.1 [M−56]⁺.

Step 2: The intermediate a14-2 (120 mg, 0.42 mmol) from the previous step was dissolved in 6 mL of dichloromethane, and 2 mL of trifluoroacetic acid was added. The mixture was reacted at room temperature for 0.5 hours. The reaction was stopped. The reaction solution was concentrated to obtain trifluoroacetate a14-3 (70 mg), yield: 91%, LC-MS: ESI-MS (m/z): 141.1 [M+H]⁺.

Step 3: Under nitrogen protection, the trifluoroacetate salt a14-3 (100 mg, 0.71 mmol) from the previous step, intermediate a1 (203 mg, 0.71 mmol) and Cs₂CO₃ (1162 mg, 3.57 mmol) were mixed in 10 mL of 1,4-dioxane. XantPhos Pd G3 (68 mg, 0.071 mmol) was added. The mixture was heated to 100° C. and reacted for 3 hours. The reaction was stopped, and filtered. The filtrate was concentrated, and separated by flash column chromatography to obtain a yellow solid a14 (70 mg), yield: 26%, LC-MS: ESI-MS (m/z): 344.1 [M+H]⁺.

### Synthesis of Intermediate a15

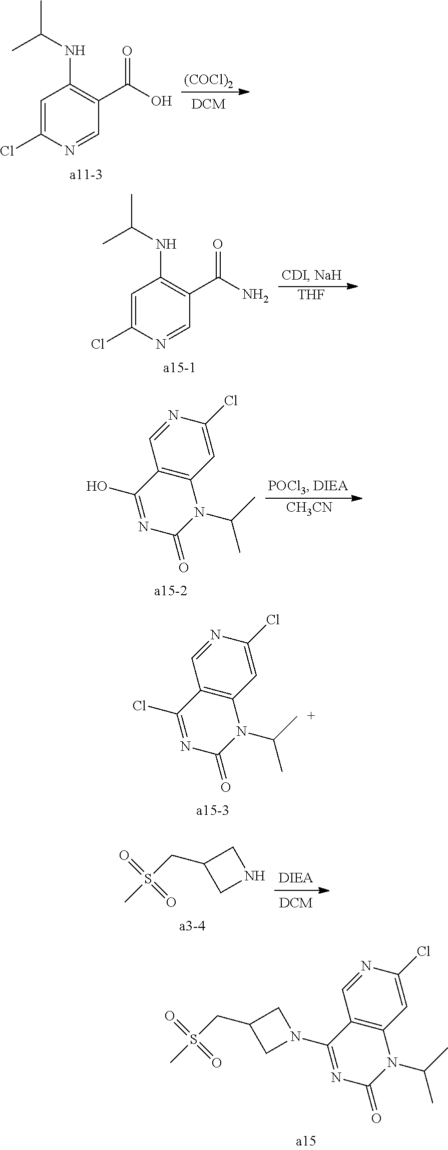

Step 1: In an ice bath, the intermediate all-3 (1.3 g, 6.10 mmol) was dissolved in 13 mL of dichloromethane, and oxalyl chloride (1.54 g, 12.1 mmol) and 3 drops of anhydrous DMF were added dropwise. The reaction solution was stirred for 1 hour. 12 mL of ammonia water was added dropwise to the reaction solution. The mixture was heated to room temperature and reacted for another 1 hour. 30 mL of ice water was added to the reaction solution to quench the reaction. The mixture was extracted with dichloromethane, concentrated, and separated by column chromatography (PE/EA=1/1) to obtain a white solid a15-1 (1.1 g), yield: 85%.

1H NMR (400 MHz, DMSO-d₆) δ 8.72 (d, J=7.8 Hz, 1H), 8.39 (s, 1H), 8.07 (s, 1H), 7.47 (s, 1H), 6.69 (s, 1H), 3.72-3.77 (m, 1H), 1.16 (d, J=6.3 Hz, 6H).

Step 2: In an ice bath, the intermediate a15-1 (1.1 g, 5.16 mmol) from the previous step was dissolved in 13 mL of anhydrous tetrahydrofuran, and NaH (1.2 g, 30.1 mmol, 60%) was added. The reaction solution was stirred for 30 minutes. CDI (2.5 g, 15.4 mmol) was added to the reaction solution. The mixture was heated to room temperature and reacted for another 1 hour. The reaction was completed by LC-MS monitoring. 50 mL of ice water was added to the reaction solution. The mixture was extracted with dichloromethane, concentrated, and separated by column chromatography (PE/EA=1/1) to obtain a white solid a15-2 (1.0 g), yield: 81%, LC-MS: ESI-MS (m/z): 240.0 [M+H]⁺.

Step 3: The intermediate a15-2 (1.0 g, 4.18 mmol) from the previous step and DIEA (530 mg, 4.18 mmol) was dissolved in 15 mL of acetonitrile, and POCl₃ (640 mg, 4.18 mmol) was added. The reaction solution was heated to 90° C. and reacted for 1 hour. The reaction was stopped, and the mixture was cooled to room temperature. The reaction solution was poured into 40 mL of ice water. The mixture was extracted with ethyl acetate, dried with anhydrous sodium sulfate, and filtered. The filtrate was concentrated, and separated by column chromatography (PE/EA=5/1) to obtain a white solid a15-3 (700 mg), yield: 65%, LC-MS: ESI-MS (m/z): 258.0 [M+H]⁺.

Step 4: In an ice bath, intermediate a3-4 (408 mg, 1.55 mmol), intermediate a15-3 (400 mg, 1.55 mmol) and DIEA (802 mg, 6.19 mmol) were dissolved in 10 mL of dichloromethane. The mixture was reacted in an ice bath for 1 hour. The reaction was stopped. 30 mL of water was added to the reaction solution. The mixture was extracted with dichloromethane, dried over anhydrous sodium sulfate, and filtered. The filtrate was concentrated, and separated by flash column chromatography to obtain a white solid a15 (300 mg), yield: 47%, LC-MS: ESI-MS (m/z): 371.0 [M+H]⁺.

Synthesis of Intermediates a16, a18-a22, a24, a27, a29, a31, a34, and a36-a38

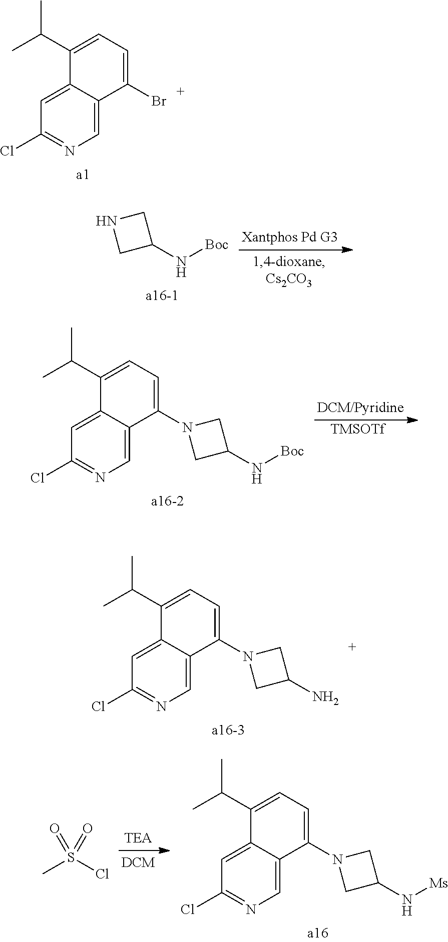

Step 1: Under nitrogen protection, compound a16-1 (304 mg, 1.76 mmol), intermediate a1 (500 mg, 1.76 mmol) and Cs₂CO₃ (2.86 g, 8.78 mmol) were mixed in 10 mL of 1,4-dioxane. XantPhos Pd G3 (166 mg, 0.17 mmol) was added. The mixture was heated to 100° C. for 2 hours. The reaction was stopped. The mixture was filtered. The filtrate was concentrated, and separated by flash column chromatography to obtain a yellow solid a16-2 (400 mg), yield: 55%, LC-MS: ESI-MS (m/z): 376.2 [M+H]⁺.

Step 2: In an ice bath, the intermediate a16-2 (120 mg, 0.32 mmol) from the previous step was dissolved in 2 mL of dichloromethane. 0.5 mL of pyridine was added, and 0.5 mL of trimethylsilyl trifluoromethanesulfonate was slowly added. The mixture was reacted at room temperature for 16 hours. The reaction was completed by LC-MS monitoring. 30 mL of aqueous saturated sodium bicarbonate solution was added to the system to quench the reaction. The mixture was extracted with dichloromethane. The organic phases were combined, washed with saturated brine, dried over anhydrous sodium sulfate, and filtered. The filtrate was concentrated, and separated by thin layer chromatography to obtain a yellow solid a16-3 (75 mg), yield: 77%, LC-MS: ESI-MS (m/z): 276.2 [M+H]⁺.

Step 3: The intermediate a16-3 (21 mg, 0.077 mmol) from the previous step and triethylamine (21 mg, 0.21 mmol) were dissolved in 2 mL of dichloromethane. Methylsulfonyl chloride (139 μL, 0.18 mmol) was added. The mixture was stirred at room temperature for 2 hours. The reaction was stopped. 10 mL of ice water was added to the reaction solution. The mixture was extracted with dichloromethane, dried with anhydrous sodium sulfate, and filtered. The filtrate was concentrated, and separated by thin layer chromatography to obtain a white solid a16 (20 mg), yield: 65%, LC-MS: ESI-MS (m/z): 354.0 [M+H]⁺.

Referring to the synthetic route of intermediate a16, the following intermediates were synthesized using a similar skeleton structure.

| | | | | | LC-MS |
|---|---|---|---|---|---|
| Replacement of structure | | | Target intermediate | | [M + H]⁺ |
| Replace MsCl in step 3 with | 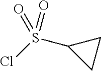 | | 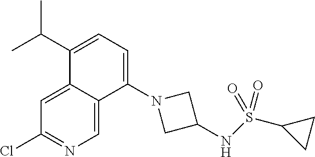 [SMILES: CC(C)c1ccc(N2CC(NS(=O)(=O)C3CC3)C2)c2cnc(Cl)cc12] | a18 | 380 |
| Replace intermediate a16-1 with | 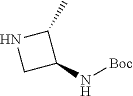 | a19-1 | 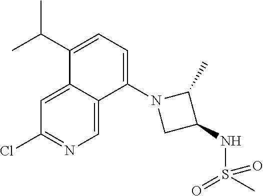 [SMILES: CC(C)c1ccc(N2C[C@H](NS(C)(=O)=O)[C@H]2C)c2cnc(Cl)cc12] | a19 | 368 |
| Replace intermediate a16-1 with | 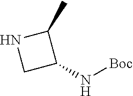 | a20-1 | 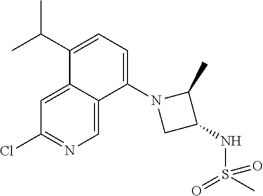 [SMILES: CC(C)c1ccc(N2C[C@@H](NS(C)(=O)=O)[C@@H]2C)c2cnc(Cl)cc12] | a20 | 368 |
| Replace MsCl in step 3 with | 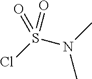 | | 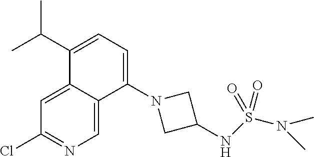 [SMILES: CC(C)c1ccc(N2CC(NS(=O)(=O)N(C)C)C2)c2cnc(Cl)cc12] | a21 | 383 |
| Replace MsCl in step 3 with | 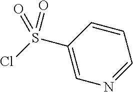 | | 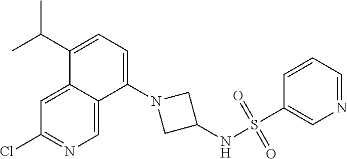 [SMILES: CC(C)c1ccc(N2CC(NS(=O)(=O)c3cccnc3)C2)c2cnc(Cl)cc12] | a22 | 417 |
| Replace intermediate a16-1 with | 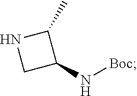 | a19-1 | 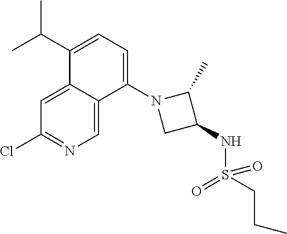 [SMILES: CCCS(=O)(=O)N[C@H]1CN(c2ccc(C(C)C)c3cc(Cl)ncc23)[C@@H]1C] | a24 | 396 |
| Replace MsCl in step 3 with | 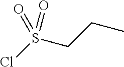 | | | | |
| Replace MsCl in step 3 with | 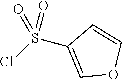 | | 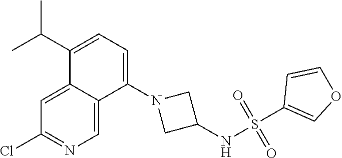 [SMILES: CC(C)c1ccc(N2CC(NS(=O)(=O)c3ccoc3)C2)c2cnc(Cl)cc12] | a27 | 406 |
| Replace MsCl in step 3 with acetyl chloride | | | 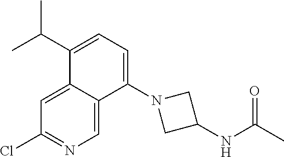 [SMILES: CC(=O)NC1CN(c2ccc(C(C)C)c3cc(Cl)ncc23)C1] | a29 | 318 |
| Replace MsCl in step 3 with | 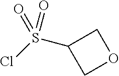 | | 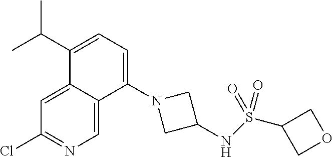 [SMILES: CC(C)c1ccc(N2CC(NS(=O)(=O)C3COC3)C2)c2cnc(Cl)cc12] | a31 | 396 |
| Replace intermediate a16-1 with | 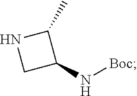 | a19-1 | 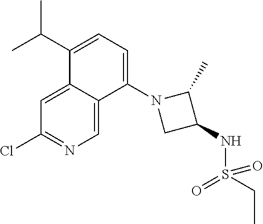 [SMILES: CCS(=O)(=O)N[C@H]1CN(c2ccc(C(C)C)c3cc(Cl)ncc23)[C@@H]1C] | a34 | 382 |

| | | | | |
|---|---|---|---|---|
| Replace MsCl in step 3 with | 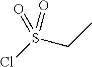 | | | |
| Replace MsCl in step 3 with | 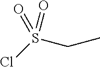 | 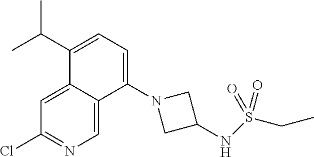 [SMILES: CCS(=O)(=O)NC1CN(c2ccc(C(C)C)c3cc(Cl)ncc23)C1] | a36 | 368 |
| Replace MsCl in step 3 with | 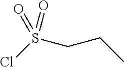 | 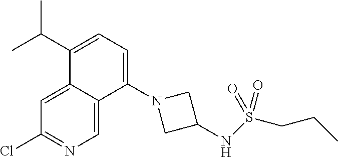 [SMILES: CCCS(=O)(=O)NC1CN(c2ccc(C(C)C)c3cc(Cl)ncc23)C1] | a37 | 382 |
| Replace MsCl in step 3 with | 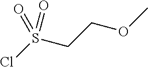 | 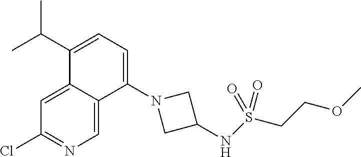 [SMILES: COCCS(=O)(=O)NC1CN(c2ccc(C(C)C)c3cc(Cl)ncc23)C1] | a38 | 399 |

Synthesis of Intermediates a23, a30, a32, a33, a35, a39, and a41

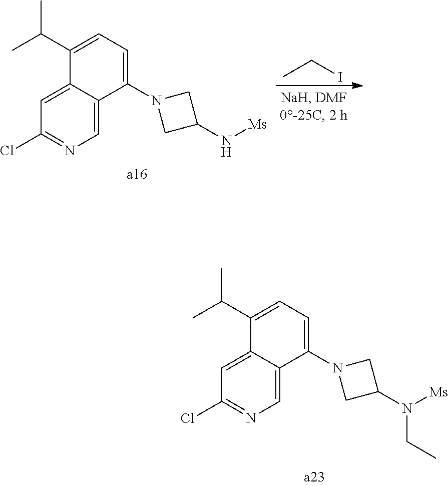

In an ice bath, intermediate a16 (40 mg, 0.11 mmol) was dissolved in 3 mL of DMF. NaH (6 mg, 0.15 mmol) and iodoethane (20 mg, 0.13 mmol) were added. The mixture was warmed to room temperature and reacted for 2 hours. The reaction was completed by LC-MS monitoring. 30 mL of aqueous saturated sodium bicarbonate solution was added to the system to quench the reaction. The mixture was extracted with dichloromethane. The organic phases were combined, washed with saturated brine, dried over anhydrous sodium sulfate, and filtered. The filtrate was concentrated, and separated by column chromatography to obtain a yellow solid a23 (40 mg), yield: 84%, LC-MS: ESI-MS (m/z): 382.1 [M+H]+

Referring to the synthetic route of intermediate a23, the following intermediates were synthesized using a similar skeleton structure.

| | | | | | LC-MS |
|---|---|---|---|---|---|
| Replacement of structure | | | Target intermediate | | [M + H]⁺ |
| Replace ethyl iodide EtI with methyl iodide MeI | | | 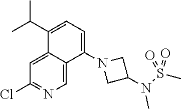 [SMILES: CC(C)c1ccc(N2CC(N(C)S(C)(=O)=O)C2)c2cnc(Cl)cc12] | a30 | 368 |
| Replace intermediate a16 with: | 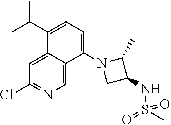 [SMILES: CC(C)c1ccc(N2C[C@H](NS(C)(=O)=O)[C@H]2C)c2cnc(Cl)cc12] | a19 | 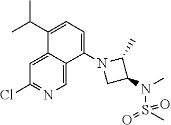 [SMILES: CC(C)c1ccc(N2C[C@H](N(C)S(C)(=O)=O)[C@H]2C)c2cnc(Cl)cc12] | a32 | 382 |
| Replace ethyl iodide EtI | | | | | |
| with methyl iodide MeI | | | | | |
| Replace intermediate a16 with: | 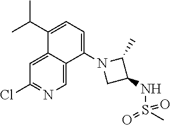 [SMILES: CC(C)c1ccc(N2C[C@H](NS(C)(=O)=O)[C@H]2C)c2cnc(Cl)cc12] | a19 | 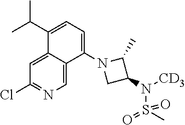 [SMILES: [2H]C([2H])([2H])N([C@H]1CN(c2ccc(C(C)C)c3cc(Cl)ncc23)[C@@H]1C)S(C)(=O)=O] | a33 | 385 |
| Replace ethyl iodide | | | | | |
| EtI with deuterated | | | | | |
| methyl iodide CD₃I | | | | | |
| Replace intermediate a16 with: | 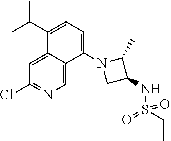 [SMILES: CCS(=O)(=O)N[C@H]1CN(c2ccc(C(C)C)c3cc(Cl)ncc23)[C@@H]1C] | a34 | 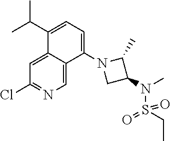 [SMILES: CCS(=O)(=O)N(C)[C@H]1CN(c2ccc(C(C)C)c3cc(Cl)ncc23)[C@@H]1C] | a35 | 396 |
| Replace ethyl iodide EtI | | | | | |
| with methyl iodide MeI | | | | | |
| Replace intermediate a16 with: | 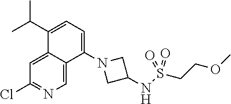 [SMILES: COCCS(=O)(=O)NC1CN(c2ccc(C(C)C)c3cc(Cl)ncc23)C1] | a38 | 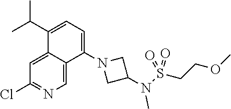 [SMILES: COCCS(=O)(=O)N(C)C1CN(c2ccc(C(C)C)c3cc(Cl)ncc23)C1] | a39 | 412 |
| Replace ethyl iodide EtI | | | | | |
| with methyl iodide MeI | | | | | |
| Replace intermediate a16 with: | 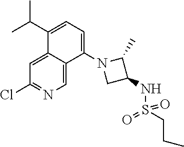 [SMILES: CCCS(=O)(=O)N[C@H]1CN(c2ccc(C(C)C)c3cc(Cl)ncc23)[C@@H]1C] | a24 | 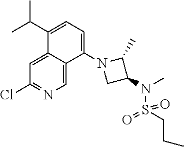 [SMILES: CCCS(=O)(=O)N(C)[C@H]1CN(c2ccc(C(C)C)c3cc(Cl)ncc23)[C@@H]1C] | a41 | 410 |
| Replace ethyl iodide EtI with | | | | | |
| methyl iodide MeI | | | | | |

### Synthesis of Intermediates a25 and a42

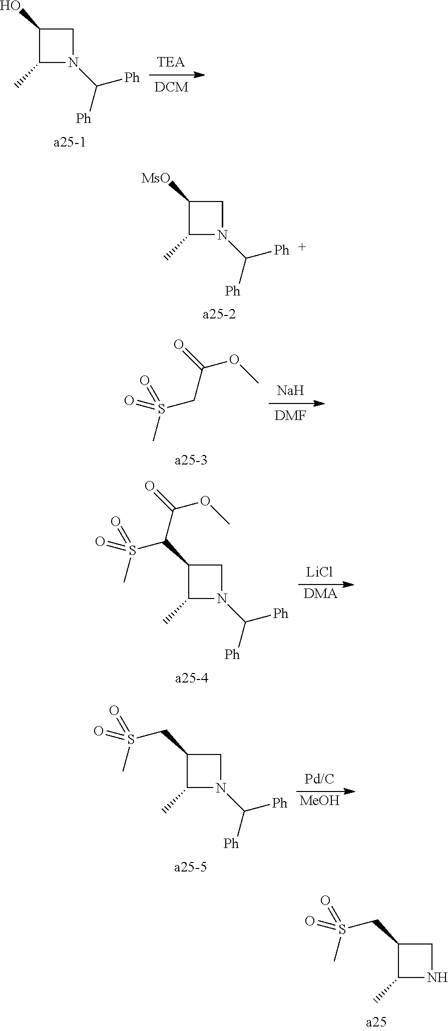

Step 1: Under nitrogen protection, the raw material a25-1 (7.0 g, 27.7 mmol) and triethylamine (4.2 g, 41.5 mmol) were dissolved in 40 mL of dichloromethane. Methylsulfonyl chloride (3.21 g, 28.0 mmol) was added. The mixture was reacted at room temperature for 4 hours. The reaction was stopped, and the solvent was removed under reduced pressure to obtain a crude product a25-2 (10.8 g).

Step 2: The crude product a25-2 (10.8 g) from the previous step and the raw material a25-3 (4.21 g, 27.7 mmol) were dissolved in 30 mL of DMF. NaH (1.11 g, 27.7 mmol) was added. The mixture was slowly heated to 80° C. and reacted for 12 hours. The reaction was completed by LC-MS monitoring. 100 mL of saturated saline solution was added to the system to quench the reaction. The mixture was extracted with dichloromethane. The organic phases were combined, washed with saturated brine, dried with anhydrous sodium sulfate, and filtered. The filtrate was concentrated to obtain a crude product a25-4 (15 g). LC-MS: ESI-MS (m/z): 388.3 [M+H]⁺.

Step 3: The crude product a25-4 (15 g) from the previous step and LiCl (2.52 g, 60.0 mmol) were dissolved in 40 mL of N,N-dimethylacetamide DMA. The mixture was heated to 150° C. and reacted for 2 hours. The reaction was stopped. 100 mL of ice water was added to the reaction solution. The mixture was extracted with dichloromethane, dried over anhydrous sodium sulfate, and filtered. The filtrate was concentrated, and separated by flash column chromatography to obtain a yellow solid a25-5 (6.42 g), with a total yield of 71% for three steps, LC-MS: ESI-MS (m/z): 330.1 [M+H]⁺.

Step 4: Under hydrogen atmosphere (2 atm), the intermediate a25-5 (6.42 g, 19.5 mmol) from the previous step and Pd/C (1.28 g, 20%) were mixed in 40 mL of methanol. 2 mL of trifluoroacetic acid was added. The reaction was allowed to react at room temperature for 16 hours. The reaction was stopped. The mixture was filtered and concentrated to obtain a yellow oil a25 (4.5 g), which was directly used in the next reaction. LC-MS: ESI-MS (m/z): 164.2 [M+H]⁺.

Referring to the synthetic route of intermediate a25, the following intermediate was synthesized using a similar skeleton structure.

| Replacement of structure | Target intermediate | LC-MS [M + H]⁺ |
|---|---|---|
| 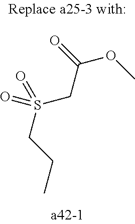 | 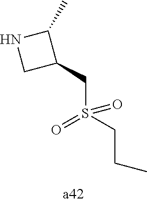 | 192 |

### Synthesis of Intermediates a26 and a43

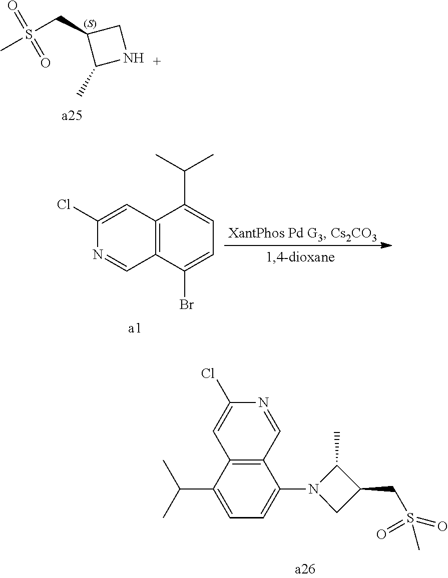

Under nitrogen protection, intermediate a25 (2.0 g, 12.3 mmol), intermediate a1 (3.47 g, 12.3 mmol) and Cs₂CO₃ (8.01 g, 24.6 mmol) were mixed in 80 mL of 1,4-dioxane. XantPhos Pd G3 (1.14 g, 1.2 mmol) was added. The mixture was heated to 100° C. and reacted for 6 hours. The reaction was stopped. The mixture was filtered. The filtrate was concentrated, and separated by flash column chromatography to obtain a brown solid a26 (970 mg), yield: 23%, LC-MS: M+H⁺=367.3.

Referring to the synthetic route of intermediate a26, the following intermediate was synthesized using a similar skeleton structure.

| Replacement of structure | Target intermediate | LC-MS [M + H]⁺ |
|---|---|---|
| Replace a25 with a42 | 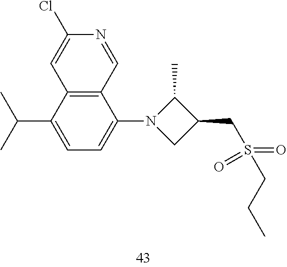 [SMILES: CCCS(=O)(=O)C[C@H]1CN(c2ccc(C(C)C)c3cc(Cl)ncc23)[C@@H]1C] | 395 |

### Synthesis of Intermediate a28

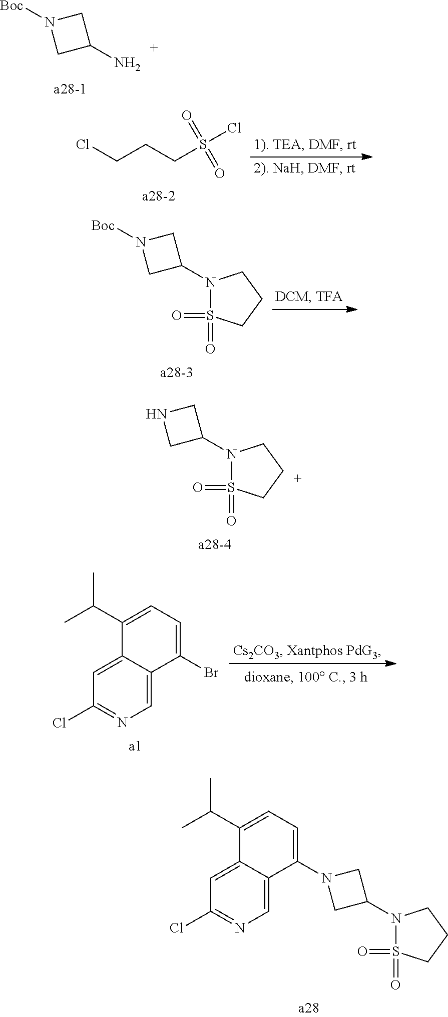 [SMILES: CC(C)(C)OC(=O)N1CC(N)C1 ; O=S(=O)(Cl)CCCCl ; CC(C)(C)OC(=O)N1CC(N2CCCS2(=O)=O)C1 ; O=S1(=O)CCCN1C1CNC1 ; CC(C)c1ccc(Br)c2cnc(Cl)cc12 ; CC(C)c1ccc(N2CC(N3CCCS3(=O)=O)C2)c2cnc(Cl)cc12]

Step 1: In an ice bath, under nitrogen protection, the raw material a28-1 (250 mg, 1.45 mmol) and triethylamine (294 mg, 2.9 mmol) were dissolved in 5 mL of DMF. 3-Chloropropylsulfonyl chloride a28-2 (282 mg, 1.59 mmol) was added. The mixture was stirred for 30 minutes, and NaH (174 mg, 4.35 mmol) was added. The mixture was warmed to room temperature and reacted for 12 hours. The reaction was stopped. 30 mL of aqueous saturated NH₄C₁ solution was added to the system. The mixture was extracted with ethyl acetate, dried over anhydrous sodium sulfate, and filtered. The filtrate was concentrated to obtain a crude product a28-3 (380 mg), yield: 83%, LC-MS: M+Na*=299.0.

Step 2: The intermediate a28-3 (220 mg, 0.72 mmol) from the previous step was dissolved in 7 mL of dichloromethane, and 3 mL of trifluoroacetic acid was added. The mixture was reacted at room temperature for 2 hours. The reaction was stopped. The solvent was removed by evaporation under reduced pressure. 10 mL of water was added, and the mixture was freeze-dried to obtain a white solid a28-4 (150 mg), yield: 65%, LC-MS: M+H⁺=177.1.

Step 3: Under nitrogen protection, the intermediate trifluoroacetate a28-4 (100 mg, 0.34 mmol), intermediate a1 (212 mg, 0.75 mmol) and Cs₂CO₃ (332 mg, 1.02 mmol) were mixed in 10 mL of 1,4-dioxane. XantPhos Pd G3 (35 mg, 0.034 mmol) was added. The mixture was heated to 100° C. and reacted for 3 hours. The reaction was stopped, and the mixture was filtered. The filtrate was concentrated, and separated by flash column chromatography to obtain a yellow solid a28 (50 mg), yield: 35%, LC-MS: M+H⁺=380.1.

### Synthesis of Intermediate a40

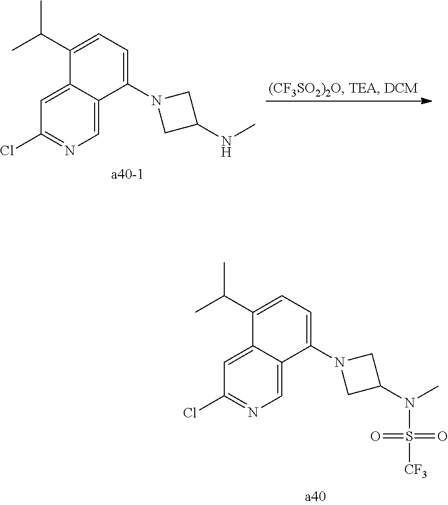 [SMILES: CNC1CN(c2ccc(C(C)C)c3cc(Cl)ncc23)C1 ; CC(C)c1ccc(N2CC(N(C)S(=O)(=O)C(F)(F)F)C2)c2cnc(Cl)cc12]

Referring to the synthetic route of intermediate a16, intermediate a40-1 was obtained. Operation steps: The intermediate 1-(3-chloro-5-isopropylisoquinolin-8-yl)-N-methylazetidin-3-amine a40-1 (10 mg, 0.035 mmol) and triethylamine (11 mg, 0.11 mmol) were dissolved in 3 mL of dichloromethane. 1.0 mL of a solution of trifluoromethanesulfonic anhydride (20 mg, 0.07 mmol) in dichloromethane was added dropwise. The mixture was reacted at room temperature for 2 hours. 30 mL of aqueous saturated sodium bicarbonate solution was added to the system to quench the reaction. The mixture was extracted with ethyl acetate, dried over anhydrous sodium sulfate, and concentrated. The crude product was separated by thin layer chromatography (DCM/MeOH, 10/1) to obtain a light yellow solid a40 (15 mg), yield: 93%, LC-MS: M+H⁺=422.1.

### Synthesis of Intermediates b1 and b2

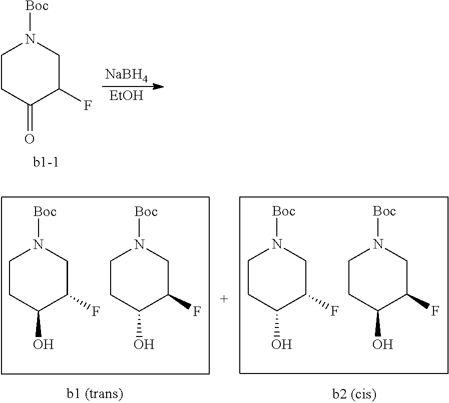

In an ice bath, under nitrogen protection, the raw material tert-butyl 3-fluoro-4-oxopiperidin-1-carboxylate intermediate b1-1 (3.0 g, 14.0 mmol) was dissolved in 50 mL of ethanol. Sodium borohydride (0.78 g, 21.0 mmol) was added. The mixture was heated to room temperature and reacted for 4 hours. The reaction was completed by LC-MS monitoring. 50 mL of aqueous saturated ammonium chloride solution was added to the reaction solution to quench the reaction. The mixture was extracted with ethyl acetate, washed with saturated brine, dried over anhydrous sodium sulfate, concentrated, and separated by flash column chromatography (PE/EA=2/1) to obtain trans intermediate b1 and cis intermediate b2 (2.52 g), yield: 90%, LCMS: ESI-MS (m/z): 164.0 [M−55]⁺.

Synthesis of Intermediates b3-b5, b10-b12, x1 and x2

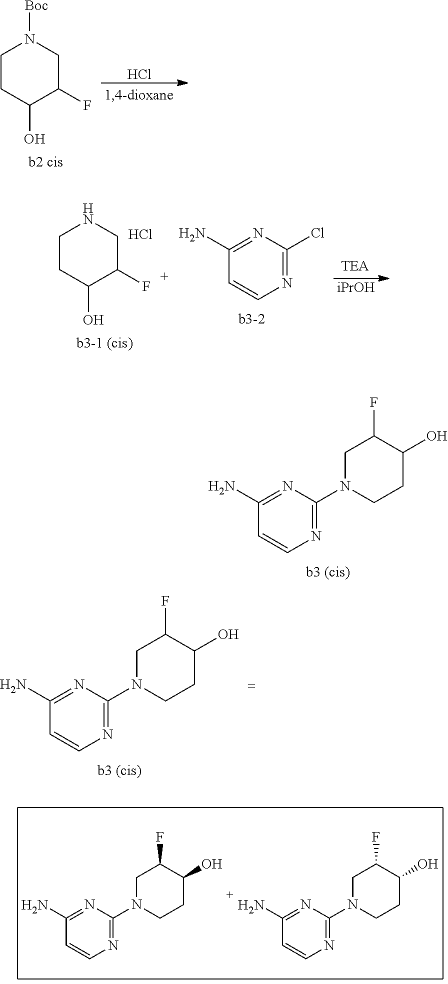

Under nitrogen protection, cis-tert-butyl 3-fluoro-4-hydroxypiperidin-1-carboxylate b2 (800 mg, 3.63 mmol) was dissolved in 5 mL of 1,4-dioxane. A 4M solution of hydrogen chloride in dioxane (4.5 mL, 18.16 mmol) was slowly added dropwise. The mixture was reacted at room temperature for 2 hours, and the solvent was removed by evaporation under reduced pressure to obtain compound b3-1 (210 mg), which was directly used in the next step.

cis-3-Fluoropiperidin-4-ol hydrochloride b3-1 (840 mg, 5.4 mmol), 2-chloropyrimidin-4-amine b3-2 (700 mg, 5.4 mmol) and triethylamine (1.63 g, 16.2 mmol) was dissolved in 20 mL of isopropanol. The reaction system was sealed and reacted at 110° C. for 18 hours. After cooling to room temperature, the reaction solution was concentrated under reduced pressure and separated by column chromatography (DCM/MeOH=21/1) to obtain a yellow solid cis-b3 (200 mg), yield: 16%, LC-MS: ESI-MS (m/z): 283.9 [M+H]⁺.

Referring to the synthetic route of compound b3, the following intermediates were synthesized using a similar skeleton structure.

| Replacement of structure | Target intermediate | LC-MS [M + H]⁺ |
|---|---|---|
| Replace b2 (cis) with b1 (trans) | 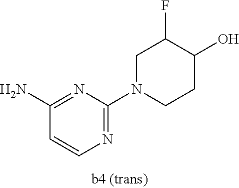 [SMILES: Nc1ccnc(N2CCC(O)C(F)C2)n1] | 213 |
| 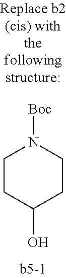 [SMILES: CC(C)(C)OC(=O)N1CCC(O)CC1] | 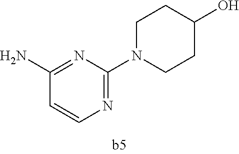 [SMILES: Nc1ccnc(N2CCC(O)CC2)n1] | 195 |
| 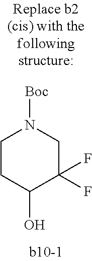 [SMILES: CC(C)(C)OC(=O)N1CCC(O)C(F)(F)C1] | 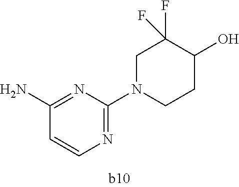 [SMILES: Nc1ccnc(N2CCC(O)C(F)(F)C2)n1] | 231 |
| 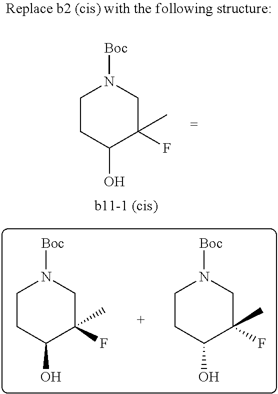 [SMILES: CC(C)(C)OC(=O)N1CCC(O)C(C)(F)C1 ; CC(C)(C)OC(=O)N1CC[C@H](O)[C@](C)(F)C1 ; CC(C)(C)OC(=O)N1CC[C@@H](O)[C@@](C)(F)C1] | 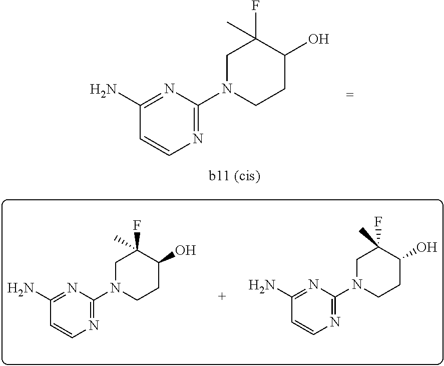 [SMILES: CC1(F)CN(c2nccc(N)n2)CCC1O ; C[C@@]1(F)CN(c2nccc(N)n2)CC[C@@H]1O ; C[C@]1(F)CN(c2nccc(N)n2)CC[C@H]1O] | 227 |
| 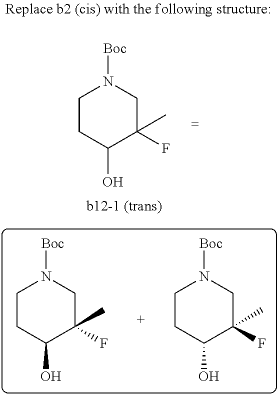 [SMILES: CC(C)(C)OC(=O)N1CCC(O)C(C)(F)C1 ; CC(C)(C)OC(=O)N1CC[C@H](O)[C@@](C)(F)C1 ; CC(C)(C)OC(=O)N1CC[C@@H](O)[C@](C)(F)C1] | 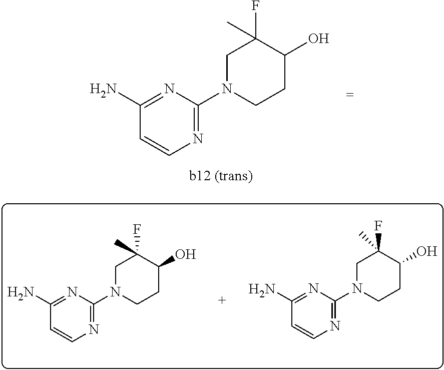 [SMILES: CC1(F)CN(c2nccc(N)n2)CCC1O ; C[C@]1(F)CN(c2nccc(N)n2)CC[C@@H]1O ; C[C@@]1(F)CN(c2nccc(N)n2)CC[C@H]1O] | 227 |
| 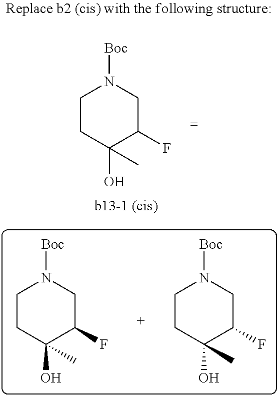 [SMILES: CC(C)(C)OC(=O)N1CCC(C)(O)C(F)C1 ; CC(C)(C)OC(=O)N1CC[C@](C)(O)[C@H](F)C1 ; CC(C)(C)OC(=O)N1CC[C@@](C)(O)[C@@H](F)C1] | 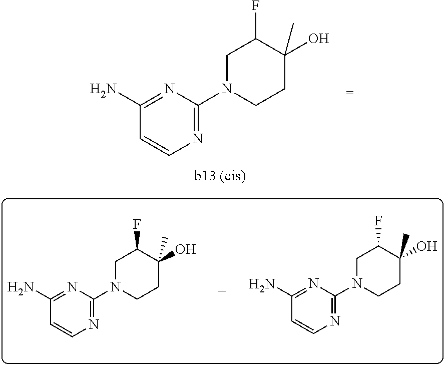 [SMILES: CC1(O)CCN(c2nccc(N)n2)CC1F ; C[C@]1(O)CCN(c2nccc(N)n2)C[C@H]1F ; C[C@@]1(O)CCN(c2nccc(N)n2)C[C@@H]1F] | 227 |
| Replace b2 (cis) with b2 chiral (3S, 4R) | 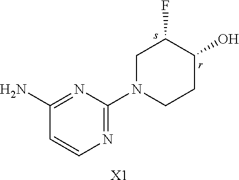 | 213 |
| Replace b2 (cis) with b2 chiral (3R, 4S) | 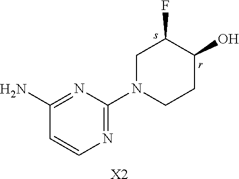 | 213 |

Synthesis of Intermediates b6-b8

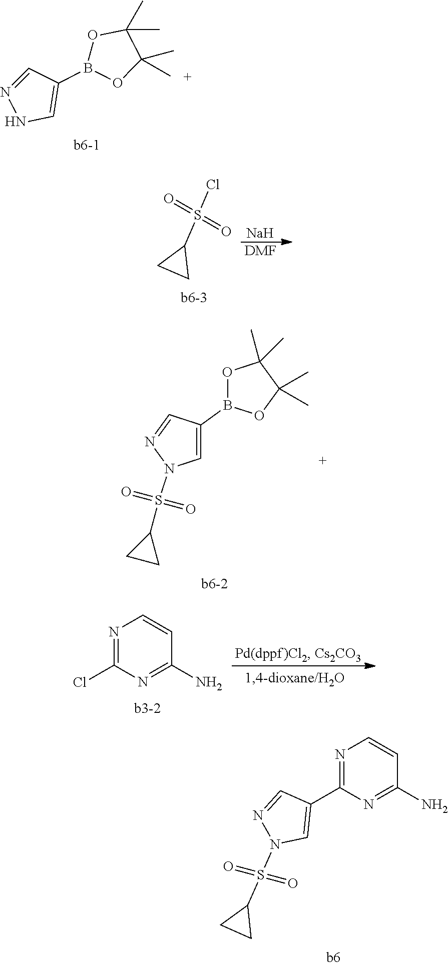

In an ice bath, the raw material 4-(4,4,5,5-tetramethyl-1,3,2-dioxaborolan-2-yl)-1H-pyrazole b6-1 (200 mg, 1.03 mmol) was dissolved in 2 mL of DMF. 60% NaH (82 mg, 2.06 mmol) was added. The mixture was stirred for 1 hour, and cyclopropanesulfonyl chloride (217 mg, 1.54 mmol) was added. The mixture was warmed to room temperature and stirred for 5 hours. 30 mL of ice water was added to the reaction solution to quench the reaction. The mixture was extracted with ethyl acetate, washed with saturated brine, dried over anhydrous sodium sulfate, and filtered. The filtrate was concentrated to obtain crude b6-2, which was directly used in the next step. LCMS: ESI-MS (m/z): 299.1 [M+H]⁺.

Under nitrogen protection, the crude product b6-2 (288 mg, 0.58 mmol) from the previous step, 2-chloropyrimidin-4-amine b3-2 (50 mg, 0.39 mmol) and Cs₂CO₃ (252 mg, 0.77 mmol) were mixed in 6 mL of a mixture of 1,4-dioxane and water (v/v=5/1). Pd(dppf)Cl₂ (57 mg, 0.077 mmol) was added. The mixture was heated to 100° C. and reacted for 6 hours. The reaction was completed by LC-MS monitoring. The mixture was filtered, and 20 mL of water was added to the filtrate. The mixture was extracted with ethyl acetate, dried over anhydrous sodium sulfate, and filtered. The filtrate was concentrated, and separated by flash column chromatography (PE/EA=2/1) to obtain compound b6 (30 mg), yield: 27%, LCMS: ESI-MS (m/z): 266.0 [M+H]⁺.

Referring to the synthetic route of compound b6, the following intermediates were synthesized using a similar skeleton structure.

| Replacement of structure | Target intermediate | LC-MS [M + H]⁺ |
|---|---|---|
| 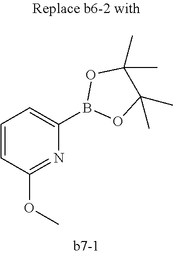 [SMILES: COc1cccc(B2OC(C)(C)C(C)(C)O2)n1] | 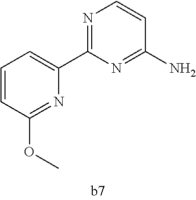 | 203 |
| 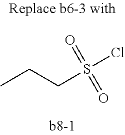 | 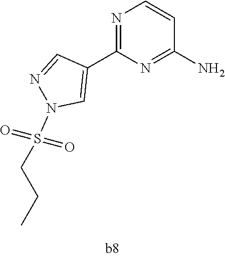 [SMILES: CCCS(=O)(=O)n1cc(-c2nccc(N)n2)cn1] | 268 |

### Synthesis of Intermediates b9, x3 and x4

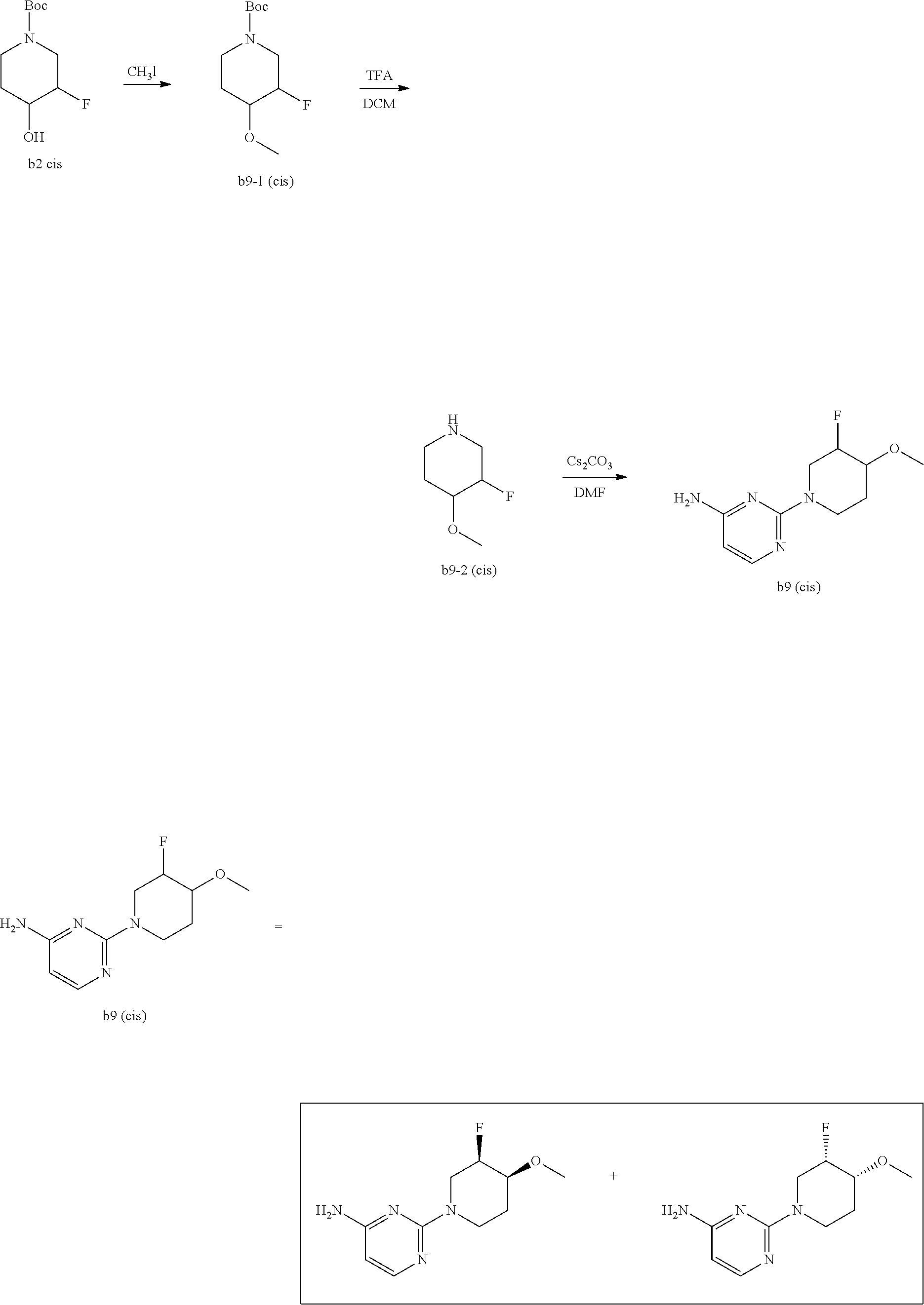

In an ice bath, cis intermediate b2 (600 mg, 2.72 mmol) was dissolved in 6 mL of anhydrous tetrahydrofuran. NaH (131 mg, 3.3 mmol) was added, and iodomethane (426 mg, 3.0 mmol) was added dropwise. The mixture was heated to room temperature and reacted for 3 hours. 20 mL of ice water was added to the system to quench the reaction. The mixture was extracted with ethyl acetate, dried over anhydrous sodium sulfate, and filtered. The filtrate was concentrated, and separated by flash column chromatography (PE/EA=5/1) to obtain a light yellow solid b9-1 (600 mg), yield: 94%, LCMS: ESI-MS (m/z): 134.0 [M−55]⁺.

Under nitrogen protection, the cis intermediate b9-1 (600 mg, 2.56 mmol) was dissolved in 5 mL of dichloromethane. 2 mL of trifluoroacetic acid was slowly added dropwise. The mixture was reacted at room temperature for 2 hours. The solvent was removed by evaporation under reduced pressure to obtain compound b9-2 (340 mg), which was directly used in the next step.

The cis trifluoroacetate b9-2 (340 mg, 1.37 mmol), 2-chloropyrimidin-4-amine b3-2 (125 mg, 0.96 mmol) and cesium carbonate (896 mg, 2.72 mmol) were mixed in 8 mL of DMF. The reaction system was reacted at 100° C. for 1 hour. After cooling to room temperature, 30 mL of water was added to the system. The mixture was extracted with ethyl acetate, dried over anhydrous sodium sulfate, and filtered. The filtrate was concentrated, and separated by column chromatography (EtOAc) to obtain a white solid cis b9 (200 mg), yield: 65%, LC-MS: ESI-MS (m/z): 227.1 [M+H]⁺.

Referring to the synthetic route of compound b9, the following intermediates were synthesized using a similar skeleton structure.

| Replacement of structure | Target intermediate | LC-MS [M + H]⁺ |
|---|---|---|
| Replace b2 (cis) with b2 chiral (3S,4R) | 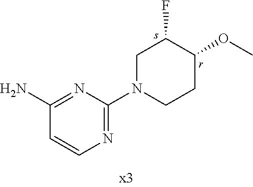 [SMILES: CO[C@@H]1CCN(c2nccc(N)n2)C[C@@H]1F] | 227 |
| Replace b2 (cis) with b2 chiral (3R,4S) | 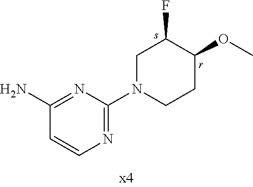 [SMILES: CO[C@H]1CCN(c2nccc(N)n2)C[C@H]1F] | 227 |

### Example 2

Preparation of target molecules P1-P5, P7-P9, P11-P19, P21-P51

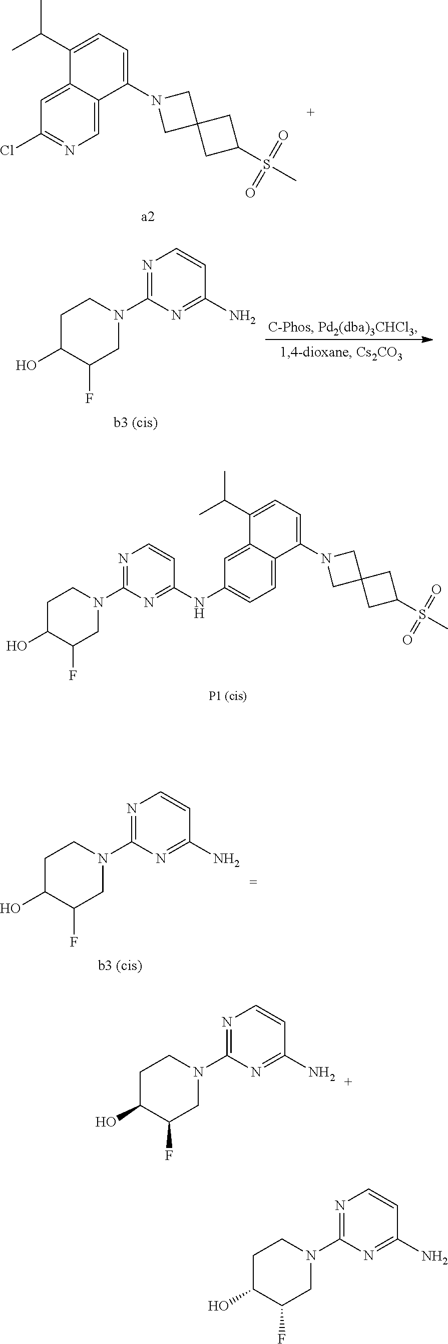 [SMILES: CC(C)c1ccc(N2CC3(CC(S(C)(=O)=O)C3)C2)c2cnc(Cl)cc12 ; Nc1ccnc(N2CCC(O)C(F)C2)n1 ; CC(C)c1ccc(N2CC3(CC(S(C)(=O)=O)C3)C2)c2ccc(Nc3ccnc(N4CCC(O)C(F)C4)n3)cc12 ; Nc1ccnc(N2CCC(O)C(F)C2)n1 ; Nc1ccnc(N2CC[C@H](O)[C@H](F)C2)n1 ; Nc1ccnc(N2CC[C@@H](O)[C@@H](F)C2)n1]

Under nitrogen protection, intermediate a2 (70 mg, 0.185 mmol), Cs₂CO₃ (180 mg, 0.55 mmol) and intermediate b3 (39 mg, 0.185 mmol) were mixed in 2 mL of 1,4-dioxane. Pd₂(dba)₃CHCl₃ (56 mg, 0.055 mmol) and C-phos (24 mg, 0.055 mmol) were added. The mixture was heated to 100° C. and reacted for 12 hours. The reaction was completed by LC-MS monitoring. The mixture was filtered. The filtrate was concentrated, and separated by HPLC preparative chromatography to obtain a yellow solid P1 (4.0 mg), yield: 4%, LCMS: ESI-MS (m/z): 555.0 [M+H]⁺.

¹HNMR (400 MHz, DMSO-d₆) δ 9.87 (s, 1H), 9.00 (s, 1H), 8.60 (s, 1H), 7.95 (d, J=5.7 Hz, 1H), 7.36 (d, J=8.0 Hz, 1H), 6.41 (d, J=5.7 Hz, 1H), 6.34 (d, J=8.1 Hz, 1H), 5.11 (d, J=5.1 Hz, 1H), 4.74-4.58 (m, 1H), 4.58-4.52 (m, 1H), 4.38-4.30 (m, 1H), 4.17 (s, 2H), 4.08 (s, J=6.7 Hz, 2H), 3.93-3.76 (m, 2H), 3.59-3.40 (m, 2H), 2.87 (s, 3H), 2.64-2.53 (m, 4H), 1.72-1.64 (m, 2H), 1.25 (dd, J=6.7, 4.6 Hz, 6H).

Referring to the synthetic route of compound P1, the following target molecules were synthesized using a similar skeleton structure (unless otherwise stated, the compounds are in cis configuration).

| | | | LC- |
|---|---|---|---|
| | | | MS |
| | | | [M + |
| Replacement of structure | Target molecules | ¹H NMR | H]⁺ |
| 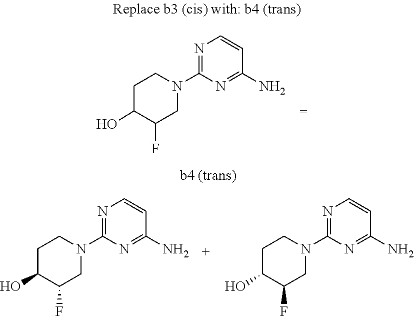 [SMILES: Nc1ccnc(N2CCC(O)C(F)C2)n1 ; Nc1ccnc(N2CC[C@H](O)[C@@H](F)C2)n1 ; Nc1ccnc(N2CC[C@@H](O)[C@H](F)C2)n1] | 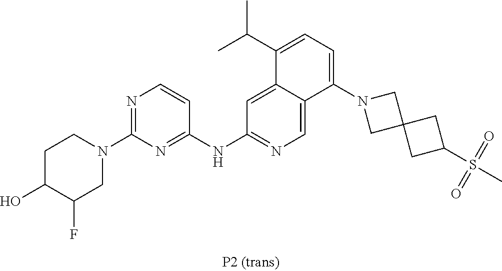 [SMILES: CC(C)c1ccc(N2CC3(CC(S(C)(=O)=O)C3)C2)c2cnc(Nc3ccnc(N4CCC(O)C(F)C4)n3)cc12] | ¹HNMR (400 MHz, DMSO-d₆) δ 9.90 (s, 1H), 9.00 (s, 1H), 8.59 (s, 1H), 7.97 (d, J = 5.6 Hz, 1H), 7.35 (d, J = 8.0 Hz, 1H), 6.44 (d, J = 5.7 Hz, 1H), 6.33 (d, J = 8.1 Hz, 1H), 5.31 (d, J = 4.7 Hz, 1H), 4.48-4.21 (m, 2H), 4.16 (s, 3H), 4.07 (s, 2H), 3.93-3.83 (m, 1H), 3.80-3.71 (m, 1H), 3.54-3.48 (m, 1H), 3.47-3.38 (m, 2H), 2.87 (s, 3H), 2.59-2.56 (m, 4H), 1.92-1.81 (m, 1H), 1.50-1.39 (m, 1H), 1.24 (dd, J = 6.6, 5.4 Hz, 6H). | 555.0 |
| 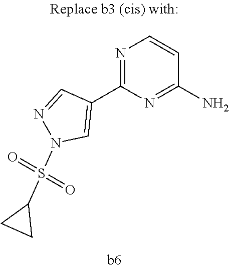 [SMILES: Nc1ccnc(-c2cnn(S(=O)(=O)C3CC3)c2)n1] | 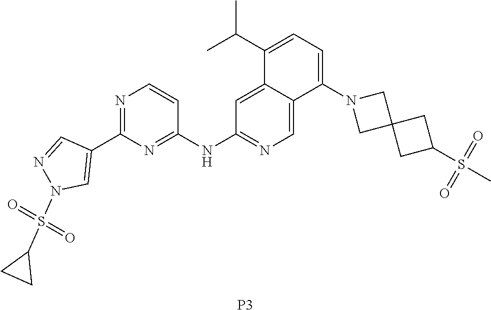 [SMILES: CC(C)c1ccc(N2CC3(CC(S(C)(=O)=O)C3)C2)c2cnc(Nc3ccnc(-c4cnn(S(=O)(=O)C5CC5)c4)n3)cc12 ; "P3"] | ¹H NMR (400 MHz, DMSO-d₆) δ 10.35 (s, 1H), 9.06 (s, 1H), 8.73 (s, 1H), 8.66 (s, 1H), 8.48 (s, 1H), 8.39 (d, J = 5.9 Hz, 1H), 7.39 (d, J = 8.0 Hz, 1H), 7.19 (s, 1H), 6.37 (d, J = 8.1 Hz, 1H), 4.19 (s, 2H), 4.11 (s, 2H), 3.94-3.85 (m, 1H), 3.58-3.48 (m, 1H), 3.24 (dd, J = 8.5, 3.8 Hz, 1H), 2.88 (s, 3H), 2.63-2.54 (m, 4H), 1.33 (d, J = 6.8 Hz, 6H), 1.31-1.21 (m, 4H). | 608.7 |
| 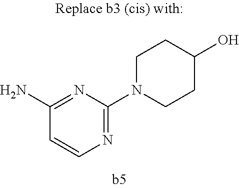 [SMILES: Nc1ccnc(N2CCC(O)CC2)n1] | 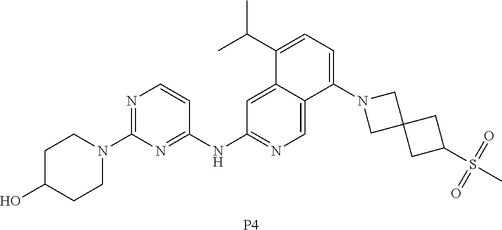 [SMILES: CC(C)c1ccc(N2CC3(CC(S(C)(=O)=O)C3)C2)c2cnc(Nc3ccnc(N4CCC(O)CC4)n3)cc12 ; "P4"] | ¹H NMR (400 MHz, DMSO-d₆) δ 9.87 (s, 1H), 9.03 (s, 1H), 8.67 (s, 1H), 7.98 (d, J = 5.6 Hz, 1H), 7.39 (d, J = 8.0 Hz, 1H), 6.39 (dd, J = 14.6, 6.9 Hz, 2H), 4.75 (d, J = 4.2 Hz, 1H), 4.40-4.32 (m, 2H), 4.21 (s, 2H), 4.12 (s, 2H), 3.97-3.88 (m, 1H), 3.80-3.72 (m, 1H), 3.46- 3.42 (m, 1H), 3.33 (s, 1H), 3.29 (s, 1H), 2.91 (s, 3H), 2.61 (dd, J = 7.9, 3.2 Hz, 4H), 1.86-1.77 (m, 2H), 1.39 (td, J = 12.9, 4.1 Hz, 2H), 1.28 (d, J = 6.8 Hz, 6H). | 661.0 |
| 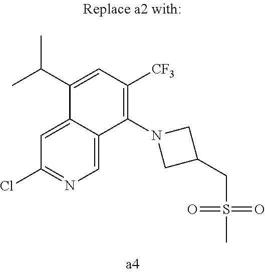 [SMILES: CC(C)c1cc(C(F)(F)F)c(N2CC(CS(C)(=O)=O)C2)c2cnc(Cl)cc12] | 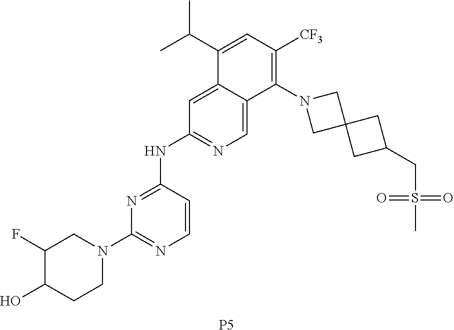 | ¹H NMR (400 MHz, DMSO-d₆) δ 10.53 (s, 1H), 9.08 (s, 1H), 8.49 (s, 1H), 7.99 (d, J = 6.2 Hz, 1H), 7.37 (s, 1H), 6.56 (s, 1H), 5.35-5.00 (m, 1H), 4.81-4.61 (m, 3H), 4.59-4.43 (m, 1H), 4.28-4.24 (m, 2H), 3.92- 3.80 (m, 2H), 3.66-3.59 (m, 1H), 3.55 (d, J = 7.5 Hz, 2H), 3.42-3.32 (m, 2H), 3.23 (dt, J = 21.4, 7.4 Hz, 1H), 2.94 (s, 3H), 1.76-1.68 (m, 2H), 1.26 (dd, J = 6.7, 4.9 Hz, 6H). | 597.2 |
| 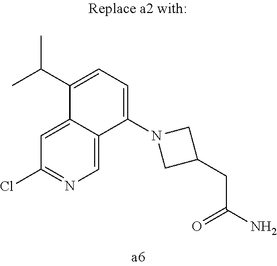 [SMILES: CC(C)c1ccc(N2CC(CC(N)=O)C2)c2cnc(Cl)cc12] | 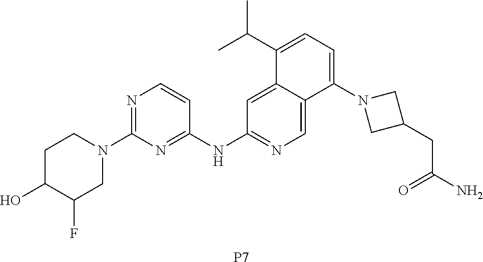 [SMILES: CC(C)c1ccc(N2CC(CC(N)=O)C2)c2cnc(Nc3ccnc(N4CCC(O)C(F)C4)n3)cc12 ; "P7"] | 1H NMR (400 MHz, MeOD) δ 9.07 (s, 1H), 8.64 (s, 1H), 7.92 (d, J = 5.7 Hz, 1H), 7.42 (d, J = 8.0 Hz, 1H), 6.47 (d, J = 8.2 Hz, 1H), 6.43 (d, J = 6.3 Hz, 1H), 4.76 (s, 1H), 4.62 (d, J = 8.2 Hz, 1H), 4.46-4.43 (m, 1H), 4.37-4.40 (m, 2H), 3.89-4.00 (m, 1H), 3.85-3.89 (m, 2H), 3.57-3.65 (m, 2H), 3.45-3.50 (m, 1H), 3.08- 3.13 (m, 1H), 2.62-2.65 (d, J = 8.2 Hz, 2H), 1.84-1.90 (m, 1H), 1.80-1.82 (m, 1H), 1.33 (dd, J = 6.7, 4.5 Hz, 6H). | 493.9 |
| 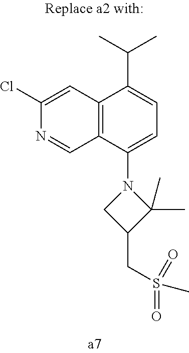 [SMILES: CC(C)c1ccc(N2CC(CS(C)(=O)=O)C2(C)C)c2cnc(Cl)cc12] | 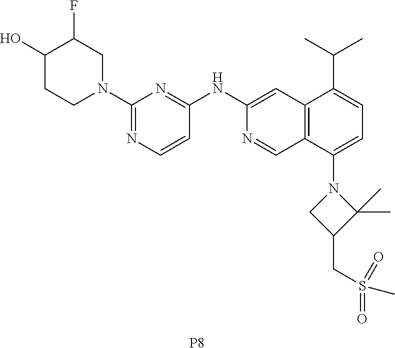 [SMILES: CC(C)c1ccc(N2CC(CS(C)(=O)=O)C2(C)C)c2cnc(Nc3ccnc(N4CCC(O)C(F)C4)n3)cc12 ; "P8"] | ¹H NMR (400 MHz, DMSO-d₆) δ 9.93 (s, 1H), 9.30 (s, 1H), 8.64 (s, 1H), 8.20 (s, 0.3 H), 7.99 (d, J = 5.7 Hz, 1H), 7.47 (d, J = 8.0 Hz, 1H), 6.87 (d, J = 8.0 Hz, 1H), 6.42 (d, J = 5.4 Hz, 1H), 5.15 (d, J = 4.7 Hz, 1H), 4.78-4.56 (m, 2H), 4.43-4.34 (m, 1H), 3.98-3.90 (m, 2H), 3.89-3.80 (m, 1H), 3.61-3.54 (m, 1H), 3.51- 3.46 (m, 2H), 3.05 (s, 3H), 3.01- 2.93 (m, 1H), 1.83-1.65 (m, 3H), 1.56 (s, 2H), 1.35-1.25 (m, 6H), 0.99 (s, 3H). | 557.2 |
| 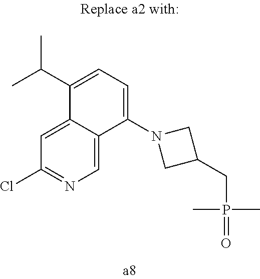 [SMILES: CC(C)c1ccc(N2CC(CP(C)(C)=O)C2)c2cnc(Cl)cc12] | 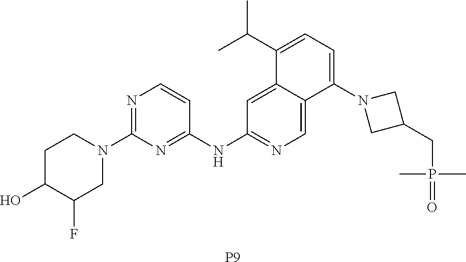 [SMILES: CC(C)c1ccc(N2CC(CP(C)(C)=O)C2)c2cnc(Nc3ccnc(N4CCC(O)C(F)C4)n3)cc12 ; "P9"] | ¹H NMR (400 MHz, DMSO-d₆) δ 11.10 (s, 1H), 9.15 (s, 1H), 8.56 (s, 1H), 8.00 (d, J = 6.9 Hz, 1H), 7.51 (d, J = 8.1 Hz, 1H), 6.72 (s, 1H), 6.51 (d, J = 8.1 Hz, 1H), 4.88 (s, 1H), 4.76 (s, 1H), 4.50 (s, 1H), 4.44-4.40 (m, 2H), 3.97 (m, 1H), 3.91-3.87 (m, 2H), 3.71 (dd, J = 30.2, 13.7 Hz, 2H), 3.50-3.40 (m, 2H), 3.15-3.10 (m, 1H), 2.13 (dd, J = 12.0, 7.7 Hz, 2H), 1.81 (d, J = 4.8 Hz, 2H), 1.42 (d, J = 13.0 Hz, 6H), 1.30 (t, J = 6.3 Hz, 6H). | 527.3 |
| 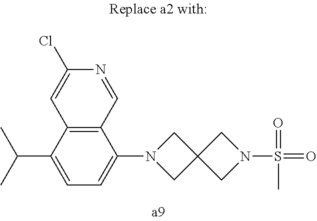 [SMILES: CC(C)c1ccc(N2CC3(C2)CN(S(C)(=O)=O)C3)c2cnc(Cl)cc12] | 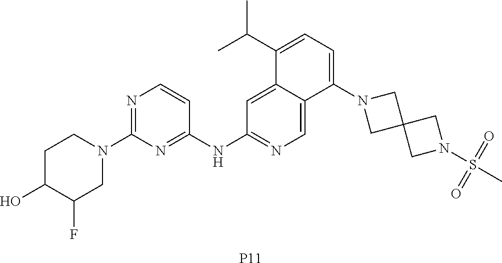 [SMILES: CC(C)c1ccc(N2CC3(C2)CN(S(C)(=O)=O)C3)c2cnc(Nc3ccnc(N4CCC(O)C(F)C4)n3)cc12 ; "P11"] | 1H NMR (400 MHz, DMSO-d₆) δ 9.92 (s, 1H), 9.05 (s, 1H), 8.65 (s, 1H), 8.07 (s, 1H), 8.00 (d, J = 5.6 Hz, 1H), 7.42 (d, J = 8.0 Hz, 1H), 6.45 (d, J = 5.6 Hz, 1H), 6.41 (d, J = 8.1 Hz, 1H), 5.15 (d, J = 4.9 Hz, 1H), 4.74 (m, 1H), 4.64 (m, 2H), 4.39 (m, 1H), 4.31 (s, 4H), 4.13 (s, 4H), 3.87 (m, 1H), 3.48 (m, 1H), 3.03 (s, 3H), 1.72 (d, J = 4.8 Hz, 2H), 1.29 (dd, J = 6.6, 4.6 Hz, 6H). | 556.2 |
| 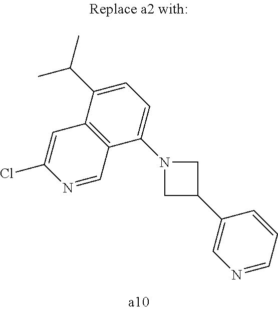 [SMILES: CC(C)c1ccc(N2CC(c3cccnc3)C2)c2cnc(Cl)cc12] | 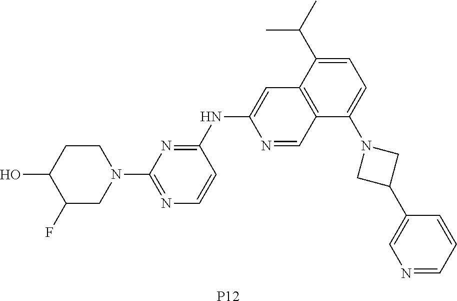 [SMILES: CC(C)c1ccc(N2CC(c3cccnc3)C2)c2cnc(Nc3ccnc(N4CCC(O)C(F)C4)n3)cc12 ; "P12"] | 1H NMR (400 MHz, DMSO-d₆) δ 9.89 (s, 1H), 9.13 (s, 1H), 8.66 (s, 2H), 8.49 (d, J = 3.7 Hz, 1H), 8.33 (s, 2H), 7.99-7.96 (m, 2H), 7.52-7.38 (m, 2H), 6.48 (dd, J = 19.8, 6.8 Hz, 2H), 4.75 (s, 1H), 4.63-4.55 (m, 3H), 4.39 (d, J = 13.0 Hz, 1H), 4.17- 4.20 (m, 2H), 4.09 (d, J = 6.4 Hz, 1H), 3.90-3.83 (m, 1H), 3.84-3.60 (m, 4H), 1.73 (d, J = 4.7 Hz, 2H), 1.23-1.33 (m, 6H). | 514.2 |
| 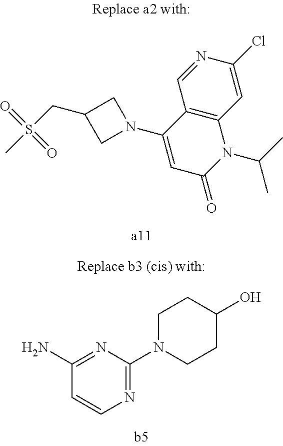 [SMILES: CC(C)n1c(=O)cc(N2CC(CS(C)(=O)=O)C2)c2cnc(Cl)cc21 ; Nc1ccnc(N2CCC(O)CC2)n1] | 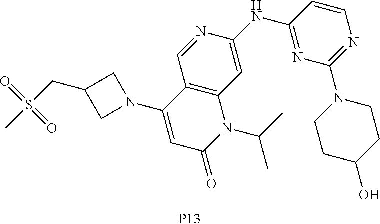 | ¹H NMR (400 MHz, DMSO-d₆) δ 9.94 (s, 1H), 8.52 (s, 1H), 8.02 (d, J = 5.6 Hz, 2H), 6.70 (s, 1H), 5.05 (s, 1H), 4.75-4.65 (m, 1H), 4.42 (t, J = 8.2 Hz, 2H), 4.30-4.18 (m, 2H), 4.08-3.98 (m, 2H), 3.75-3.65 (m, 1H), 3.55 (d, J = 7.3 Hz, 2H), 3.28- 3.16 (m, 4H), 2.97 (s, 3H), 1.80- 1.70 (m, 2H), 1.48 (d, J = 6.8 Hz, 6H), 1.36-1.28 (m, 2H). | 528.2 |
| 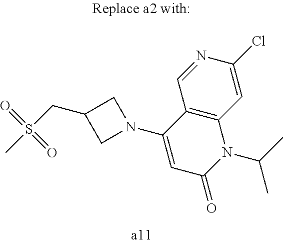 [SMILES: CC(C)n1c(=O)cc(N2CC(CS(C)(=O)=O)C2)c2cnc(Cl)cc21] | 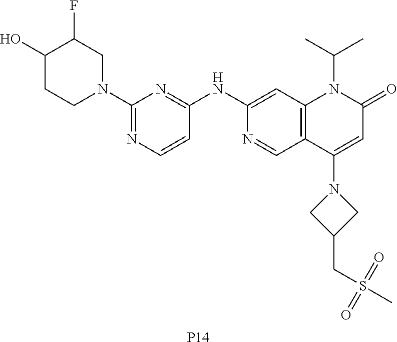 [SMILES: CC(C)n1c(=O)cc(N2CC(CS(C)(=O)=O)C2)c2cnc(Nc3ccnc(N4CCC(O)C(F)C4)n3)cc21 ; "P14"] | ¹H NMR (400 MHz, MeOD) δ 8.66 (s, 1H), 7.86 (d, J = 7.1 Hz, 1H), 7.76 (s, 1H), 7.11 (s, 1H), 5.18 (s, 1H), 4.69-4.37 (m, 4H), 4.27-4.17 (m, 1H), 4.16-4.09 (m, 2H), 3.98-3.77 (m, 2H), 3.61 (dd, J = 28.6, 14.2 Hz, 1H), 3.51 (d, J = 7.5 Hz, 2H), 3.46- 3.28 (m, 3H), 2.92 (s, 3H), 2.56 (s, 1H), 1.87-1.80 (m, 2H), 1.52 (dd, J = 6.9, 3.7 Hz, 6H). | 546.8 |
| 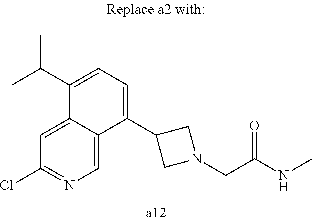 [SMILES: CNC(=O)CN1CC(c2ccc(C(C)C)c3cc(Cl)ncc23)C1] | 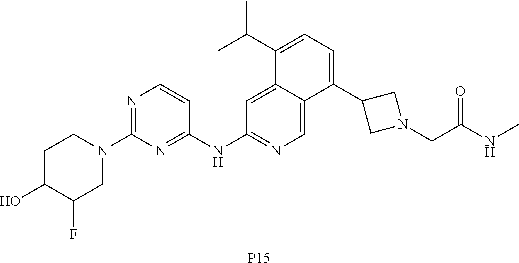 [SMILES: CNC(=O)CN1CC(c2ccc(C(C)C)c3cc(Nc4ccnc(N5CCC(O)C(F)C5)n4)ncc23)C1 ; "P15"] | ¹H NMR (400 MHz, MeOD) δ 9.07 (s, 1H), 8.80 (brs, 1H), 7.86 (d, J = 7.2 Hz, 1H), 7.74 (d, J = 7.5 Hz, 1H), 7.55 (s, 1H), 6.76 (brs, 1H), 5.02- 5.00 (m, 2H), 4.84-4.71 (m, 2H), 4.51 (brs, 3H), 4.32 (s, 1H), 4.21- 3.94 (m, 2H), 3.69-3.65 (m, 2H), 3.57-3.39 (m, 1H), 2.77 (s, 3H), 2.04-1.89 (m, 2H), 1.40 (t, J = 6.5 Hz, 6H). | 508.3 |
| 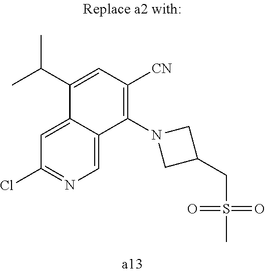 [SMILES: CC(C)c1cc(C#N)c(N2CC(CS(C)(=O)=O)C2)c2cnc(Cl)cc12] | 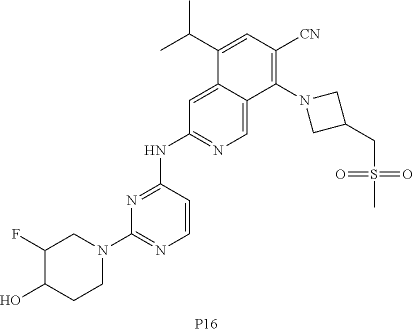 | ¹H NMR (400 MHz, DMSO-d₆) δ 10.09 (s, 1H), 9.07 (s, 1H), 8.56 (s, 1H), 8.04 (d, J = 5.6 Hz, 1H), 7.31 (s, 1H), 6.52 (d, J = 5.6 Hz, 1H), 5.13 (d, J = 5.0 Hz, 1H), 4.88 (t, J = 8.5 Hz, 2H), 4.74 (s, 1H), 4.61-4.55 (m, 1H), 4.50-4.44 (m, 2H), 4.39-4.33 (m, 1H), 3.91-3.83 (m, 1H), 3.626- 3.61 (m, 2H), 3.58-3.49 (m, 1H), 3.41-3.34 (m, 2H), 3.01 (s, 3H), 1.75-1.68 (m,, 2H), 1.28 (dd, J = 6.5, 4.6 Hz, 6H). | 554.2 |
| 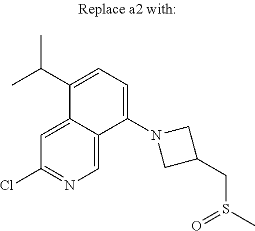 [SMILES: CC(C)c1ccc(N2CC(CS(C)=O)C2)c2cnc(Cl)cc12] | 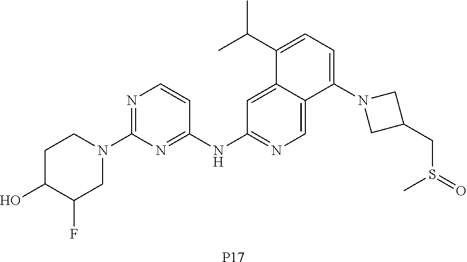 | ¹H NMR (400 MHz, DMSO-d₆) δ 9.90 (s, 1H), 9.03 (s, 1H), 8.60 (s, 1H), 7.96 (d, J = 5.7 Hz, 1H), 7.39 (d, J = 8.0 Hz, 1H), 6.57-6.29 (m, 2H), 5.12 (s, 1H), 4.77-4.59 (m, 1H), 4.59-4.52 (m, 1H), 4.35 (t, J = 7.0 Hz, 3H), 3.97-3.91 (m, 2H), 3.89-3.76 (m, 1H), 3.61-3.49 (m, 1H), 3.45 (dd, J = 13.5, 7.0 Hz, 1H), 3.22-3.16 (m, 2H), 3.06 (dd, J = 16.1, 10.3 Hz, 1H), 2.56 (s, 3H), 1.76- 1.67 (m, 2H), 1.62 (dd, J = 14.9, 7.7 Hz, 1H), 1.26 (t, J = 6.0 Hz, 6H). | 513.2 |
| 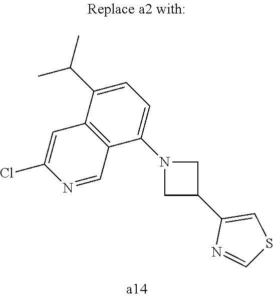 [SMILES: CC(C)c1ccc(N2CC(c3cscn3)C2)c2cnc(Cl)cc12] | 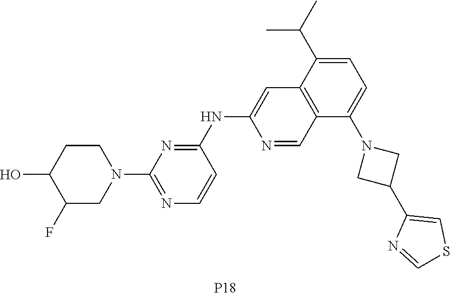 | ¹H NMR (400 MHz, DMSO-d₆) δ 9.90 (s, 1H), 9.14-9.07 (m, 2H), 8.65 (s, 1H), 7.99 (d, J = 5.6 Hz, 1H), 7.67 (d, J = 1.9 Hz, 1H), 7.43 (d, J = 7.9 Hz, 1H), 6.47 (dd, J = 11.7, 6.8 Hz, 2H), 5.16 (s, 1H), 4.75 (s, 1H), 4.61 (d, J = 10.2 Hz, 4H), 4.39 (m, J = 13.5 Hz, 1H), 4.30-4.21 (m, 3H), 3.87 (d, J = 25.6 Hz, 2H), 3.60 (m, J = 11.7 Hz, 1H), 3.53-3.49 (m, 1H), 1.72 (d, J = 4.8 Hz, 2H), 1.31 (dd, J = 6.6, 4.6 Hz, 6H). | 520.2 |
| 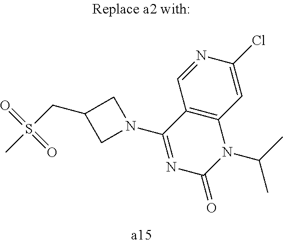 [SMILES: CC(C)n1c(=O)nc(N2CC(CS(C)(=O)=O)C2)c2cnc(Cl)cc21] | 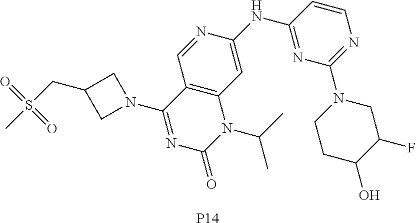 | ¹H NMR (400 MHz, DMSO-d₆) δ 10.37 (s, 1H), 8.57 (s, 1H), 8.07 (d, J = 5.8 Hz, 1H), 7.95 (s, 1H), 6.73 (s, 1H), 4.76-4.57 (m, 3H), 4.54-4.43 (m, 2H), 4.32-4.17 (m, 2H), 3.88- 3.74 (m, 2H), 3.58 (d, J = 7.5 Hz, 3H), 3.52-3.41 (m, 1H), 3.33-3.22 (m, 2H), 2.98 (s, 3H), 1.70-1.63 (m, 2H), 1.47 (dd, J = 6.8, 4.6 Hz, 6H). | 547.0 |
| 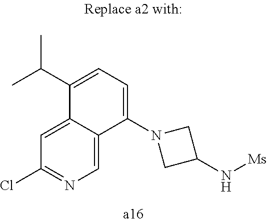 [SMILES: CC(C)c1ccc(N2CC(NS(C)(=O)=O)C2)c2cnc(Cl)cc12] | 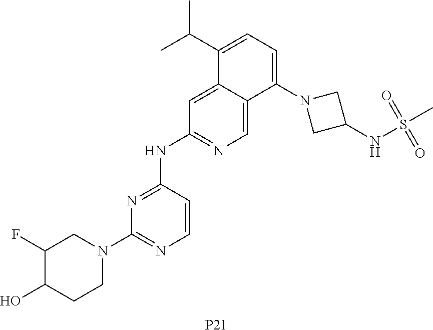 | ¹H NMR (400 MHz, CD₃OD) δ 9.08 (s, 1H), 8.65 (s, 1H), 8.21 (s, 1H), 7.92 (d, J = 5.8 Hz, 1H), 7.45 (d, J = 7.9 Hz, 1H), 6.53 (d, J = 7.9 Hz, 1H), 6.37 (d, J = 5.8 Hz, 1H), 4.78-4.60 (m, 2H), 4.48 (dd, J = 10.2, 6.4 Hz, 3H), 4.39 (dd, J = 9.6, 4.1 Hz, 1H), 4.02-3.94 (m, 1H), 3.91 (d, J = 4.0 Hz, 2H), 3.64 (dd, J = 25.5, 10.8 Hz, 1H), 3.59-3.54 (m, 1H), 3.49-3.38 (m, 1H), 1.92 (dd, J = 19.9, 10.6 Hz, 1H), 1.83 (dd, J = 10.2, 6.1 Hz, 1H), 1.34 (t, J = 6.1 Hz, 6H). | 530.2 |
| 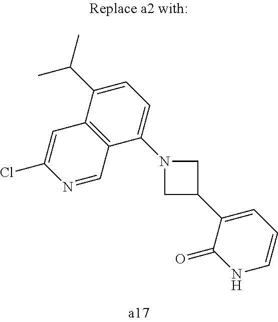 [SMILES: CC(C)c1ccc(N2CC(c3ccc[nH]c3=O)C2)c2cnc(Cl)cc12] | 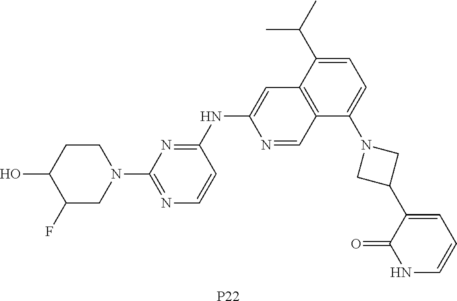 | ¹H NMR (400 MHz, DMSO-d₆) δ 9.91 (s, 1H), 9.38 (s, 1H), 8.64 (s, 1H), 7.99 (d, J = 5.6 Hz, 1H), 7.96 (d, J = 5.1 Hz, 1H), 7.77 (d, J = 7.0 Hz, 1H), 7.37 (d, J = 8.0 Hz, 1H), 6.90 (dd, J = 7.2, 5.2 Hz, 2H), 6.58 (d, J = 8.2 Hz, 1H), 6.44 (d, J = 5.6 Hz, 1H), 5.15 (d, J = 4.9 Hz, 1H), 4.75 (s, 1H), 4.75-4.63 (m, 3H), 4.49 (dd, J = 9.2, 5.1 Hz, 1H), 4.41-4.38 (m, 1H), 4.01-3.96 (m, 1H), 3.91- 3.84 (m, 1H), 3.60 (d, J = 13.8 Hz, 1H), 3.49-3.45 (m, 2H), 3.61-3.35 (m, 2H), 1.72 (d, J = 5.2 Hz, 2H), 1.36-1.19 (m, 6H). | 529.8 |
| 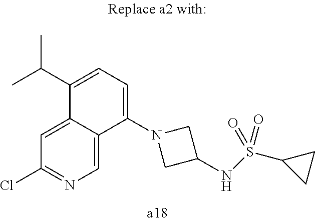 [SMILES: CC(C)c1ccc(N2CC(NS(=O)(=O)C3CC3)C2)c2cnc(Cl)cc12] | 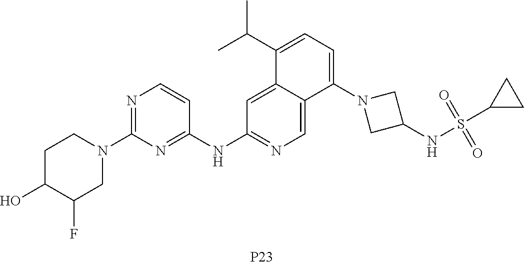 | ¹H NMR (400 MHz, DMSO-d₆) δ 9.93 (s, 1H), 9.02 (s, 1H), 8.60 (s, 1H), 7.96 (d, J = 5.8 Hz, 1H), 7.83 (d, J = 9.1 Hz, 1H), 7.40 (d, J = 7.9 Hz, 1H), 6.43 (d, J = 8.0 Hz, 2H), 5.12 (s, 1H), 4.75-4.58 (m, 1H), 4.58-4.52 (m, 1H), 4.49 (t, J = 7.4 Hz, 2H), 4.37-4.27 (m, 2H), 3.90 (t, J = 6.8 Hz, 2H), 3.87-3.77 (m, 1H), 3.61-3.51 (m, 1H), 3.45 (dd, J = 13.6, 6.9 Hz, 2H), 2.55-2.52 (m, 1H), 1.75-1.66 (m, 2H), 1.26 (t, J = 5.6 Hz, 6H), 0.99-0.94 (m, 2H), 0.93-0.88 (m, 2H). | 556.2 |
| 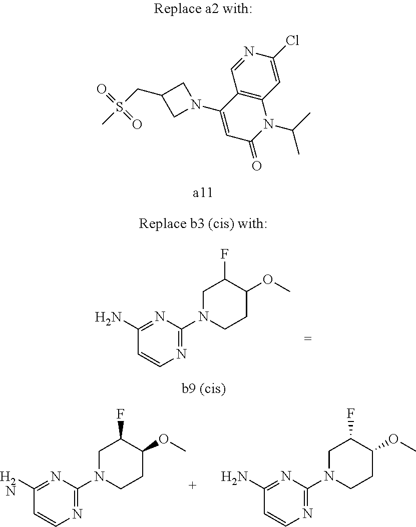 [SMILES: CC(C)n1c(=O)cc(N2CC(CS(C)(=O)=O)C2)c2cnc(Cl)cc21 ; COC1CCN(c2nccc(N)n2)CC1F ; CO[C@H]1CCN(c2nccc(N)n2)C[C@H]1F ; CO[C@@H]1CCN(c2nccc(N)n2)C[C@@H]1F] | 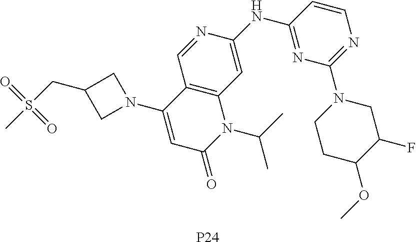 | ¹H NMR (400 MHz, DMSO-d₆) δ 10.85 (s, 1H), 8.59 (s, 1H), 8.03 (d, J = 6.4 Hz, 1H), 7.86 (s, 1H), 7.71- 7.57 (m, 1H), 5.13 (s, 1H), 5.01 (d, J = 48.9 Hz, 1H), 4.61-4.99 (m, 1H), 4.44 (t, J = 8.2 Hz, 2H), 4.33-4.23 (m, 1H), 4.18 (t, J = 6.5 Hz, 1H), 4.07-4.00 (m, 2H), 3.82-3.75 (m, 3H), 3.55 (d, J = 7.4 Hz, 2H), 3.50- 3.43 (m, 1H), 3.29-3.13 (m, 2H), 2.97 (s, 3H), 1.90-1.80 (m, 1H), 1.76-1.66 (m, 1H), 1.64-1.54 (m, 1H), 1.48 (dd, J = 6.2, 4.0 Hz, 6H). | 560.2 |
| 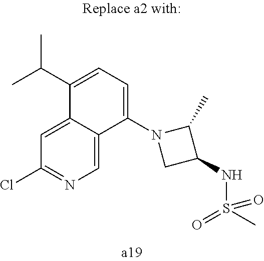 [SMILES: CC(C)c1ccc(N2C[C@H](NS(C)(=O)=O)[C@H]2C)c2cnc(Cl)cc12] | 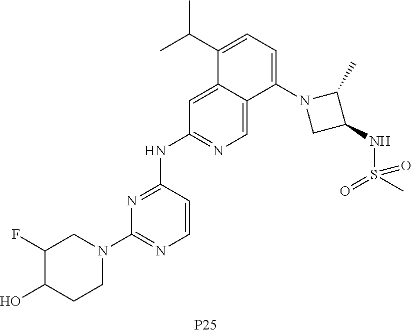 | ¹H NMR (400 MHz, DMSO-d₆) δ 9.95 (s, 1H), 9.05 (s, 1H), 8.67 (s, 1H), 8.00 (d, J = 5.6 Hz, 1H), 7.78 (d, J = 8.6 Hz, 1H), 7.44 (d, J = 8.0 Hz, 1H), 6.62 (d, J = 8.2 Hz, 1H), 6.44 (d, J = 5.6 Hz, 1H), 5.16 (s, 1H), 4.80-4.76 (m, 2H), 4.62-4.60 (m, 2H), 4.40-4.37 (m, 1H), 4.17-4.11 (m, 1H), 3.97-3.84 (m, 2H), 3.58-3.47 (m, 3H), 2.96 (s, 3H), 1.77-1.67 (m, 2H), 1.42 (d, J = 6.1 Hz, 3H), 1.35- 1.18 (m, 6H). | 544.0 |
| 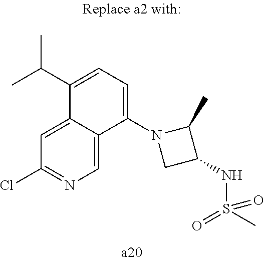 [SMILES: CC(C)c1ccc(N2C[C@@H](NS(C)(=O)=O)[C@@H]2C)c2cnc(Cl)cc12] | 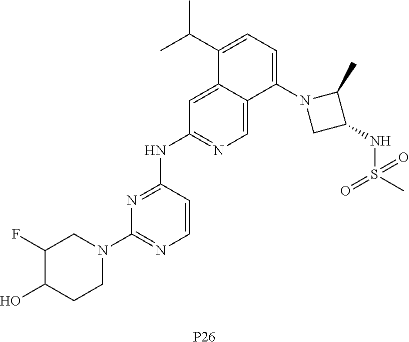 | ¹H NMR (400 MHz, DMSO-d₆) δ 9.92 (s, 1H), 9.06 (s, 1H), 8.67 (s, 1H), 8.20 (s, 1H), 8.00 (d, J = 5.6 Hz, 1H), 7.76 (d, J = 8.5 Hz, 1H), 7.44 (d, J = 8.0 Hz, 1H), 6.62 (d, J = 8.1 Hz, 1H), 6.45 (d, J = 5.7 Hz, 1H), 5.14 (s, 1H), 4.80-4.74 (m, 1H), 4.59- 4.52 (m, 1H), 4.35-4.41 (m, 1H), 4.17-4.11 (m, 1H), 3.96-3.88 (m, 2H), 3.64-3.57 (m, 1H), 3.57- 3.48 (m, 3H), 2.96 (s, 3H), 1.76-1.70 (m, 2H), 1.42 (d, J = 6.1 Hz, 3H), 1.34-1.27 (m, 6H). | 544.0 |
| 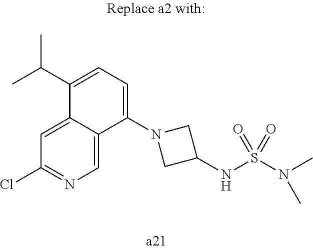 [SMILES: CC(C)c1ccc(N2CC(NS(=O)(=O)N(C)C)C2)c2cnc(Cl)cc12] | 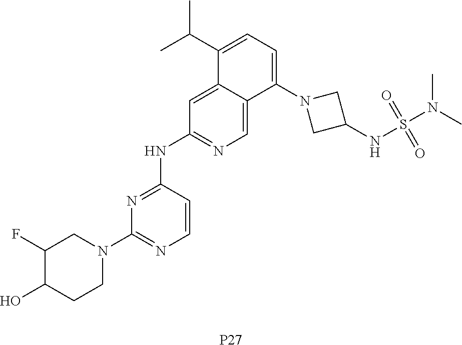 | ¹H NMR (400 MHz, DMSO-d₆) δ 9.91 (s, 1H), 9.05 (s, 1H), 8.65 (s, 1H), 8.17 (s, 1H), 8.00 (d, J = 5.6 Hz, 1H), 7.94 (d, J = 8.8 Hz, 1H), 7.42 (d, J = 8.0 Hz, 1H), 6.46 (d, J = 8.0 Hz, 2H), 5.16 (s, 1H), 4.75 (s, 1H), 4.62 (s, 1H), 4.49 (d, J = 7.5 Hz, 2H), 4.39 (t, J = 13.9 Hz, 1H), 4.32-4.27 (m, 1H), 3.89-3.84 (m, 1H), 3.59 (d, J = 13.8 Hz, 1H), 3.45-3.53 (m, 2H), 2.69 (s, 6H), 1.72 (d, J = 4.7 Hz 2H), 1.30 (dd, J = 6.7, 4.6 Hz, 6H). | 559.1 |
| 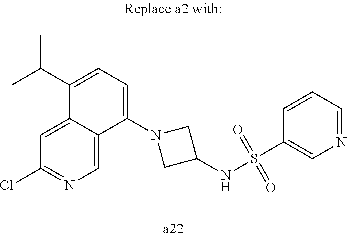 [SMILES: CC(C)c1ccc(N2CC(NS(=O)(=O)c3cccnc3)C2)c2cnc(Cl)cc12] | 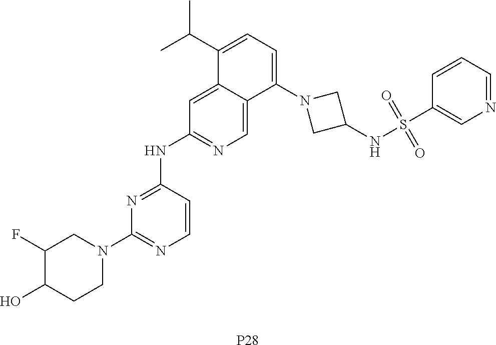 | ¹H NMR (400 MHz, DMSO-d₆) δ 9.91 (s, 1H), 8.97 (d, J = 1.9 Hz, 1H), 8.87 (s, 1H), 8.85-8.82 (m, 1H), 8.65 (d, J = 6.6 Hz, 1H), 8.58 (s, 1H), 8.21 (d, J = 8.1 Hz, 1H), 7.95 (d, J = 5.7 Hz, 1H), 7.65 (dd, J = 8.0, 4.9 Hz, 1H), 7.35 (d, J = 8.0 Hz, 1H), 6.41 (d, J = 4.5 Hz, 1H), 6.32 (d, J = 8.0 Hz, 1H), 5.11 (s, 1H), 4.71 (s, 1H), 4.56-4.53 (m, 1H), 4.34-4.27 (m, 5H), 3.83 (d, J = 24.4 Hz, 1H), 3.65 (s, 2H), 3.60-3.48 (m, 1H), 3.47-3.42 (m, 1H), 1.68 (d, J = 4.5 Hz, 2H), 1.24 (dd, J = 6.3, 5.0 Hz, 6H). | 593.0 |
| 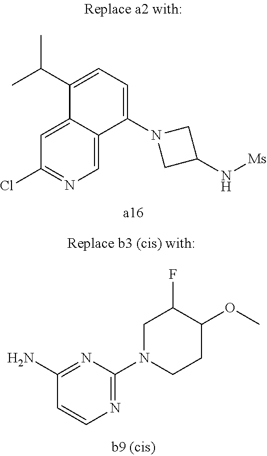 [SMILES: CC(C)c1ccc(N2CC(NS(C)(=O)=O)C2)c2cnc(Cl)cc12 ; COC1CCN(c2nccc(N)n2)CC1F] | 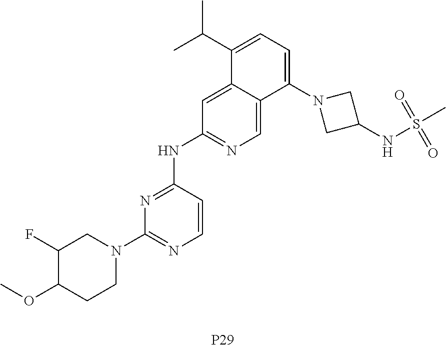 | ¹H NMR (400 MHz, DMSO-d₆) δ 9.93 (s, 1H), 9.05 (s, 1H), 8.63 (s, 1H), 8.00 (d, J = 5.7 Hz, 1H), 7.84 (d, J = 8.6 Hz, 1H), 7.43 (d, J = 8.0 Hz, 1H), 6.49-6.44 (m, 2H), 5.01 (s, 1H), 4.89 (s, 1H), 4.76-4.70 (m, 1H), 4.57-4.47 (m, 3H), 4.39-4.34 (m, 1H), 3.93-3.89 (m, 2H), 3.67-3.44 (m, 4H), 3.37 (s, 3H), 2.97 (s, 3H), 1.80-1.72 (m, 2H), 1.30 (dd, J = 6.7, 4.5 Hz, 6H). | 544.0 |
| 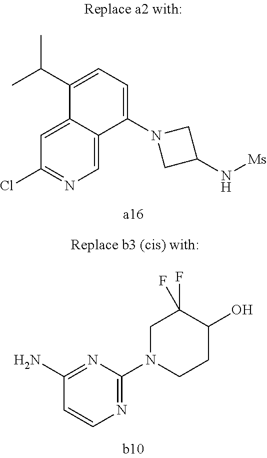 [SMILES: CC(C)c1ccc(N2CC(NS(C)(=O)=O)C2)c2cnc(Cl)cc12 ; Nc1ccnc(N2CCC(O)C(F)(F)C2)n1] | 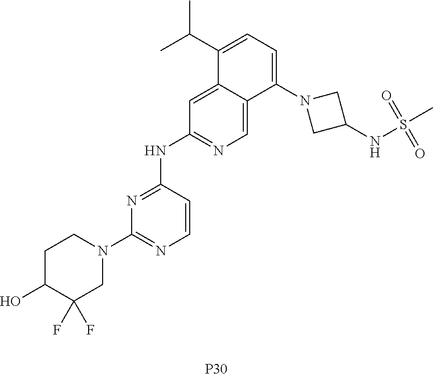 | ¹H NMR (400 MHz, DMSO-d₆) δ 9.92 (s, 1H), 9.01 (s, 1H), 8.55 (s, 1H), 8.21 (s, 1H), 7.99 (d, J = 5.7 Hz, 1H), 7.79 (d, J = 7.0 Hz, 1H), 7.39 (d, J = 8.0 Hz, 1H), 6.50 (d, J = 5.6 Hz, 1H), 6.42 (d, J = 8.1 Hz, 1H), 5.71 (s, 1H), 4.52-4.46 (m, 2H), 4.37- 4.24 (m, 2H), 4.10-4.03 (m, 1H), 4.0-3.92 (m, 1H), 3.90-3.85 (m, 2H), 3.75-3.68 (m, 1H), 3.51-3.40 (m, 2H), 2.93 (s, 3H), 1.90-1.80 (m, 1H), 1.2-1.68 (m, 1H), 1.25 (d, J = 6.7 Hz, 6H). | 547.9 |
| 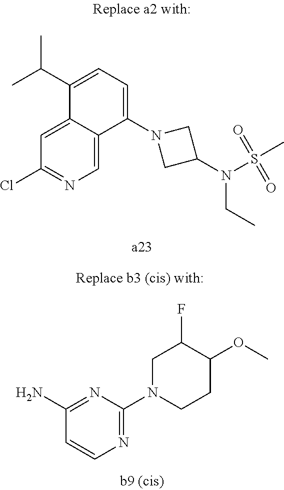 [SMILES: CCN(C1CN(c2ccc(C(C)C)c3cc(Cl)ncc23)C1)S(C)(=O)=O ; COC1CCN(c2nccc(N)n2)CC1F] | 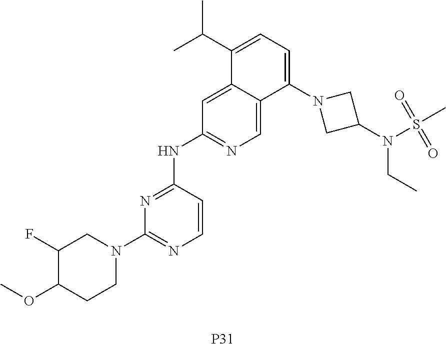 | ¹H NMR DMSO (400 MHz, DMSO- d₆) δ 10.00 (s, 1H), 9.05 (s, 1H), 8.58 (s, 1H), 7.96 (d, J = 5.9 Hz, 1H), 7.41 (d, J = 8.0 Hz, 1H), 6.47 (m, 2H), 4.93 (d, J = 49.5 Hz, 1H), 4.64 (m, 2H), 4.43 (m, 3H), 4.08 (m, 2H), 3.64-3.49 (m, 2H), 3.44-3.48 (m, 2H), 3.31-3.22 (m, 5H), 2.94 (s, 3H), 1.85-1.65 (m, 2H), 1.26 (dd, J = 6.7, 4.8 Hz, 6H), 1.17 (t, J = 7.1 Hz, 3H). | 572.1 |
| 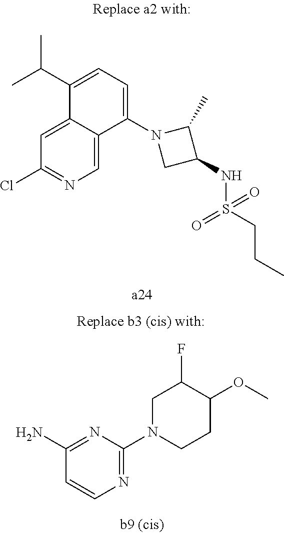 [SMILES: CCCS(=O)(=O)N[C@H]1CN(c2ccc(C(C)C)c3cc(Cl)ncc23)[C@@H]1C ; "a24" ; COC1CCN(c2nccc(N)n2)CC1F] | 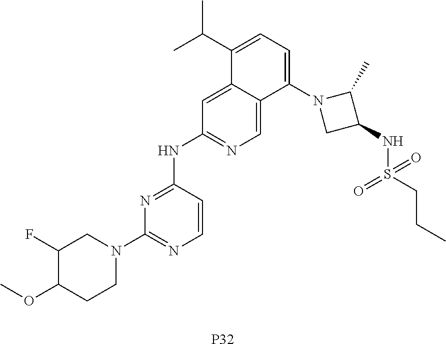 | ¹H NMR (400 MHz, DMSO-d₆) δ 9.96 (s, 1H), 9.06 (s, 1H), 8.65 (s, 1H), 8.00 (d, J = 5.7 Hz, 1H), 7.80 (d, J = 8.7 Hz, 1H), 7.44 (d, J = 8.0 Hz, 1H), 6.62 (d, J = 8.2 Hz, 1H), 6.47 (d, J = 5.6 Hz, 1H), 5.01 (s, 1H), 4.88 (s, 1H), 4.74-4.79 (m, 2H), 4.49 (m, 1 H), 4.15-4.10 (m, 1H), 3.85- 3.95 (m, 2 H), 3.60-3.65 (m, 1H), 3.47-3.55 (m, 3H), 3.46-3.40 (m, 1H), 3.37 (s, 3H), 3.01 (dd, J = 8.8, 5.6 Hz, 2H), 1.85-1.80 (m, 1H), 1.72- 1.65 (m, 3H), 1.41 (d, J = 6.1 Hz, 3H), 1.33-1.28 (m, 6H), 0.99 (t, J = 7.4 Hz, 3H). | 586.3 |
| 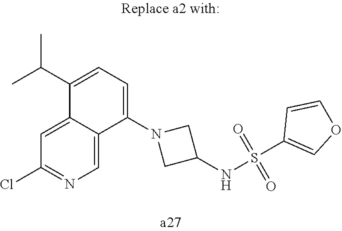 [SMILES: CC(C)c1ccc(N2CC(NS(=O)(=O)c3ccoc3)C2)c2cnc(Cl)cc12] | 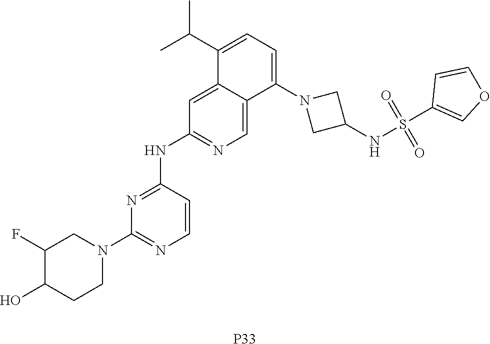 | ¹H NMR (400 MHz, DMSO-d₆) δ 9.91 (s, 1H), 8.97 (s, 1H), 8.63 (s, 1H), 8.42 (d, J = 8.1 Hz 1H), 7.99 (d, J = 5.6 Hz, 2H), 7.91 (s, 1H), 7.75- 7.63 (m, 1H), 7.41 (d, J = 7.8 Hz, 1H), 6.80 (s, 1H), 6.53-6.35 (m, 2H), 5.14 (s, 1H), 4.75 (s, 1H), 4.63-4.59 (m, 1H), 4.45-4.19 (m, 4H), 3.94- 3.72 (m, 3H), 3.65-3.39 (m, 3H), 1.76-1.66 (m, 2H), 1.30-1.24 (m, 6H). | 582.0 |
| 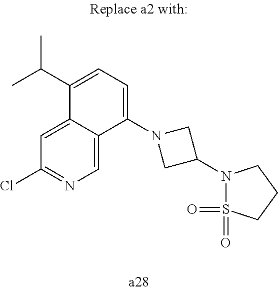 [SMILES: CC(C)c1ccc(N2CC(N3CCCS3(=O)=O)C2)c2cnc(Cl)cc12] | 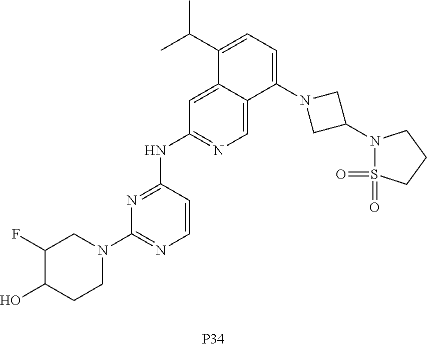 | ¹H NMR (400 MHz, DMSO-d₆) δ 9.92 (s, 1H), 9.06 (s, 1H), 8.65 (s, 1H), 8.00 (d, J = 5.6 Hz, 1H), 7.42 (d, J = 8.0 Hz, 1H), 6.50-6.37 (m, 2H), 5.16 (d, J = 5.1 Hz, 1H), 4.74 (s, 1H), 4.62-4.64 (m, 1H), 4.38- 4.30 (m, 4H), 4.23-4.21 (m, 2H), 3.87 (d, J = 19.1 Hz, 2H), 3.58-3.47 (m, 2H), 3.43 (t, J = 6.6 Hz, 2H), 3.31-3.25 (m, 2H), 2.33-2.21 (m, 2H), 1.73-1.71 (m, J = 4.7 Hz, 2H), 1.30 (dd, J = 6.7, 4.6 Hz, 6H). | 556.0 |
| 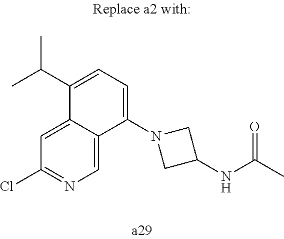 [SMILES: CC(=O)NC1CN(c2ccc(C(C)C)c3cc(Cl)ncc23)C1] | 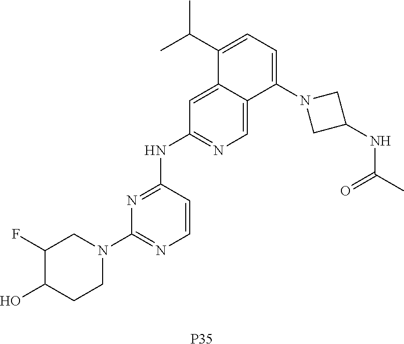 | ¹H NMR (400 MHz, DMSO-d₆) δ 9.92 (s, 1H), 9.05 (s, 1H), 8.65 (s, 1H), 8.56 (d, J = 7.0 Hz, 1H), 7.99 (d, J = 5.6 Hz, 1H), 7.42 (d, J = 8.0 Hz, 1H), 6.50-6.38 (m, 2H), 5.16 (s, 1H), 4.75 (s, 1H), 4.62-4.59 (m, 2H), 4.49-4.33 (m, 3H), 4.07-4.01 (m, 1H), 3.93-3.78 (m, 3H), 3.59 (d, J = 13.2 Hz, 1H), 3.54-3.39 (m, 3H), 1.85 (s, 3H), 1.75-1.65 (m, 2H), 1.30 (dd, J = 6.6, 4.7 Hz, 6H). | 494.1 |
| 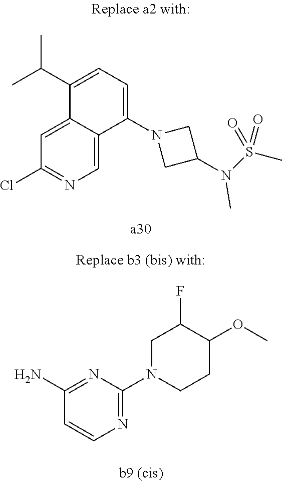 [SMILES: CC(C)c1ccc(N2CC(N(C)S(C)(=O)=O)C2)c2cnc(Cl)cc12 ; COC1CCN(c2nccc(N)n2)CC1F] | 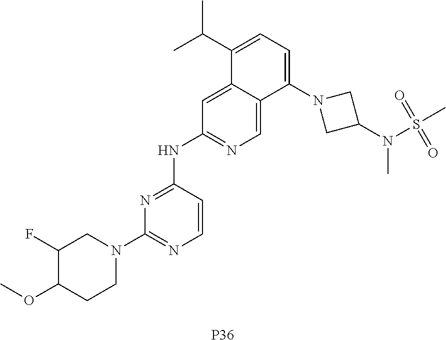 | ¹H NMR (400 MHz, DMSO-d₆) δ 9.90 (s, 1H), 9.08 (s, 1H), 8.62 (s, 1H), 8.00 (d, J = 5.4 Hz, 1H), 7.43 (d, J = 7.8 Hz, 1H), 6.46 (d, J = 8.0 Hz, 2H), 5.01 (s, 1H), 4.88 (s, 1H), 4.67-4.63 (m, 2H), 4.48-4.44 (m, 1H), 4.40-4.37 (m, 2H), 4.22-4.17 (m, 3H), 3.62-3.58 (m, 1H), 3.50- 3.46 (m, 2H), 3.37 (s, 3H), 2.95 (s, 3H), 2.93 (s, 3H), 1.81-1.76 (m, 2H), 1.30-1.23 (m, 6H). | 558.3 |
| 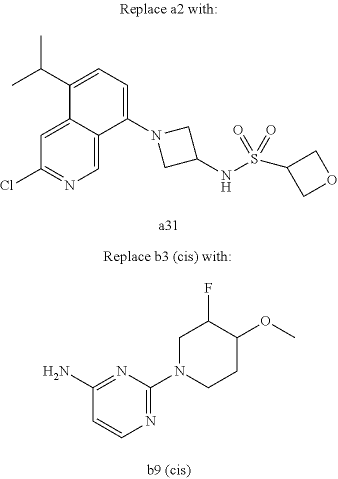 [SMILES: CC(C)c1ccc(N2CC(NS(=O)(=O)C3COC3)C2)c2cnc(Cl)cc12 ; COC1CCN(c2nccc(N)n2)CC1F] | 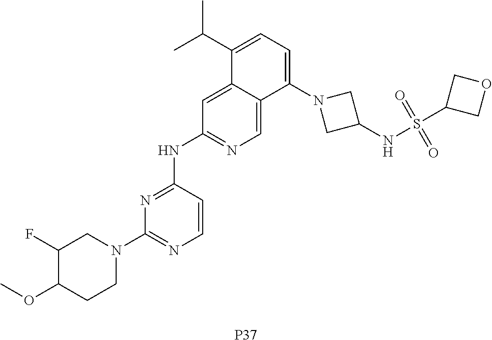 | ¹H NMR (400 MHz, DMSO-d₆) δ 9.85 (s, 1H), 8.99 (s, 1H), 8.58 (s, 1H), 8.20 (s, 1H), 7.96 (d, J = 5.7 Hz, 1H), 7.38 (d, J = 8.0 Hz, 1H), 6.43 (dd, J = 15.3, 6.9 Hz, 2H), 4.99- 4.93 (m, 1H), 4.86-4.76 (m, 2H), 4.73-4.56 (m, 4H), 4.46 (t, J = 7.5 Hz, 3H), 4.35 (s, 1H), 3.81 (t, J = 6.9 Hz, 2H), 3.63-3.37 (m, 3H), 3.33 (s, 3H), 3.24-3.22 (m, 1H), 1.81-1.68 (m, J = 32.1, 14.8, 7.1 Hz, 2H), 1.26 (dd, J = 6.7, 4.3 Hz, 6H). | 586.0 |
| 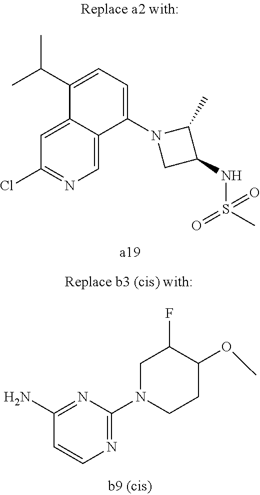 [SMILES: CC(C)c1ccc(N2C[C@H](NS(C)(=O)=O)[C@H]2C)c2cnc(Cl)cc12 ; COC1CCN(c2nccc(N)n2)CC1F] | 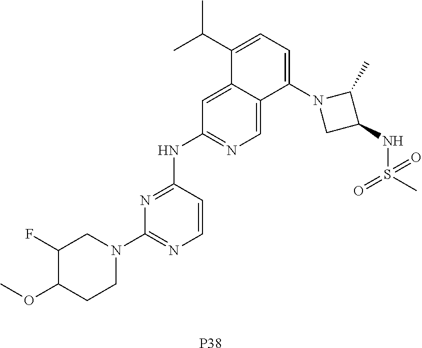 | ¹H NMR (400 MHz, DMSO-d₆) δ 9.96 (s, 1H), 9.06 (s, 1H), 8.65 (s, 1H), 8.00 (d, J = 5.5 Hz, 1H), 7.77 (d, J = 8.5 Hz, 1H), 7.44 (d, J = 7.8 Hz, 1H), 6.62 (d, J = 7.9 Hz, 1H), 6.46 (d, J = 5.3 Hz, 1H), 5.01 (s, 1H), 4.89 (s, 1H), 4.77-4.74 (m, 2H), 4.49 (d, J = 12.7 Hz, 1H), 4.17-4.10 (m, 1H), 3.95-3.90 (m, 1H), 3.62- 3.49 (m, 4H), 3.37 (s, 3H), 2.96 (s, 3H), 1.85-1.72 (m, 2H), 1.42 (d, J = 5.8 Hz, 3H), 1.32-1.31 (m, 6H). | 558.1 |
| 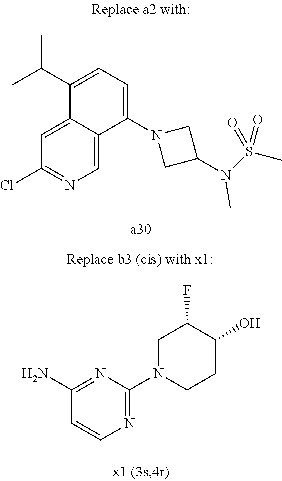 [SMILES: CC(C)c1ccc(N2CC(N(C)S(C)(=O)=O)C2)c2cnc(Cl)cc12 ; Nc1ccnc(N2CC[C@@H](O)[C@@H](F)C2)n1] | 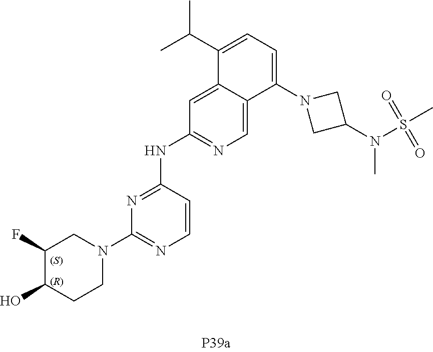 | ¹H NMR (400 MHz, DMSO-d₆) δ 9.90 (s, 1H), 9.09 (s, 1H), 8.64 (s, 1H), 8.00 (d, J = 5.8 Hz, 1H), 7.44 (d, J = 8.0 Hz, 1H), 6.48 (d, J = 8.0 Hz, 2H), 5.16 (d, J = 5.1 Hz, 1H), 4.76 (s, 1H), 4.69-4.58 (m, 2H), 4.42-4.38 (m, 3H), 4.24-4.19 (m, 2H), 3.91-3.79 (m, 1H), 3.63-3.49 (m, 3H), 2.96 (s, 3H), 2.93 (s, 3H), 1.73 (d, J = 4.8 Hz, 2H), 1.30 (dd, J = 6.6, 4.8 Hz, 6H). | 544.2 |
| 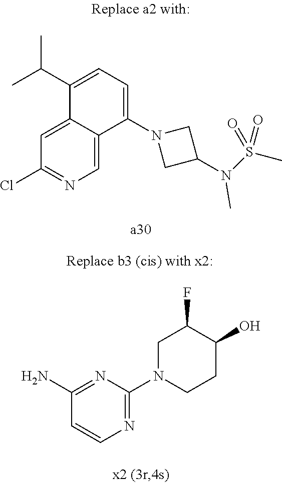 [SMILES: CC(C)c1ccc(N2CC(N(C)S(C)(=O)=O)C2)c2cnc(Cl)cc12 ; Nc1ccnc(N2CC[C@H](O)[C@H](F)C2)n1] | 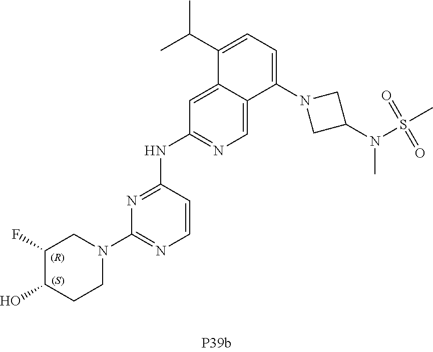 | The same as above | 544.2 |
| 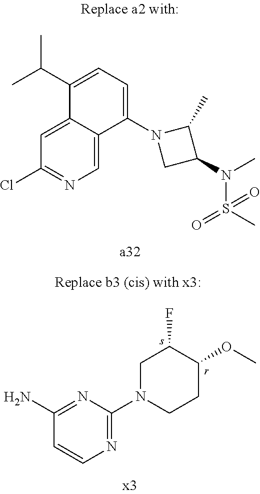 [SMILES: CC(C)c1ccc(N2C[C@H](N(C)S(C)(=O)=O)[C@H]2C)c2cnc(Cl)cc12 ; CO[C@@H]1CCN(c2nccc(N)n2)C[C@@H]1F] | 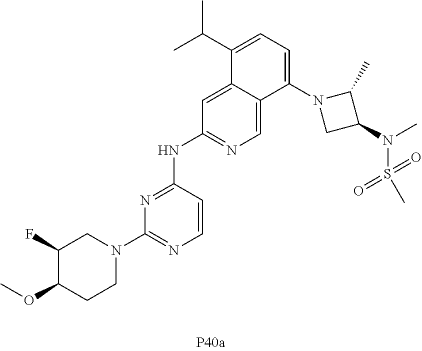 [SMILES: CO[C@@H]1CCN(c2nccc(Nc3cc4c(C(C)C)ccc(N5C[C@H](N(C)S(C)(=O)=O)[C@H]5C)c4cn3)n2)C[C@@H]1F] | ¹H NMR (400 MHz, DMSO-d₆) δ 9.95 (s, 1H), 9.08 (s, 1H), 8.65 (s, 1H), 8.00 (d, J = 5.7 Hz, 1H), 7.45 (d, J = 8.0 Hz, 1H), 6.66 (d, J = 8.1 Hz, 1H), 6.47 (d, J = 5.6 Hz, 1H), 5.00 (s, 1H), 4.88 (s, 1H), 4.74 (s, 1H), 4.64 (t, J = 7.5 Hz, 1H), 4.52- 4.41 (m, 2H), 4.24-4.18 (m, 1H), 3.81 (t, J = 7.2 Hz, 1H), 3.66-3.49 (m, 3H), 3.37 (s, 3H), 3.27-3.25 (m, 1H), 2.95 (s, 3H), 2.82 (s, 3H), 1.84-1.81 (m, J = 20.0, 16.6 Hz, 2H), 1.40 (d, J = 6.0 Hz, 3H), 1.37-1.27 (m, 6H). | 572.3 |
| 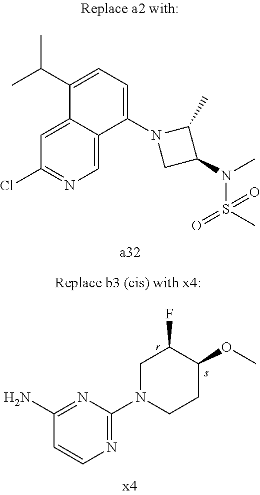 [SMILES: CC(C)c1ccc(N2C[C@H](N(C)S(C)(=O)=O)[C@H]2C)c2cnc(Cl)cc12 ; CO[C@H]1CCN(c2nccc(N)n2)C[C@H]1F] | 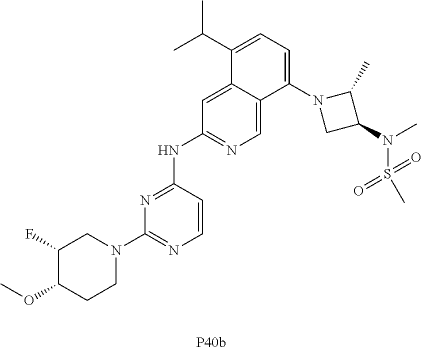 [SMILES: CO[C@H]1CCN(c2nccc(Nc3cc4c(C(C)C)ccc(N5C[C@H](N(C)S(C)(=O)=O)[C@H]5C)c4cn3)n2)C[C@H]1F] | The same as above | 572.3 |
| 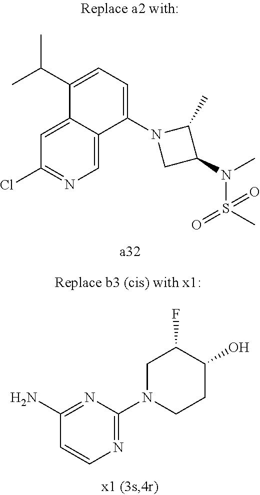 [SMILES: CC(C)c1ccc(N2C[C@H](N(C)S(C)(=O)=O)[C@H]2C)c2cnc(Cl)cc12 ; Nc1ccnc(N2CC[C@@H](O)[C@@H](F)C2)n1] | 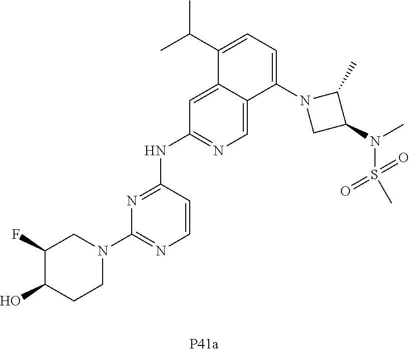 [SMILES: CC(C)c1ccc(N2C[C@H](N(C)S(C)(=O)=O)[C@H]2C)c2cnc(Nc3ccnc(N4CC[C@@H](O)[C@@H](F)C4)n3)cc12] | ¹H NMR (400 MHz, DMSO-d₆) δ 9.96 (s, 1H), 9.09 (s, 1H), 8.67 (s, 1H), 8.00 (d, J = 5.6 Hz, 1H), 7.46 (d, J = 7.7 Hz, 1H), 6.68 (d, J = 7.5 Hz, 1H), 6.49 (s, 1H), 5.18 (s, 1H), 4.77 (s, 1H), 4.65-4.63 (m, 2H), 4.43-4.39 (m, 2H), 4.27-4.12 (m, 2H), 3.84-3.81 (m, 2H), 3.68-3.48 (m, 2H), 2.95 (s, 3H), 2.82 (s, 3H), 1.74-1.64 (m, 2H), 1.49-1.13 (m, 9H). | 558.1 |
| 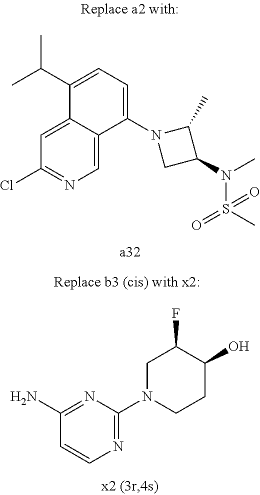 [SMILES: CC(C)c1ccc(N2C[C@H](N(C)S(C)(=O)=O)[C@H]2C)c2cnc(Cl)cc12 ; Nc1ccnc(N2CC[C@H](O)[C@H](F)C2)n1] | 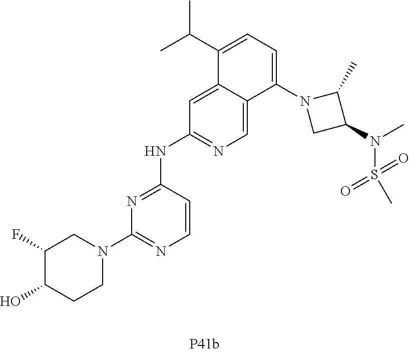 [SMILES: CC(C)c1ccc(N2C[C@H](N(C)S(C)(=O)=O)[C@H]2C)c2cnc(Nc3ccnc(N4CC[C@H](O)[C@H](F)C4)n3)cc12] | ¹H NMR (400 MHz, DMSO-d₆) δ 9.95 (s, 1H), 9.08 (s, 1H), 8.68 (s, 1H), 8.00 (d, J = 5.7 Hz, 1H), 7.45 (d, J = 8.0 Hz, 1H), 6.66 (d, J = 8.1 Hz, 1H), 6.45 (d, J = 5.7 Hz, 1H), 5.16 (d, J = 5.1 Hz, 1H), 4.77-4.61 (m, 3H), 4.45-4.36 (m, 2H), 4.24- 4.20 (m, 1H), 3.94-3.84 (m, 1H), 3.84-3.78 (m, 1H), 3.62-3.54 (m, 1H), 3.51 (d, J = 6.2 Hz, 1H), 3.40- 3.35 (m, 1H), 2.95 (s, 3H), 2.82 (s, 3H), 1.77-1.69 (m, 2H), 1.40 (d, J = 6.0 Hz, 3H), 1.31 (dd, J = 11.6, 6.8 Hz, 6H). | 558.1 |
| 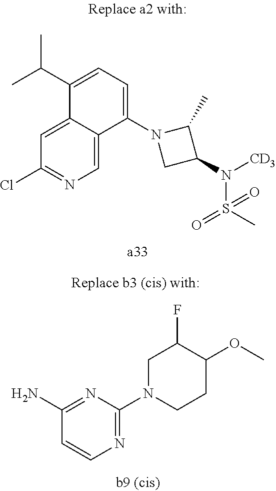 [SMILES: [2H]C([2H])([2H])N([C@H]1CN(c2ccc(C(C)C)c3cc(Cl)ncc23)[C@@H]1C)S(C)(=O)=O ; COC1CCN(c2nccc(N)n2)CC1F] | 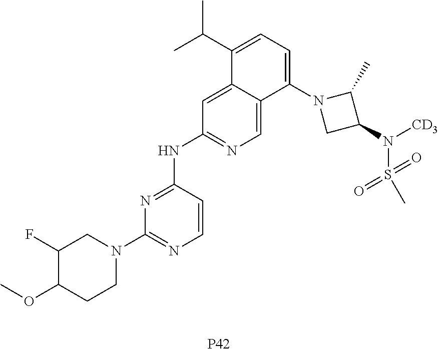 | ¹H NMR (400 MHz, DMSO-d₆) δ 10.12 (s, 1H), 9.10 (s, 1H), 8.64 (s, 1H), 8.00 (d, J = 5.8 Hz, 1H), 7.46 (d, J = 7.9 Hz, 1H), 6.68 (d, J = 8.0 Hz, 1H), 6.52 (s, 1H), 4.97 (d, J = 48.8 Hz, 1H), 4.77-4.68 (m, 1H), 4.65 (t, J = 7.4 Hz, 1H), 4.51-4.40 (m, 2H), 4.20 (dd, J = 13.5, 6.8 Hz, 1H), 3.81 (t, J = 7.1 Hz, 1H), 3.67- 3.59 (m, 1H), 3.59-3.53 (m, 1H), 3.53-3.42 (m, 2H), 3.37 (s, 3H), 2.95 (s, 3H), 1.89-1.81 (m, 1H), 1.78-1.70 (m, 1H), 1.40 (d, J = 6.0 Hz, 3H), 1.31 (dd, J = 11.9, 6.7 Hz, 6H). | 575.1 |
| 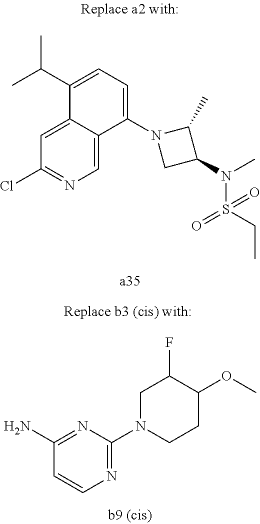 [SMILES: CCS(=O)(=O)N(C)[C@H]1CN(c2ccc(C(C)C)c3cc(Cl)ncc23)[C@@H]1C ; COC1CCN(c2nccc(N)n2)CC1F] | 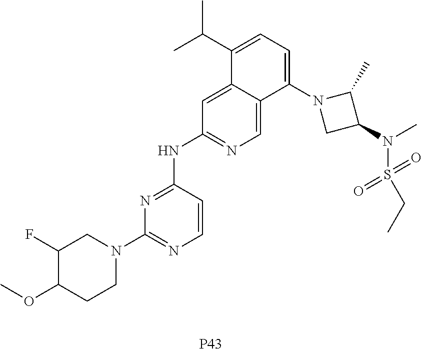 [SMILES: CCS(=O)(=O)N(C)[C@H]1CN(c2ccc(C(C)C)c3cc(Nc4ccnc(N5CCC(OC)C(F)C5)n4)ncc23)[C@@H]1C ; "P43"] | ¹H NMR (400 MHz, DMSO-d₆) δ 9.96 (s, 1H), 9.08 (s, 1H), 8.65 (s, 1H), 8.00 (d, J = 5.6 Hz, 1H), 7.44 (d, J = 8.0 Hz, 1H), 6.67 (d, J = 8.1 Hz, 1H), 6.47 (d, J = 5.5 Hz, 1H), 4.95 (dd, J = 49.6, 1.3 Hz, 1H), 4.79- 4.69 (m, 1H), 4.62 (t, J = 7.4 Hz, 1H), 4.53-4.41 (m, 2H), 4.31 (q, J = 6.9 Hz, 1H), 3.86 (t, J = 7.1 Hz, 1H), 3.67-3.57 (m, 1H), 3.57-3.48 (m, 2H), 3.35-3.24 (m, 4H), 3.12 (q, J = 7.3 Hz, 2H), 2.86 (s, 3H), 1.87-1.78 (m, 1H), 1.78-1.68 (m, 1H), 1.39 (d J = 6.0 Hz, 3H), 1.31 (dd, J = 11.0, 6.3 Hz, 6H), 1.21 (t, J = 7.3 Hz, 3H). | 585.9 |
| 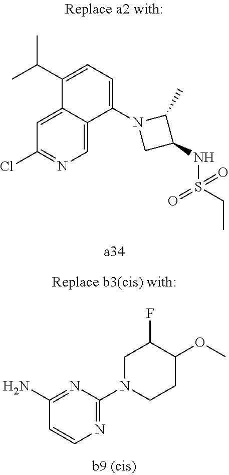 [SMILES: CCS(=O)(=O)N[C@H]1CN(c2ccc(C(C)C)c3cc(Cl)ncc23)[C@@H]1C ; COC1CCN(c2nccc(N)n2)CC1F] | 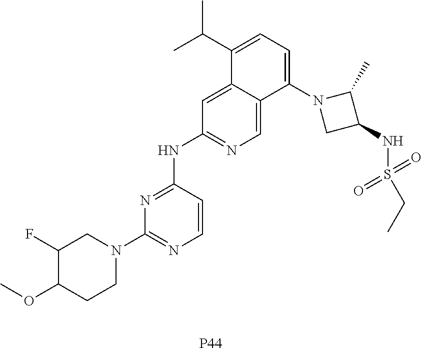 | ¹H NMR (400 MHz, DMSO-d₆) δ 10.28 (s, 1H), 9.09 (s, 1H), 8.63 (s, 1H), 8.00 (d, J = 6.0 Hz, 1H), 7.81 (d, J = 8.7 Hz, 1H), 7.47 (d, J = 8.0 Hz, 1H), 6.66 (d, J = 8.1 Hz, 1H), 6.55 (s, 1H), 4.99 (d, J = 48.6 Hz, 1H), 4.77 (t, J = 7.2 Hz, 1H), 4.74- 4.66 (m, 1H), 4.52-4.40 (m, 1H), 4.17-4.10 (m, 1H), 3.89 (dt, J = 14.3, 7.1 Hz, 1H), 3.68-3.59 (m, 1H), 3.59-3.42 (m, 4H), 3.37 (s, 3H), 3.04 (q, J = 7.3 Hz, 2H), 1.90- 1.81 (m, 1H), 1.79-1.70 (m, 1H), 1.41 (d, J = 6.1 Hz, 3H), 1.31 (dd, J = 10.3, 4.7 Hz, 6H), 1.21 (t, J = 7.3 Hz, 3H). | 572.2 |
| 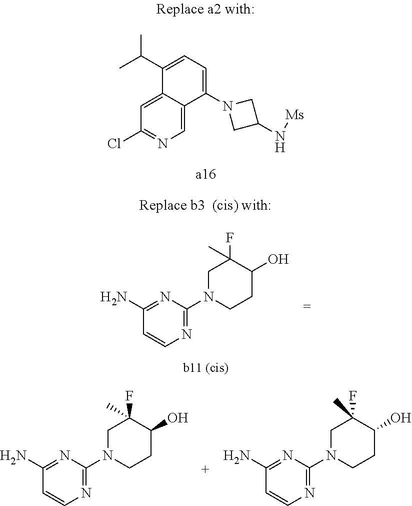 [SMILES: CC(C)c1ccc(N2CC(NS(C)(=O)=O)C2)c2cnc(Cl)cc12 ; CC1(F)CN(c2nccc(N)n2)CCC1O ; C[C@@]1(F)CN(c2nccc(N)n2)CC[C@@H]1O ; C[C@]1(F)CN(c2nccc(N)n2)CC[C@H]1O] | 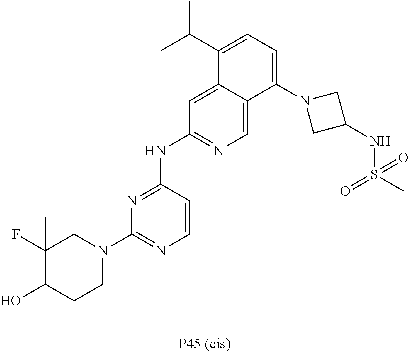 [SMILES: CC(C)c1ccc(N2CC(NS(C)(=O)=O)C2)c2cnc(Nc3ccnc(N4CCC(O)C(C)(F)C4)n3)cc12 ; "P45 (cis)"] | ¹H NMR (400 MHz, DMSO-d₆) δ 10.42 (s, 1H), 9.09 (s, 1H), 8.56 (s, 1H), 7.99 (d, J = 6.2 Hz, 1H), 7.84 (d, J = 8.5 Hz, 1H), 7.47 (d, J = 8.0 Hz, 1H), 6.61 (s, 1H), 6.50 (d, J = 8.0 Hz, 1H), 5.12 (s, 1H), 4.62 (d, J = 2.6 Hz, 1H), 4.54 (td, J = 7.1, 3.3 Hz, 2H), 4.37 (dd, J = 14.3, 7.1 Hz, 1H), 3.93 (t, J = 5.7 Hz, 2H), 3.65-3.60 (m, 1H), 3.58-3.54 (m, 1H), 3.51- 3.46 (m, 2H), 3.24-3.19 (m, 1H), 2.97 (s, 3H), 1.83-1.71 (m, 2H), 1.38 (d, J = 21.3 Hz, 3H), 1.30 (dd, J = 6.4, 4.8 Hz, 6H). | 543.8 |
| 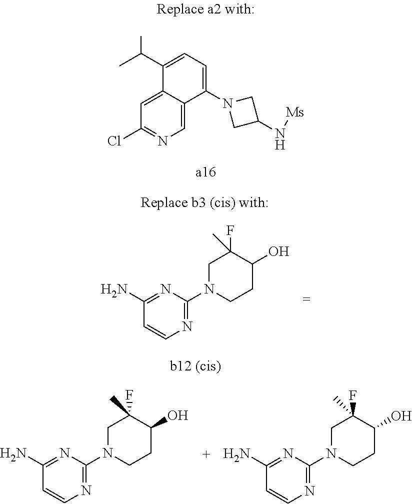 [SMILES: CC(C)c1ccc(N2CC(NS(C)(=O)=O)C2)c2cnc(Cl)cc12 ; CC1(F)CN(c2nccc(N)n2)CCC1O ; C[C@]1(F)CN(c2nccc(N)n2)CC[C@@H]1O ; C[C@@]1(F)CN(c2nccc(N)n2)CC[C@H]1O] | 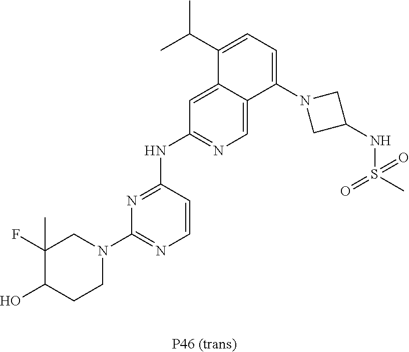 [SMILES: CC(C)c1ccc(N2CC(NS(C)(=O)=O)C2)c2cnc(Nc3ccnc(N4CCC(O)C(C)(F)C4)n3)cc12 ; "P46 (trans)"] | ¹H NMR (400 MHz, DMSO-d₆) δ 9.90 (s, 1H), 9.04 (s, 1H), 8.63 (s, 1H), 8.31 (s, 1H), 7.99 (d, J = 5.6 Hz, 1H), 7.85 (d, J = 8.5 Hz, 1H), 7.43 (d, J = 8.0 Hz, 1H), 6.46 (t, J = 6.2 Hz, 2H), 5.36 (s, 1H), 4.53 (t, J = 7.2 Hz, 2H), 4.36 (dd, J = 14.5, 7.1 Hz, 1H), 3.97-3.86 (m, 5H), 3.80-3.69 (m, 1H), 3.58-3.44 (m, 2H), 2.97 (s, 3H), 1.99-1.83 (m, 1H), 1.54 (td, J = 11.9, 6.1 Hz, 1H), 1.38-1.17 (m, 9H). | 543.8 |
| 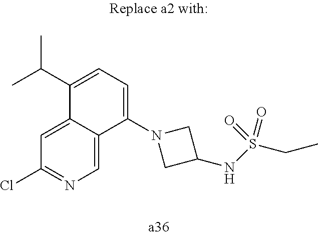 [SMILES: CCS(=O)(=O)NC1CN(c2ccc(C(C)C)c3cc(Cl)ncc23)C1] | 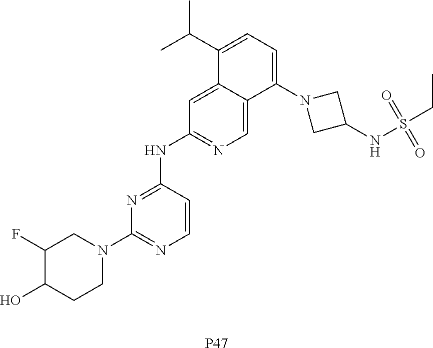 [SMILES: CCS(=O)(=O)NC1CN(c2ccc(C(C)C)c3cc(Nc4ccnc(N5CCC(O)C(F)C5)n4)ncc23)C1 ; "P47"] | ¹H NMR (400 MHz, DMSO-d₆) δ 10.15 (s, 1H), 9.06 (s, 1H), 8.63 (s, 1H), 8.00 (d, J = 5.9 Hz, 1H), 7.86 (d, J = 8.6 Hz, 1H), 7.45 (d, J = 8.0 Hz, 1H), 6.53 (s, 1H), 6.48 (d, J = 8.0 Hz, 1H), 5.18 (s, 1H), 4.72 (d, J = 50.7 Hz, 1H), 4.63-4.55 (m, 1H), 4.52 (t, J = 7.4 Hz, 2H), 4.40-4.30 (m, 2H), 3.95-3.83 (m, 2H), 3.67- 3.57 (m, 1H), 3.56-3.44 (m, 2H), 3.05 (dd, J = 14.6, 7.3 Hz, 2H), 1.78- 1.70 (m, 2H), 1.30 (dd, J = 6.0, 5.4 Hz, 6H), 1.22 (t, J = 7.3 Hz, 3H). | 544.2 |
| 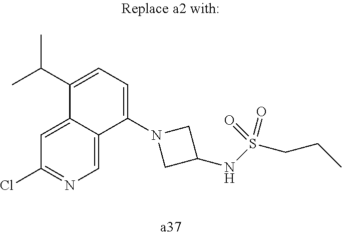 [SMILES: CCCS(=O)(=O)NC1CN(c2ccc(C(C)C)c3cc(Cl)ncc23)C1] | 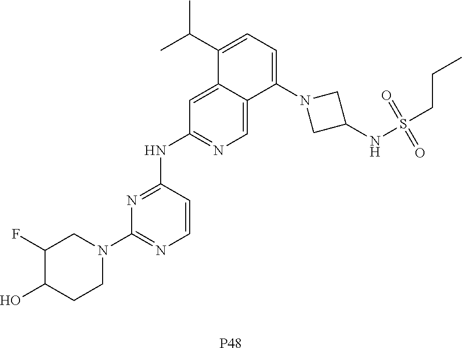 [SMILES: CCCS(=O)(=O)NC1CN(c2ccc(C(C)C)c3cc(Nc4ccnc(N5CCC(O)C(F)C5)n4)ncc23)C1 ; "P48"] | ¹H NMR (400 MHz, DMSO-d₆) δ 9.93 (s, 1H), 9.04 (s, 1H), 8.65 (s, 1H), 8.14 (s, 0.2H), 8.00 (d, J = 5.7 Hz, 1H), 7.87 (d, J = 8.7 Hz, 1H), 7.43 (d, J = 8.0 Hz, 1H), 6.50-6.42 (m, 2H), 5.16 (d, J = 4.6 Hz, 1H), 4.77-4.54 (m, 1H), 4.63-4.57 (m, 1H), 4.51 (t, J = 7.4 Hz, 2H), 4.41- 4.31 (m, 2H), 3.92-3.82 (m, 3H), 3.64-3.56 (m, 1H), 3.56-3.45 (m, 2H), 3.05-2.99 (m, 2H), 1.76-1.64 (m, 4H), 1.30 (dd, J = 6.5, 4.8 Hz, 6H), 1.00 (t, J = 7.4 Hz, 3H). | 557.9 |
| 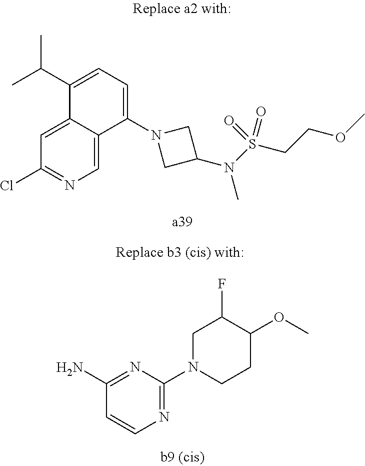 [SMILES: COCCS(=O)(=O)N(C)C1CN(c2ccc(C(C)C)c3cc(Cl)ncc23)C1 ; COC1CCN(c2nccc(N)n2)CC1F] | 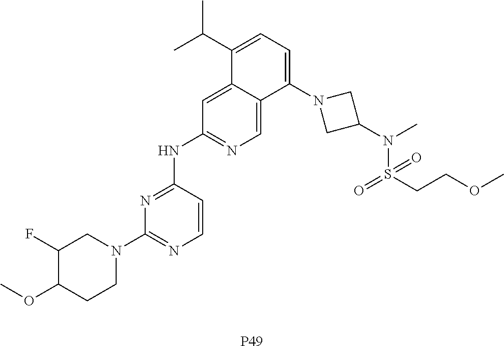 | ¹H NMR (400 MHz, DMSO-d₆) δ 9.92 (s, 1H), 9.08 (s, 1H), 8.63 (s, 1H), 8.00 (d, J = 5.6 Hz, 1H), 7.43 (d, J = 8.0 Hz, 1H), 6.54-6.40 (m, 2H), 4.94 (d, J = 51.4 Hz, 1H), 4.79- 4.71 (m, 2H), 4.50 (d, J = 12.9 Hz, 1H), 4.40-4.35 (m, 2H), 4.27-4.20 (m, 2H), 3.68-3.64 (m, 2H), 3.564- 3.55 (m, 1H), 3.54-3.42 (m, 2H), 3.42-3.37 (m, 2H), 3.37 (s, 3H), 3.36-3.34 (m, 1H), 3.30 (s, 3H), 2.96 (s, 3H), 1.86-1.78 (m, 1H), 1.74- 1.66 (m, 1H), 1.38-1.16 (m, 6H). | 602.0 |
| 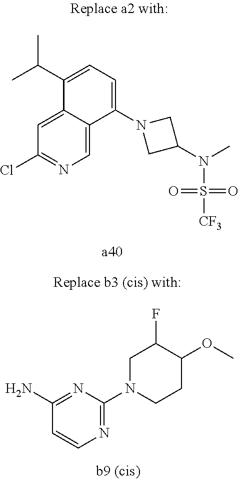 [SMILES: CC(C)c1ccc(N2CC(N(C)S(=O)(=O)C(F)(F)F)C2)c2cnc(Cl)cc12 ; COC1CCN(c2nccc(N)n2)CC1F] | 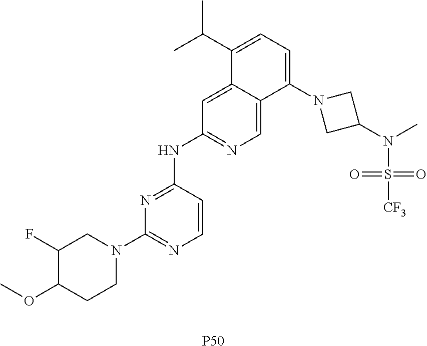 | ¹H NMR (400 MHz, DMSO-d₆) δ 9.91 (s, 1H), 9.09 (s, 1H), 8.63 (s, 1H), 8.39 (s, 1H), 8.01 (d, J = 5.6 Hz, 1H), 7.43 (d, J = 8.0 Hz, 1H), 6.55- 6.39 (m, 2H), 5.03-4.87 (m, 2H), 4.73-4.70 (m, 1H), 4.53-4.47 (m, 1H), 4.43 (d, J = 6.2 Hz, 4H), 3.66- 3.57 (m, 1H), 3.54-3.41 (m, 3H), 3.37 (s, 3H), 3.30-3.22 (m, 3H), 1.85-1.78 (m, 1H), 1.74-1.66 (m, 1H), 1.40-1.18 (m, 6H). | 612.2 |
| 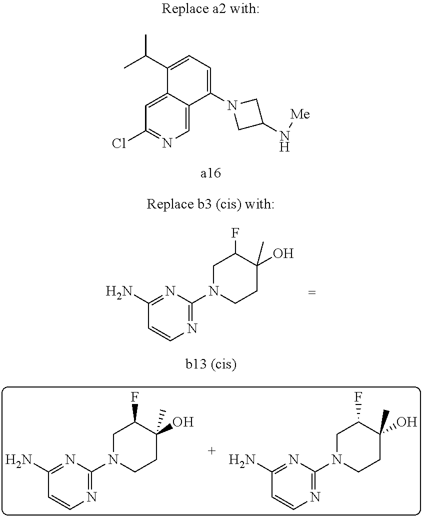 [SMILES: CNC1CN(c2ccc(C(C)C)c3cc(Cl)ncc23)C1 ; CC1(O)CCN(c2nccc(N)n2)CC1F ; C[C@]1(O)CCN(c2nccc(N)n2)C[C@H]1F ; C[C@@]1(O)CCN(c2nccc(N)n2)C[C@@H]1F] | 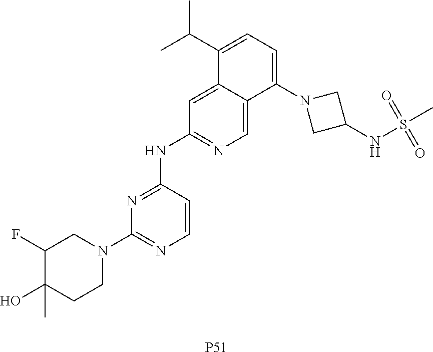 | ¹H NMR (400 MHz, DMSO-d₆) δ 9.93 (s, 1H), 9.04 (s, 1H), 8.65 (s, 1H), 8.33 (s, 1H), 8.01 (d, J = 5.7 Hz 1H), 7.84 (d, J = 8.7 Hz, 1H), 7.42 (d, J = 8.0 Hz, 1H), 6.46 (dd, J = 12.0, 6.7 Hz, 2H), 4.87 (s, 1H), 4.52 (t, J = 8.1 Hz, 2H), 4.48-4.25 (m, 3H), 4.21-4.17 (m, 1H), 3.91 (t, J = 8.1 Hz, 2H), 3.64-3.59 (m, 1H), 3.52-3.46 (m, 2H), 2.97 (s, 3H), 1.72- 1.68 (m, 1H), 1.60-1.56 (m, 1H), 1.33-1.26 (m, 6H), 1.26 (s, 3H). | 544.2 |

### Example 3

### Preparation of Target Molecule P6

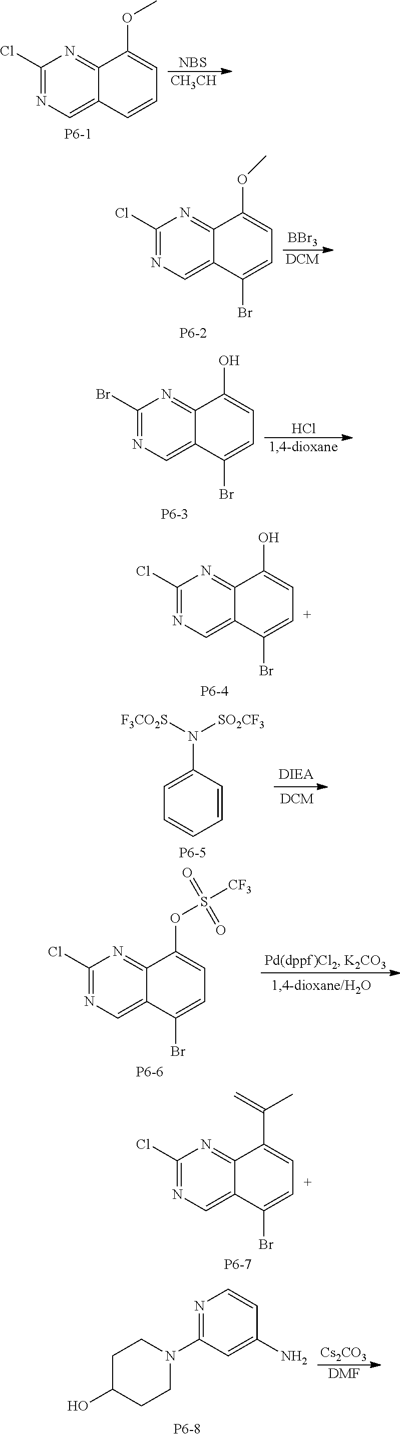

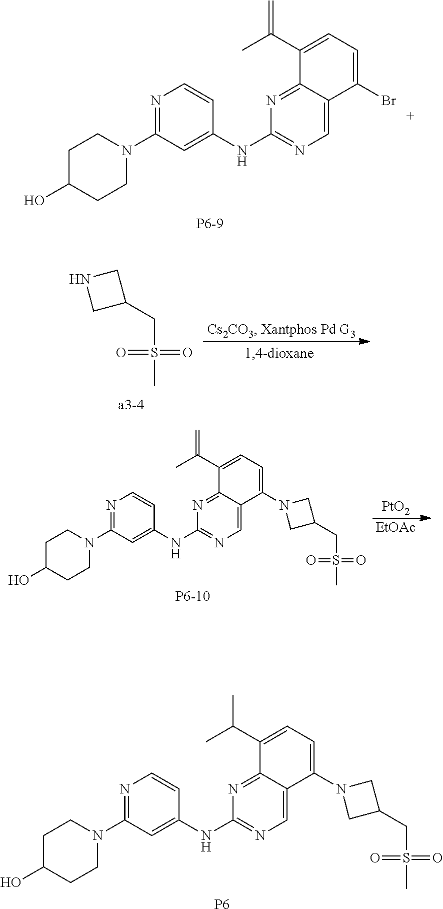

2-Chloro-8-methoxyquinazoline P6-1 (2.0 g, 10.3 mmol) was dissolved in 50 mL of acetonitrile, and N-bromosuccinimide (3.67 g, 20.6 mmol) was added. The mixture was stirred at room temperature for 2 hours. The reaction was completed by LC-MS monitoring. The solvent was removed by evaporation under reduced pressure, and 100 mL of water was added to the system. The mixture was extracted with ethyl acetate, dried over anhydrous sodium sulfate, and filtered. The filtrate was concentrated under reduced pressure, and separated by column chromatography (PE/EA=1/10) to obtain 5-bromo-2-chloro-8-methoxyquinazoline P6-2 (2.85 g), yield: 80%, LCMS: ESI-MS (m/z): 272.9 [M+H]⁺.

¹H NMR (400 MHz, CDCl₃) δ 9.54 (s, 1H), 7.78 (d, J=8.4 Hz, 1H), 7.15 (d, J=8.4 Hz, 1H), 4.08 (s, 3H).

The intermediate P6-2 (2.0 g, 10.3 mmol) from the previous step was dissolved in 30 mL of dichloromethane, and boron tribromide (2.74 g, 10.9 mmol) was added. The mixture was reacted at room temperature for 12 hours. The reaction was completed by LC-MS monitoring. Aqueous saturated sodium bicarbonate solution was slowly added to the reaction solution to quench the reaction. The mixture was extracted with dichloromethane, dried over anhydrous sodium sulfate, and filtered. The filtrate was concentrated under reduced pressure, and separated by column chromatography (DCM/MeOH=20/1) to obtain 2,5-dibromo-8-hydroxyquinazoline P6-3 (2.8 g), yield: 81%, LCMS: ESI-MS (m/z): 302.8 [M+H]⁺.

The intermediate P6-3 (2.8 g, 9.2 mmol) from the previous step was dissolved in 46 mL of a solution of 1M hydrogen chloride in 1,4-dioxane and the mixture was reacted at room temperature for 1 hour. The reaction was completed by LC-MS monitoring. The reaction solution was concentrated under reduced pressure, and aqueous saturated sodium bicarbonate solution was slowly added to the reaction solution to adjust the pH to about 8. The solution was extracted with dichloromethane, dried over anhydrous sodium sulfate, and filtered. The filtrate was concentrated under reduced pressure to obtain a crude product P6-4 (2.7 g), LCMS: ESI-MS (m/z): 258.8 [M+H]⁺.

The crude product P6-4 (2.7 g) from the previous step and DIEA (2.7 g, 20.8 mmol) were dissolved in 30 mL of dichloromethane, and N-phenylbistrifluoromethylsulfonamide P6-5 (5.57 g, 15.6 mmol) was added. The mixture was allowed to react at room temperature for 12 hours. The reaction was completed by LC-MS monitoring. The reaction solution was concentrated under reduced pressure. 100 mL of water was added to the reaction solution. The mixture was extracted with dichloromethane, dried over anhydrous sodium sulfate, and filtered. The filtrate was concentrated under reduced pressure, and separated by column chromatography (PE/EA=1/1) to obtain compound P6-6 (3.2 g), yield: 71%, LCMS: ESI-MS (m/z): 392.8 [M+H]⁺.

Under nitrogen protection, the intermediate P6-6 (1.0 g, 2.6 mmol) from the previous step, K₂CO₃ (706 mg, 5.2 mmol) and isopropenylboronic acid pinacol ester a1-3 (515 mg, 3.1 mmol) were dissolved in 11 mL of a mixed solution of 1,4-dioxane and water (v/v=10/1). Pd(dppf)Cl₂ (187 mg, 0.25 mmol) was added. The mixture was heated to 45° C. for 2 hours. The reaction was completed by LC-MS monitoring. The mixture was filtered. The filtrate was concentrated, and separated by column chromatography (PE/EA=10/1) to obtain a brown oil P6-7 (100 mg), yield: 8%, LCMS: ESI-MS (m/z): 282.8 [M+H]⁺.

The intermediate P6-7 (100 mg, 0.21 mmol) from the previous step and Cs₂CO₃ (649 mg, 0.63 mmol) were dissolved in 5 mL of DMF. 1-(4-aminopyridin-2-yl)piperidin-4-ol P6-8 (49 mg, 0.25 mmol) was added. The mixture was heated to 110° C. and reacted for 12 hours. The reaction was completed by LC-MS monitoring. After cooling to room temperature, 50 mL of water was added to the reaction solution. The mixture was extracted with dichloromethane, dried over anhydrous sodium sulfate, and filtered. The filtrate was concentrated under reduced pressure, and separated by column chromatography (DCM/MeOH=20/1) to obtain compound P6-9 (40 mg), yield: 39%, LCMS: ESI-MS (m/z): 439.7 [M+H]⁺.

Under nitrogen protection, the intermediate P6-9 (40 mg, 0.091 mmol) from the previous step, Cs₂CO₃ (89 mg, 0.27 mmol) and intermediate a3-4 (36 mg, 0.14 mmol) were dissolved in 2 mL of 1,4-dioxane. XantPhos PdG3 (17 mg, 0.018 mmol) was added. The mixture was heated to 100° C. and reacted for 6 hours. The reaction was completed by LC-MS monitoring. The mixture was filtered. The filtrate was concentrated, and separated by HPLC preparative chromatography to obtain a yellow solid P6-10 (30 mg), yield: 59%, LCMS: ESI-MS (m/z): 509.1 [M+H]⁺.

The intermediate P6-10 (20 mg, 0.04 mmol) from the previous step was dissolved in 5 mL of ethyl acetate, and platinum dioxide (45 mg, 0.19 mmol) was added. The mixture was reacted at room temperature for 6 hours under a hydrogen atmosphere. The reaction solution was filtered. The filtrate was concentrated, and separated by HPLC preparative chromatography to obtain the target compound P6 (1.0 mg), yield: 5%, LC-MS: ESI-MS (m/z): 510.9 [M+H]⁺.

### Example 4

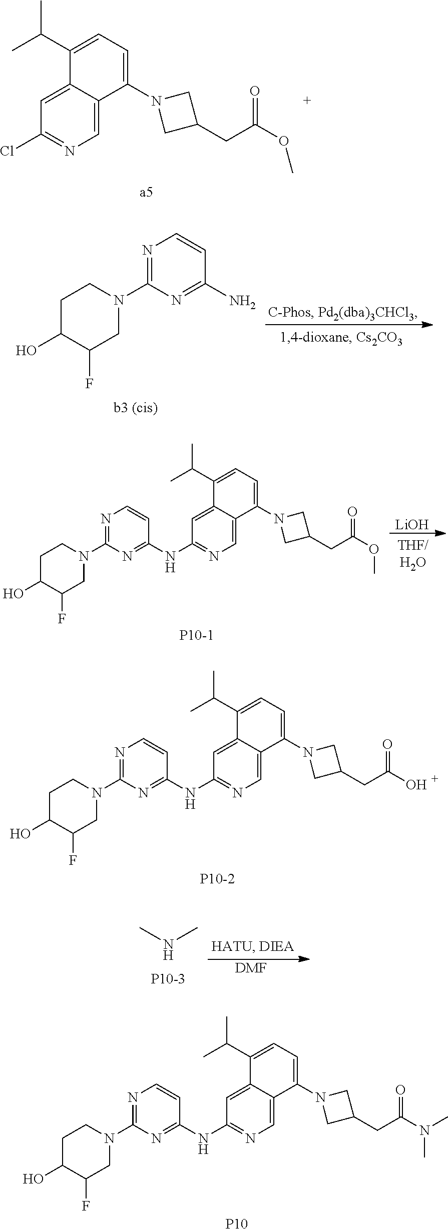

Under nitrogen protection, intermediate a5 (100 mg, 0.30 mmol), Cs₂CO₃ (196 mg, 0.60 mmol) and intermediate b3 cis (64 mg, 0.30 mmol) were mixed in 3 mL of 1,4-dioxane. Pd₂(dba)₃CHCl₃ (31 mg, 0.03 mmol) was added. The mixture was heated to 100° C. and reacted for 16 hours. The reaction was completed by LC-MS monitoring. The mixture was filtered. The filtrate was concentrated, and separated by flash column chromatography (DCM/MeOH=10/1) to obtain a yellow solid P10-1 (100 mg), yield: 59%, LCMS: ESI-MS (m/z): 508.9 [M+H]⁺.

The intermediate P10-I (100 mg, 0.60 mmol) from the previous step was dissolved in 1 mL of tetrahydrofuran, and 3 N aqueous LiOH solution (0.5 mL) was added. The mixture was reacted at room temperature for 3 hours. The reaction was stopped. The reaction solution was concentrated under reduced pressure. Acetic acid was added to the system to adjust the pH to about 6. 10 mL of water was added. The mixture was extracted with ethyl acetate and concentrated under reduced pressure to obtain crude product P10-2 (50 mg), yield: 23%, LCMS: ESI-MS (m/z): 495.1 [M+H]⁺.

The crude product P10-I (10 mg, 0.039 mmol) and a solution of dimethylamine in tetrahydrofuran (1 mL, 1M) were dissolved in 1 mL of DMF. DIEA (16 mg, 0.12 mmol) and HATU (3.0 mg, 0.06 mmol) were added. The mixture was stirred at room temperature for 16 hours. 5 mL of water was added to the system. The mixture was extracted with ethyl acetate, concentrated under reduced pressure, and separated by thin layer chromatography to obtain compound P10 (1.5 mg), yield: 24%. [cis configuration]

¹H NMR (400 MHz, DMSO-d₆) δ 9.08 (s, 1H), 8.41 (s, 1H), 7.92 (d, J=8.0 Hz, 1H), 7.42 (d, J=8.0 Hz, 1H), 6.39 (d, J=8.2 Hz, 1H), 6.33 (d, J=6.3 Hz, 1H), 4.86 (s, 1H), 4.66-4.56 (m, 1H), 4.32-4.40 (m, 3H), 3.89-4.00 (m, 1H), 3.85-3.89 (m, 2H), 3.57-3.65 (m, 2H), 3.45-3.50 (m, 1H), 3.10 (s, 3H), 3.09-3.13 (m, 3H), 2.89 (s, 3H), 2.60 (d, J=6.0 Hz, 2H), 1.84-1.90 (m, 1H), 1.80-1.82 (m, 1H), 1.33 (dd, J=6.7, 4.5 Hz, 6H).

Referring to the synthetic route of compound P10, the following target molecule was synthesized using a similar skeleton structure (unless otherwise stated, the compound is in cis configuration).

| Replacement | | | | LC-MS |
|---|---|---|---|---|
| of structure | Target molecule | | ¹H NMR | [M + H]⁺ |
| Replace P10- 3 with: MeNH₂(HCl) | 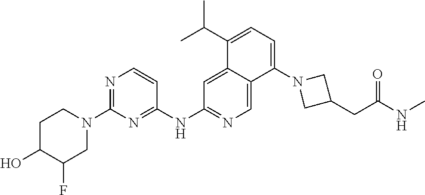 [SMILES: CNC(=O)CC1CN(c2ccc(C(C)C)c3cc(Nc4ccnc(N5CCC(O)C(F)C5)n4)ncc23)C1] | P20 | ¹H NMR (400 MHz, CD₃OD) δ 9.06 (s, 1H), 8.64 (s, 1H), 7.93 (d, J = 5.9 Hz, 1H), 7.43 (d, J = 8.0 Hz, 1H), 6.46 (d, J = 8.0 Hz, 1H), 6.34 (d, J = 5.8 Hz, 1H), 4.78 − 4.58 (m, 2H), 4.45 − 4.35 (m, 1H), 4.29 (t, J = 7.5 Hz, 2H), 3.96 (dd, J = 22.7, 7.2 Hz, 1H), 3.83 (t, J = 6.5 Hz, 2H), 3.70 − 3.52 (m, 2H), 3.43 (t, J = 9.1 Hz, 1H), 3.09 (dt, J = 13.5, 6.8 Hz, 1H), 2.71 (s, 3H), 2.60 (d, J = 7.7 Hz, 2H), 1.94 − 1.78 (m, 2H), 1.35 − 1.31 (m, 6H). | 508.2 |

### Example 5

Preparation of target molecules H1-H2, H4-H5, H12, H23, H25, and H33-H34

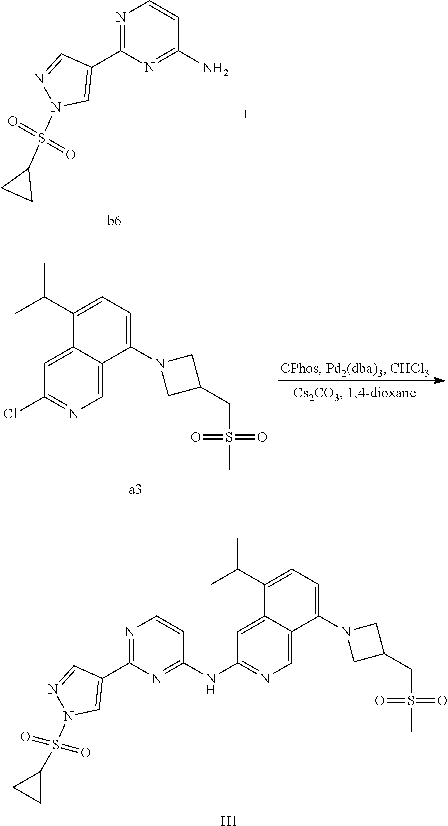 [SMILES: Nc1ccnc(-c2cnn(S(=O)(=O)C3CC3)c2)n1 ; CC(C)c1ccc(N2CC(CS(C)(=O)=O)C2)c2cnc(Cl)cc12 ; CC(C)c1ccc(N2CC(CS(C)(=O)=O)C2)c2cnc(Nc3ccnc(-c4cnn(S(=O)(=O)C5CC5)c4)n3)cc12 ; "H1"]

Under nitrogen protection, intermediate a3 (35 mg, 0.10 mmol), Cs₂CO₃ (48 mg, 0.15 mmol) and intermediate b6 (32 mg, 0.12 mmol) were mixed in 3 mL of 1,4-dioxane. Pd₂(dba)₃CHCl₃ (10 mg, 0.01 mmol) and C-phos (4.5 mg, 0.01 mmol) were added. The mixture was heated to 100° C. and reacted for 12 hours. The reaction was completed by LC-MS monitoring. The mixture was filtered. The filtrate was concentrated, and separated by HPLC preparative chromatography to obtain a yellow solid H1 (2.7 mg), yield: 5%, LCMS: ESI-MS (m/z): 582.1 [M+H]⁺.

¹H NMR (400 MHz, DMSO-d₆) δ 10.37 (d, J=8.4 Hz, 1H), 9.10 (s, 1H), 8.77 (s, 1H), 8.70 (s, 1H), 8.51 (s, 1H), 8.43 (d, J=5.9 Hz, 1H), 7.45 (d, J=8.0 Hz, 1H), 7.25 (s, 1H), 6.45 (d, J=8.1 Hz, 1H), 4.41 (t, J=7.7 Hz, 2H), 3.98 (t, J=6.9 Hz, 2H), 3.64-3.52 (m, 3H), 3.31-3.23 (m, 2H), 3.02 (s, 3H), 1.37 (d, J=6.8 Hz, 6H), 1.30 (ddd, J=21.4, 16.3, 9.3 Hz, 4H).

Referring to the synthetic route of compound H1, the following target molecules were synthesized using a similar skeleton structure.

| Replacement of | | | |
|---|---|---|---|
| structure | | Target molecule | |
| Replace intermediate b6 with: 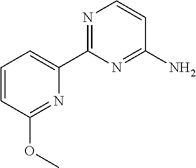 | b7 | 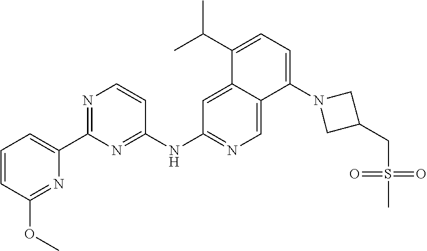 [SMILES: COc1cccc(-c2nccc(Nc3cc4c(C(C)C)ccc(N5CC(CS(C)(=O)=O)C5)c4cn3)n2)n1] | H2 |
| Replace intermediate a3 with: 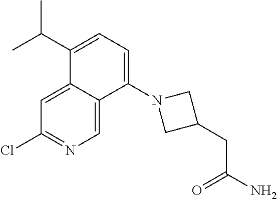 [SMILES: CC(C)c1ccc(N2CC(CC(N)=O)C2)c2cnc(Cl)cc12] | a6 | 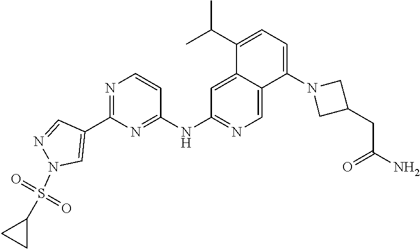 [SMILES: CC(C)c1ccc(N2CC(CC(N)=O)C2)c2cnc(Nc3ccnc(-c4cnn(S(=O)(=O)C5CC5)c4)n3)cc12] | H4 |
| Replace intermediate a3 with: 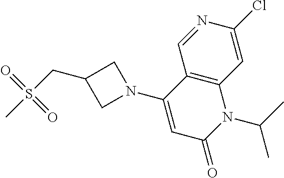 [SMILES: CC(C)n1c(=O)cc(N2CC(CS(C)(=O)=O)C2)c2cnc(Cl)cc21] | a11 | 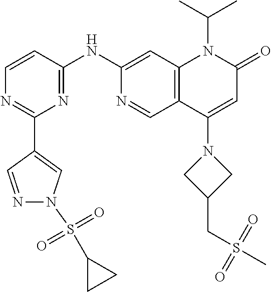 [SMILES: CC(C)n1c(=O)cc(N2CC(CS(C)(=O)=O)C2)c2cnc(Nc3ccnc(-c4cnn(S(=O)(=O)C5CC5)c4)n3)cc21] | H5 |
| Replace intermediate a3 with: 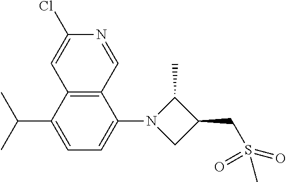 [SMILES: CC(C)c1ccc(N2C[C@H](CS(C)(=O)=O)[C@H]2C)c2cnc(Cl)cc12] | a26 | 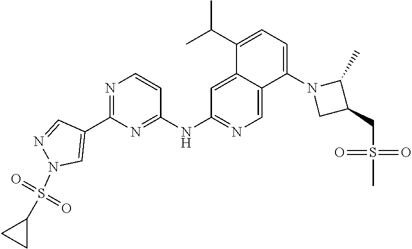 [SMILES: CC(C)c1ccc(N2C[C@H](CS(C)(=O)=O)[C@H]2C)c2cnc(Nc3ccnc(-c4cnn(S(=O)(=O)C5CC5)c4)n3)cc12] | H12 |
| Replace intermediate a3 with: 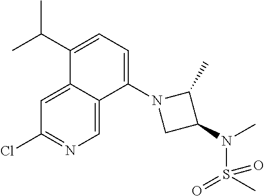 [SMILES: CC(C)c1ccc(N2C[C@H](N(C)S(C)(=O)=O)[C@H]2C)c2cnc(Cl)cc12] | a32 | 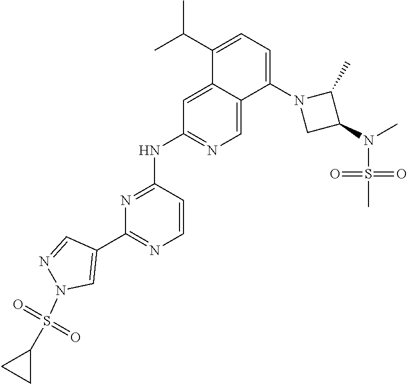 [SMILES: CC(C)c1ccc(N2C[C@H](N(C)S(C)(=O)=O)[C@H]2C)c2cnc(Nc3ccnc(-c4cnn(S(=O)(=O)C5CC5)c4)n3)cc12] | H23 |
| Replace intermediate a3 with: 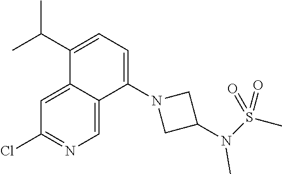 [SMILES: CC(C)c1ccc(N2CC(N(C)S(C)(=O)=O)C2)c2cnc(Cl)cc12] | a30 | 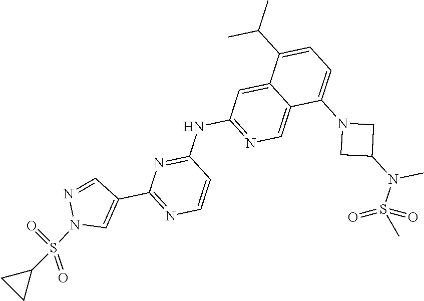 [SMILES: CC(C)c1ccc(N2CC(N(C)S(C)(=O)=O)C2)c2cnc(Nc3ccnc(-c4cnn(S(=O)(=O)C5CC5)c4)n3)cc12] | H25 |
| Replace intermediate a3 with: 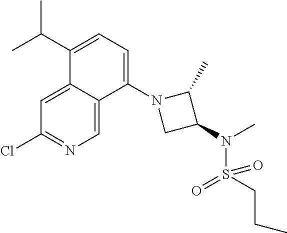 [SMILES: CCCS(=O)(=O)N(C)[C@H]1CN(c2ccc(C(C)C)c3cc(Cl)ncc23)[C@@H]1C] | a41 | 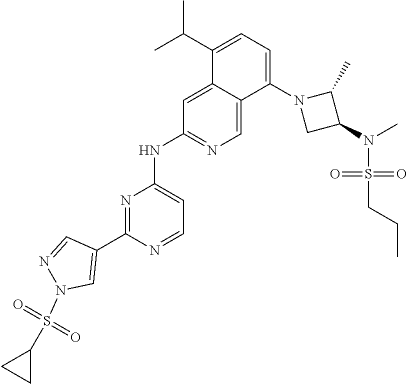 [SMILES: CCCS(=O)(=O)N(C)[C@H]1CN(c2ccc(C(C)C)c3cc(Nc4ccnc(-c5cnn(S(=O)(=O)C6CC6)c5)n4)ncc23)[C@@H]1C] | H33 |
| Replace intermediate a3 with: 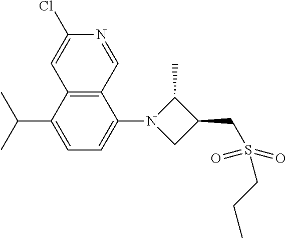 [SMILES: CCCS(=O)(=O)C[C@H]1CN(c2ccc(C(C)C)c3cc(Cl)ncc23)[C@@H]1C] | 43 | 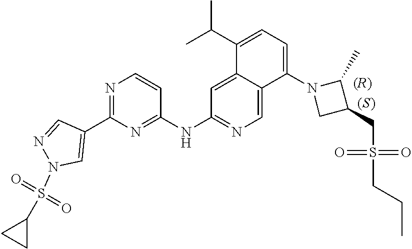 | H34 |

| | | | LC-MS |
|---|---|---|---|
| Replacement of structure | | ¹H NMR | [M + H]⁺ |
| Replace | | ¹H NMR (400 MHz, DMSO-d₆) δ | 519.2 |
| intermediate | | 10.38 (s, 1H), 9.10 (s, 1H), 8.86 (s, | |
| b6 with: | | 1H), 8.56 (d, J = 5.8 Hz, 1H), 8.28 | |
| | | (s, 1H), 8.00 (d, J = 6.7 Hz, 1H), | |
| 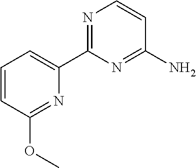 | b7 | 7.93 − 7.87 (m, 1H), 7.44 − 7.32 (m, 2H), 7.00 (d, J = 7.5 Hz, 1H), 6.42 (d, J = 8.0 Hz, 1H), 4.41 (t, J = 7.7 Hz, 2H), 4.08 − 3.89 (m, 5H), 3.58 (m, 3H), 3.28 − 3.24 (m, 1H), 3.02 (s, 3H), 1.28 (d, J = 6.8 Hz, 6H). | |
| Replace | | ¹H NMR (400 MHz, DMSO-d₆) δ | 547.0 |
| intermediate | | 10.37 (s, 1H), 9.11 (s, 1H), 8.76 (s, | |
| a3 with: | | 1H), 8.69 (s, 1H), 8.51 (s, 1H), 8.43 | |
| | | (d, J = 5.9 Hz, 1H), 7.43 (d, J = 8.0 | |
| 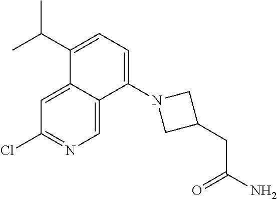 [SMILES: CC(C)c1ccc(N2CC(CC(N)=O)C2)c2cnc(Cl)cc12] | a6 | Hz, 1H), 7.38 (s, 1H), 7.23 (s, 1H), 6.86 (s, 1H), 6.41 (d, J = 8.1 Hz, 1H), 4.32 (t, J = 7.6 Hz, 2H), 3.84 (t, J = 6.6 Hz, 2H), 3.61 − 3.50 (m, 1H), 3.31 − 3.26 (m, 1H), 3.07 − 2.98 (m, 1H), 2.49 − 2.47 (m, 2H), 1.37 (d, J = 6.8 Hz, 6H), 1.34 − 1.21 (m, 4H). | |
| Replace | | ¹H NMR (400 MHz, DMSO-d₆) δ | 598.8 |
| intermediate | | 10.43 (s, 1H), 8.63 (d, J = 8.5 Hz, | |
| a3 with: | | 1H), 8.57 (s, 1H), 8.47 (d, J = 5.9 | |
| | | Hz, 1H), 8.42 (s, 1H), 7.94 (s, 1H), | |
| 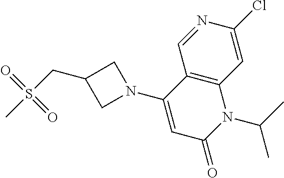 [SMILES: CC(C)n1c(=O)cc(N2CC(CS(C)(=O)=O)C2)c2cnc(Cl)cc21] | a11 | 7.67 (s, 1H), 5.43 − 5.18 (m, 1H), 5.09 (s, 1H), 4.44 (t, J = 8.3 Hz, 2H), 4.05 (dd, J = 8.1, 6.4 Hz, 2H), 3.55 (d, J = 7.4 Hz, 2H), 3.27 − 3.14 (m, 2H), 2.97 (s, 3H), 1.51 (d, J = 7.1 Hz, 6H), 1.35 − 1.28 (m, 2H), 1.25 − 1.20 (m, 2H). | |
| Replace | | ¹H NMR (400 MHz, DMSO-d₆) δ | 596.2 |
| intermediate | | 10.42 (s, 1H), 9.11 (s, 1H), 8.80 (s, | |
| a3 with: | | 1H), 8.70 (s, 1H), 8.52 (s, 1H), 8.43 | |
| | | (d, J = 5.9 Hz, 1H), 8.38 (s, 1H), | |
| 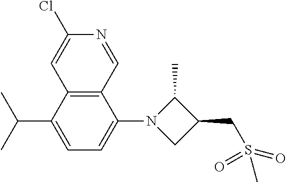 [SMILES: CC(C)c1ccc(N2C[C@H](CS(C)(=O)=O)[C@H]2C)c2cnc(Cl)cc12] | a26 | 7.46 (d, J = 8.0 Hz, 1H), 7.23 (s, 1H), 6.60 (d, J = 8.1 Hz, 1H), 4.69 (t, J = 7.4 Hz, 1H), 4.24 − 4.17 (m, 1H), 3.66 (t, J = 7.1 Hz, 1H), 3.62 − 3.47 (m, 4H), 3.00 (s, 3H), 2.90 (dd, J = 14.1, 7.2 Hz, 1H), 1.43 (d, J = 6.0 Hz, 3H), 1.38 (dd, J = 6.6, 2.4 Hz, 6H), 1.35 − 1.28 (m, 2H), 1.28 − 1.23 (m, 2H). | |
| Replace | | ¹H NMR (400 MHz, DMSO-d₆) δ | 611.3 |
| intermediate | | 10.42 (s, 1H), 9.13 (s, 1H), 8.81 (s, | |
| a3 with: | | 1H), 8.70 (s, 1H), 8.52 (s, 1H), 8.44 | |
| | | (d, J = 5.9 Hz, 1H), 7.48 (d, J = 8.0 | |
| 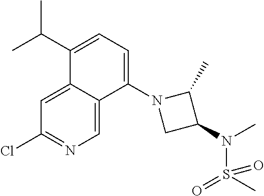 [SMILES: CC(C)c1ccc(N2C[C@H](N(C)S(C)(=O)=O)[C@H]2C)c2cnc(Cl)cc12] | a32 | Hz, 1H), 7.24 (s, 1H), 6.70 (d, J = 8.1 Hz, 1H), 4.66 (t, J = 7.3 Hz, 1H), 4.48 − 4.43 (m, 1H), 4.24 − 4.19 (m, 1H), 3.83 (t, J = 7.1 Hz, 1H), 3.62 − 3.56 (m, 1H), 3.29 − 3.27(m,1H), 2.96 (s, 3H), 2.83 (s, 3H), 1.42 −1.35 (m, 9H), 1.34 − 1.16 (m, 4H). | |
| Replace | | ¹H NMR (400 MHz, DMSO-d₆) δ | 597.0 |
| intermediate | | 10.37 (s, 1H), 9.14 (s, 1H), 8.76 (s, | |
| a3 with: | | 1H), 8.69 (s, 1H), 8.51 (s, 1H), 8.44 | |
| | | (d, J = 5.9 Hz, 1H), 8.40 (s, 1H), 7.46 | |
| 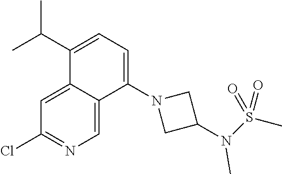 [SMILES: CC(C)c1ccc(N2CC(N(C)S(C)(=O)=O)C2)c2cnc(Cl)cc12] | a30 | (d, J = 8.0 Hz, 1H), 7.26 (d, J = 4.7 Hz, 1H), 6.50 (d, J = 8.0 Hz, 1H), 4.71 − 4.64 (m, 1H), 4.42 (t, J = 7.9 Hz, 2H), 4.26 − 4.21 (m, 2H), 3.60 − 3.56 (m, 1H), 3.27 − 3.26 (m, 1H), 2.96 (s, 3H), 2.94 (s, 3H), 1.40 − 1.35 (m, 6H), 1.34 − 1.25 (m, 4H). | |
| Replace | | ¹H NMR (400 MHz, DMSO-d₆) δ | 639.8 |
| intermediate | | 10.36 (s, 1H), 9.09 (s, 1H), 8.68 (s, | |
| a3 with: | | 1H), 8.56 (s, 1H), 8.47 (s, 1H), 8.40 | |
| | | (d, J = 5.9 Hz, 1H), 7.35 (d, J = 7.8 | |
| 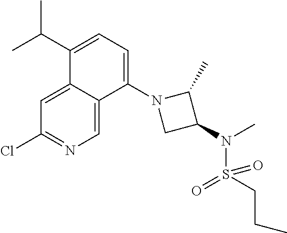 [SMILES: CCCS(=O)(=O)N(C)[C@H]1CN(c2ccc(C(C)C)c3cc(Cl)ncc23)[C@@H]1C] | a41 | Hz, 1H), 7.25 (s, 1H), 6.60 (d, J = 8.0 Hz, 1H), 4.61 (t, J = 7.5 Hz, 1H), 4.43 (t, J = 6.1 Hz, 1H), 4.28 − 4.23 (m, 1H), 3.82 (t, J = 7.2 Hz, 1H), 3.25 − 3.20 (m, 2H), 3.06 − 3.01 (m, 2H), 2.91 (t, J = 7.4 Hz, 2H), 2.82 (s, 3H), 1.69 − 1.64 (m, 3H), 1.35 (d, J = 6.0 Hz, 6H), 0.97 (t, J = 7.4 Hz, 4H), 0.88 (t, J = 7.3 Hz, 3H). | |
| Replace | | ¹H NMR (400 MHz, DMSO-d₆) δ | 624.8 |
| intermediate | | 10.40 (s, 1H), 9.11 (s, 1H), 8.79 (s, | |
| a3 with: | | 1H), 8.70 (s, 1H), 8.51 (s, 1H), 8.43 | |
| | | (d, J = 5.9 Hz, 1H), 7.46 (d, J = 8.0 | |
| 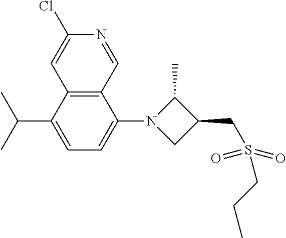 [SMILES: CCCS(=O)(=O)C[C@H]1CN(c2ccc(C(C)C)c3cc(Cl)ncc23)[C@@H]1C] | 43 | Hz, 1H), 7.23 (s, 1H), 6.60 (d, J = 8.2 Hz, 1H), 4.68 (t, J = 7.4 Hz, 1H), 4.23 − 4.18 (m, 1H), 3.68 − 3.48 (m, 5H), 3.30 − 3.26 (m, 1H), 3.08 (dd, J = 9.0, 6.7 Hz, 2H), 2.92 − 2.87(m, 1H), 1.72 (dd, J = 15.4, 7.5 Hz, 2H), 1.43 (d, J = 6.0 Hz, 3H), 1.38 (dd, J = 6.8, 2.2 Hz, 6H), 1.30 − 1.19 (m, 1H), 1.00 (t, J = 7.4 Hz, 3H). | |

### Example 6

### Preparation of Target Molecules H3, H16-H11, H13-H22, H24, and H26-H32

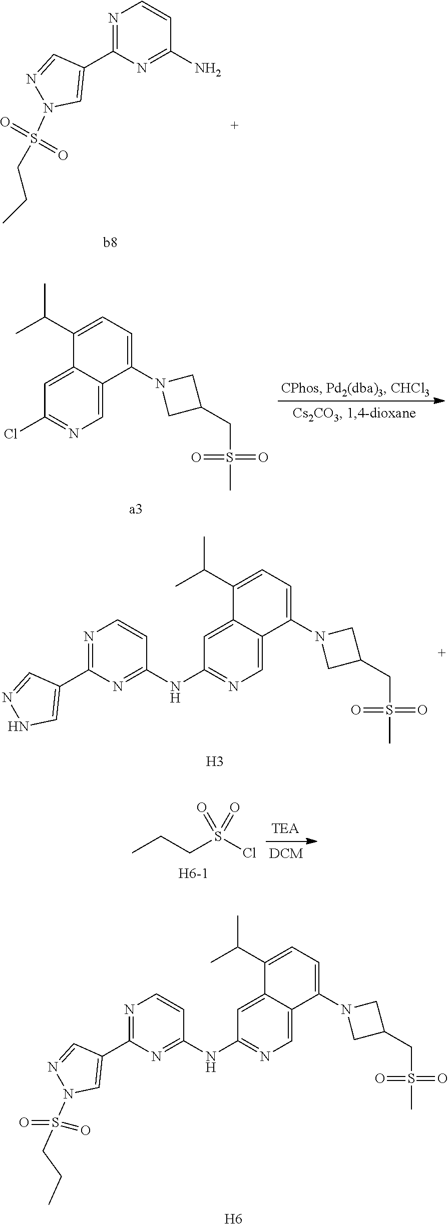

Under nitrogen protection, intermediate a3 (9 mg, 0.026 mmol), Cs₂CO₃ (17 mg, 0.052 mmol) and intermediate b8 (7 mg, 0.026 mmol) were mixed in 1.5 mL of 1,4-dioxane. Pd₂(dba)₃CHCl₃ (81 mg, 0.008 mmol) and C-phos (3.0 mg, 0.008 mmol) were added. The mixture was heated to 100° C. and reacted for 20 hours. The reaction was completed by LC-MS monitoring. The mixture was filtered. The filtrate was concentrated, and separated by HPLC preparative chromatography to obtain a yellow solid H3 (5.0 mg), yield: 44%, LCMS: ESI-MS (m/z): 437.3 [M+H]⁺.

In an ice bath, compound H3 (5 mg, 0.011 mmol) was dissolved in 1.5 mL of dichloromethane. Triethylamine (3 mg, 0.021 mmol) and n-propylsulfonyl chloride (3 mg, 0.021 mmol) were added. The mixture was warmed to room temperature and reacted for 1 hour. The reaction was stopped. 10 mL of water was added to the reaction solution. The mixture was extracted with ethyl acetate, concentrated, and separated by HPLC preparative chromatography to obtain compound H6 (2.1 mg), LCMS: ESI-MS (m/z): 584.0 [M+H]⁺.

1H NMR (400 MHz, DMSO-d₆) δ 10.43 (s, 1H), 9.11 (s, 1H), 8.77 (s, 1H), 8.70 (s, 1H), 8.53 (s, 1H), 8.44 (d, J=5.9 Hz, 1H), 7.46 (d, J=8.0 Hz, 1H), 7.25 (bs, 1H), 6.45 (d, J=8.1 Hz, 1H), 4.43-4.39 (m, 2H), 3.98-3.82 (m, 2H), 3.586-3.52 (m, 2H), 3.60-3.57 (m, 3H), 3.29-3.26 (m, 1H), 3.02 (s, 3H), 1.64-1.58 (m, 2H), 1.38 (d, J=6.8 Hz, 6H), 0.96-0.90 (m, 3H).

Referring to the synthetic route of compound H6, the following target molecules were synthesized using a similar skeleton structure.

| Replacement of | | | |
|---|---|---|---|
| structure | | Target molecule | |
| Replace compound H6-1 with: 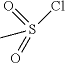 | | 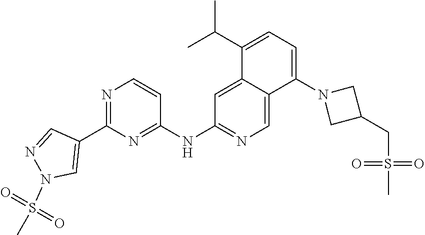 [SMILES: CC(C)c1ccc(N2CC(CS(C)(=O)=O)C2)c2cnc(Nc3ccnc(-c4cnn(S(C)(=O)=O)c4)n3)cc12] | H7 |
| Replace compound H6-1 with: Tf₂O | | 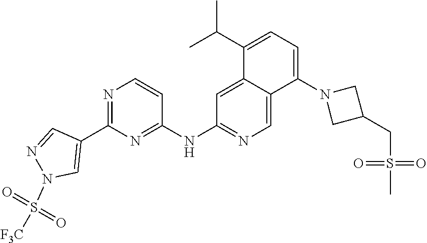 [SMILES: CC(C)c1ccc(N2CC(CS(C)(=O)=O)C2)c2cnc(Nc3ccnc(-c4cnn(S(=O)(=O)C(F)(F)F)c4)n3)cc12] | H8 |
| Replace compound H6-1 with: 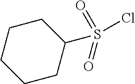 | | 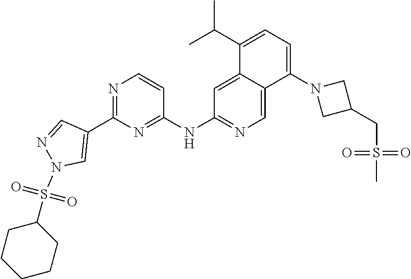 [SMILES: CC(C)c1ccc(N2CC(CS(C)(=O)=O)C2)c2cnc(Nc3ccnc(-c4cnn(S(=O)(=O)C5CCCCC5)c4)n3)cc12] | H9 |
| Replace compound H6-1 with: 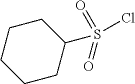 | | 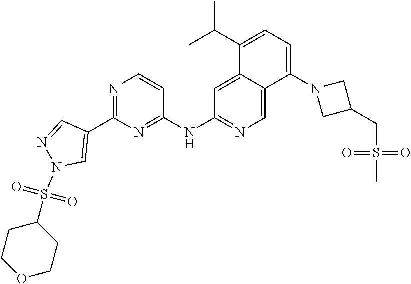 [SMILES: CC(C)c1ccc(N2CC(CS(C)(=O)=O)C2)c2cnc(Nc3ccnc(-c4cnn(S(=O)(=O)C5CCOCC5)c4)n3)cc12] | H10 |
| Replace compound H6-1 with: 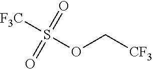 [SMILES: O=S(=O)(OCC(F)(F)F)C(F)(F)F] | | 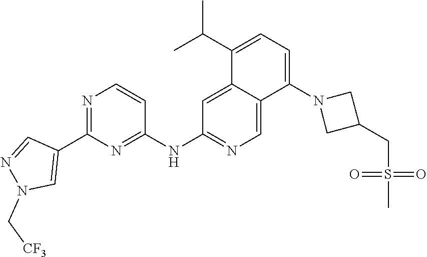 [SMILES: CC(C)c1ccc(N2CC(CS(C)(=O)=O)C2)c2cnc(Nc3ccnc(-c4cnn(CC(F)(F)F)c4)n3)cc12] | H11 |
| Replace compound H6-1 with: 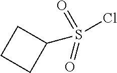 | | 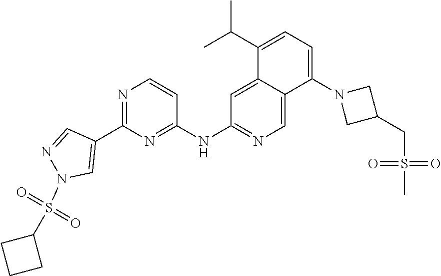 [SMILES: CC(C)c1ccc(N2CC(CS(C)(=O)=O)C2)c2cnc(Nc3ccnc(-c4cnn(S(=O)(=O)C5CCC5)c4)n3)cc12] | H13 |
| Replace compound H6-1 with: 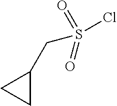 | | 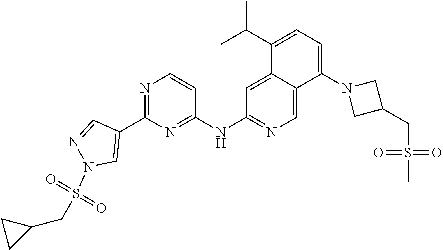 [SMILES: CC(C)c1ccc(N2CC(CS(C)(=O)=O)C2)c2cnc(Nc3ccnc(-c4cnn(S(=O)(=O)CC5CC5)c4)n3)cc12] | H14 |
| Replace compound H6-1 with: 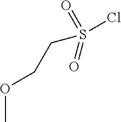 | | 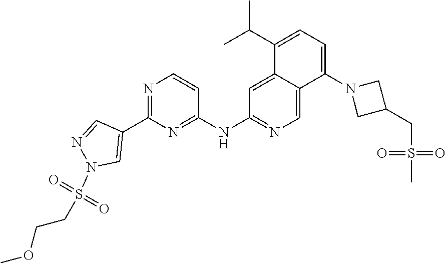 [SMILES: COCCS(=O)(=O)n1cc(-c2nccc(Nc3cc4c(C(C)C)ccc(N5CC(CS(C)(=O)=O)C5)c4cn3)n2)cn1] | H15 |
| Replace compound H6-1 with: 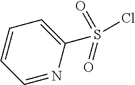 | | 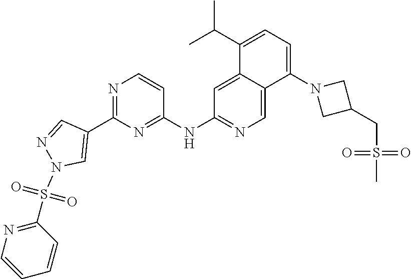 [SMILES: CC(C)c1ccc(N2CC(CS(C)(=O)=O)C2)c2cnc(Nc3ccnc(-c4cnn(S(=O)(=O)c5ccccn5)c4)n3)cc12] | H16 |
| Replace compound H6-1 with: 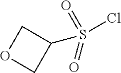 | | 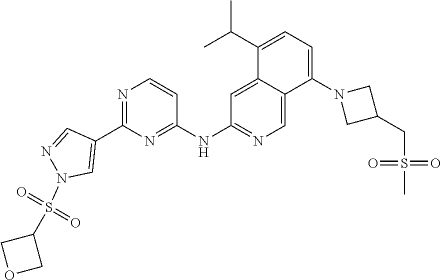 [SMILES: CC(C)c1ccc(N2CC(CS(C)(=O)=O)C2)c2cnc(Nc3ccnc(-c4cnn(S(=O)(=O)C5COC5)c4)n3)cc12] | H17 |
| Replace compound H6-1 with: 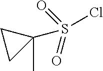 | | 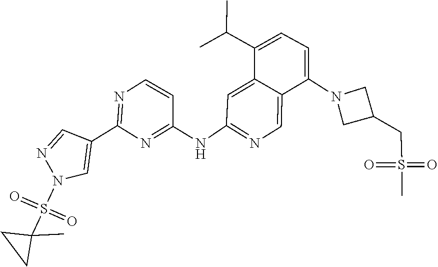 [SMILES: CC(C)c1ccc(N2CC(CS(C)(=O)=O)C2)c2cnc(Nc3ccnc(-c4cnn(S(=O)(=O)C5(C)CC5)c4)n3)cc12] | H18 |
| Replace compound H6-1 with: 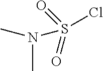 | | 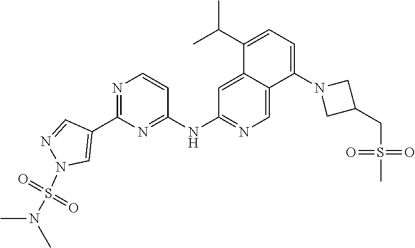 [SMILES: CC(C)c1ccc(N2CC(CS(C)(=O)=O)C2)c2cnc(Nc3ccnc(-c4cnn(S(=O)(=O)N(C)C)c4)n3)cc12] | H19 |
| Replace compound H6-1 with: 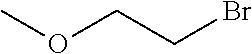 | | 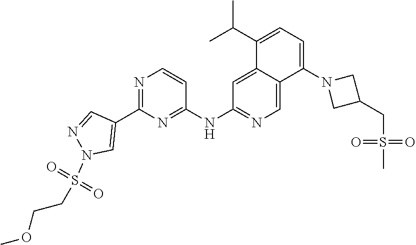 [SMILES: COCCS(=O)(=O)n1cc(-c2nccc(Nc3cc4c(C(C)C)ccc(N5CC(CS(C)(=O)=O)C5)c4cn3)n2)cn1] | H20 |
| Replace intermediate a3 with: 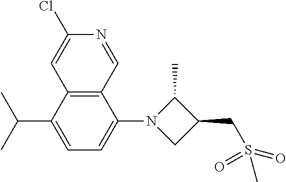 [SMILES: CC(C)c1ccc(N2C[C@H](CS(C)(=O)=O)[C@H]2C)c2cnc(Cl)cc12] | a26 | 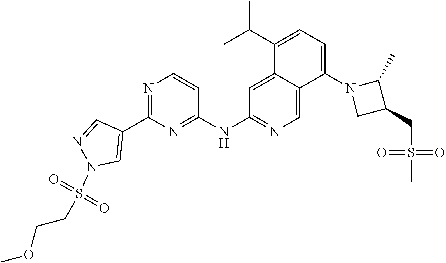 [SMILES: COCCS(=O)(=O)n1cc(-c2nccc(Nc3cc4c(C(C)C)ccc(N5C[C@H](CS(C)(=O)=O)[C@H]5C)c4cn3)n2)cn1] | H21 |

| Replace compound H6-1 with: 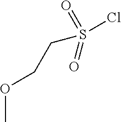 | | | |
|---|---|---|---|
| Replace intermediate a3 with: 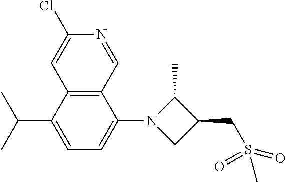 [SMILES: CC(C)c1ccc(N2C[C@H](CS(C)(=O)=O)[C@H]2C)c2cnc(Cl)cc12] Replace compound H6-1 with: | a26 | 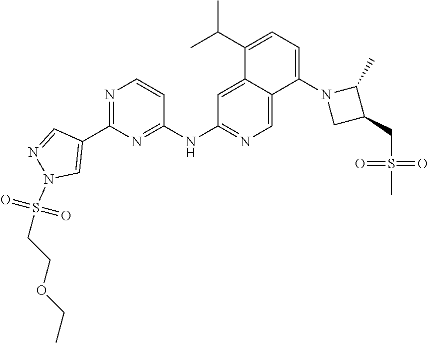 [SMILES: CCOCCS(=O)(=O)n1cc(-c2nccc(Nc3cc4c(C(C)C)ccc(N5C[C@H](CS(C)(=O)=O)[C@H]5C)c4cn3)n2)cn1] | H22 |
| 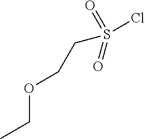 | | | |
| Replace compound H6-1 with: 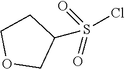 | | 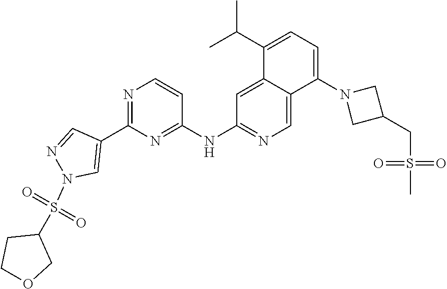 [SMILES: CC(C)c1ccc(N2CC(CS(C)(=O)=O)C2)c2cnc(Nc3ccnc(-c4cnn(S(=O)(=O)C5CCOC5)c4)n3)cc12] | H24 |
| Replace compound H6-1 with: 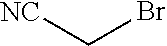 | | 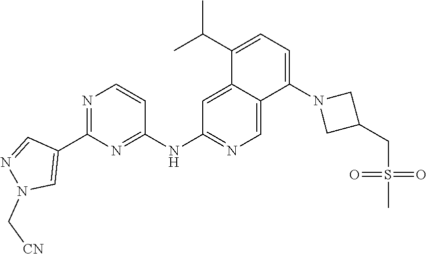 [SMILES: CC(C)c1ccc(N2CC(CS(C)(=O)=O)C2)c2cnc(Nc3ccnc(-c4cnn(CC#N)c4)n3)cc12] | H26 |
| Replace compound H6-1 with: 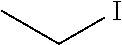 | | 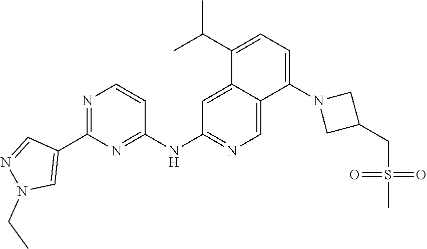 [SMILES: CCn1cc(-c2nccc(Nc3cc4c(C(C)C)ccc(N5CC(CS(C)(=O)=O)C5)c4cn3)n2)cn1] | H27 |
| Replace intermediate a3 with: 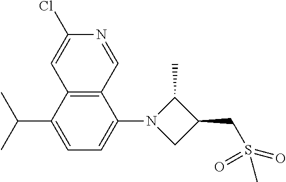 [SMILES: CC(C)c1ccc(N2C[C@H](CS(C)(=O)=O)[C@H]2C)c2cnc(Cl)cc12] | a26 | 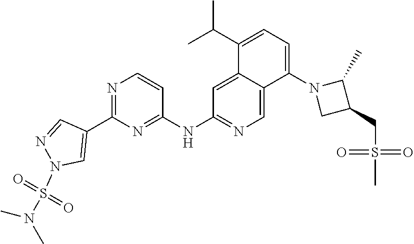 [SMILES: CC(C)c1ccc(N2C[C@H](CS(C)(=O)=O)[C@H]2C)c2cnc(Nc3ccnc(-c4cnn(S(=O)(=O)N(C)C)c4)n3)cc12] | H28 |
| Replace compound H6-1 with: 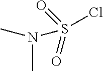 | | | |
| Replace compound H6-1 with: Methyl iodide MeI | | 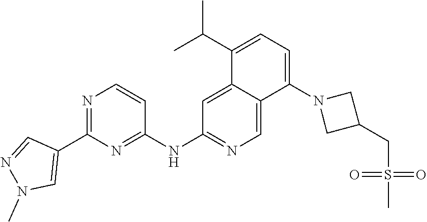 [SMILES: CC(C)c1ccc(N2CC(CS(C)(=O)=O)C2)c2cnc(Nc3ccnc(-c4cnn(C)c4)n3)cc12] | H29 |
| Replace intermediate a3 with: 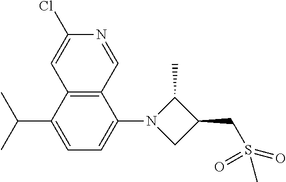 [SMILES: CC(C)c1ccc(N2C[C@H](CS(C)(=O)=O)[C@H]2C)c2cnc(Cl)cc12] | a26 | 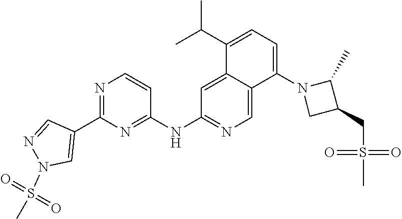 [SMILES: CC(C)c1ccc(N2C[C@H](CS(C)(=O)=O)[C@H]2C)c2cnc(Nc3ccnc(-c4cnn(S(C)(=O)=O)c4)n3)cc12] | H30 |
| Replace | | | |
| compound H6-1 | | | |
| with: | | | |
| 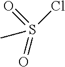 | | | |
| Replace intermediate a3 with: 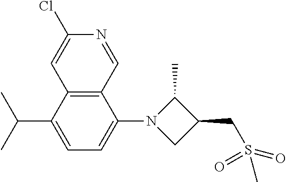 [SMILES: CC(C)c1ccc(N2C[C@H](CS(C)(=O)=O)[C@H]2C)c2cnc(Cl)cc12] Replace compound H6-1 with: | a26 | 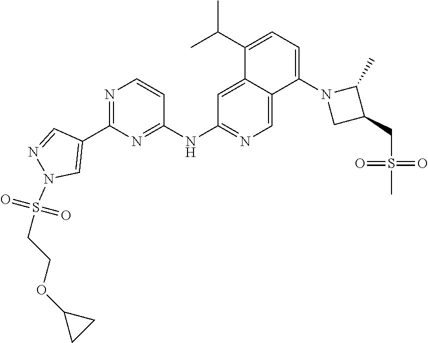 [SMILES: CC(C)c1ccc(N2C[C@H](CS(C)(=O)=O)[C@H]2C)c2cnc(Nc3ccnc(-c4cnn(S(=O)(=O)CCOC5CC5)c4)n3)cc12] | H31 |
| 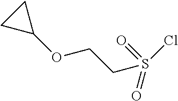 | | | |
| Replace intermediate a3 with: 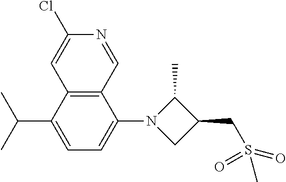 [SMILES: CC(C)c1ccc(N2C[C@H](CS(C)(=O)=O)[C@H]2C)c2cnc(Cl)cc12] Replace compound H6-1 with: | a26 | 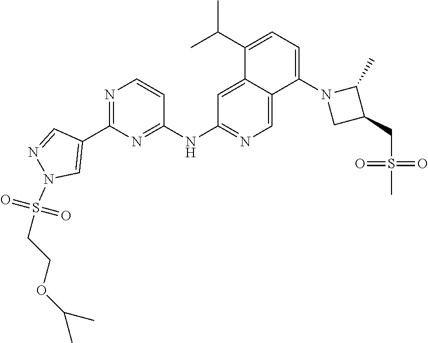 [SMILES: CC(C)OCCS(=O)(=O)n1cc(-c2nccc(Nc3cc4c(C(C)C)ccc(N5C[C@H](CS(C)(=O)=O)[C@H]5C)c4cn3)n2)cn1] | H32 |

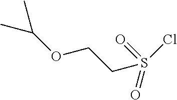

| Replacement of | | | LC-MS |
|---|---|---|---|
| structure | | ¹H NMR | [M + H]⁺ |
| Replace | | HNMR (400 MHz, DMSO-d₆) δ | 555.9 |
| compound H6-1 | | 10.38 (s, 1H), 9.10 (s, 1H), 8.79 (s, | |
| with: | | 1H), 8.69 (s, 1H), 8.50 (s, 1H), 8.43 | |
| | | (d, J = 5.9 Hz, 1H), 7.45 (d, J = 8.0 | |
| 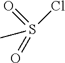 | | Hz, 1H), 7.24 (s, 1H), 6.45 (d, J = 8.1 Hz, 1H), 4.39-4.41 (m, 2H), 3.98-4.01 (m, 2H), 3.69 (s, 3H), 3.58-3.60 (m, 3H), 3.30 − 3.25 (m, 1H), 3.02 (s, 3H), 1.38 (d, J = 6.8 Hz, 6H). | |
| Replace | | 1H NMR (400 MHz, DMSO-d₆) δ | 609.9 |
| compound H6-1 | | 10.47 (s, 1H), 9.11 (s, 1H), 8.96 (s, | |
| with: Tf₂O | | 1H), 8.84 (s, 1H), 8.72 (s, 1H), 8.48 | |
| | | (d, J = 5.9 Hz, 1H), 8.25 (s, 2H), 7.45 | |
| | | (d, J = 8.0 Hz, 1H), 7.33 (s, 1H), 6.45 | |
| | | (d, J = 8.1 Hz, 1H), 4.39-4.43 (m, | |
| | | 2H), 4.01 − 3.96 (m, 2H), 3.60 (d, J = | |
| | | 7.4 Hz, 2H), 3.55 (d, J = 6.4 Hz, 1H), | |
| | | 3.28 − 3.26 (m, 1H), 3.02 (s, 3H), 1.35 | |
| | | (d, J = 7.9 Hz, 6H). | |
| Replace | | 1H NMR (400 MHz, DMSO-d₆) δ | 614.0 |
| compound H6-1 | | 10.40 (s, 1H), 9.10 (s, 1H), 8.77 (s, | |
| with: | | 1H), 8.66 (s, 1H), 8.53 (s, 1H), 8.44 | |
| | | (d, J = 5.9 Hz, 1H), 7.45 (d, J = 8.0 | |
| 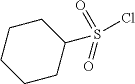 | | Hz, 1H), 7.24 (s, 1H), 6.45 (d, J = 8.1 Hz, 1H), 4.39-4.43 (m, 2H), 3.96-4.00 (m, 2H), 3.91 − 3.84 (m, 1H), 3.59 (d, J = 8.0 Hz, 2H), 3.56 (d, J = 6.8 Hz, 1H), 3.28-3.31 (m, 1H), 3.02 (s, 3H), 1.90 (d, J = 10.0 Hz, 2H), 1.77 (d, J = | |
| | | 13.2 Hz, 2H), 1.59 (d, J = 13.0 Hz, | |
| | | 1H), 1.44 (dd, J = 12.4, 3.1 Hz, 2H), | |
| | | 1.38 (d, J = 6.8 Hz, 6H), 1.32 − 1.24 | |
| | | (m, 2H), 1.15 − 1.08 (m, 1H). | |
| Replace | | ¹H NMR (400 MHz, DMSO-d₆) δ | 626.0 |
| compound H6-1 | | 10.40 (s, 1H), 9.10 (s, 1H), 8.76 (s, | |
| with: | | 1H), 8.68 (s, 1H), 8.55 (s, 1H), 8.44 | |
| | | (d, J = 5.9 Hz, 1H), 7.46 (d, J = 8.0 | |
| 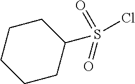 | | Hz, 1H), 7.24 (s, 1H), 6.45 (d, J = 8.1 Hz, 1H), 4.41 (t, J = 7.7 Hz, 2H), 4.24 − 4.20 (m, 1H), 3.98 (t, J = 6.9 Hz, 2H), 3.91 (dd, J = 11.6, 3.3 Hz, 2H), 3.60 (d, J = 7.4 Hz, 2H), 3.57 − 3.53 (m, 1H), 3.32 − 3.26 (m, 3H), 3.02 (s, | |
| | | 3H), 1.78 − 1.65 (m, 4H), 1.38 (d, J = | |
| | | 6.8 Hz, 6H). | |
| Replace | | ¹H NMR (400 MHz, DMSO-d₆) δ | 560.0 |
| compound H6-1 | | 10.27 (s, 1H), 9.10 (s, 1H), 8.67 (s, | |
| with: | | 1H), 8.44 (s, 1H), 8.38 (d, J = 5.9 Hz, | |
| | | 1H), 8.20 (s, 1H), 7.45 (d, J = 8.0 Hz, | |
| 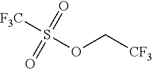 [SMILES: O=S(=O)(OCC(F)(F)F)C(F)(F)F] | | 1H), 7.23 (s, 1H), 6.44 (d, J = 8.1 Hz, 1H), 5.31-5.24 (m, 2H), 4.41 (t, J = 7.7 Hz, 2H), 3.98 (t, J = 6.9 Hz, 2H), 3.60 (d, J = 7.3 Hz, 2H), 3.58-3.55 (m, 1H), 3.29-3.27 (m, 1H), 3.02 (s, | |
| | | 3H), 1.34 (d, J = 6.8 Hz, 6H). | |
| Replace | | ¹H NMR (400 MHz, DMSO-d₆) δ | 596.0 |
| compound H6-1 | | 0.36 (s, 1H), 9.10 (s, 1H), 8.77 (s, | |
| with: | | 1H), 8.68 (s, 1H), 8.51 (s, 1H), 8.43 | |
| | | (d, J = 5.9 Hz, 1H), 7.45 (d, J = 8.0 | |
| 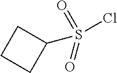 | | Hz, 1H), 7.24 (d, J = 5.2 Hz, 1H), 6.45 (d, J = 8.1 Hz, 1H), 4.65-4.60 (m, 1H), 4.43-4.39 (m, 2H), 3.98-3.97 (m, 2H), 3.58 (d, J = 7.4 Hz, 3H), 3.27 (m, 1H), 3.02 (s, 3H), 2.34-2.28 (m, | |
| | | 2H), 2.00-1.93 (m, 2H), 1.38 (d, J = | |
| | | 6.8 Hz, 6H). | |
| Replace | | ¹H NMR (400 MHz, DMSO-d₆) δ | 596.1 |
| compound H6-1 | | 10.39 (s, 1H), 9.10 (s, 1H), 8.79 (s, | |
| with: | | 1H), 8.70 (s, 1H), 8.52 (s, 1H), 8.43 | |
| | | (d, J = 5.9 Hz, 1H), 7.45 (d, J = 8.1 | |
| 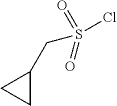 | | Hz, 1H), 7.23 (s, 1H), 6.45 (d, J = 8.0 Hz, 1H), 4.41 (t, J = 7.6 Hz, 2H), 3.98 (t, J = 6.9 Hz, 2H), 3.82 (d, J = 7.1 Hz, 2H), 3.58 (t, J = 9.2 Hz, 4H), 3.31 3.27 (m, 1H), 3.02 (s, 3H), 1.40 − 1.37 (m, 6H), 0.92 − 0.83 (m, 1H), 0.46 (d, J = 4.7 Hz, 2H), 0.12 (d, J = | |
| | | 5.9 Hz, 2H). | |
| Replace | | ¹H NMR (400 MHz, DMSO-d₆) δ | 600.1 |
| compound H6-1 | | 10.38 (s, 1H), 9.10 (s, 1H), 8.78 (s, | |
| with: | | 1H), 8.65 (s, 1H), 8.49 (s, 1H), 8.43 | |
| | | (d, J = 5.9 Hz, 1H), 7.46 (d, J = 7.9 | |
| 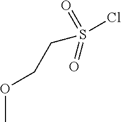 | | Hz, 1H), 7.23 (s, 1H), 6.45 (d, J = 8.1 Hz, 1H), 4.41 (t, J = 7.6 Hz, 2H), 4.10 (t, J = 5.4 Hz, 2H), 3.98 (t, J = 6.9 Hz, 2H), 3.69 (t, J = 5.4 Hz, 2H), 3.60 (d, J = 7.4 Hz, 3H), 3.33- 3.28 (m, 1H), 3.09 (s, 3H), 3.02 (s, 3H), 1.39 (d, J = 6.8 Hz, 6H). | |
| Replace | | ¹H NMR (400 MHz, DMSO-d₆) δ | 619.0 |
| compound H6-1 | | 10.38 (s, 1H), 9.25 (d, J = 2.0 Hz, | |
| with: | | 1H), 9.10 (s, 1H), 8.98 (dd, J = 4.8, | |
| | | 1.5 Hz, 1H), 8.94 (s, 1H), 8.74 (s, | |
| 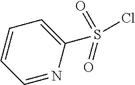 | | 1H), 8.52 − 8.49 (m, 1H), 8.48 (s, 1H), 8.42 (d, J = 5.9 Hz, 1H), 7.75 (dd, J = 7.7, 4.8 Hz, 1H), 7.46 (d, J = 8.0 Hz, 1H), 7.26 (s, 1H), 6.45 (d, J = 8.1 Hz, 1H), 4.41 (t, J = 7.7 Hz, 2H), 3.98 (t, J = 6.9 Hz, 2H), 3.61 (t, J = 7.0 Hz, | |
| | | 3H), 3.31 − 3.28 (m, 1H), 3.02 (s, 3H), | |
| | | 1.38 (d, J = 6.8 Hz, 6H). | |
| Replace | | ¹H NMR (400 MHz, DMSO-d₆) δ | 598.0 |
| compound H6-1 | | 10.38 (s, 1H), 9.10 (s, 1H), 8.77 (d, | |
| with: | | J = 14.3 Hz, 2H), 8.54 (s, 1H), 8.44 (d, | |
| | | J = 5.9 Hz, 1H), 7.45 (d, J =7.9 Hz, | |
| 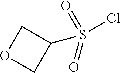 | | 1H), 7.26 (s, 1H), 6.45 (d, J = 8.0 Hz, 1H), 4.92 (t, J = 7.8 Hz, 2H), 4.89 (d, J = 5.9 Hz, 2H), 4.41 (t, J = 7.8 Hz, 2H), 3.98 (t, J = 7.8 Hz, 2H), 3.60 (d, J = 7.4 Hz, 3H), 3.02 (s, 3H), 1.37 (d, J = 6.8 Hz, 6H). | |
| Replace | | ¹H NMR (400 MHz, DMSO-d₆) δ | 596.1 |
| compound H6-1 | | 10.39 (s, 1H), 9.10 (s, 1H), 8.77 (s, | |
| with: | | 1H), 8.70 (s, 1H), 8.54 (s, 1H), 8.44 | |
| | | (d, J = 5.9 Hz, 1H), 7.45 (d, J = 8.0 | |
| 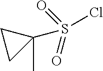 | | Hz, 1H), 7.25 (s, 1H), 6.45 (d, J = 8.1 Hz, 1H), 4.41 (t, J = 7.7 Hz, 2H), 3.98 (t, J = 6.9 Hz, 3H), 3.58 (dd, J = 15.2, 7.3 Hz, 4H), 3.31 − 3.28 (m, 1H), 3.02 (s, 4H), 1.61 (d, J = 4.6 Hz, 2H), 1.38 (d, J = 6.0 Hz, 9H), 1.24 − 1.21 (m, 2H). | |
| Replace | | ¹H NMR (400 MHz, DMSO-d₆) δ | 585.0 |
| compound H6-1 | | 10.36 (s, 1H), 9.10 (s, 1H), 8.78 (s, | |
| with: | | 1H), 8.64 (s, 1H), 8.46 (s, 1H), 8.42 | |
| | | (d, J = 5.9 Hz, 1H), 7.45 (d, J = 8.0 | |
| 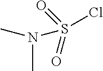 | | Hz, 1H), 7.23 (s, 1H), 6.45 (d, J = 8.1 Hz, 1H), 4.41 (t, J = 7.7 Hz, 2H), 3.98 (t, J = 6.8 Hz, 2H), 3.58 (dd, J = 12.3, 7.0 Hz, 3H), 3.31 − 3.28 (m, 1H), 3.02 (s, 3H), 2.94 (s, 6H), 1.37 (d, J = 6.8 Hz, 6H). | |
| Replace | | ¹H NMR (400 MHz, DMSO-d₆) δ | 536.1 |
| compound H6-1 | | 10.19 (s, 1H), 9.09 (s, 1H), 8.77 (s, | |
| with: | | 1H), 8.35 (d, J = 5.8 Hz, 1H), 8.28 (s, | |
| | | 1H), 8.09 (s, 1H), 7.45 (d, J = 8.0 Hz, | |
| 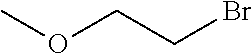 | | 1H), 7.14 (d, J = 5.6 Hz, 1H), 6.43 (d, J = 8.1 Hz, 1H), 4.41 (t, J = 7.7 Hz, | |
| | | 2H), 3.98 (t, J = 6.9 Hz, 2H), 3.74 (t, | |
| | | J = 5.2 Hz, 3H),3.60 (d, J =7.4Hz, | |
| | | 3H), 3.31 − 3.28 (m, 1H), 3.24 (s, 3H), | |
| | | 3.02 (s, 3H), 1.36 (d, J = 6.8 Hz, 6H). | |
| Replace | | ¹H NMR (400 MHz, DMSO-d₆) δ | 614.1 |
| intermediate a3 | | 10.43 (s, 1H), 9.11 (s, 1H), 8.81 (s, | |
| with: | | 1H), 8.66 (s, 1H), 8.50 (s, 1H), 8.43 | |
| | | (d, J = 5.9 Hz, 1H), 7.47 (d, J = 8.0 Hz, | |
| 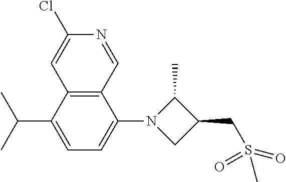 [SMILES: CC(C)c1ccc(N2C[C@H](CS(C)(=O)=O)[C@H]2C)c2cnc(Cl)cc12] | a26 | 1H), 7.21 (s, 1H), 6.60 (d, J = 8.0 Hz, 1H), 4.70 (t, J = 7.8 Hz, 1H), 4.21 (t, J = 7.8 Hz, 1H), 4.10 (t, J = 5.5 Hz, 3H), 3.69 (t, J = 5.5 Hz, 3H), 3.56- 3.54 (m, 5H), 3.09 (s, 3H), 3.00 (s, 3H), 1.43 (d, J = 6.0 Hz, 3H), 1.39 (d, J = 6.7 Hz, 6H). | |
| Replace | | | |
| compound H6-1 | | | |
| with: | | | |
| 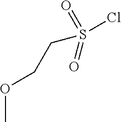 | | | |
| Replace | | ¹H NMR (400 MHz, DMSO-d₆) δ | 628.0 |
| intermediate a3 | | 10.41 (s, 1H), 9.11 (s, 1H), 8.82 (s, | |
| with: | | 1H), 8.64 (s, 1H), 8.48 (s, 1H), 8.42 | |
| | | (d, J = 5.8 Hz, 1H), 7.47 (d, J = 7.9 | |
| 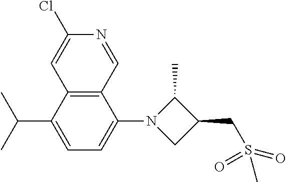 [SMILES: CC(C)c1ccc(N2C[C@H](CS(C)(=O)=O)[C@H]2C)c2cnc(Cl)cc12] | a26 | Hz, 1H), 7.19 (s,1H), 6.60 (d, J = 8.0 Hz, 1H), 4.70 − 4.67(m, 1H), 4.23 − 4.18 (m, 1H), 4.12-4.05 (m, 2H), 3.76 − 3.70 (m, 2H), 3.68 − 3.66 (m, 1H), 3.58-3.52 (m, 2H), 3.26 − 3.20 (m, 2H), 3.17 − 3.16 (m, 1H), 3.00 (s, 3H), 2.91 − 2.89 (m, 1H), 1.55 − 1.29 (m, 9H), 0.84 (t, J = 6.8 Hz, 3H). | |
| Replace | | | |
| compound H6-1 | | | |
| with: | | | |
| 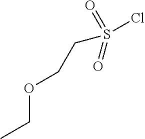 | | | |
| Replace | | ¹H NMR (400 MHz, DMSO-d₆) δ | 612.0 |
| compound H6-1 | | 10.40 (s, 1H), 9.10 (s, 1H), 8.76 − 8.73 | |
| with: | | (m, 2H), 8.54 (s, 1H), 8.44 (d, J = 5.9 | |
| | | Hz, 1H), 7.45 (d, J = 8.0 Hz, 1H), 7.25 | |
| 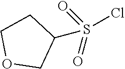 | | (s, 1H), 6.45 (d, J = 8.1 Hz, 1H), 4.74 − 4.69 (m, 1H), 4.41 (t, J = 7.7 Hz, 2H), 4.22 − 4.18 (m, 1H), 4.02 − 3.88 (m, 4H), 3.80 − 3.74 (m, 1H), 3.67 − 3.63 (m, 4H), 3.02 (s, 3H), 2.325 − 2.28 (m, 2H), 1.37 (dd, J = 6.7, 2.7 Hz, 6H). | |
| Replace | | ¹H NMR (400 MHz, DMSO-d₆) δ | 517.9 |
| compound H6-1 | | 10.25 (s, 1H), 9.10 (s, 1H), 8.70 (s, 1H), | |
| with: | | 8.48 (s, 1H), 8.44 (s, 1H), 8.38 (d, J = | |
| | | 5.8 Hz, 1H), 8.19 (s, 1H), 7.45 (d, J = | |
| 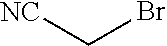 | | 7.9 Hz, 1H), 7.22 (s, 1H), 6.44 (d, J = 8.0 Hz, 1H), 5.60 (s, 2H), 4.41 (t, J = | |
| | | 7.5 Hz, 2H), 3.98 (t, J = 6.8 Hz, 3H), | |
| | | 3.60 (d, J = 7.3 Hz, 2H), 3.58 − 3.54 (m, | |
| | | 1H), 3.30 − 3.27 (m, 1H), 3.02 (s, 3H), | |
| | | 1.36 (d, J = 6.7 Hz, 6H). | |
| Replace | | ¹H NMR (400 MHz, DMSO-d₆) δ | 506.2 |
| compound H6-1 | | 10.19 (s, 1H), 9.09 (s, 1H), 8.78 (s, | |
| with: | | 1H), 8.36 − 8.30 (m, 2H), 8.08 (s, 1H), | |
| | | 7.45 (d, J = 8.0 Hz, 1H), 7.12 (d, J = | |
| 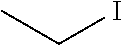 | | 4.8 Hz, 1H), 6.43 (d, J = 8.0 Hz, 1H), 4.41 (t, J = 7.5 Hz, 2H), 4.23 (dd, J = | |
| | | 14.3, 7.1 Hz, 2H), 3.98 (t, J = 6.7 Hz, | |
| | | 2H), 3.64 − 3.56 (m, 3H), 3.30 − 3.25 | |
| | | (m, 1H), 3.02 (s, 3H), 1.44 (t, J = 7.2 | |
| | | Hz, 3H), 1.36 (d, J = 6.7 Hz, 6H). | |
| Replace | | ¹H NMR (400 MHz, DMSO-d₆) δ | 599.8 |
| intermediate a3 | | 10.39 (s, 1H), 9.07 (s, 1H), 8.76 (s, | |
| with: | | 1H), 8.61 (s, 1H), 8.43 − 8.36 (m, 2H), | |
| | | 7.42 (d, J = 8.0 Hz, 1H), 7.18 (s, 1H), | |
| 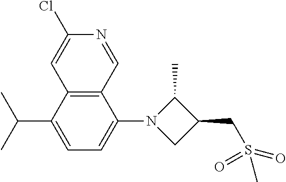 [SMILES: CC(C)c1ccc(N2C[C@H](CS(C)(=O)=O)[C@H]2C)c2cnc(Cl)cc12] | a26 | 6.56 (d, J = 8.1 Hz, 1H), 4.65 (t, J = 7.4 Hz, 1H), 4.20 − 4.13 (m, 1H), 3.62 (t, J = 7.1 Hz, 1H), 3.56 − 3.50 (m, 3H), 2.96 (s, 3H), 2.90 (s, 6H), 2.87 − 2.83 (m, 1H), 1.39 (d, J = 6.0 Hz, 3H), 1.34 (dd, J = 6.6, 2.8 Hz, 6H). | |
| Replace | | | |
| compound H6-1 | | | |
| with: | | | |
| 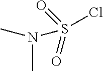 | | | |
| Replace | | ¹H NMR (400 MHz, DMSO-d₆) δ | 492.1 |
| compound H6-1 | | 10.32 (s, 1H), 9.06 (s, 1H), 8.74 (s, | |
| with: | | 1H), 8.36 − 8.27 (m, 2H), 8.05 (s, 1H), | |
| Methyl iodide | | 7.42 (d, J = 8.0 Hz, 1H), 7.11 (s, 1H), | |
| Mel | | 6.41 (d, J = 8.0 Hz, 1H), 4.37 (t, J = | |
| | | 7.6 Hz, 2H), 3.96 − 3.92 (m, 2H), 3.91 | |
| | | (s, 3H), 3.56 (d, J = 7.2 Hz, 3H), 3.27 − | |
| | | 3.23 (m, 1H), 2.98 (s, 3H), 1.32 (d, J = | |
| | | 6.7 Hz, 6H). | |
| Replace | | ¹H NMR (400 MHz, DMSO-d₆) δ | 570.0 |
| intermediate a3 | | 10.41 (s, 1H), 8.81 (s, 1H), 8.69 (s, 1H), | |
| with: | | 8.69 (s, 1H), 8.51 (s, 1H), 8.43 (d, J = | |
| | | 5.9 Hz, 1H), 7.46 (d, J = 8.0 Hz, 1H), | |
| 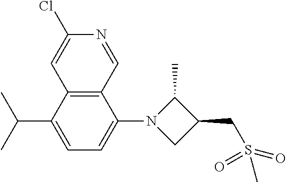 [SMILES: CC(C)c1ccc(N2C[C@H](CS(C)(=O)=O)[C@H]2C)c2cnc(Cl)cc12] | a26 | 7.21 (d, J = 4.6 Hz, 1H), 6.60 (d, J = 8.1 Hz, 1H), 4.69 (t, J = 7.5 Hz, 1H), 4.24 − 4.18 (m, 1H), 3.69 (s, 3H), 3.67 − 3.62 (m, 1H), 3.54 (ddd, J = 22.6, 13.0, 7.5 Hz, 3H), 3.00 (s, 3H), 2.90 (dd, J = 14.5, 7.2 Hz, 1H), 1.43 (d, J = 6.0 Hz, 3H), 1.38 (dd, J = 6.6, 2.8 Hz, 6H). | |
| Replace | | | |
| compound H6-1 | | | |
| with: | | | |
| 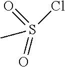 | | | |
| Replace | | ¹H NMR (400 MHz, DMSO-d₆) δ | 640.0 |
| intermediate a3 | | 10.62 (s, 1H), 9.13 (s, 1H), 8.75 (d, J = | |
| with: | | 18.2 Hz, 1H), 8.69 (s, 1H), 8.51 (s, 1H), | |
| | | 8.45 (d, J = 6.0 Hz, 1H), 7.48 (d, J = 8.0 | |
| 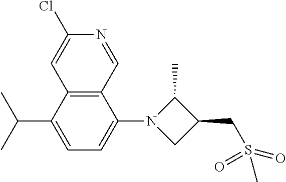 [SMILES: CC(C)c1ccc(N2C[C@H](CS(C)(=O)=O)[C@H]2C)c2cnc(Cl)cc12] | a26 | Hz, 1H), 7.26 (s, 1H), 6.62 (d, J = 8.2 Hz, 1H), 4.70 (t, J = 7.5 Hz, 1H), 4.22 (t, J = 6.6 Hz, 1H), 4.11 (t, J = 5.3 Hz, 2H), 3.77 (t, J = 5.3 Hz, 2H), 3.67 (t, J = 7.2 Hz, 1H), 3.61 − 3.50 (m, 3H), 3.17 (dt, J = 8.9, 4.6 Hz, 1H), 3.00 (s, 3H), 2.95 − 2.87 (m, 1H), 1.44 (d, J = 6.1 Hz, 3H), 1.39 (dd, J = 6.7, 3.3 Hz, 6H), 0.29 (t, J = 4.6 Hz, 4H). | |
| Replace | | | |
| compound H6-1 | | | |
| with: | | | |
| 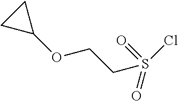 | | | |
| Replace intermediate a3 with: 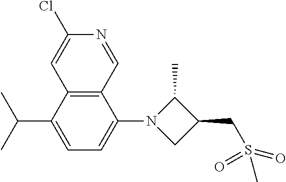 [SMILES: CC(C)c1ccc(N2C[C@H](CS(C)(=O)=O)[C@H]2C)c2cnc(Cl)cc12] | a26 | ¹H NMR (400 MHz, DMSO-d₆) δ 10.39 (s, 1H), 9.11 (s, 1H), 8.81 (s, 1H), 8.64 (s, 1H), 8.48 (s, 1H), 8.42 (d, J = 5.7 Hz, 1H), 7.47 (d, J = 7.9 Hz, 1H), 7.20 (s, 1H), 6.60 (d, J = 7.9 Hz, 1H), 4.71 − 4.66 (m, 1H), 4.23 − 4.18 (m, 1H), 4.05- 4.03 (m, J = 4.6 Hz, 2H), 3.74 − 3.69 (m, 3H), 3.57 − 3.49 (m, 4H), 2.95-2.89 (m, 1H), 3.00 (s, 3H), 1.45 − 1.38 (m, 6H), 1.23 (s, 3H), 0.86 (d, J = 5.9 Hz, 6H). | 642.1 |

Replace
compound H6-1
with:

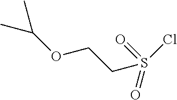

### Example 7

The inhibitory effect of the compounds of the present disclosure on kinase EGFR activity:

The MSA (Mobility Shift Assay) method was used to detect the effects of small molecule inhibitors on the kinase activity of EGFR wild type and its mutants (EGFR^{wt}, EGFR-L858R, EGFR-L858R-T790M-C797S, EGFR-L858R-C797S).

10 μL of kinase solution at 2.5 times the final concentration was added to the compound wells and positive control Max wells; 10 μL of 1x kinase buffer was added to the negative control Min wells. The mixture was centrifuged at 1000 rpm for 30 seconds, and the reaction plate was shaken to mix the solution well and then incubated at room temperature for 10 minutes. A mixed solution of ATP and kinase substrate at 5/3 times the final concentration was prepared in 1x kinase buffer. 15 μL of a mixed solution of ATP and substrate at 5/3 times the final concentration was added to initiate the reaction. The 384-well plate was centrifuged at 1000 rpm for 30 seconds, and shaken to mix the solution well and then incubated at room temperature for the corresponding time. 30 μL of stop detection solution was added to stop the kinase reaction, and the mixture was centrifuged at 1000 rpm for 30 seconds, and then to mixed well by shaking. The conversion rate was read using Caliper EZ Reader.

Calculation formula:

$$\% Inhibition = \frac{Conversion \% \_max - Conversion \% \_sample}{Conversion \% \_max - Conversion \% \_min} \times 100$$

wherein: Conversion %_sample is the conversion rate reading of the sample; Conversion %_min is the mean of the negative control wells, representing the conversion rate reading of the wells without enzyme activity; Conversion %_max is the mean of the positive control wells, representing the conversion rate reading of the wells without compound inhibition.

### Fitting Dose-Effect Curve:

The log (inhibitor) vs. response —Variable slope of the analysis software GraphPad Prism 5 was used to fit the dose-effect curve, where the log value of the concentration was used as the X-axis and the percentage inhibition rate was used as the Y-axis, to obtain the IC₅₀ value of each compound on the enzyme activity.

The calculation formula is Y=Bottom+(Top-Bottom)/(1+10\hat{ }((Log IC₅₀−X)*Hill Slope))

TABLE 1
Inhibitory test results of compounds on
EGFR (wild-type), EGFR (L858R), EGFR
(L858R/C797S) and EGFR (L858R/T790M/C797S)

| | EGFR | EGFR | EGFR (L858R/ | EGFR (L858R/ |
|---|---|---|---|---|
| Com- | wt/ | (L858R)/ | C797S)/ | T790M/C797S)/ |
| pound | IC₅₀/nM | IC₅₀/nM | IC₅₀/nM | IC₅₀/nM |
| P1 | >100 | 2.78 | 58.3 | 0.47 |
| P2 | >100 | 43.0 | >100 | 6.7 |
| P3 | 17.9 | 5.8 | 10.6 | 0.71 |
| P4 | >100 | 43.3 | >100 | 4.6 |
| P5 | >100 | >100 | >100 | 7.1 |
| P6 | >100 | 45.2 | 89.8 | 6.1 |
| P7 | 50.5 | 14.0 | 22.0 | 0.21 |
| P8 | >100 | 67.8 | 83.3 | 0.97 |
| P9 | >100 | >100 | >100 | 3.4 |
| P10 | >100 | 43.2 | 41.0 | 0.56 |
| P11 | >100 | >100 | >100 | 4.8 |
| P12 | >100 | >100 | >100 | 1.9 |
| P13 | >100 | 31.2 | 34.5 | 1.0 |
| P14 | 80.7 | 9.5 | 11.8 | 0.12 |
| P15 | >100 | 98.9 | >100 | 1.7 |
| P16 | >100 | N.D. | 24.1 | 0.28 |
| P17 | >100 | 73.4 | >100 | 1.6 |
| P18 | >100 | N.D. | >100 | 9.9 |
| P19 | >100 | >100 | >100 | 3.4 |
| P20 | 90.9 | 20.5 | 43.1 | 0.42 |
| P21 | 8.4 | 1.9 | 2.3 | 0.06 |
| P22 | >100 | >100 | >100 | 3.2 |
| P23 | 33.8 | 7.8 | 8.2 | 0.11 |
| P24 | 41.2 | N.D. | 10.4 | 0.13 |
| P25 | 5.1 | 0.77 | 1.22 | 0.08 |
| P26 | >100 | >100 | >100 | 30.9 |
| P28 | 64 | 13 | 27 | 0.34 |
| P29 | 8.4 | 1.3 | 2.5 | 0.07 |
| P30 | 18.1 | 3.5 | 4.8 | 0.10 |
| P39a | 9.4 | 1.1 | 2.0 | 0.05 |
| P45 | 20.7 | 4.1 | 7.6 | 0.18 |
| P46 | 41.8 | 6.2 | 9.3 | 0.61 |
| P51 | 13.4 | 2.5 | 4.2 | 0.12 |
| H1 | 3.9 | 1.6 | 1.5 | 0.4 |
| H2 | >100 | >100 | >100 | 71.6 |
| H3 | 11.7 | 5.6 | 4.7 | 0.7 |
| H4 | 6.1 | 2.1 | 2.9 | 0.3 |
| H5 | 10.2 | 4.6 | 1.9 | 0.43 |
| H6 | 5.2 | 1.5 | 2.2 | 0.3 |
| H7 | 2.4 | N.D. | 0.77 | 0.14 |
| H8 | >100 | N.D. | 6.4 | 3.5 |
| H9 | >100 | N.D. | 12.0 | 6.3 |
| H10 | 30.3 | 8.1 | 7.2 | 0.34 |
| H11 | 24.3 | 5.8 | 5.1 | 0.44 |
| H12 | 1.9 | 0.51 | 0.60 | 0.43 |
| H13 | 11.4 | 1.8 | 2.5 | 0.91 |
| H18 | 7.3 | 2.6 | 5.0 | 0.60 |
| H19 | 7.4 | 2.9 | 4.6 | 0.40 |
| Control | 30.2 | 7.1 | 11.0 | 0.19 |
| A | | | | |

N.D. = Not Detected

The above results show that the molecules of the present disclosure have a good inhibitory effect on osimertinib-resistant mutations in EGFR, and have a weaker inhibition on wild-type EGFR, thus having high selectivity.

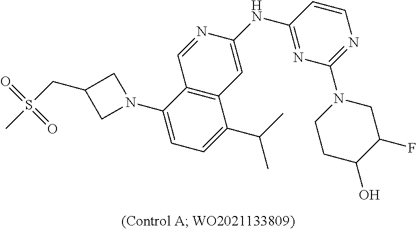 [SMILES: CC(C)c1ccc(N2CC(CS(C)(=O)=O)C2)c2cnc(Nc3ccnc(N4CCC(O)C(F)C4)n3)cc12]

### Example 8

Promega CellTiter-Glo reagent was used to detect the effects of small molecule inhibitors on the proliferation of five Ba/F3 cell lines (Ba/F3-FL-EGFR, Ba/F3-FL-EGFR-L858R, Ba/F3-FL-EGFR-L858R-T790M-C797S, Ba/F3-TEL-EGFR-L858R-C797S and Ba/F3-FL-EGFR-del19-C797S).

The cell lines were cultured in an incubator at 37° C. and 5% CO₂. Cells were passaged regularly and cells in the logarithmic phase were used for plating. 95 μL of cell suspension was added to each well of the cell plate, and culture medium without cells (containing 0.1% DMSO) was added to the Min control wells. Substance addition for compound detection cell plate: 5 μL of 20× compound working solution was added to the cell culture plate as shown in the table below. 50 μL of DMSO-cell culture medium mixture was added to the Max control, with the final DMSO concentration of 0.1%. 5 μL of DMSO-cell culture medium mixture was added to the Max control. The final DMSO concentration was 0.1%.

The compounds to be tested were added as per the table below

| 1 × Compound | | | | | | | | | | |
|---|---|---|---|---|---|---|---|---|---|---|
| solution | C-1 | C-2 | C-3 | C-4 | C-5 | C-6 | C-7 | C-8 | C-9 | C-10 |
| Concentration | 10.0000 | 2.5000 | 0.6250 | 0.1563 | 0.0391 | 0.0098 | 0.0024 | 0.00061 | 0.00015 | 10.0000 |
| (μM) | | | | | | | | | | |
| C1 to C9 (μL) | 5 | 5 | 5 | 5 | 5 | 5 | 5 | 5 | 5 | 5 |
| Culture medium | 95 | 95 | 95 | 95 | 95 | 95 | 95 | 95 | 95 | 95 |
| (μL) | | | | | | | | | | |

The culture plate was incubated in a 37° C., 5% CO₂ incubator for 72 hours. Cell viability was detected by CellTiter-Glo luminescence assay. Data analysis:

The cell proliferation inhibition rate (Inhibition Rate) data was processed using the following formula:

$$Inhibition Rate ( Inh \% ) = 100 - ( {RLU}_{Drug} - {RLU}_{Min} ) / ( {RLU}_{Max} - {RLU}_{Min} ) * 100 \% .$$

wherein: RLU_{Drug} represents the relative luminescence unit of the cells with the addition of drug, RLU_{Min} represents the luminescence unit of the culture medium, and RLU_{Max} represents the relative luminescence unit of the cells with the addition of DMSO.

The inhibition rates corresponding to different concentrations of compounds were calculated in EXCEL, and then the inhibition rate curve was plotted using GraphPad Prism software and related parameters including the maximum and minimum inhibition rates of cells and IC₅₀ values were calculated.

TABLE 2
Antiproliferative inhibitory effects of representative
compounds on BaF3 cells transfected with kinase
EGFRwt, EGFR(L858R), EGFR(L858R/C797S),
EGFR(L858R/T790M/C797S) and EGFR(del19/C797S)

| | | | Ba/F3- | Ba/F3-FL- | |
|---|---|---|---|---|---|
| | | | TEL- | EGFR- | Ba/F3-FL- |
| | | Ba/F3-FL- | EGFR- | L858R- | EGFR- |
| | Ba/F3-FL- | EGFR- | L858R- | T790M- | del19- |
| Com- | EGFR | L858R | C797S | C797S | C797S |
| pound | IC₅₀/nM | IC₅₀/nM | IC₅₀/nM | IC₅₀/nM | IC₅₀/nM |
| P1 | 1425 | 656 | 389 | 340 | N.D. |
| P3 | 889 | 55 | 43 | 248 | N.D. |
| P7 | 7377 | 771 | 271 | 554 | N.D. |
| P21 | 1511 | 76 | 38.6 | 36 | 205 |
| P24 | 3436 | 128 | 63 | 57 | 283 |
| P25 | 618 | 42 | 15 | 11 | 71 |
| P27 | 2198 | 169 | 74 | 64 | 226 |
| P29 | 1088 | 41 | 13 | 13 | 202 |
| P30 | 2339 | 111 | 48 | 47 | 200 |
| P31 | 1461 | 86 | 19 | 18 | 295 |
| P32 | 565 | 22 | 16 | 8.9 | 123 |
| P33 | 1504 | 218 | 115 | 91 | 351 |
| P34 | 903 | 206 | 83 | 78 | 234 |
| P35 | 8761 | 2518 | 1695 | 1659 | 3121 |
| P36 | 1086 | 33 | 8.3 | 14 | 159 |
| P37 | 1142 | 83 | 33 | 34 | 339 |
| P38 | 540 | 15 | 7.0 | 5.4 | 76 |
| P39a | 670 | 18 | 14 | 8.8 | 52 |
| P39b | 760 | 24 | 16 | 14 | 60 |
| P40a | 421 | 20 | 5.8 | 2.5 | 53 |
| P40b | 465 | 25 | 5.9 | 4.8 | 81 |
| P41a | 693 | 22 | 7.1 | 4.6 | 57 |
| P41b | 464 | 15 | 5.6 | 3.7 | 35 |
| P42 | 761 | 16 | 6.6 | 5.8 | 90 |
| P43 | 593 | 36 | 9.8 | 5.6 | 142 |
| P44 | 775 | 21 | 10 | 6.7 | 110 |
| P45 | 2777 | 181 | 61 | 38 | 265 |
| P46 | 3107 | 208 | 88 | 271 | 308 |
| P47 | 1610 | 113 | 38 | 42 | 179 |
| P48 | 1167 | 76 | 29 | 31 | 144 |
| P49 | 1072 | 121 | 36 | 38 | 316 |
| P50 | 6625 | 1891 | 542 | 720 | 3510 |
| P51 | 1748 | 86 | 28 | 63 | 181 |
| H1 | 101 | 5.9 | 3.1 | 16 | 10 |
| H3 | 854 | N.D. | 188 | 462 | 216 |
| H4 | 628 | 46 | 39 | 146 | N.D. |
| H6 | 559 | N.D. | 40 | 90 | 48 |
| H7 | 365 | 13 | 11 | 25 | 12 |
| H10 | 1082 | 94 | 54 | 219 | 101 |
| H11 | >10000 | 40 | 37 | 123 | 97 |
| H12 | 209 | 7.7 | 5.8 | 16 | 4.3 |
| H13 | 640 | 44 | 18 | 158 | 23 |
| H14 | 721 | 71 | 28 | 136 | 34 |
| H15 | 148 | 5.7 | 4.3 | 8.6 | 6.6 |
| H16 | 1359 | 235 | 122 | 652 | 264 |
| H17 | 858 | 56 | 33 | 127 | 58 |
| H18 | 658 | 73 | 46 | 150 | 69 |
| H19 | 559 | 64 | 36 | 123 | 57 |
| H20 | 2113 | 43 | 22 | 188 | 53 |
| H21 | 76 | 2.9 | 1.8 | 4.4 | 4.3 |
| H22 | 229 | 17 | 13 | 14 | 14 |
| H23 | 121 | 3.7 | 4.3 | 10 | 3.1 |
| H24 | 458 | 42 | 12 | 94 | 20 |
| H25 | 208 | 14 | 9.6 | 26 | 11 |
| H26 | 1421 | 74 | 56 | 336 | 128 |
| H27 | >10000 | 49 | 21 | 322 | 53 |
| H28 | 595 | 50 | 23 | 68 | 48 |
| H29 | 739 | 91 | 34 | 235 | 59 |
| H30 | 259 | 10 | 8.9 | 21 | 9.2 |
| H31 | 503 | 50 | 55 | 64 | 39 |
| H32 | 3292 | 510 | 226 | 397 | 340 |
| H33 | 202 | 14 | 5.9 | 13 | 7.1 |
| H34 | 656 | 65 | 28 | 73 | 37 |
| Control A | 2022 | 176 | 52 | 38 | N.D. |
| Osimertinib | 13 | 1.5 | 2135 | 1747 | 906 |

N.D. = Not Detected

The above results show that the compounds of the present disclosure have good anti-proliferative effects on L858R-related mutations and osimertinib-resistant mutations in EGFR, and have weaker inhibitory effects on wild-type EGFR. Some compounds are significantly superior to the control molecules.

### Example 9

Promega CellTiter-Glo reagent was used to detect the effects of small molecule inhibitors on the proliferation of three Ba/F3 cell lines (Ba/F3-FL-EGFR-de119, Ba/F3-FL-EGFR-de119-T790M, Ba/F3-FL-EGFR-de119-T790M-C797S).

The cell lines were cultured in an incubator at 37° C. and 5% CO₂. Cells were passaged regularly and cells in the logarithmic phase were used for plating. 95 μL of cell suspension was added to each well of the cell plate, and culture medium without cells (containing 0.1% DMSO) was added to the Min control wells. Substance addition for compound detection cell plate: 5 μL of 20× compound working solution was added to the cell culture plate. 5 μL of DMSO-cell culture medium mixture was added to the Max control, with the final DMSO concentration of 0.1%. 5 μL of DMSO-cell culture medium mixture was added to the Max control. The final DMSO concentration was 0.1%.

The culture plate was incubated in a 37° C., 5% CO₂ incubator for 72 hours. Cell viability was detected by CellTiter-Glo luminescence assay. Data analysis:

The cell proliferation inhibition rate (Inhibition Rate) data was processed using the following formula:

Inhibition Rate(Inh%)=100−(RLU_{Drug}−RLU_{Min})/(RLU_{Max}−RLU_{Min})*100%.

wherein: RLU_{Drug} represents the relative luminescence unit of the cells with the addition of drug, RLU_{Min} represents the luminescence unit of the culture medium, and RLU_{Max} represents the relative luminescence unit of the cells with the addition of DMSO.

The inhibition rates corresponding to different concentrations of compounds were calculated in EXCEL, and then the inhibition rate curve was plotted using GraphPad Prism software and related parameters including the maximum and minimum inhibition rates of cells and IC₅₀ values were calculated.

TABLE 3
Antiproliferative inhibitory effects of representative compounds
on BaF3 cells transfected with kinase EGFR (del19),
EGFR(del19/T790M) and EGFR(del19/T790M/C797S)

| | Ba/F3-FL- | Ba/F3-FL-EGFR- | Ba/F3-FL-EGFR- |
|---|---|---|---|
| | EGFR-Del19 | del19/T790M | del19-T790M- |
| Compound | (E746-A750) | IC₅₀/nM | C797S IC₅₀/nM |
| H12 | 15 | 5.1 | 7.8 |
| H23 | 3.1 | 0.3 | 3.1 |
| H30 | 15 | 5.1 | 7.8 |
| P39a | 41 | 4.4 | 8.4 |
| P41a | 54 | 3.2 | 5.9 |
| P41b | 36 | 2.3 | 3.7 |
| Osimertinib | 5.8 | 2.0 | 1002 |

The above results indicate that the compounds of the present disclosure have good anti-proliferative effects on del19-related mutations and osimertinib-resistant mutations in EGFR.

### Example 10

The liver microsome stability test of the compounds was as follows:

The compounds of the present disclosure were subjected to a liver microsome stability test. The compounds to be tested were co-incubated with liver microsomes of different species with or without the addition of NADPH. The final concentration of the compounds to be tested in the test system was 1 μM. The final concentration of NADPH was 1 mM, and the final concentration of liver microsomes was 0.5 mg/ml. The compound concentrations in the incubation supernatant at different time points within 60 minutes were detected and pharmacokinetic parameters (such as clearance Clint) were calculated.

This result indicates that the molecules of the present disclosure have good metabolic stability (especially in the human body).

| | Human | Mouse |
|---|---|---|
| | Clint/ | Clint/ |
| Compound | (mL/min/kg) | (mL/min/kg) |
| P21 | 13.6 | 396 |
| P29 | 23.4 | 440 |
| P30 | <8.6 | 230 |
| P39a | <8.6 | 231 |
| H1 | <8.6 | 174 |
| H7 | 21.3 | 151 |
| H12 | 14.2 | 196 |
| H19 | 17.2 | 61.8 |
| H23 | <8.6 | 448 |
| H30 | 11.7 | 145 |

### Example 11

In vivo efficacy assay in BALB/c nude mice. Details were as follows:

BaF3 (del19/T790M1C797S) tumor cells were cultured and inoculated into 6-8 weeks old female BALB/c nude mice (weighing about 20 g), with all mice being subcutaneously inoculated. The mice were cultured in an SPF-grade experimental environment, and all mice had free access to commercially certified standard diets. When the average tumor volume of the mice grew to about 150 mm³, the test compound was orally administered daily. The dosage was as follows: blank group vehicle (DMSO/Solutol/H₂O, 5/10/85). The dosage of the administration group was 100 mg/kg, twice a day; the dosage of the osimertinib group was 25 mg/kg, once a day. The tumor volume was measured three times a week with a two-dimensional caliper, and the animals were weighed every day. After 13 consecutive days of administration, the inhibition rate (TGI/100%) was calculated based on the final tumor volume. The volume calculation formula is: V=1/2a*b², where a represents the long diameter of the tumor, and b represents the short diameter of the tumor.

| Test drug | Dosage | | Tumor volume (mm³) | TGI |
|---|---|---|---|---|
| Blank group | 0 | | 3870 | 0% |
| Osimertinib | 25 | mg/kg, QD | 3110 | 20% |
| H12 | 100 | mg/kg^{$}, BID | 1341 | 68% |
| H23 | 100 | mg/kg^{$}, BID | 28 | 103% |
| P39a | 100 | mg/kg, BID | 945 | 79% |
| P41a | 100 | mg/kg, BID | 693 | 85% |

^{$}means that the dosage was 50 mg/kg for the first 4 days and 100 mg/kg starting from the 5th day.

The results show that the molecules of the present disclosure have good in vivo efficacy, have excellent inhibitory effects on osimertinib-resistant tumor cells, can overcome drug resistance, and bring clinical benefits.

1. A compound of formula (I), or a pharmaceutically acceptable salt, isotopic variant, tautomer, stereoisomer, prodrug, polymorph, hydrate or solvate thereof: 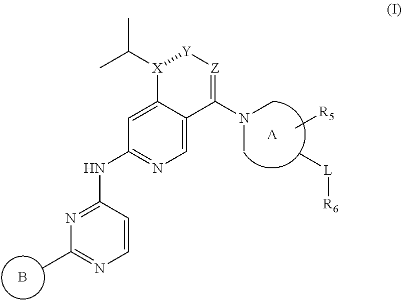
wherein: 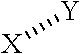
represents 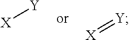
wherein when 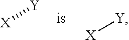
X is selected from CH and N, and Y is selected from CH₂, C═O and C═S; and when 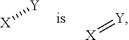
X is C, and Y is CH;
Z is selected from CH and N;
ring A is 4- to 8-membered heterocyclyl;
ring B is selected from 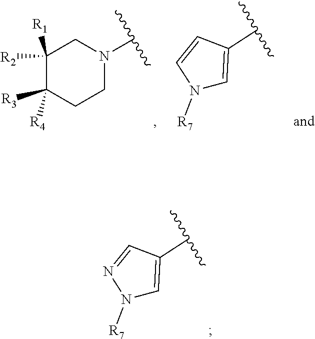
L is selected from a chemical bond, —S(═O)(=M)-, —N(R_{N})—S(═O)(=M)-, —S(═O)(=M)-N(R_{N})—, —N(R_{N})—C(═O)—, —C(═O)—N(R_{N})—, —CH(R_{N})—C(═O)—, —C(═O)—CH(R_{N})—, —CH(R_{N})—S(═O)(=M)- and —S(═O)(=M)-CH(R_{N})—;
R₁ and R₂ are independently selected from H, halogen, C₁₋₆ alkyl and C₁₋₆ haloalkyl;
R₃ and R₄ are independently selected from H, ORₐ, C₁₋₆ alkyl and C₁₋₆ haloalkyl;
R₅ is selected from H, halogen, C₁₋₆ alkyl and C₁₋₆ haloalkyl;
R₆ is selected from —C₀₋₆ alkylene-ORₐ, —C₀₋₆ alkylene-NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl;
R₇ is selected from H, —C₁₋₆ alkylene-CN, —C₁₋₆ alkylene-ORₐ, —C₁₋₆ alkylene-NR_{b}R_{c}, C₁₋₆ alkyl, C₁₋₆ haloalkyl and —S(═O)(=M)-R₈;
wherein M is O or NH;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
R₈ is selected from —C₀₋₆ alkylene-ORₐ, —C₀₋₆ alkylene-NR_{b}R_{c}, C₁₋₆ alkyl, —C₁₋₆ alkylene-C₃₋₆ cycloalkyl, C₁₋₆ haloalkyl, C₃₋₆ cycloalkyl, 4- to 6-membered heterocyclyl, C₆₋₁₀ aryl and 5- to 6-membered heteroaryl, which are optionally substituted with one or more Rₐ;
Rₐ, R_{b} and R_{c} are independently selected from H, C₁₋₆ alkyl, C₁₋₆ haloalkyl, C₃₋₆ cycloalkyl and 4 to 6-membered heterocyclyl; or R_{b} and R_{c} together with the nitrogen atom to which they are attached form 4- to 7-membered heterocyclyl;
each group in X, Y, Z, ring A, ring B, L, R₁-R₈, R_{N}, Rₐ, R_{b} and R_{c} may be substituted by one or more deuterium atoms, up to full deuteration;
provided that, when L is —CH(R_{N})—S(═O)(=M)-, and the ring where X, Y and Z are located is a benzene ring, B is

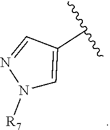

2. The compound of formula (I) according to claim 1, or a pharmaceutically acceptable salt, isotopic variant, tautomer, stereoisomer, prodrug, polymorph, hydrate or solvate thereof, wherein the compound has one or more of the following definitions:
i) wherein the ring where X, Y and Z are located is selected from: 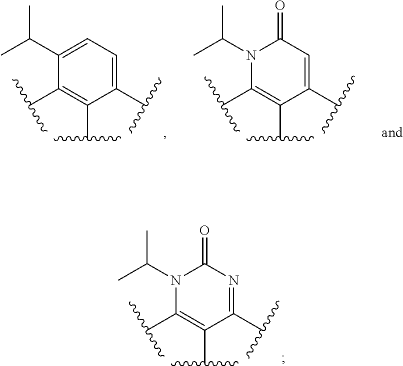 [SMILES: *c1ccc(C(C)C)c(*)c1* ; *c1cc(=O)n(C(C)C)c(*)c1* ; *c1nc(=O)n(C(C)C)c(*)c1*]
ii) wherein ring A is selected from: 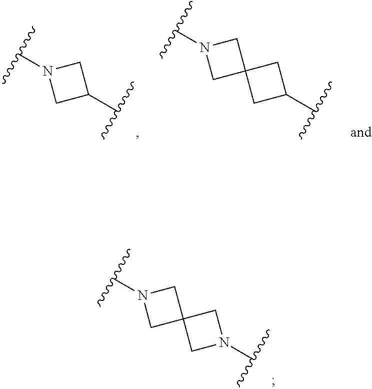 [SMILES: *C1CN(*)C1 ; *C1CC2(C1)CN(*)C2 ; *N1CC2(C1)CN(*)C2]
iii) wherein ring B is 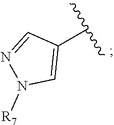
iv) wherein ring B is 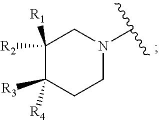
v) wherein L is selected from —S(═O)(=M)-, —N(R_{N})—S(═O)(=M)-, —N(R_{N})—C(═O)—, —CH(R_{N})—C(═O)— and —CH(R_{N})—S(═O)(=M)-; alternatively, L is —N(R_{N})—S(═O)(=M)- or —CH(R_{N})—S(═O)(=M)-;
vi) wherein one of R₁ and R₂ is halogen, and the other is selected from H, halogen and C₁₋₆ alkyl;
alternatively, one of R₁ and R₂ is F, and the other is selected from H, F and Me: alternatively, one of R₁ and R₂ is F, and the other is H:
vii) wherein one of R₃ and R₄ is ORₐ, and the other is selected from H and C₁₋₆ alkyl;
alternatively, one of R₃ and R₄ is OH or OMe, and the other is selected from H and Me; or
viii) wherein R₅ is selected from H and Me.

3.-9. (canceled)

10. The compound of formula (I) according to claim 1, or a pharmaceutically acceptable salt, isotopic variant, tautomer, stereoisomer, prodrug, polymorph, hydrate or solvate thereof, having the following structures: 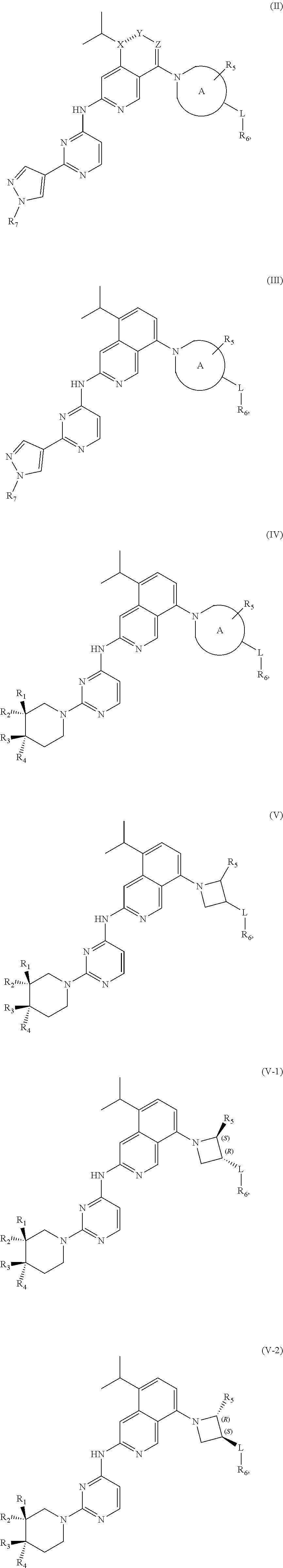 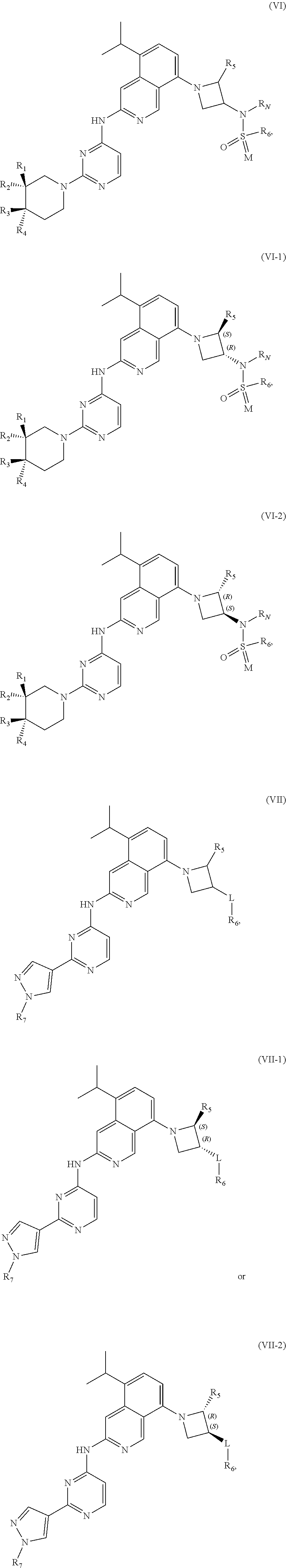
wherein each group is as defined in claim 1.

11. The compound of claim 10, or a pharmaceutically acceptable salt, isotopic variant, tautomer, stereoisomer, prodrug, polymorph, hydrate or solvate thereof, which is a compound of formula (II): 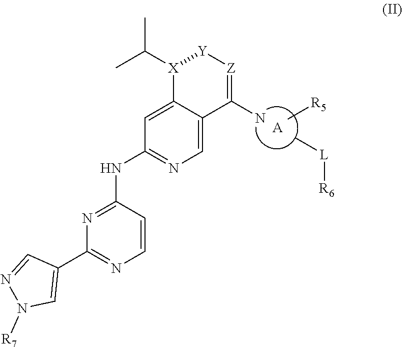
wherein: 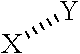
represents 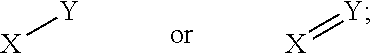
wherein when 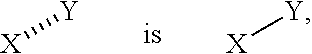
X is selected from CH and N, and Y is selected from CH₂, C═O and C═S; and when 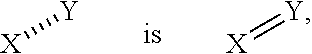
X is C, and Y is CH;
Z is selected from CH and N;
ring A is 4- to 8-membered heterocyclyl;
L is selected from —S(═O)(=M)-, —N(R_{N})—S(═O)(=M)-, —S(═O)(=M)-N(R_{N})—, —N(R_{N})—C(═O)—, —C(═O)—N(R_{N})—, —CH(R_{N})—C(═O)—, —C(═O)—CH(R_{N})—, —CH(R_{N})—S(═O)(=M)- and —S(═O)(=M)-CH(R_{N})—;
R₅ is selected from H, halogen, C₁₋₆ alkyl and C₁₋₆ haloalkyl;
R₆ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl;
R₇ is selected from H, C₁₋₆ alkyl, C₁₋₆ haloalkyl and —S(═O)(=M)-R₈;
wherein M is O or NH;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
R₈ is selected from ORₐ, NR_{b}R_{c}, C₁₋₆ alkyl, —CH₂—C₃₋₆ cycloalkyl, C₁₋₆ haloalkyl, C₃₋₆ cycloalkyl, 4- to 6-membered heterocyclyl, C₆₋₁₀ aryl and 5- to 6-membered heteroaryl, which are optionally substituted with one or more Rₐ;
Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl.

12. The compound of claim 10, or a pharmaceutically acceptable salt, isotopic variant, tautomer, stereoisomer, prodrug, polymorph, hydrate or solvate thereof, which is a compound of formula (III): 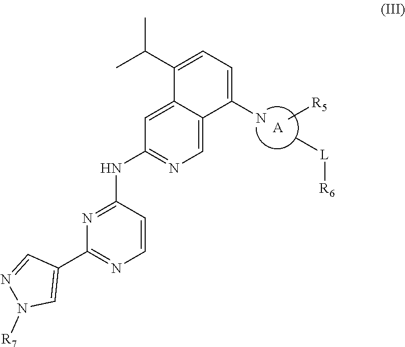
wherein:
ring A is 4- to 8-membered heterocyclyl;
L is selected from —S(═O)(=M)-, —N(R_{N})—S(═O)(=M)-, —S(═O)(=M)-N(R_{N})—, —N(R_{N})—C(═O)—, —C(═O)—N(R_{N})—, —CH(R_{N})—C(═O)—, —C(═O)—CH(R_{N})—, —CH(R_{N})—S(═O)(=M)- and —S(═O)(=M)-CH(R_{N})—;
R₅ is selected from H, halogen, C₁₋₆ alkyl and C₁₋₆ haloalkyl;
R₆ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl;
R₇ is selected from H, C₁₋₆ alkyl, C₁₋₆ haloalkyl and —S(═O)(=M)-R₈;
wherein M is O or NH;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
R₈ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl, which are optionally substituted with one or more Rₐ;
Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl.

13. The compound of claim 12, or a pharmaceutically acceptable salt, isotopic variant, tautomer, stereoisomer, prodrug, polymorph, hydrate or solvate thereof,
wherein:
ring A is selected from 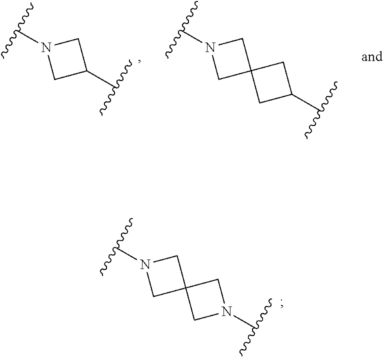 [SMILES: *C1CN(*)C1 ; *C1CC2(C1)CN(*)C2 ; *N1CC2(C1)CN(*)C2]
L is selected from —S(═O)(=M)-, —N(R_{N})—S(═O)(=M)-, —S(═O)(=M)-N(R_{N})—, —N(R_{N})—C(═O)—, —C(═O)—N(R_{N})—, —CH(R_{N})—C(═O)—, —C(═O)—CH(R_{N})—, —CH(R_{N})—S(═O)(=M)- and —S(═O)(=M)-CH(R_{N})—;
R₅ is selected from H and Me;
R₆ is selected from Me, Et, Pr, CH₂CF₃, cyclopropyl, OH, NH₂, NHMe, NMe₂, furan-2-yl and pyridin-4-yl;
R₇ is selected from H, trifluoroethyl and —S(═O)(=M)-R₈;
wherein M is O or NH;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
R₈ is selected from Me, Et, Pr, CH₂CF₃, cyclopropyl, OH, NH₂, NHMe, NMe₂, pyran-4-yl, furan-2-yl and pyridin-4-yl;
Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl,
alternatively, wherein:
ring A is 4- to 8-membered heterocyclyl:
L is selected from —S(═O)(=M)-, —CH(R_{N})—C(═O)— and —CH(R_{N})—S(═O)(=M)-;
R₅ is H, C₁₋₆ alkyl or C₁₋₆ haloalkyl;
R₆ is selected from ORₐ, NR_{b}R_{c}, C₁₋₆ alkyl, C₁₋₆ haloalkyl, C₃₋₆ cycloalkyl and 4- to 6-membered heterocyclyl;
R₇ is selected from H, trifluoroethyl and —S(═O)(=M)-R₈;
wherein M is O or NH;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
R₈ is selected from ORₐ, NR_{b}R_{c}, C₁₋₆ alkyl, C₁₋₆ haloalkyl, C₃₋₆ cycloalkyl and 4- to 6-membered heterocyclyl;
Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl;
alternatively, wherein:
ring A is 4- to 8-membered heterocyclyl;
L is selected from —S(═O)(=M)-, —CH(R_{N})—C(═O)— and —CH(R_{N})—S(═O)(=M)-;
R₅ is selected from H and C₁₋₆ alkyl;
R₆ is selected from NR_{b}R_{c}, C₁₋₆ alkyl and C₁₋₆ haloalkyl;
R₇ is selected from H, trifluoroethyl and —S(═O)(=M)-R₈;
wherein M is O or NH;
R_{N} is selected from H, C₁₋₆ alkyl and C₁₋₆ haloalkyl;
R₈ is selected from C₁₋₆ alkyl, C₁₋₆ haloalkyl, C₃₋₆ cycloalkyl and pyran-4-yl;
R_{b} and R_{c} are independently selected from H, C₁₋₆ alkyl and C₁₋₆ haloalkyl;
alternatively, wherein:
ring A is selected from 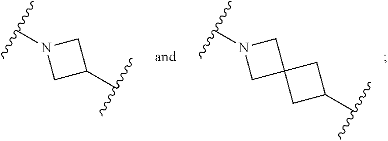 [SMILES: *C1CN(*)C1 ; *C1CC2(C1)CN(*)C2]
L is selected from —S(═O)(=M)-, —CH(R_{N})—C(═O)— and —CH(R_{N})—S(═O)(=M)-;
R₅ is H;
R₆ is selected from Me and NH₂;
R₇ is selected from H and —S(═O)(=M)-R₈;
wherein M is O;
R_{N} is selected from H, C₁₋₆ alkyl and C₁₋₆ haloalkyl;
R₈ is selected from Me, Et, Pr, cyclopropyl and pyran-4-yl.

14.-16. (canceled)

17. The compound of claim 10, or a pharmaceutically acceptable salt, isotopic variant, tautomer, stereoisomer, prodrug, polymorph, hydrate or solvate thereof, which is a compound of formula (IV): 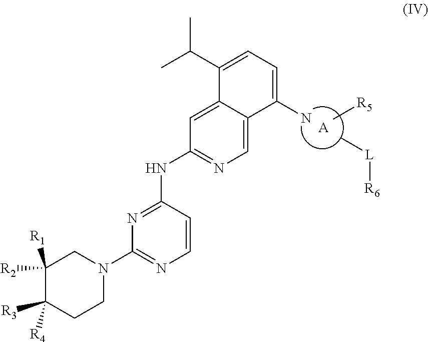
wherein:
ring A is 4- to 8-membered heterocyclyl;
L is selected from —S(═O)(=M)-, —N(R_{N})—S(═O)(=M)-, —S(═O)(=M)-N(R_{N})—, —N(R_{N})—C(═O)—, —C(═O)—N(R_{N})—, —CH(R_{N})—C(═O)— and —C(═O)—CH(R_{N})—;
R₁ and R₂ are independently selected from H, halogen, C₁₋₆ alkyl and C₁₋₆ haloalkyl;
R₃ and R₄ are independently selected from H, ORₐ, C₁₋₆ alkyl and C₁₋₆ haloalkyl;
R₅ is selected from H, halogen, C₁₋₆ alkyl and C₁₋₆ haloalkyl;
R₆ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl;
wherein M is O or NH;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl.

18. The compound of claim 17, or a pharmaceutically acceptable salt, isotopic variant, tautomer, stereoisomer, prodrug, polymorph, hydrate or solvate thereof, wherein:
ring A is selected from 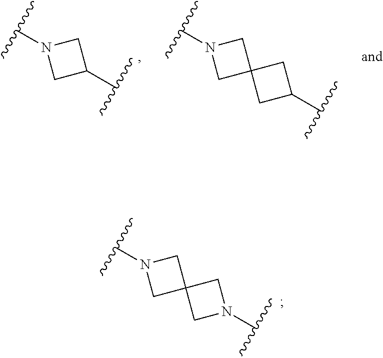 [SMILES: *C1CN(*)C1 ; *C1CC2(C1)CN(*)C2 ; *N1CC2(C1)CN(*)C2]
L is selected from —S(═O)(=M)-, —N(R_{N})—S(═O)(=M)-, —S(═O)(=M)-N(R_{N})—, —N(R_{N})—C(═O)—, —C(═O)—N(R_{N})—, —CH(R_{N})—C(═O)— and —C(═O)—CH(R_{N})—;
R₁ and R₂ are independently selected from H, halogen, C₁₋₆ alkyl and C₁₋₆ haloalkyl;
R₃ and R₄ are independently selected from H, ORₐ, C₁₋₆ alkyl and C₁₋₆ haloalkyl;
R₅ is selected from H, C₁₋₆ alkyl and C₁₋₆ haloalkyl;
R₆ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl;
wherein M is O or NH;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl.

19. The compound of claim 10, or a pharmaceutically acceptable salt, isotopic variant, tautomer, stereoisomer, prodrug, polymorph, hydrate or solvate thereof, which is a compound of formula (V), (V-1) or (V-2): 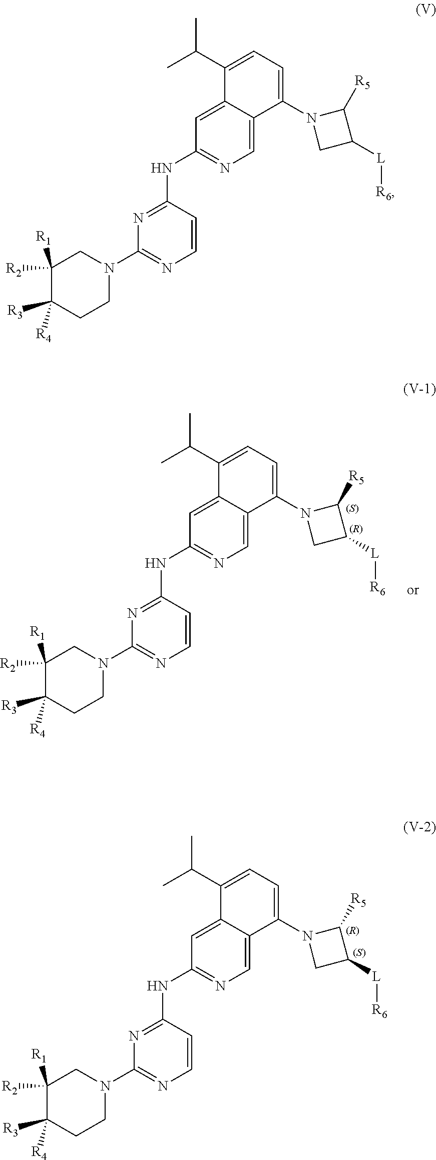
wherein:
L is selected from —S(═O)(=M)-, —N(R_{N})—S(═O)(=M)-, —S(═O)(=M)-N(R_{N})—, —N(R_{N})—C(═O)—, —C(═O)—N(R_{N})—, —CH(R_{N})—C(═O)— and —C(═O)—CH(R_{N})—;
R₁ and R₂ are independently selected from H, halogen, C₁₋₆ alkyl and C₁₋₆ haloalkyl;
R₃ and R₄ are independently selected from H, ORₐ, C₁₋₆ alkyl and C₁₋₆ haloalkyl;
R₅ is selected from H, halogen, C₁₋₆ alkyl and C₁₋₆ haloalkyl;
R₆ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl;
wherein M is O or NH;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl.

20. The compound of claim 19, or a pharmaceutically acceptable salt, isotopic variant, tautomer, stereoisomer, prodrug, polymorph, hydrate or solvate thereof, wherein:
L is selected from —S(═O)(=M)-, —N(R_{N})—S(═O)(=M)-, —N(R_{N})—C(═O)— and —CH(R_{N})—C(═O)—;
one of R₁ and R₂ is halogen, and the other is selected from H, halogen and C₁₋₆ alkyl;
one of R₃ and R₄ is ORₐ, and the other is selected from H and C₁₋₆ alkyl;
R₅ is selected from H, C₁₋₆ alkyl and C₁₋₆ haloalkyl;
R₆ is selected from ORₐ, NR_{b}R_{c}, C₁₋₆ alkyl, C₁₋₆ haloalkyl, C₃₋₆ cycloalkyl and 5- to 6-membered heteroaryl;
wherein M is O or NH;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl.

21. The compound of claim 10, or a pharmaceutically acceptable salt, isotopic variant, tautomer, stereoisomer, prodrug, polymorph, hydrate or solvate thereof, which is a compound of formula (VI), (VI-1) or (VI-2): 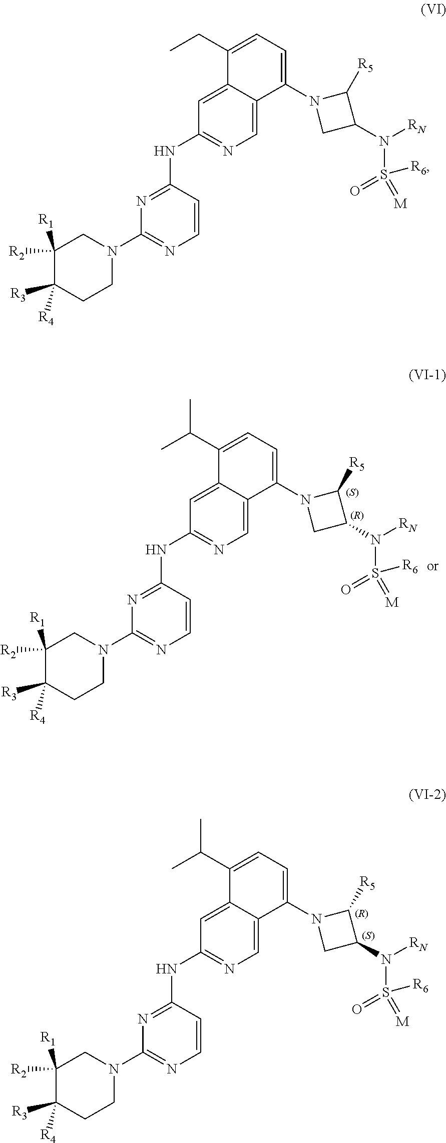
wherein:
M is O or NH;
R₁ and R₂ are independently selected from H, halogen, C₁₋₆ alkyl and C₁₋₆ haloalkyl;
R₃ and R₄ are independently selected from H, ORₐ, C₁₋₆ alkyl and C₁₋₆ haloalkyl;
R₅ is selected from H, halogen, C₁₋₆ alkyl and C₁₋₆ haloalkyl;
R₆ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
wherein Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl.

22. The compound of claim 21, or a pharmaceutically acceptable salt, isotopic variant, tautomer, stereoisomer, prodrug, polymorph, hydrate or solvate thereof,
wherein:
M is O or NH;
one of R₁ and R₂ is halogen, and the other is selected from H, halogen and C₁₋₆ alkyl;
one of R₃ and R₄ is ORₐ, and the other is selected from H and C₁₋₆ alkyl;
R₅ is selected from H and C₁₋₆ alkyl;
R₆ is selected from ORₐ, NR_{b}R_{c}, C₁₋₆ alkyl, C₁₋₆ haloalkyl, C₃₋₆ cycloalkyl and 5- to 6-membered heteroaryl;
R_{N} is selected from H and C₁₋₆ alkyl;
or R_{N} and R₆ are linked to form C₁₋₆ alkylene;
wherein Rₐ, R_{b} and R_{c} are independently selected from H, C₁₋₆ alkyl and C₁₋₆ haloalkyl,
alternatively, wherein:
M is O;
one of R₁ and R₂ is F, and the other is selected from H, F and Me:
one of R₃ and R₄ is OH or OMe, and the other is selected from H and Me:
R₅ is selected from H and Me:
R₆ is selected from Me, Et, Pr, CH₂CF₃, cyclopropyl, NMe₂, furan-2-yl and pyridin-4-yl;
R_{N} is selected from H and Me;
or R_{N} and R₆ are linked to form C₁₋₄ alkylene:
alternatively, wherein:
M is O or NH;
one of R₁ and R₂ is selected from halogen, C₁₋₆ alkyl and C₁₋₆ haloalkyl, and the other is H;
one of R₃ and R₄ is selected from ORₐ, C₁₋₆ alkyl and C₁₋₆ haloalkyl, and the other is H:
R₅ is selected from H, halogen, C₁₋₆ alkyl and C₁₋₆ haloalkyl;
R₆ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl:
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene:
wherein Rₐ, R_{b} and R_{c} are independently selected from H, C₁₋₆ alkyl and C₁₋₆ haloalkyl: or R_{b} and R_{c} together with the nitrogen atom to which they are attached form 4- to 7-membered heterocyclyl;
alternatively, wherein:
M is O or NH;
one of R₁ and R₂ is selected from halogen and C₁₋₆ alkyl, and the other is H:
one of R₃ and R₄ is ORₐ, and the other is H:
R₅ is selected from H, C₁₋₆ alkyl and C₁₋₆ haloalkyl;
R₆ is selected from C₁₋₆ alkyl, C₁₋₆ haloalkyl, C₃₋₆ cycloalkyl and 4- to 6-membered heterocyclyl;
R_{N} is selected from H, C₁₋₆ alkyl and C₁₋₆ haloalkyl:
wherein Rₐ is selected from H, C₁₋₆ alkyl and C₁₋₆ haloalkyl;
alternatively, wherein:
M is O;
one of R₁ and R₂ is halogen (alternatively F), and the other is H:
one of R₃ and R₄ is ORₐ, and the other is H;
R₅ is selected from H and C₁₋₄ alkyl;
R₆ is selected from C₁₋₄ alkyl and C₁₋₄ haloalkyl;
R_{N} is selected from H, C₁₋₄ alkyl and C₁₋₄ haloalkyl; alternatively C₁₋₄ alkyl:
wherein Rₐ is selected from H and C₁₋₄ alkyl.

23.-26. (canceled)

27. The compound of claim 10, or a pharmaceutically acceptable salt, isotopic variant, tautomer, stereoisomer, prodrug, polymorph, hydrate or solvate thereof, which is a compound of formula (VII), (VII-1) or (VII-2): 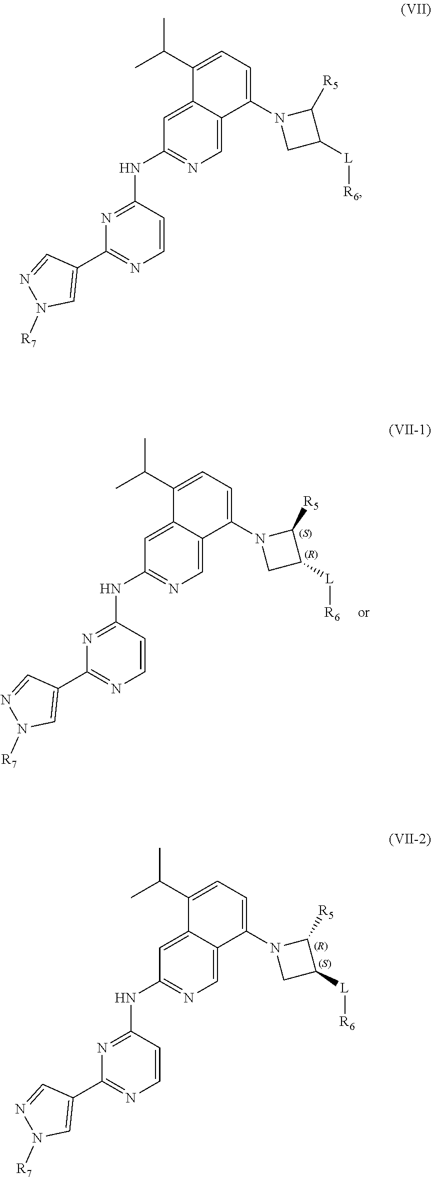
wherein:
L is selected from —S(═O)(=M)-, —N(R_{N})—S(═O)(=M)-, —S(═O)(=M)-N(R_{N})—, —N(R_{N})—C(═O)—, —C(═O)—N(R_{N})—, —CH(R_{N})—C(═O)—, —C(═O)—CH(R_{N})—, —CH(R_{N})—S(═O)(=M)- and —S(═O)(=M)-CH(R_{N})—;
R₅ is selected from H, halogen, C₁₋₆ alkyl and C₁₋₆ haloalkyl;
R₆ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl;
R₇ is selected from H, C₁₋₆ alkyl, C₁₋₆ haloalkyl and —S(═O)(=M)-R₈;
wherein M is O or NH;
R_{N} is selected from H, C₁₋₆ alkyl and C₁₋₆ haloalkyl;
or R_{N} and R₆ are linked to form C₁₋₆ alkylene, C₂₋₆ alkenylene or C₂₋₆ alkynylene;
R₈ is selected from ORₐ, NR_{b}R_{c}, C₁₋₆ alkyl, C₁₋₆ haloalkyl, C₃₋₆ cycloalkyl, 4- to 6-membered heterocyclyl, C₆₋₁₀ aryl and 5- to 6-membered heteroaryl, which are optionally substituted with one or more Rₐ;
Rₐ, R_{b} and R_{c} are independently selected from H, C₁₋₆ alkyl and C₁₋₆ haloalkyl; or R_{b} and R_{c} together with the nitrogen atom to which they are attached form 4- to 7-membered heterocyclyl.

28. The compound of claim 27, or a pharmaceutically acceptable salt, isotopic variant, tautomer, stereoisomer, prodrug, polymorph, hydrate or solvate thereof,
wherein:
L is selected from —N(R_{N})—S(═O)(=M)- and —CH(R_{N})—S(═O)(=M)-;
R₅ is selected from C₁₋₆ alkyl and C₁₋₆ haloalkyl;
R₆ is selected from C₁₋₆ alkyl, C₁₋₆ haloalkyl, C₃₋₆ cycloalkyl and 4- to 6-membered heterocyclyl;
R₇ is —S(═O)(=M)-R₈;
wherein M is O or NH;
R_{N} is selected from H, C₁₋₆ alkyl and C₁₋₆ haloalkyl;
R₈ is selected from C₁₋₆ alkyl, C₁₋₆ haloalkyl, C₃₋₆ cycloalkyl and 4- to 6-membered heterocyclyl, which are optionally substituted with one or more Rₐ;
Rₐ is selected from H, C₁₋₆ alkyl and C₁₋₆ haloalkyl,
alternatively, wherein:
L is selected from —N(R_{N})—S(═O)(=M)- and —CH₂—S(═O)(=M)-;
R₅ is selected from C₁₋₆ alkyl and C₁₋₆ haloalkyl;
R₆ is selected from C₁₋₆ alkyl and C₁₋₆ haloalkyl;
R₇ is —S(═O)(=M)-R₈:
wherein M is O or NH;
R_{N} is selected from C₁₋₆ alkyl and C₁₋₆ haloalkyl;
R₈ is selected from C₁₋₆ alkyl, C₁₋₆ haloalkyl, C₃₋₆ cycloalkyl and 4- to 6-membered heterocyclyl;
alternatively, wherein:
L is selected from —N(R_{N})—S(═O)₂— and —CH₂—S(═O)₂—;
R₅ is selected from C₁₋₆ alkyl and C₁₋₆ haloalkyl;
R₆ is selected from C₁₋₆ alkyl and C₁₋₆ haloalkyl;
R₇ is —S(═O)₂—R₈;
wherein R_{N} is selected from C₁₋₆ alkyl and C₁₋₆ haloalkyl;
R₈ is selected from C₁₋₆ alkyl, C₁₋₆ haloalkyl and C₃₋₆ cycloalkyl:
alternatively, wherein:
L is selected from —N(R_{N})—S(═O)₂— and —CH₂—S(═O)₂—;
R₅ is C₁₋₄ alkyl;
R₆ is C₁₋₄ alkyl;
R₇ is —S═O)₂—R₈;
wherein R_{N} is C₁₋₄ alkyl;
R₈ is selected from C₁₋₄ alkyl and C₃₋₆ cycloalkyl.

29.-31. (canceled)

32. A compound, or a tautomer, stereoisomer, prodrug, crystalline form, pharmaceutically acceptable salt, hydrate or solvate thereof, wherein the compound is selected from:

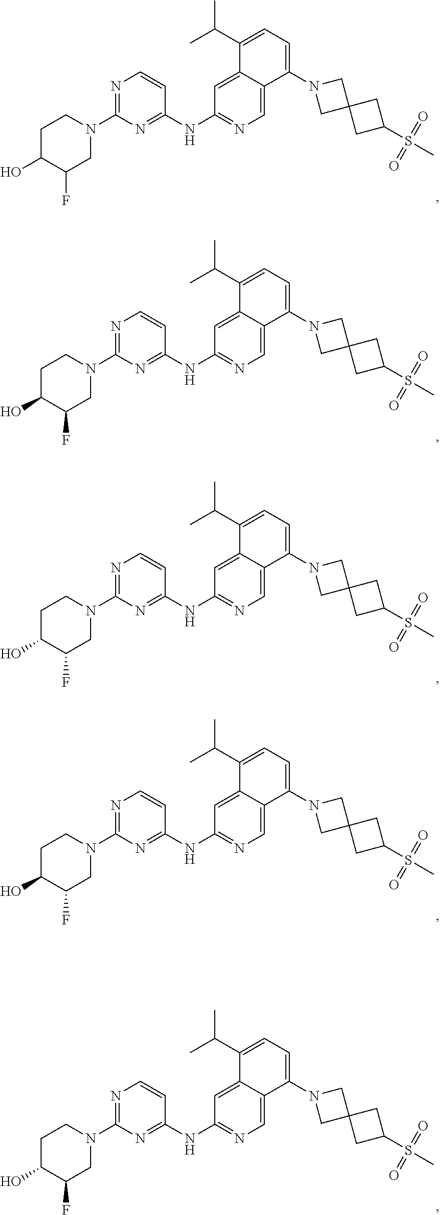 [SMILES: CC(C)c1ccc(N2CC3(CC(S(C)(=O)=O)C3)C2)c2cnc(Nc3ccnc(N4CCC(O)C(F)C4)n3)cc12 ; CC(C)c1ccc(N2CC3(CC(S(C)(=O)=O)C3)C2)c2cnc(Nc3ccnc(N4CC[C@H](O)[C@H](F)C4)n3)cc12 ; CC(C)c1ccc(N2CC3(CC(S(C)(=O)=O)C3)C2)c2cnc(Nc3ccnc(N4CC[C@@H](O)[C@@H](F)C4)n3)cc12 ; CC(C)c1ccc(N2CC3(CC(S(C)(=O)=O)C3)C2)c2cnc(Nc3ccnc(N4CC[C@H](O)[C@@H](F)C4)n3)cc12 ; CC(C)c1ccc(N2CC3(CC(S(C)(=O)=O)C3)C2)c2cnc(Nc3ccnc(N4CC[C@@H](O)[C@H](F)C4)n3)cc12]

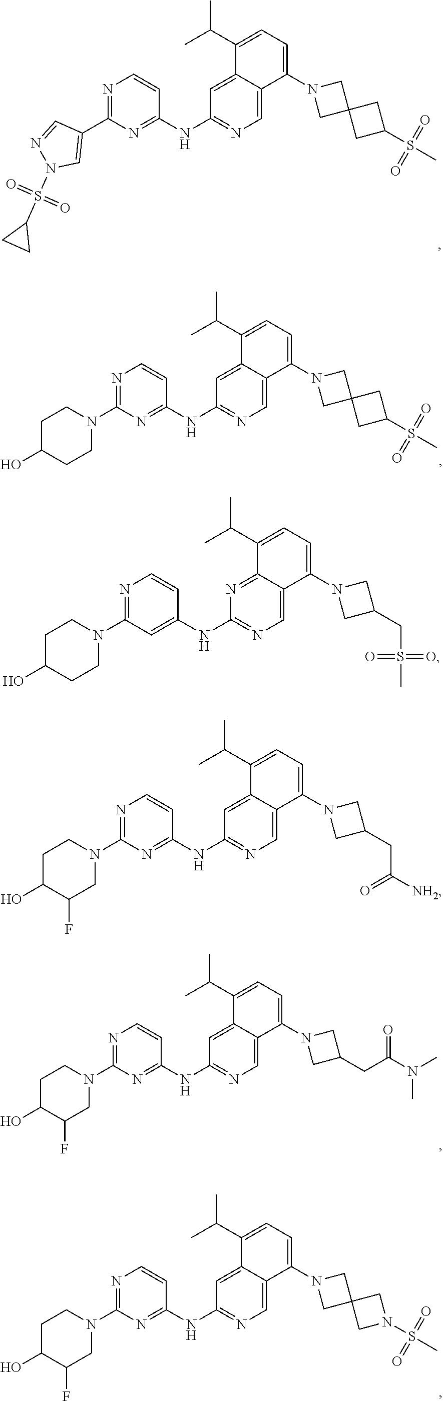 [SMILES: CC(C)c1ccc(N2CC3(CC(S(C)(=O)=O)C3)C2)c2cnc(Nc3ccnc(-c4cnn(S(=O)(=O)C5CC5)c4)n3)cc12 ; CC(C)c1ccc(N2CC3(CC(S(C)(=O)=O)C3)C2)c2cnc(Nc3ccnc(N4CCC(O)CC4)n3)cc12 ; CC(C)c1ccc(N2CC(CS(C)(=O)=O)C2)c2cnc(Nc3ccnc(N4CCC(O)CC4)c3)nc12 ; CC(C)c1ccc(N2CC(CC(N)=O)C2)c2cnc(Nc3ccnc(N4CCC(O)C(F)C4)n3)cc12 ; CC(C)c1ccc(N2CC(CC(=O)N(C)C)C2)c2cnc(Nc3ccnc(N4CCC(O)C(F)C4)n3)cc12 ; CC(C)c1ccc(N2CC3(C2)CN(S(C)(=O)=O)C3)c2cnc(Nc3ccnc(N4CCC(O)C(F)C4)n3)cc12]

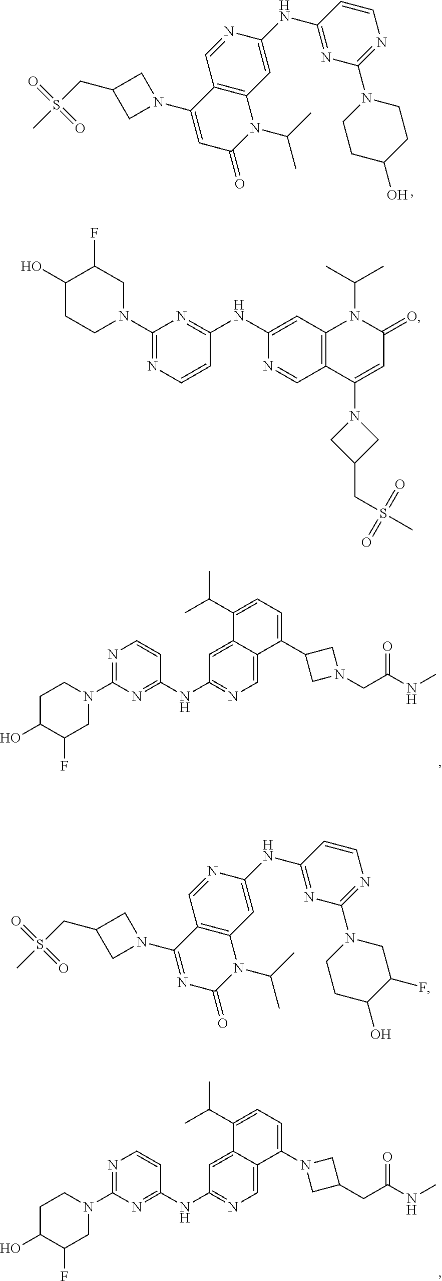

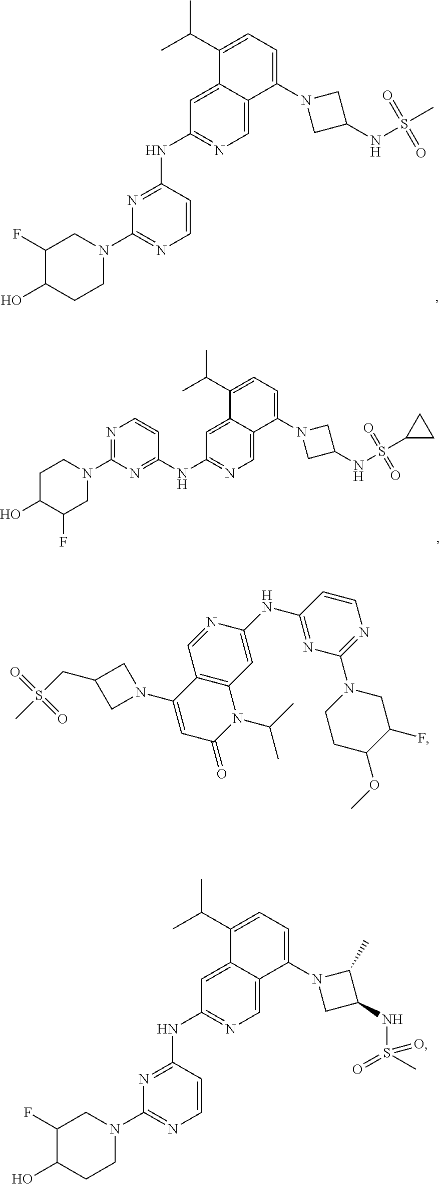

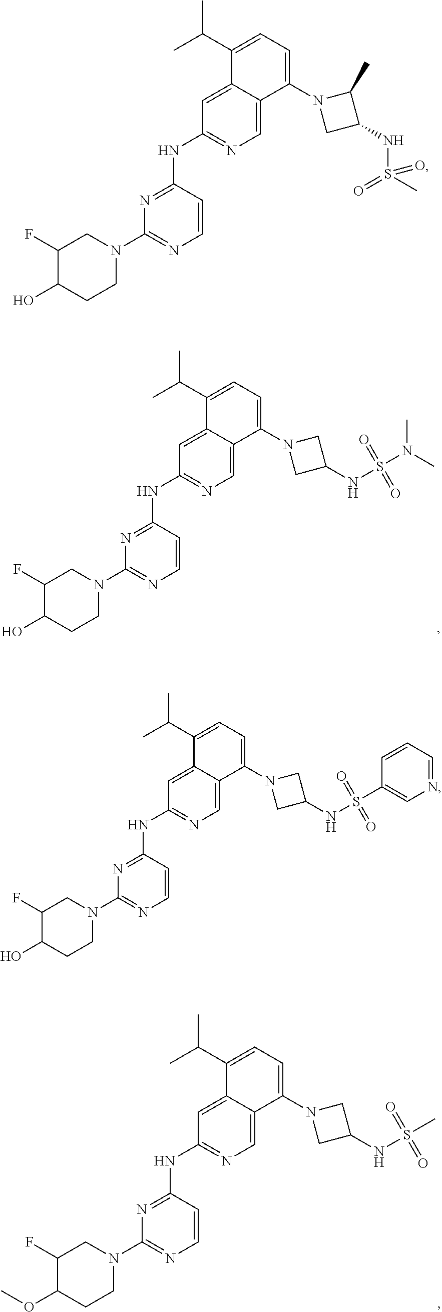

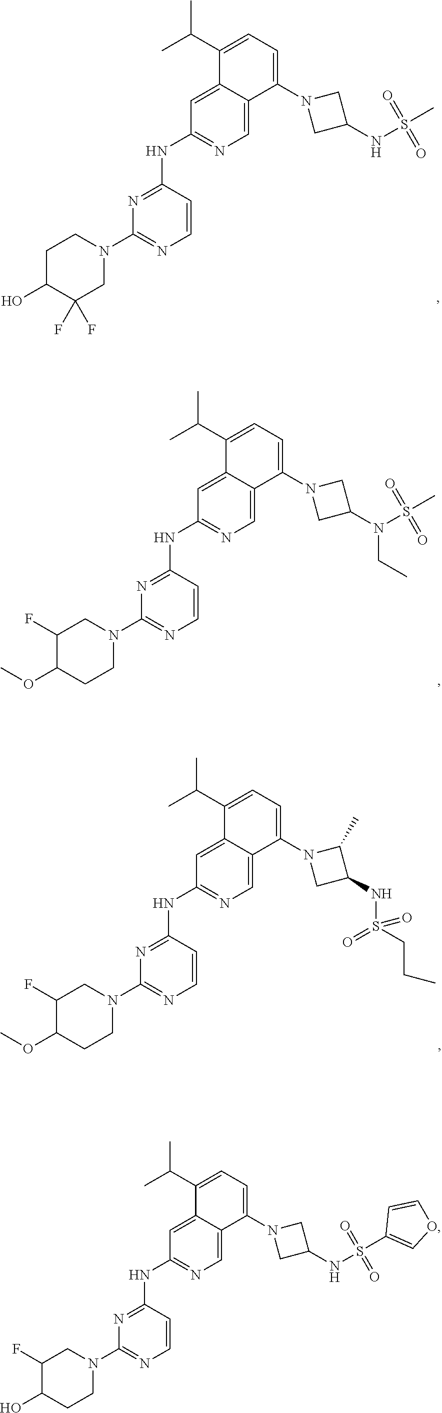

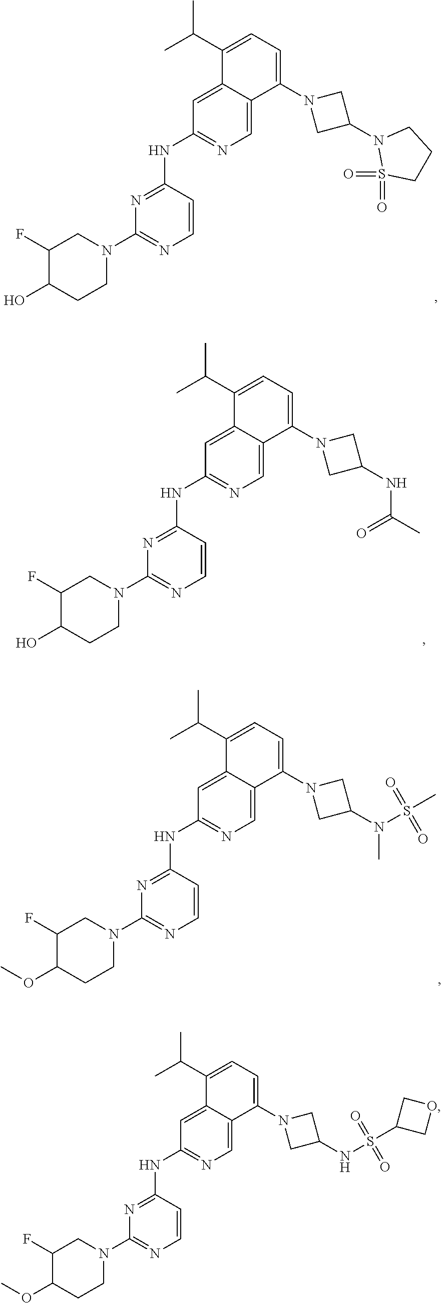

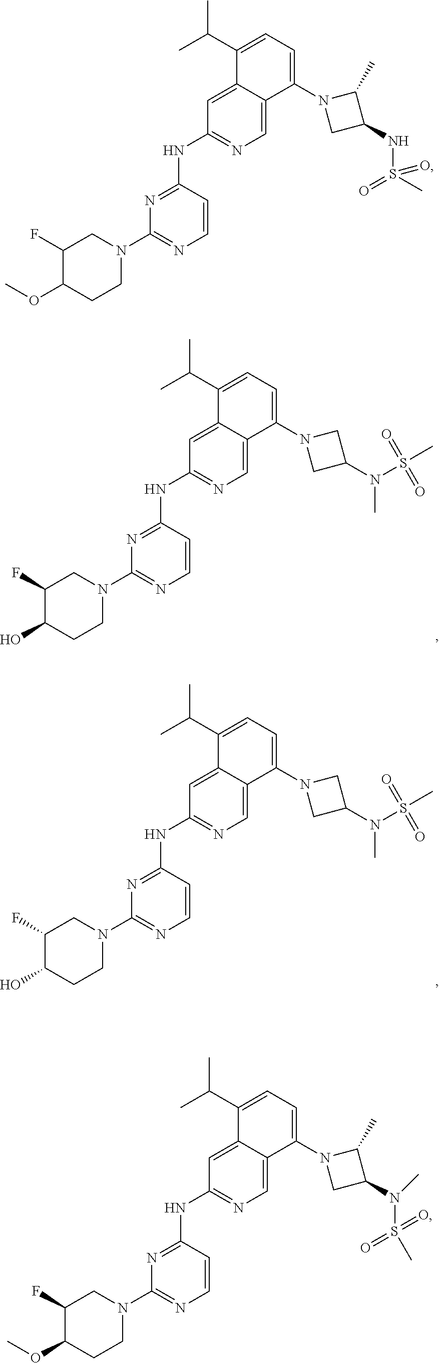

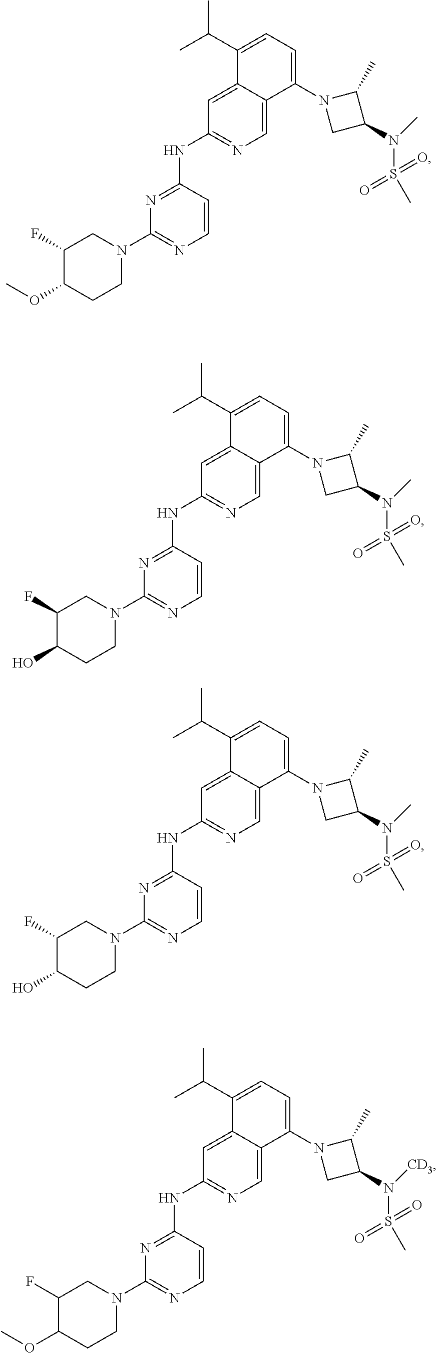 [SMILES: CO[C@H]1CCN(c2nccc(Nc3cc4c(C(C)C)ccc(N5C[C@H](N(C)S(C)(=O)=O)[C@H]5C)c4cn3)n2)C[C@H]1F ; CC(C)c1ccc(N2C[C@H](N(C)S(C)(=O)=O)[C@H]2C)c2cnc(Nc3ccnc(N4CC[C@@H](O)[C@@H](F)C4)n3)cc12 ; CC(C)c1ccc(N2C[C@H](N(C)S(C)(=O)=O)[C@H]2C)c2cnc(Nc3ccnc(N4CC[C@H](O)[C@H](F)C4)n3)cc12 ; [2H]C([2H])([2H])N([C@H]1CN(c2ccc(C(C)C)c3cc(Nc4ccnc(N5CCC(OC)C(F)C5)n4)ncc23)[C@@H]1C)S(C)(=O)=O]

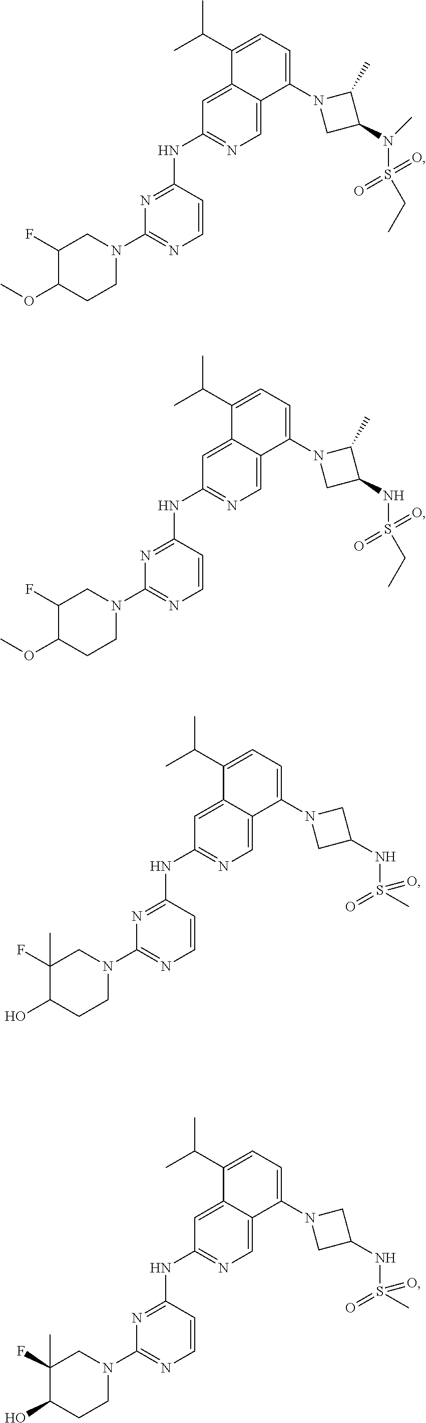 [SMILES: CCS(=O)(=O)N(C)[C@H]1CN(c2ccc(C(C)C)c3cc(Nc4ccnc(N5CCC(OC)C(F)C5)n4)ncc23)[C@@H]1C ; CCS(=O)(=O)N[C@H]1CN(c2ccc(C(C)C)c3cc(Nc4ccnc(N5CCC(OC)C(F)C5)n4)ncc23)[C@@H]1C ; CC(C)c1ccc(N2CC(NS(C)(=O)=O)C2)c2cnc(Nc3ccnc(N4CCC(O)C(C)(F)C4)n3)cc12 ; CC(C)c1ccc(N2CC(NS(C)(=O)=O)C2)c2cnc(Nc3ccnc(N4CC[C@@H](O)[C@@](C)(F)C4)n3)cc12]

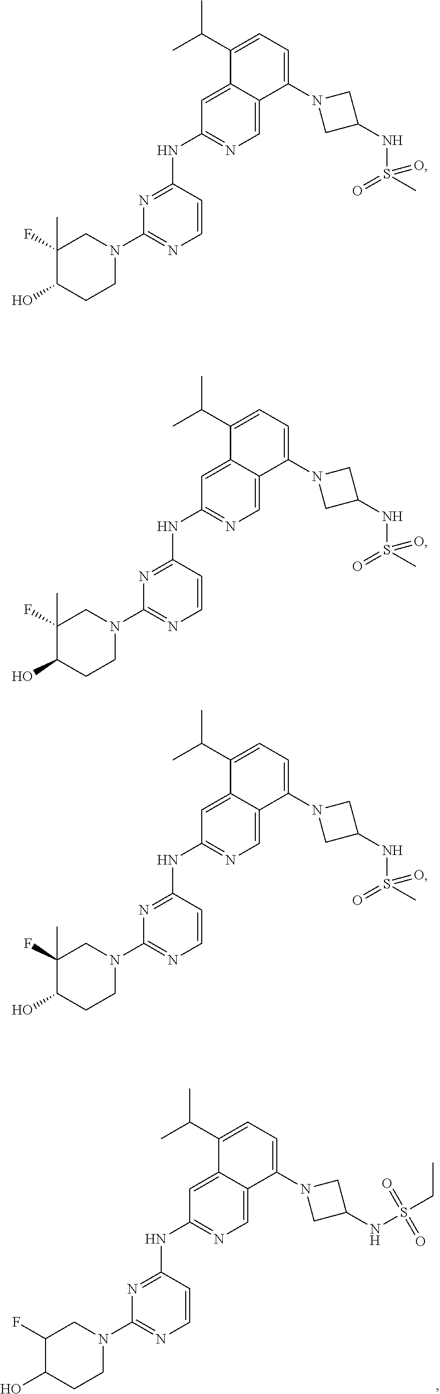 [SMILES: CC(C)c1ccc(N2CC(NS(C)(=O)=O)C2)c2cnc(Nc3ccnc(N4CC[C@H](O)[C@](C)(F)C4)n3)cc12 ; CC(C)c1ccc(N2CC(NS(C)(=O)=O)C2)c2cnc(Nc3ccnc(N4CC[C@@H](O)[C@](C)(F)C4)n3)cc12 ; CC(C)c1ccc(N2CC(NS(C)(=O)=O)C2)c2cnc(Nc3ccnc(N4CC[C@H](O)[C@@](C)(F)C4)n3)cc12 ; CCS(=O)(=O)NC1CN(c2ccc(C(C)C)c3cc(Nc4ccnc(N5CCC(O)C(F)C5)n4)ncc23)C1]

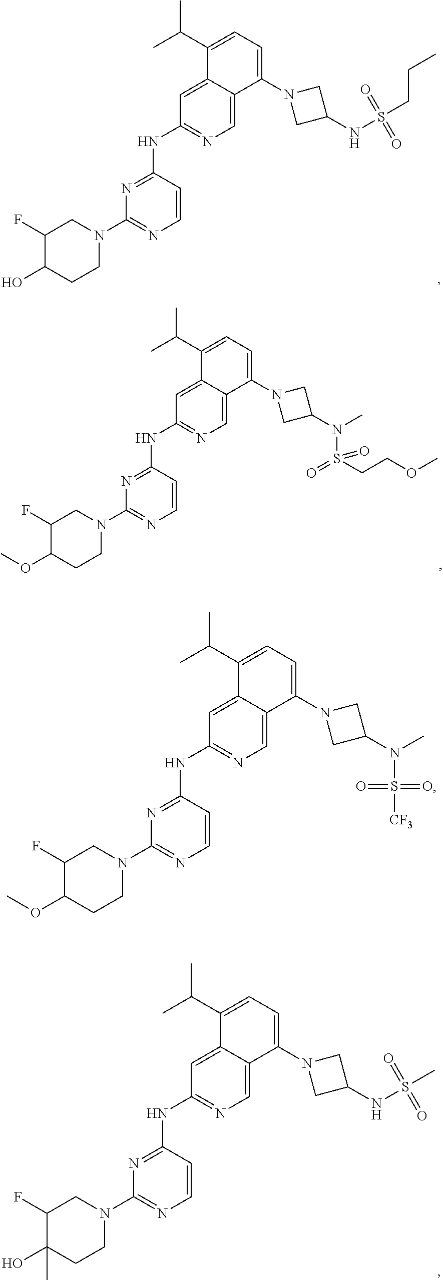

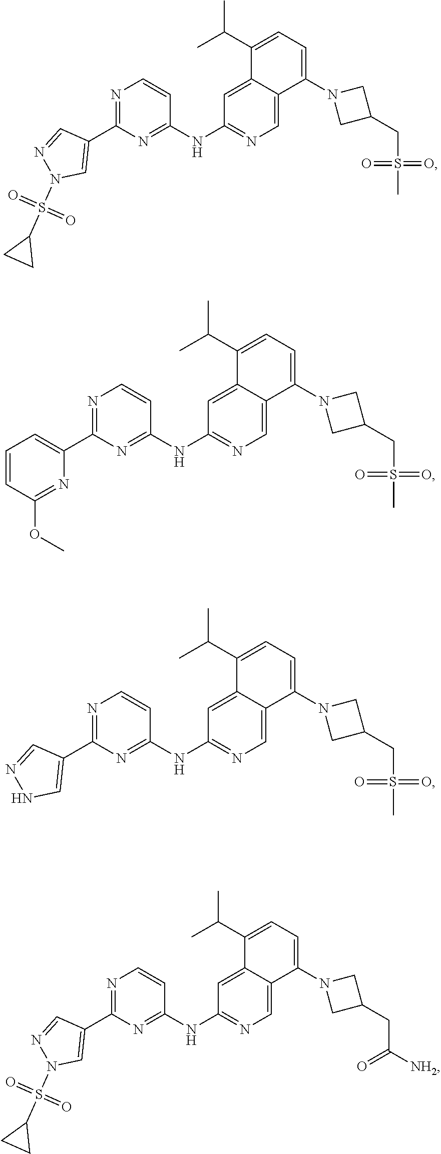 [SMILES: CC(C)c1ccc(N2CC(CS(C)(=O)=O)C2)c2cnc(Nc3ccnc(-c4cnn(S(=O)(=O)C5CC5)c4)n3)cc12 ; COc1cccc(-c2nccc(Nc3cc4c(C(C)C)ccc(N5CC(CS(C)(=O)=O)C5)c4cn3)n2)n1 ; CC(C)c1ccc(N2CC(CS(C)(=O)=O)C2)c2cnc(Nc3ccnc(-c4cn[nH]c4)n3)cc12 ; CC(C)c1ccc(N2CC(CC(N)=O)C2)c2cnc(Nc3ccnc(-c4cnn(S(=O)(=O)C5CC5)c4)n3)cc12]

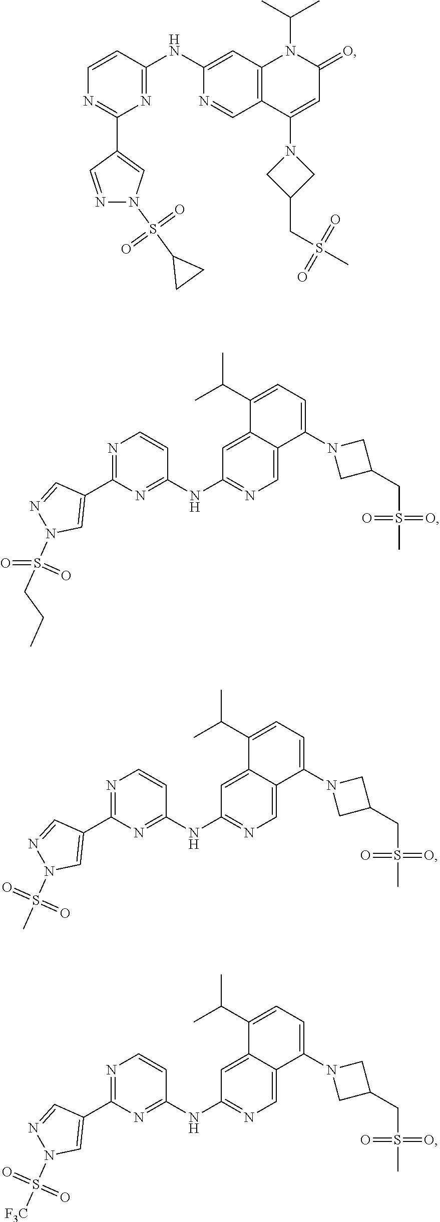 [SMILES: CC(C)n1c(=O)cc(N2CC(CS(C)(=O)=O)C2)c2cnc(Nc3ccnc(-c4cnn(S(=O)(=O)C5CC5)c4)n3)cc21 ; CCCS(=O)(=O)n1cc(-c2nccc(Nc3cc4c(C(C)C)ccc(N5CC(CS(C)(=O)=O)C5)c4cn3)n2)cn1 ; CC(C)c1ccc(N2CC(CS(C)(=O)=O)C2)c2cnc(Nc3ccnc(-c4cnn(S(C)(=O)=O)c4)n3)cc12 ; CC(C)c1ccc(N2CC(CS(C)(=O)=O)C2)c2cnc(Nc3ccnc(-c4cnn(S(=O)(=O)C(F)(F)F)c4)n3)cc12]

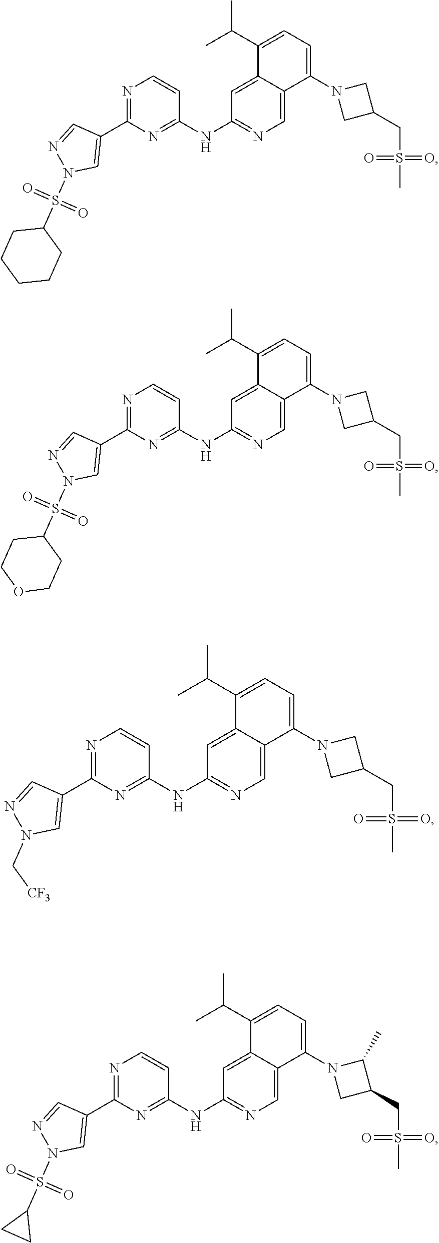 [SMILES: CC(C)c1ccc(N2CC(CS(C)(=O)=O)C2)c2cnc(Nc3ccnc(-c4cnn(S(=O)(=O)C5CCCCC5)c4)n3)cc12 ; CC(C)c1ccc(N2CC(CS(C)(=O)=O)C2)c2cnc(Nc3ccnc(-c4cnn(S(=O)(=O)C5CCOCC5)c4)n3)cc12 ; CC(C)c1ccc(N2CC(CS(C)(=O)=O)C2)c2cnc(Nc3ccnc(-c4cnn(CC(F)(F)F)c4)n3)cc12 ; CC(C)c1ccc(N2C[C@H](CS(C)(=O)=O)[C@H]2C)c2cnc(Nc3ccnc(-c4cnn(S(=O)(=O)C5CC5)c4)n3)cc12]

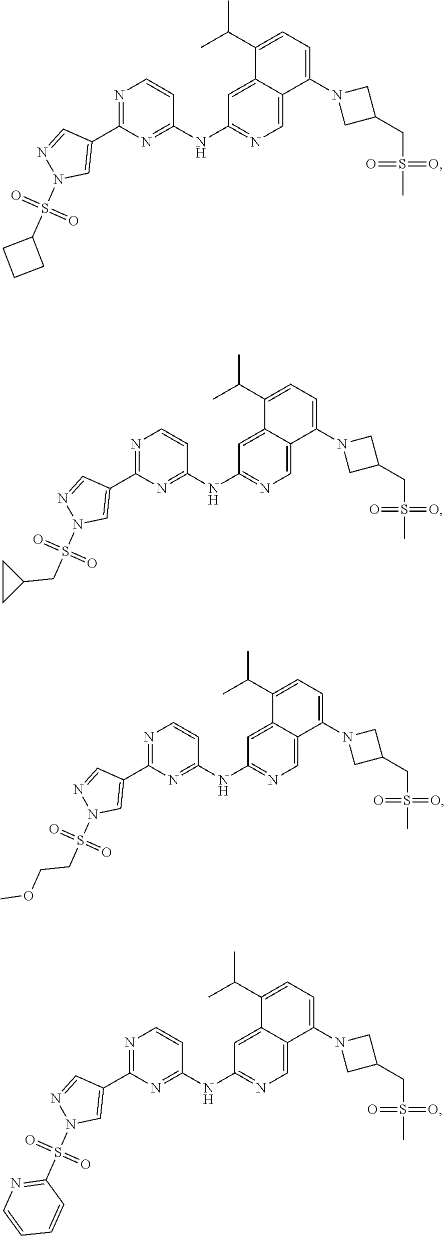 [SMILES: CC(C)c1ccc(N2CC(CS(C)(=O)=O)C2)c2cnc(Nc3ccnc(-c4cnn(S(=O)(=O)C5CCC5)c4)n3)cc12 ; CC(C)c1ccc(N2CC(CS(C)(=O)=O)C2)c2cnc(Nc3ccnc(-c4cnn(S(=O)(=O)CC5CC5)c4)n3)cc12 ; COCCS(=O)(=O)n1cc(-c2nccc(Nc3cc4c(C(C)C)ccc(N5CC(CS(C)(=O)=O)C5)c4cn3)n2)cn1 ; CC(C)c1ccc(N2CC(CS(C)(=O)=O)C2)c2cnc(Nc3ccnc(-c4cnn(S(=O)(=O)c5ccccn5)c4)n3)cc12]

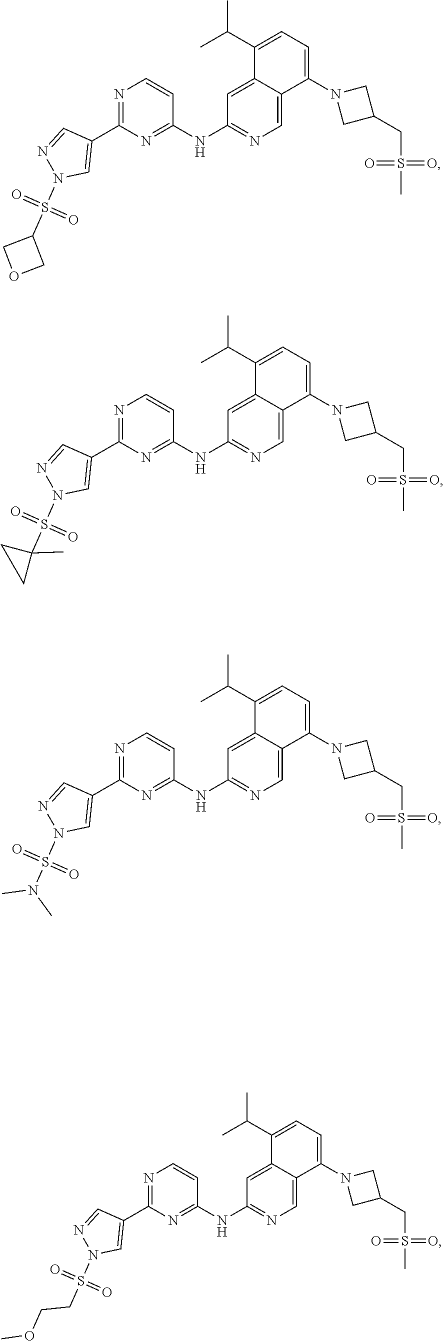 [SMILES: CC(C)c1ccc(N2CC(CS(C)(=O)=O)C2)c2cnc(Nc3ccnc(-c4cnn(S(=O)(=O)C5COC5)c4)n3)cc12 ; CC(C)c1ccc(N2CC(CS(C)(=O)=O)C2)c2cnc(Nc3ccnc(-c4cnn(S(=O)(=O)C5(C)CC5)c4)n3)cc12 ; CC(C)c1ccc(N2CC(CS(C)(=O)=O)C2)c2cnc(Nc3ccnc(-c4cnn(S(=O)(=O)N(C)C)c4)n3)cc12 ; COCCS(=O)(=O)n1cc(-c2nccc(Nc3cc4c(C(C)C)ccc(N5CC(CS(C)(=O)=O)C5)c4cn3)n2)cn1]

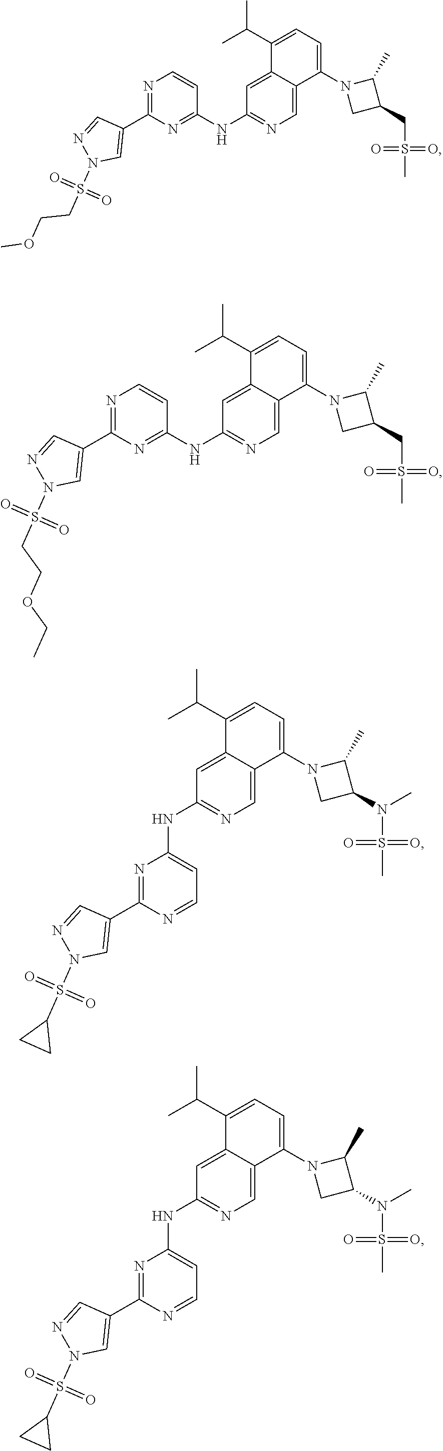 [SMILES: COCCS(=O)(=O)n1cc(-c2nccc(Nc3cc4c(C(C)C)ccc(N5C[C@H](CS(C)(=O)=O)[C@H]5C)c4cn3)n2)cn1 ; CCOCCS(=O)(=O)n1cc(-c2nccc(Nc3cc4c(C(C)C)ccc(N5C[C@H](CS(C)(=O)=O)[C@H]5C)c4cn3)n2)cn1 ; CC(C)c1ccc(N2C[C@H](N(C)S(C)(=O)=O)[C@H]2C)c2cnc(Nc3ccnc(-c4cnn(S(=O)(=O)C5CC5)c4)n3)cc12 ; CC(C)c1ccc(N2C[C@@H](N(C)S(C)(=O)=O)[C@@H]2C)c2cnc(Nc3ccnc(-c4cnn(S(=O)(=O)C5CC5)c4)n3)cc12]

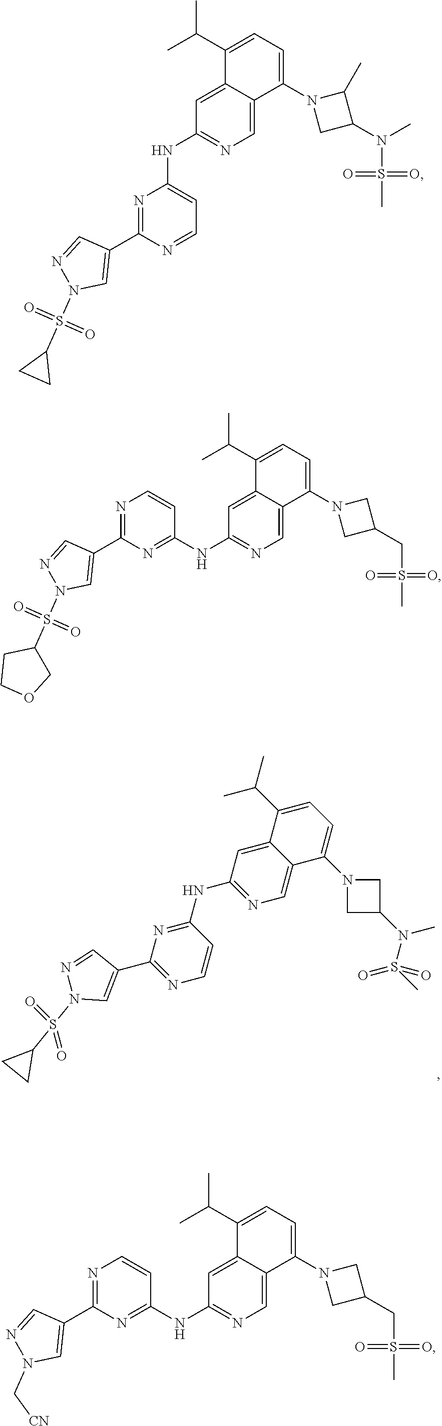 [SMILES: CC(C)c1ccc(N2CC(N(C)S(C)(=O)=O)C2C)c2cnc(Nc3ccnc(-c4cnn(S(=O)(=O)C5CC5)c4)n3)cc12 ; CC(C)c1ccc(N2CC(CS(C)(=O)=O)C2)c2cnc(Nc3ccnc(-c4cnn(S(=O)(=O)C5CCOC5)c4)n3)cc12 ; CC(C)c1ccc(N2CC(N(C)S(C)(=O)=O)C2)c2cnc(Nc3ccnc(-c4cnn(S(=O)(=O)C5CC5)c4)n3)cc12 ; CC(C)c1ccc(N2CC(CS(C)(=O)=O)C2)c2cnc(Nc3ccnc(-c4cnn(CC#N)c4)n3)cc12]

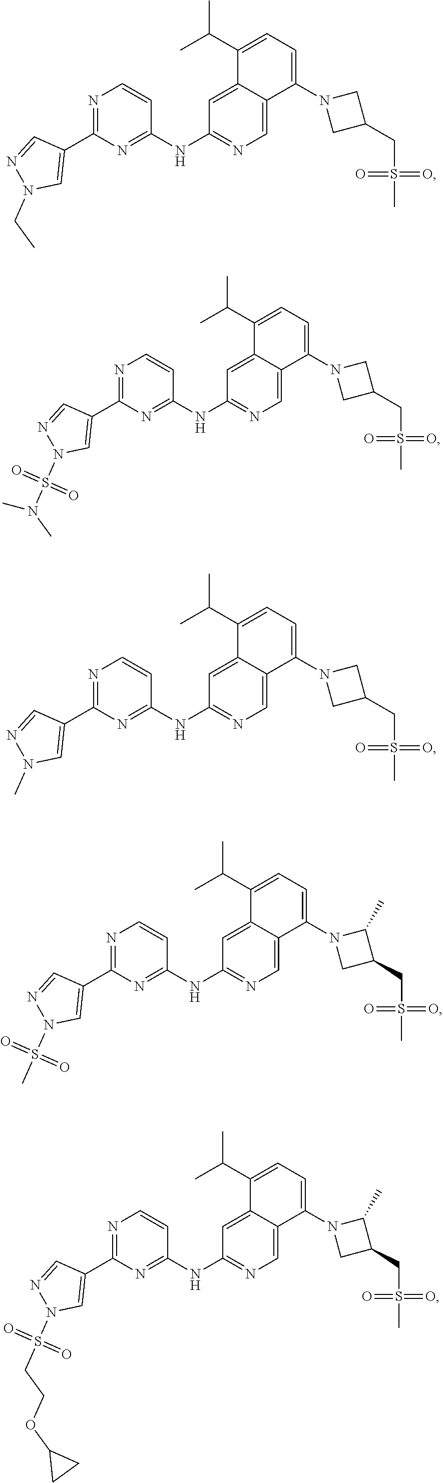 [SMILES: CCn1cc(-c2nccc(Nc3cc4c(C(C)C)ccc(N5CC(CS(C)(=O)=O)C5)c4cn3)n2)cn1 ; CC(C)c1ccc(N2CC(CS(C)(=O)=O)C2)c2cnc(Nc3ccnc(-c4cnn(S(=O)(=O)N(C)C)c4)n3)cc12 ; CC(C)c1ccc(N2CC(CS(C)(=O)=O)C2)c2cnc(Nc3ccnc(-c4cnn(C)c4)n3)cc12 ; CC(C)c1ccc(N2C[C@H](CS(C)(=O)=O)[C@H]2C)c2cnc(Nc3ccnc(-c4cnn(S(C)(=O)=O)c4)n3)cc12 ; CC(C)c1ccc(N2C[C@H](CS(C)(=O)=O)[C@H]2C)c2cnc(Nc3ccnc(-c4cnn(S(=O)(=O)CCOC5CC5)c4)n3)cc12]

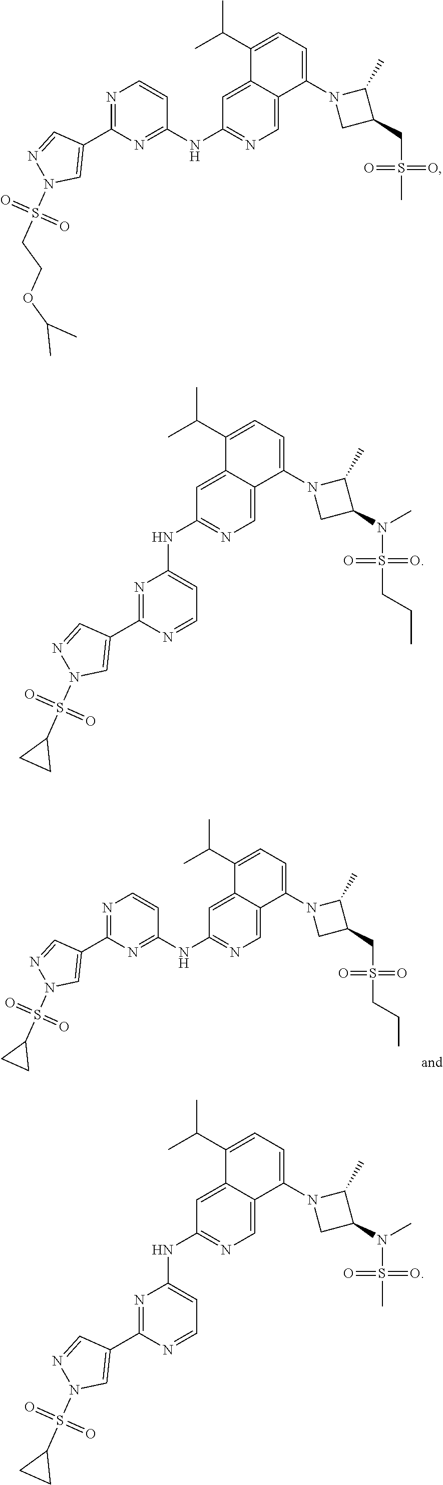 [SMILES: CC(C)OCCS(=O)(=O)n1cc(-c2nccc(Nc3cc4c(C(C)C)ccc(N5C[C@H](CS(C)(=O)=O)[C@H]5C)c4cn3)n2)cn1 ; CCCS(=O)(=O)N(C)[C@H]1CN(c2ccc(C(C)C)c3cc(Nc4ccnc(-c5cnn(S(=O)(=O)C6CC6)c5)n4)ncc23)[C@@H]1C ; CCCS(=O)(=O)C[C@H]1CN(c2ccc(C(C)C)c3cc(Nc4ccnc(-c5cnn(S(=O)(=O)C6CC6)c5)n4)ncc23)[C@@H]1C ; CC(C)c1ccc(N2C[C@H](N(C)S(C)(=O)=O)[C@H]2C)c2cnc(Nc3ccnc(-c4cnn(S(=O)(=O)C5CC5)c4)n3)cc12]

33. A pharmaceutical composition comprising the compound according to claim 1, or a pharmaceutically acceptable salt, isotopic variant, tautomer, stereoisomer, prodrug polymorph, hydrate or solvate thereof, and a pharmaceutically acceptable excipient; alternatively, further comprising other therapeutic agent.

34. (canceled)

35. A method for treating and/or preventing a disease mediated by EGFR protein and mutants thereof in a subject, the method comprising administering to the subject the compound according to claim 1, or a pharmaceutically acceptable salt, isotopic variant, tautomer, stereoisomer, prodrug, polymorph, hydrate or solvate therefor.

36. (canceled)

37. The method according to claim 35, wherein the EGFR mutant is selected from one or more of L858R mutant, del19 mutant, T790M mutant, and C797S mutant; optionally, the disease is selected from lung cancer, colon cancer, urothelial carcinoma, breast cancer, prostate cancer, brain cancer, ovarian cancer, gastric cancer, pancreatic cancer, head and neck cancer, bladder cancer and mesothelioma, alternatively non-small cell lung cancer.

38. A method for treating and/or preventing a disease mediated by EGFR protein and mutants thereof in a subject, the method comprising administering to the subject the pharmaceutical composition according to claim 33.

39. The method according to claim 38, wherein the EGFR mutant is selected from one or more of L858R mutant, del19 mutant, T790M mutant, and C797S mutant; optionally, the disease is selected from lung cancer, colon cancer, urothelial carcinoma, breast cancer, prostate cancer, brain cancer, ovarian cancer, gastric cancer, pancreatic cancer, head and neck cancer, bladder cancer and mesothelioma, alternatively non-small cell lung cancer.
